(19) United States
(12) Patent Application Publication
(10) Pub. No.: US 2023/0226183 A1
(43) Pub. Date: Jul. 20, 2023

(54) COMPOSITIONS AND METHODS FOR ALTERING GAMMA DELTA T CELL ACTIVITY

(71) Applicant: THE REGENTS OF THE UNIVERSITY OF CALIFORNIA, Oakland, CA (US)

(72) Inventors: Murad R. MAMEDOV, Oakland, CA (US); Alexander MARSON, Oakland, CA (US)

(73) Assignee: THE REGENTS OF THE UNIVERSITY OF CALIFORNIA, Oakland, CA (US)

(21) Appl. No.: 17/758,866

(22) PCT Filed: Jan. 15, 2021
(86) PCT No.: PCT/US2021/013736

§ 371 Date: Jul. 14, 2022

Related U.S. Application Data
(60) Provisional application No. 62/961,563, filed on Jan. 15, 2020.

(51) Int. Cl. A61K 39/00; C12N 15/90

(52) U.S. Cl. CPC A61K 39/4633; A61K 39/4611; C12N 15/907; C12N 2310/20

(57) ABSTRACT

Provided herein are compositions and methods for altering sensitivity of target cells to killing by γδ T cells.

### PRIOR RELATED APPLICATION

This application claims the benefit of U.S. Provisional Application No. 62/961,563 filed on Jan. 15, 2020, which is hereby incorporated by reference in its entirety.

### INCORPORATION BY REFERENCE OF SEQUENCE LISTING

This application contains a Sequence Listing in computer readable form (filename: 1325433.txt; Size 1.42 MB; created Oct. 26, 2022); which is incorporated by reference in its entirety and forms part of the disclosure.

### BACKGROUND OF THE INVENTION

Gamma delta (γδ) T cells play a role in regulating the immune response. In some cases, for example, in some cancers and infections, γδ T cells kill tumor cells and infected cells, respectively. In other cases, for example, in bone disorders or autoimmune diseases, γδ T cells exert undesirable proinflammatory effects. Methods for enhancing cell killing by γδ T cells for the treatment of cancer or decreasing cell killing by γδ T cells for the treatment of bone disorders or autoimmune diseases have great therapeutic potential.

### SUMMARY OF THE INVENTION

The present invention is directed to compositions and methods for altering sensitivity i.e., increasing or decreasing sensitivity, of target cells to killing by γδ T cells. The inventors have identified cellular factors that influence γδ T cell cytotoxicity against target cells. γδ T cell cytotoxicity can be increased by increasing expression and/or activity of one or more of these cellular factors. γδ T cell cytotoxicity can be decreased by decreasing expression and/or activity of one or more of these cellular factors. In some examples, γδ T cell cytotoxicity is decreased or reduced to treat a bone disorder or an autoimmune disorder. In other examples, γδ T cell cytotoxicity is increased to treat cancer.

Provided herein is a method of increasing sensitivity of a target cell to killing by a γδ T cell, comprising: increasing expression and/or activity of one or more cellular factors set forth in Table 1, in the target cell.

In some embodiments, the sensitivity of the target cell is increased in the presence of the γδ T cell. In some embodiments, the target cell is a cancer cell. In some embodiments, the target cell is a cell that is infected with an infectious agent.

In some embodiments, the increasing expression and/or activity of the one or more cellular factors in Table 1 comprises contacting the target cell with an agent selected from the group consisting of an antibody, a small molecule, a polypeptide, siRNA, microRNA or a drug.

In some embodiments, the increasing expression comprises increasing expression of the cellular factor of Table 1, or increasing expression of a polynucleotide encoding the cellular factor of Table 1.

Also provided is a method of decreasing sensitivity of a target cell to killing by a γδ T cell, comprising: inhibiting expression and/or activity of one or more cellular factors set forth in Table 1, in the target cell. In some embodiments, the sensitivity of the target cell is decreased in the presence of the γδ T cell.

In some embodiments, the decreasing expression and/or activity of the one or more cellular factors in Table 1 comprises contacting the target cell with an agent is selected from the group consisting of an antibody, a small molecule, a polypeptide, siRNA, microRNA, or a drug.

In some embodiments, the decreasing expression comprises reducing expression of the cellular factor, or reducing expression of a polynucleotide encoding the cellular factor.

In some embodiments, the antibody used to increase or decrease activity and/or expression of a cellular factor of Table 1 is a bispecific antibody, wherein the bispecific antibody has specificity for an epitope of the one or more cellular factors of Table 1 expressed by the target cell and specificity for an epitope on the γδ T cell.

In some embodiments, the target cell is ex vivo, in vitro or in vivo. In some embodiments, the sensitivity of the target cell is increased or decreased in a human. In some embodiments, the human has cancer, a bone disorder, an autoimmune disorder or an infectious disease.

In some embodiments, the γδ T cell is a Vγ9Vδ2 T cell. In some embodiments, the method further comprises administering γδ T cells to the human. In some embodiments, the γδ T cells are autologous γδ T cells or allogeneic γδ T cells. In some embodiments, the γδ T cells comprise a heterologous cell-surface protein that binds to a cellular factor set forth in Table 1.

Also provided is a method of increasing sensitivity of target cells to killing by a γδ T cell in a subject in need thereof, comprising increasing expression and/or activity of one or more cellular factors set forth in Table 1, in the target cells of the subject. In some embodiments, the subject has cancer or an infectious disease.

Further provided is a method of decreasing sensitivity of target cells to killing by a γδ T cell in a subject in need thereof, comprising decreasing expression and/or activity of one or more cellular factors set forth in Table 1, in the target cells of the subject. In some embodiments, the subject has a bone disorder, a metabolic disorder or an autoimmune disease.

In some embodiments, the method further comprises administering γδ T cells to the subject. In some embodiments, the γδ T cells are Vγ9Vδ2 T cells. In some embodiments, the γδ T cells are autologous γδ T cells or allogeneic γδ T cells.

Also provided is a γδ T cell comprising a heterologous cell-surface ligand that binds to a cellular factor set forth in Table 1. In some embodiments, the γδ T cell is a Vγ9Vδ2 T cell. In some embodiments, the cell-surface ligand is an antibody.

### BRIEF DESCRIPTION OF THE DRAWINGS

The present application includes the following figure. The figure is intended to illustrate certain embodiments and/or features of the compositions and methods, and to supplement any description(s) of the compositions and methods. The figure does not limit the scope of the compositions and methods, unless the written description expressly indicates that such is the case.

FIG. 1 provides aggregate data of gRNA enrichment among Daudi cells that survived the co-culture with expanded Vγ9Vδ2 T cells from three different healthy donors. The labeled data points above the dashed lines are positively enriched genes with a false-discovery rate (FDR) of less than 0.1. The biological functions of the genes are described in Table 5.

### Definitions

As used in this specification and the appended claims, the singular forms “a,” “an,” and “the” include plural reference unless the context clearly dictates otherwise.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” can refer to the segment of DNA involved in producing or encoding a polypeptide chain. It may include regions preceding and following the coding region (leader and trailer) as well as intervening sequences (introns) between individual coding segments (exons).

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

The term “increasing sensitivity” refers to increasing the target cell's responsiveness to killing by a γδ T cell. An increase in sensitivity for example, can be an increase of at least 10%, as compared to a reference control level, or an increase of least about 20%, or at least about 30%, or at least about 40%, or at least about 50%, or at least about 60%, or at least about 70%, or at least about 80%, or at least about 90%, or at least about 100%, or at least about 200%, or at least about 300% or at least about 400%. An increase in sensitivity of a target cell can be an increase of at least about 20%, or at least about 30%, or at least about 40%, or at least about 50%, or at least about 60%, or at least about 70%, or at least about 80%, or at least about 90%, or at least about 100%, or at least about 200%, or at least about 300% or at least about 400% as compared to a target cell where expression and/or activity of one or more cellular factors set forth in Table 1 is not increased. An increase in sensitivity can result in an increase in target cell killing of at least 10%, as compared to a reference control level, or an increase of least about 20%, or at least about 30%, or at least about 40%, or at least about 50%, or at least about 60%, or at least about 70%, or at least about 80%, or at least about 90%, or at least about 100%, or at least about 200%, or at least about 300% or at least about 400%.

The term “decreasing sensitivity” refers to decreasing the target cell's responsiveness to killing by a γδ T cell. A decrease in sensitivity for example, can be a decrease of at least 10%, as compared to a reference control level, or a decrease of least about 20%, or at least about 30%, or at least about 40%, or at least about 50%, or at least about 60%, or at least about 70%, or at least about 80%, or at least about 90%, or at least about 100%, or at least about 200%, or at least about 300% or at least about 400%. A decrease in sensitivity of a target cell can be a decrease of at least about 20%, or at least about 30%, or at least about 40%, or at least about 50%, or at least about 60%, or at least about 70%, or at least about 80%, or at least about 90%, or at least about 100%, or at least about 200%, or at least about 300% or at least about 400% as compared to a target cell where expression and/or activity of one or more cellular factors set forth in Table 1 is not decreased. An decrease in sensitivity can result in a decrease in target cell killing of at least 10%, as compared to a reference control level, or a decrease of least about 20%, or at least about 30%, or at least about 40%, or at least about 50%, or at least about 60%, or at least about 70%, or at least about 80%, or at least about 90%, or at least about 100%, or at least about 200%, or at least about 300% or at least about 400%.

The term “increasing expression” or “overexpression” refers to increasing the expression of a gene or protein. An increase in expression, for example, can be an increase in the amount of mRNA or protein expressed in a target cell, of at least 10%, as compared to a reference control level, or an increase of least about 20%, or at least about 30%, or at least about 40%, or at least about 50%, or at least about 60%, or at least about 70%, or at least about 80%, or at least about 90%, or at least about 100%, or at least about 200%, or at least about 300% or at least about 400%. Various methods for overexpression are known to those of skill in the art, and include, but are not limited to, stably or transiently introducing a heterologous polynucleotide encoding a protein (i.e., a cellular factor set forth in Table 1) to be overexpressed into the cell or inducing overexpression of an endogenous gene encoding the protein in the cell.

The term “inhibiting expression” refers to inhibiting or reducing the expression of a gene product, e.g., RNA or protein. As used throughout, the term “cellular factor” refers to a protein that is directly or indirectly involved in γδ T cell activity, for example, in γδ T cell cytotoxicity against a target cell. To inhibit or reduce the expression of a gene, the sequence and/or structure of the gene may be modified such that the gene would not be transcribed (for DNA) or translated (for RNA), or would not be transcribed or translated to produce a functional protein, for example, a polypeptide or protein encoded by a gene set forth in Table 1. Various methods for inhibiting or reducing expression are described in detail further herein. Some methods may introduce nucleic acid substitutions, additions, and/or deletions into the wild-type gene. Some methods may also introduce single or double strand breaks into the gene. To inhibit or reduce the expression of a protein, one may inhibit or reduce the expression of the gene or polynucleotide encoding the protein. In other embodiments, one may target the protein directly to inhibit or reduce the protein's expression using, e.g., an antibody or a protease. “Inhibited” expression refers to a decrease by at least 10% as compared to a reference control level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample).

As used herein the phrase “heterologous” refers to what is not found in nature. The term “heterologous sequence” refers to a sequence not normally found in a given cell in nature. As such, a heterologous nucleotide or protein sequence may be: (a) foreign to its host cell (i.e., is exogenous to the cell); (b) naturally found in the host cell (i.e., endogenous) but present at an unnatural quantity in the cell (i.e., greater or lesser quantity than naturally found in the host cell); or (c) be naturally found in the host cell but positioned outside of its natural locus.

“Treating” refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; or making the final point of degeneration less debilitating.

A “promoter” is defined as one or more a nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

As used herein, the term “complementary” or “complementarity” refers to specific base pairing between nucleotides or nucleic acids. Complementary nucleotides are, generally, A and T (or A and U), and G and C. The guide RNAs described herein can comprise sequences, for example, DNA targeting sequences that are perfectly complementary or substantially complementary (e.g., having 1-4 mismatches) to a genomic sequence.

As used throughout, by subject is meant an individual. For example, the subject is a mammal, such as a primate, and, more specifically, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical uses and formulations are contemplated herein. The term does not denote a particular age or sex. Thus, adult and newborn subjects, whether male or female, are intended to be covered. As used herein, patient or subject may be used interchangeably and can refer to a subject afflicted with a disease or disorder.

As used throughout, the term “targeted nuclease” refers to nuclease that is targeted to a specific DNA sequence in the genome of a cell to produce a strand break at that specific DNA sequence. The strand break can be single-stranded or double-stranded. Targeted nucleases include, but are not limited to, a Cas nuclease, a TAL-effector nuclease and a zinc finger nuclease.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep 24; 110(39):15644-9; Sampson et al., Nature. 2013 May 9; 497(7448):254-7; and Jinek, et al., Science. 2012 Aug 17; 337(6096):816-21. Variants of any of the Cas9 nucleases provided herein can be optimized for efficient activity or enhanced stability in the host cell. Thus, engineered Cas9 nucleases are also contemplated.

As used throughout, a guide RNA (gRNA) sequence is a sequence that interacts with a site-specific or targeted nuclease and specifically binds to or hybridizes to a target nucleic acid within the genome of a cell, such that the gRNA and the targeted nuclease co-localize to the target nucleic acid in the genome of the cell. Each gRNA includes a DNA targeting sequence or protospacer sequence of about 10 to 50 nucleotides in length that specifically binds to or hybridizes to a target DNA sequence in the genome. For example, the targeting sequence may be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the gRNA comprises a crRNA sequence and a transactivating crRNA (tracrRNA) sequence. In some embodiments, the gRNA does not comprise a tracrRNA sequence.

As used herein, the term “Cas9” refers to an RNA-mediated nuclease (e.g., of bacterial or archeal orgin, or derived therefrom). Exemplary RNA-mediated nucleases include the foregoing Cas9 proteins and homologs thereof. Other RNA-mediated nucleases include Cpf1 (See, e.g., Zetsche et al., Cell, Volume 163, Issue 3, p759-771, 22 Oct. 2015) and homologs thereof. Similarly, as used herein, the term “Cas9 ribonucleoprotein” complex and the like refers to a complex between the Cas9 protein and a guide RNA, the Cas9 protein and a crRNA, the Cas9 protein and a trans-activating crRNA (tracrRNA), or a combination thereof (e.g., a complex containing the Cas9 protein, a tracrRNA, and a crRNA guide RNA). It is understood that in any of the embodiments described herein, a Cas9 nuclease can be subsitututed with a Cpf1 nuclease or any other guided nuclease.

As used herein, the phrase “modifying” refers to inducing a structural change in the sequence of the genome at a target genomic region in a cell, for example, a target cell or a γδ T cell. For example, the modifying can take the form of inserting a nucleotide sequence into the genome of the cell. Such modifying can be performed, for example, by inducing a double stranded break within a target genomic region, or a pair of single stranded nicks on opposite strands and flanking the target genomic region. Methods for inducing single or double stranded breaks at or within a target genomic region include the use of a Cas9 nuclease domain, or a derivative thereof, and a guide RNA, or pair of guide RNAs, directed to the target genomic region. “Modifying” can also refer to altering the expression of a gene or protein in a γδ T cell, for example inhibiting expression of a gene or protein or overexpressing a protein in a γδ T cell.

As used herein, the phrase “introducing” in the context of introducing a nucleic acid or a complex comprising a nucleic acid, for example, an RNP complex, refers to the translocation of the nucleic acid sequence or the RNP complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

### DETAILED DESCRIPTION OF THE INVENTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### I. Introduction

γδ T cells are a specialized subset of T cells in peripheral blood that are part of both the adaptive and innate immune system. Due to their donor-unrestricted nature (i.e., they are not MHC-restricted) and their ability to kill a wide variety of tumor cells, these cells are strong cell therapy candidates. However, little is known about the factors that γδ T cells recognize on target cells. The inventors have identified cellular factors that are involved in γδ T cell cytotoxicity against target cells.

### II. Methods and Compositions

As described herein, the disclosure provides compositions and methods directed to altering sensitivity of a target cell to killing by a γδ T cell. The disclosure provides methods of increasing sensitivity of target cells to killing by γδ T cells by increasing the expression and/or activity of one or more cellular factors on the target cell. Compositions and methods directed to decreasing sensitivity of a target cell to killing by γδ T cells, by decreasing the expression and/or activity of one or more cellular factors on the target cell are also provided. The disclosure also features compositions comprising modified γδ T cells and populations of modified γδ T cells that express a cell-surface ligand, that binds to a cellular factor set forth herein.

Examples of cellular factors whose expression and/or activity may be altered to increase or decrease sensitivity of a target cell to killing by a γδ T cell in the methods described herein include, but are not limited to, the cellular factors set forth in Table 1. National Center for Biotechnology Information (NCBI) Gene (formerly Entrez Gene) ID for each of the cellular factors and their splice variants, if applicable, are provided in Table 2. Amino acid sequences encoding the cellular factors, and their splice variants, if applicable, are are also provided in Table 2. All of the nucleotide and protein sequences set forth under each NCBI Gene ID are hereby incorporated in their entireties by this reference. The protein sequence of any of the cellular factors set forth herein can comprise, consist of or consist essentially of any of the amino acid sequences listed in Table 2. Any of the amino acid sequences listed in Table 2 having an X as a first amino acid also include the amino acid sequence that does not have an X as the first amino acid. In some embodiments, sensitivity of a a population of target cells to killing by a γδ T cell is increased or decreased. In some embodiments, the present invention provides a method of increasing sensitivity of a target cell to killing by a γδ T cell, comprising: increasing expression and/or activity of one or more cellular factors set forth in Table 1, in some embodiments, the present invention provides a method of decreasing sensitivity of a target cell to killing by a γδ T cell, comprising: decreasing expression and/or activity of one or more cellular factors set forth in Table 1, In some embodiments, the sensitivity of the cell is increased or decreased in the presence of the γδ T cell or a population of γδ T cells. Methods for measuring an increase or a decrease in the sensitivity of a target cell to killing by γδ T cells are available to those of skill in the art. See, for example Vollenweider et al. (1993). Heterogeneous binding and killing behaviour of human gamma/delta-TCR+ lymphokine-activated killer cells against K562 and Daudi cells. Cancer Immunology, Immunotherapy: CII, 36(5), 331-336; Burjanadzé et al. (2007). In vitro expansion of gamma delta T cells with anti-myeloma cell activity by Phosphostim and IL-2 in patients with multiple myeloma. British Journal of Haematology, 139(2), 206-216; and D'Asaroet al. (2010). V gamma 9V delta 2 T lymphocytes efficiently recognize and kill zoledronate-sensitized, imatinib-sensitive, and imatinib-resistant chronic myelogenous leukemia cells. The Journal of Immunology, 184(6), 3260-3268.

In some embodiments, expression of an amino acid sequence having at least about 80%, 85%, 90%, 95% or 99% identity to an amino acid sequence set forth in Table 1 is increased or decreased. It is understood that, when referring to one or more cellular factors set forth in Table 1, this can be the protein, i.e., the cellular factor, or the polynucleotide encoding the cellular factor.

In the methods provided herein, γδ T cell activity, for example target cell killing, can be increased (for example, when sensitivity to cell killing is increased in a target cell) or decreased (for example, when sensitivity to cell killing is decreased in a target cell). To increase the activity of γδ T cells refers to any treatment or manipulation of target cells or γδ T cells which results in an increase (i.e., enhancement, upregulation, induction, stimulation) in the number, activation, biological activity and/or survivability of the γδ T cells. Therefore, increasing the activity of γδ T cells can be accomplished by increasing the number of γδ T cells in a subject (i.e., by causing the cells to proliferate/expand or by recruiting additional γδ T cells to a site), by increasing the activation of γδ T cells in a subject, by decreasing the proximity of γδ T cells to a target cell, by increasing biological activity of γδ T cells (e.g., effector functions or other activities of the cell) in an animal and/or by increasing the ability of γδ T cells to survive in a subject. To decrease the activity of γδ T cells refers to any treatment or manipulation of target cells or γδ T cells which results in a decrease (i.e., reduction, downregulation, inhibition) in the number, activation, biological activity and/or survivability of the γδ T cells. Therefore, decreasing the activity of γδ T cells can be accomplished by decreasing the number of γδ T cells in a subject, by decreasing the activation of γδ T cells in a subject, by decreasing biological activity of γδ T cells (e.g., effector functions or other activities of the cell) in a subject and/or by decreasing the ability of γδ T cells to survive in a subject.

TABLE 1

| | | Falde- | | Log2 | |
|---|---|---|---|---|---|
| | | discover | | Fold | |
| Gene ID | p-value | Rate | Rank | Change | Localization |
| BTN3A1 | 2.75E−07 | 0.00033 | 1 | 2.0651 | Surface |
| SPIB | 2.75E−07 | 0.00033 | 2 | 1.2994 | Intracellular |
| IRF1 | 2.75E−07 | 0.00033 | 3 | 1.6107 | Intracellular |
| FBXW7 | 2.75E−07 | 0.00033 | 4 | 1.1113 | Intracellular |
| BTN2A1 | 2.75E−07 | 0.00033 | 5 | 1.7442 | Surface |
| RFXAP | 2.75E−07 | 0.00033 | 6 | 1.5428 | Intracellular |
| ACAT2 | 2.75E−07 | 0.00033 | 7 | 1.9893 | Intracellular |
| NLRC5 | 2.75E−07 | 0.00033 | 8 | 0.86688 | Intracellular |
| SQLE | 2.75E−07 | 0.00033 | 9 | 0.83862 | Intracellular |
| BTN3A2 | 2.75E−07 | 0.00033 | 10 | 1.5982 | Surface |
| SPI1 | 2.75E−07 | 0.00033 | 11 | 1.2165 | Intracellular |
| IRF9 | 2.75E−07 | 0.00033 | 12 | 0.96081 | Intracellular |
| GGNBP2 | 2.75E−07 | 0.00033 | 13 | 1.0078 | Intracellular |
| IRF8 | 2.75E−07 | 0.00033 | 14 | 1.0578 | Intracellular |
| SIRT1 | 2.75E−07 | 0.00033 | 15 | 1.0665 | Intracellular |
| GLTSCR1 | 8.25E−07 | 0.000782 | 16 | 0.7409 | Intracellular |
| MYLIP | 8.25E−07 | 0.000782 | 17 | 0.86979 | Intracellular |
| CREBBP | 8.25E−07 | 0.000782 | 18 | 0.8983 | Intracellular |
| MGA | 8.25E−07 | 0.000782 | 19 | 0.82215 | Intracellular |
| ZHX2 | 1.37E−06 | 0.001179 | 20 | 0.71898 | Intracellular |
| ZBTB7A | 1.37E−06 | 0.001179 | 21 | 0.54501 | Intracellular |
| RCE1 | 3.02E−06 | 0.002475 | 22 | 0.67358 | Intracellular |
| SLC25A19 | 4.12E−06 | 0.003229 | 23 | 0.73648 | Intracellular |
| CSNK2A1 | 6.32E−06 | 0.004744 | 24 | 1.1232 | Intracellular |
| NPRL2 | 1.07E−05 | 0.007723 | 25 | 0.80111 | Intracellular |
| LRRC8A | 1.40E−05 | 0.009711 | 26 | 0.61842 | Surface |
| TSPYL2 | 1.51E−05 | 0.010084 | 27 | 0.6468 | Intracellular |
| STAT2 | 1.84E−05 | 0.011846 | 28 | 0.97544 | Intracellular |
| JAK1 | 2.28E−05 | 0.014169 | 29 | 0.81642 | Intracellular |
| GLA | 2.45E−05 | 0.014686 | 30 | 0.64116 | Intracellular |
| FTSJ1 | 2.78E−05 | 0.016129 | 31 | 0.51345 | Intracellular |
| THY1 | 4.32E−05 | 0.024288 | 32 | 0.58375 | Surface |
| HDAC4 | 4.87E−05 | 0.026553 | 33 | 0.55804 | Intracellular |
| FOXP1 | 8.77E−05 | 0.038517 | 34 | 0.57003 | Intracellular |
| PDS5B | 5.17E−05 | 0.027373 | 35 | 0.79912 | Intracellular |
| AFF3 | 5.91E−05 | 0.03041 | 36 | 0.69448 | Intracellular |
| S1PR2 | 6.24E−05 | 0.031216 | 37 | 0.76215 | Surface |
| HPS3 | 6.74E−05 | 0.03278 | 38 | 0.57092 | Intracellular |
| STAT1 | 7.78E−05 | 0.036868 | 39 | 0.68125 | Intracellular |
| XK | 8.61E−05 | 0.038517 | 40 | 0.51888 | Surface |
| HMGCR | 8.77E−05 | 0.038517 | 41 | 0.67715 | Intracellular |
| MTA2 | 9.27E−05 | 0.039722 | 42 | 0.76151 | Intracellular |
| MPC2 | 9.71E−05 | 0.04064 | 43 | 0.75121 | Intracellular |
| XPNPEP1 | 0.00010915 | 0.044667 | 44 | 0.74729 | Intracellular |
| GFI1 | 0.00012675 | 0.050474 | 45 | 0.48516 | Intracellular |
| PRKD3 | 0.00012894 | 0.050474 | 46 | 0.62809 | Intracellular |
| ADAM22 | 0.00014764 | 0.056562 | 47 | 0.6288 | Surface |
| ICAM1 | 0.00016249 | 0.060953 | 48 | 1.1527 | Surface |
| LRRC8C | 0.00016964 | 0.062336 | 49 | 0.68614 | Surface |
| PRKCE | 0.00017458 | 0.062871 | 50 | 0.55031 | Intracellular |
| ACSL3 | 0.00019053 | 0.065975 | 51 | 0.60161 | Intracellular |
| KCNG2 | 0.00021857 | 0.074257 | 52 | 0.48822 | Unknown |
| SULT1A2 | 0.00018338 | 0.064745 | 53 | 0.41917 | Intracellular |
| MTMR8 | 0.00022627 | 0.075449 | 54 | 0.67626 | Intracellular |
| CPSF7 | 0.00023782 | 0.076221 | 55 | 0.52652 | Intracellular |
| SGMS1 | 0.00023837 | 0.076221 | 56 | 0.81844 | Intracellular |
| EFHC2 | 0.00024387 | 0.076221 | 57 | 0.65609 | Intracellular |
| MOSPD1 | 0.00024552 | 0.076221 | 58 | 0.64724 | Intracellular |
| ZNF699 | 0.00026036 | 0.07896 | 59 | 1.0114 | Intracellular |
| ZC3H12A | 0.00026311 | 0.07896 | 60 | 0.59232 | Intracellular |
| IFNAR1 | 0.00027686 | 0.080033 | 61 | 1.2274 | Surface |
| LRBA | 0.00028126 | 0.080033 | 62 | 0.67749 | Intracellular |
| DDIT4 | 0.00028676 | 0.080033 | 63 | 0.60138 | Intracellular |
| CDK5 | 0.00029226 | 0.080033 | 64 | 0.32866 | Intracellular |
| ZNF687 | 0.00029281 | 0.080033 | 65 | 0.66561 | Intracellular |
| SLC37A3 | 0.00029336 | 0.080033 | 66 | 0.72918 | Intracellular |
| PNKD | 0.00048086 | 0.11637 | 67 | 0.46774 | Intracellular |
| G2E3 | 0.00030435 | 0.081794 | 68 | 0.85742 | Intracellular |
| FAM122A | 0.00032855 | 0.086998 | 69 | 0.49139 | Intracellular |
| AMMECR1 | 0.00036154 | 0.094346 | 70 | 0.53388 | Intracellular |
| CARKD | 0.00038683 | 0.098661 | 71 | 0.44109 | Intracellular |
| GSPT2 | 0.00038903 | 0.098661 | 72 | 0.57598 | Intracellular |

TABLE 2

| | | NCBI gene ID | | | |
|---|---|---|---|---|---|
| Gene/protein | | (formerly Entrez | | | |
| ID | Gene description | (gene) | Length | Amino acid sequence | SEQ ID NO: |
| GGNBP2 | gametogenetin binding | 79893 | 698 | MARLVAVCRDGEEEFPFERRQIPLYIDDTLTMVMEFPDNVLNLDGH | 1. |
| | protein 2 | | | QNNGAQLKQFIQRHGMLKQQDLSIAMVVTSREVLSALSQLVPCVG | |
| | [Source:HGNC | | | CRRSVERLFSQLVESGNPALEPLTVGPKGVLSVTRSCMTDAKKLYT | |
| | Symbol; Acc:HGNC: | | | LFYVHGSKLNDMIDAIPKSKKNKRCQLHSLDTHKPKPLGGCWMDV | |
| | 19357] | | | WELMSQECRDEVVLIDSSCLLETLETYLRKHRFCTDCKNKVLRAY | |
| | | | | NILIGELDCSKEKGYCAALYEGLRCCPHERHIHVCCETDFIAHLLGR | |
| | | | | AEPEFAGGRRERHAKTIDIAQEEVLTCLGIHLYERLHRIWQKLRAEE | |
| | | | | QTWQMLFYLGVDALRKSFEMTVEKVQGISRLEQLCEEFSEEERVRE | |
| | | | | LKQEKKRQKRKNRRKNKCVCDIPTPLQTADEKEVSQEKETDFIENS | |
| | | | | SCKACGSTEDGNTCVEVIVTNENTSCTCPSSGNLLGSPKIKKGLSPH | |
| | | | | CNGSDCGYSSSMEGSETGSREGSDVACTEGICNHDEHGDDSCVHH | |
| | | | | CEDKEDDGDSCVECWANSEENDTKGKNKKKKKKSKILKCDEHIQK | |
| | | | | LGSCITDPGNRETSGNTMHTVFHRDKTKDTHPESCCSSEKGGQPLP | |
| | | | | WFEHRKNVPQFAEPTETLFGPDSGKGAKSLVELLDESECTSDEEIFIS | |
| | | | | QDEIQSFMANNQSFYSNREQYRQHLKEKFNKYCRLNDHKRPICSG | |
| | | | | WLTTAGAN* | |
| GGNBP2 | gametogenetin binding | 79893 | 134 | XDTKGKNKKKKKKSKILKCDEHIQKLGSCITDPGNRETSGNTMHTV | 2. |
| | protein 2 | | | FHRDKTKDTHPESCCSSEKGGQPLPWFEHRKNVPQFAEPTETLFGP | |
| | [Source:HGNC | | | DSGKGAKSLVELLVSIPCGLTVHNCLGTGWMWEGIVFEGLR* | |
| | Symbol; Acc:HGNC: | | | | |
| | 19357] | | | | |
| GGNBP2 | gametogenetin binding | 79893 | 158 | MARLVAVCRDGEEEFPFERRQIPLYIDDTLTMVMEFPDNVLNLDGH | 3. |
| | protein 2 | | | QNNGAQLKQFIQRHGMLKQQDLSIAMVVTSREVLSALSQLVPCVG | |
| | [Source:HGNC | | | CRRSVERLFSQLVESGNPALEPLTVGPKGVLSVTRSCMTDAKKLYT | |
| | Symbol; Acc:HGNC: | | | LFYVHGSKLNDMIDAIPKSKK | |
| | 19357] | | | | |
| GGNBP2 | gametogenetin binding | 79893 | 69 | MARLVAVCRDGEEEFPFERRQIPLYIDDTLTVSGPGRAGPAAPLAA | 4. |
| | protein 2 | | | VSPPPPPPGRALRTGERVCCNS* | |
| | [Source:HGNC | | | | |
| | Symbol; Acc:HGNC: | | | | |
| | 19357] | | | | |
| PDS5B | PDS5 cohesin | 23047 | 1448 | MAHSKTRTNDGKITYPPGVKEISDKISKEEMVRRLKMVVKTFMDM | 5. |
| | associated factor B | | | DQDSEEEKELYLNLALHLASDFFLKHPDKDVRLLVACCLADIFRIY | |
| | [Source:HGNC | | | APEAPYTSPDKLKDIFMFITRQLKGLEDTKSPQFNRYFYLLENIAWV | |
| | Symbol; Acc:HGNC: | | | KSYNICFELEDSNEIFTQLYRTLFSVINNGHNQKVHMHMVDLMSSII | |
| | 20418] | | | CEGDTVSQELLDTVLVNLVPAHKNLNKQAYDLAKALLKRTAQAIE | |
| | | | | PYITNFFNQVLMLGKTSISDLSEHVFDLILELYNIDSHLLLSVLPQLEF | |
| | | | | KLKSNDNEERLQVVKLLAKMFGAKDSELASQNKPLWQCYLGRFN | |
| | | | | DIHVPIRLECVKFASHCLMNHPDLAKDLTEYLKVRSHDPEEAIRHD | |
| | | | | VIVSIVTAAKKDILLVNDHLLNFVRERTLDKRWRVRKEAMMGLAQ | |
| | | | | IYKKYALQSAAGKDAAKQIAWIKDKLLHIYYQNSIDDRLLVERIFA | |
| | | | | QYMVPHNLETTERMKCLYYLYATLDLNAVKALNEMWKCQNLLR | |
| | | | | HQVKDLLDLIKQPKTDASVKAIFSKVMVITRNLPDPGKAQDFMKKF | |
| | | | | TQVLEDDEKIRKQLEVLVSPTCSCKQAEGCVREITKKLGNPKQPTN | |
| | | | | PFLEMIKFLLERIAPVHIDTESISALIKQVNKSIDGTADDEDEGVPTD | |
| | | | | QAIRAGLELLKVLSFTHPISFHSAETFESLLACLKMDDEKVAEAALQ | |
| | | | | IFKNTGSKIEEDFPHIRSALLPVLHHKSKKGPPRQAKYAIHCIHAIFSS | |
| | | | | KETQFAQIFEPLHKSLDPSNLEHLITPLVTIGHIALLAPDQFAAPLKSL | |
| | | | | VATFIVKDLLMNDRLPGKKTTKLWVPDEEVSPETMVKIQAIKMMV | |
| | | | | RWLLGMKNNHSKSGTSTLRLLTTILHSDGDLTEQGKISKPDMSRLR | |
| | | | | LAAGSAIVKLAQEPCYHEIITLEQYQLCALAINDECYQVRQVFAQK | |
| | | | | LHKGLSRLRLPLEYMAICALCAKDPVKERRAHARQCLVKNINVRR | |
| | | | | EYLKQHAAVSEKLLSLLPEYVVPYTIHLLAHDPDYVKVQDIEQLKD | |
| | | | | VKECLWFVLEILMAKNENNSHAFIRKMVENIKQTKDAQGPDDAKM | |
| | | | | NEKLYTVCDVAMNIIMSKSTTYSLESPKDPVLPARFFTQPDKNFSNT | |
| | | | | KNYLPPEMKSFFTPGKPKTTNVLGAVNKPLSSAGKQSQTKSSRMET | |
| | | | | VSNASSSSNPSSPGRIKGRLDSSEMDHSENEDYTMSSPLPGKKSDKR | |
| | | | | DDSDLVRSELEKPRGRKKTPVTEQEEKLGMDDLTKLVQEQKPKGS | |
| | | | | QRSRKRGHTASESDEQQWPEEKRLKEDILENEDEQNSPPKKGKRGR | |
| | | | | PPKPLGGGTPKEEPTMKTSKKGSKKKSGPPAPEEEEEEERQSGNTEQ | |
| | | | | KSKSKQHRVSRRAQQRAESPESSAIESTQSTPQKGRGRPSKTPSPSQ | |
| | | | | PKKNVRVGRSKQAATKENDSSEEVDVFQGSSPVDDIPQEETEEEEV | |
| | | | | STVNVRRRSAKRERR* | |
| PDS5B | PDS5 cohesin | 23047 | 1392 | MAHSKTRTNDGKITYPPGVKEISDKISKEEMVRRLKMVVKTFMDM | 6. |
| | associated factor B | | | DQDSEEEKELYLNLALHLASDFFLKHPDKDVRLLVACCLADIFRIY | |
| | [Source:HGNC | | | APEAPYTSPDKLKDIFMFITRQLKGLEDTKSPQFNRYFYLLENIAWV | |
| | Symbol; Acc:HGNC: | | | KSYNICFELEDSNEIFTQLYRTLFSVINNGHNQKVHMHMVDLMSSII | |
| | 20418] | | | CEGDTVSQELLDTVLVNLVPAHKNLNKQAYDLAKALLKRTAQAIE | |
| | | | | PYITNFFNQVLMLGKTSISDLSEHVFDLILELYNIDSHLLLSVLPQLEF | |
| | | | | KLKSNDNEERLQVVKLLAKMFGAKDSELASQNKPLWQCYLGRFN | |
| | | | | DIHVPIRLECVKFASHCLMNHPDLAKDLTEYLKVRSHDPEEAIRHD | |
| | | | | VIVSIVTAAKKDILLVNDHLLNFVRERTLDKRWRVRKEAMMGLAQ | |
| | | | | IYKKYALQSAAGKDAAKQIAWIKDKLLHIYYQNSIDDRLLVERIFA | |
| | | | | QYMVPHNLETTERMKCLYYLYATLDLNAVKALNEMWKCQNLLR | |
| | | | | HQVKDLLDLIKQPKTDASVKAIFSKVMVITRNLPDPGKAQDFMKKF | |
| | | | | TQVLEDDEKIRKQLEVLVSPTCSCKQAEGCVREITKKLGNPKQPTN | |
| | | | | PFLEMIKFLLERIAPVHIDTESISALIKQVNKSIDGTADDEDEGVPTD | |
| | | | | QAIRAGLELLKVLSFTHPISFHSAETFESLLACLKMDDEKVAEAALQ | |
| | | | | IFKNTGSKIEEDFPHIRSALLPVLHHKSKKGPPRQAKYAIHCIHAIFSS | |
| | | | | KETQFAQIFEPLHKSLDPSNLEHLITPLVTIGHIALLAPDQFAAPLKSL | |
| | | | | VATFIVKDLLMNDRLPGKKTTKLWVPDEEVSPETMVKIQAIKMMV | |
| | | | | RWLLGMKNNHSKSGTSTLRLLTTILHSDGDLTEQGKISKPDMSRLR | |
| | | | | LAAGSAIVKLAQEPCYHEIITLEQYQLCALAINDECYQVRQVFAQK | |
| | | | | LHKGLSRLRLPLEYMAICALCAKDPVKERRAHARQCLVKNINVRR | |
| | | | | EYLKQHAAVSEKLLSLLPEYVVPYTIHLLAHDPDYVKVQDIEQLKD | |
| | | | | VKECLWFVLEILMAKNENNSHAFIRKMVENIKQTKDAQGPDDAKM | |
| | | | | NEKLYTVCDVAMNIIMSKSTTYSLESPKDPVLPARFFTQPDKNFSNT | |
| | | | | KNYLPPEMKSFFTPGKPKTTNVLGAVNKPLSSAGKQSQTKSSRMET | |
| | | | | VSNASSSSNPSSPGRIKGRLDSSEMDHSENEDYTMSSPLPGKKSDKR | |
| | | | | DDSDLVRSELEKPRGRKKTPVTEQEEKLGMDDLTKLVQEQKPKGS | |
| | | | | QRSRKRGHTASESDEQQWPEEKRLKEDILENEDEQNSPPKKGKRGR | |
| | | | | PPKPLGGGTPKEEPTMKTSKKGSKKKSGPPAPEEEEEEERQSGNTEQ | |
| | | | | KSKSKQHRVSRRAQQRAESPESSAIESTQSTPQKGRGRPSKTPSPSQ | |
| | | | | PKKNV* | |
| PDS5B | PDS5 cohesin | 23047 | 302 | IKQTKDAQGPDDAKMNEKLYTVCDVAMNIIMSKSTTYSLESPKDP | 7. |
| | associated factor B | | | VLPARFFTQPDKNFSNTKNYLPPEMKSFFTPGKPKTTNVLGAVNKP | |
| | [Source:HGNC | | | LSSAGKQSQTKSSRMETVSNASSSSNPSSPGRIKGRLDSSEMDHSEN | |
| | Symbol; Acc:HGNC: | | | EDYTMSSPLPGKKSDKRDDSDLSELEKPRGRKKTPVTEQEEKLGM | |
| | 20418] | | | DDLTKLVQEQKPKGSQRSRKRGHTASESDEQQWPEEKRLKEDILEN | |
| | | | | EDEQNSPPKKGKRGRPPKPLGGGTPKEEPTMKTSKKGSKKKSGPPA | |
| | | | | PEEEEEEERQSGNTEQKSKSKQHRVSR | |
| IRF9 | interferon regulatory | 10379 | 210 | EGDTGGPAVWKTRLRCALNKSSEFKEVPERGRMDVAEPYKVYQLL | 8. |
| | factor 9 [Source:HGNC | | | PPGIVSGQPGTQKVPSKRQHSSVSSERKEEEDAMQNCTLSPSVLQDS | |
| | Symbol; Acc:HGNC: | | | LNNEEEGASGGAVHSDIGSSSSSSSPEPQEVTDTTEAPFQGDQRSLEF | |
| | 6131] | | | LLPPEPDLVRYFQGLGPPPKFQVTLNFWEESHGSSHTPQNLITVKME | |
| | | | | QAFARYLLEQTPEQQAAILSLV* | |
| IRF9 | interferon regulatory | 10379 | 394 | MASGRARCTRKLRNWVVEQVESGQFPGVCWDDTAKTMFRIPWKH | 9. |
| | factor 9 [Source:HGNC | | | AGKQDFREDQDAAFFKAWAIFKGKYKEGDTGGPAVWKTRLRCAL | |
| | Symbol; Acc:HGNC: | | | NKSSEFKEVPERGRMDVAEPYKVYQLLPPGIVSGQPGTQKVPSKRQ | |
| | 6131] | | | HSSVSSERKEEEDAMQNCTLSPSVLQDSLNNEEEGASGGAVHSDIG | |
| | | | | SSSSSSSPEPQEVTDTTEAPFQGDQRSLEFLLPPEPDYSLLLTFIYNGR | |
| | | | | VVGEAQVQSLDCRLVAEPSGSESSMEQVLFPKPGPLEPTQRLLSQLE | |
| | | | | RGILVASNPRGLFVQRLCPIPISWNAPQAPPGPGPHLLPSNECVELFR | |
| | | | | TAYFCRDLVRYFQGLGPPPKFQVTLNFWEESHGSSHTPQNLITVKM | |
| | | | | EQAFARYLLEQTPEQQAAILSLV* | |
| IRF9 | interferon regulatory | 10379 | 51 | MASGRARCTRKLRNWVVEQVESGQFPGVCWDDTAKTMFRIPWKH | 10. |
| | factor 9 [Source:HGNC | | | AGLGNI* | |
| | Symbol; Acc:HGNC: | | | | |
| | 6131] | | | | |
| IRF9 | interferon regulatory | 10379 | 354 | MDVAEPYKVYQLLPPGIVSGQPGTQKVPSKRQHSSVSSERKEEEDA | 11. |
| | factor 9 [Source:HGNC | | | MQNCTLSPSVLQDSLNNEEEGASGGAVHSDIGSSSSSSSPEPQEVTD | |
| | Symbol; Acc:HGNC: | | | TTEAPFQGDQRSLEFLLPPEPDYSLLLTFIYNGRVVGEAQVQSLDCR | |
| | 6131] | | | LVAEPSGSESSMEQVLFPKPGPLEPTQRLLSQLERGILVASNPRGLFV | |
| | | | | QRLCPIPISWNAPQAPPGPGPHLLPSNECVELFRTAYFCRDPPCSLC | |
| | | | | MALLRLCVAYPVWPYAQPGSYLAALLQPRQWKRWFRLGQVLSGP | |
| | | | | GPPTEVPGNTEFLGREPWLQPYSTESYHSEDGAGLCPILAGADSRA | |
| | | | | AGSHSVPGVEPGGPIFHLTSLFFLSPLK* | |
| IRF9 | interferon regulatory | 10379 | 267 | MASGRARCTRKLRNWVVEQVESGQFPGVCWDDTAKTMFRIPWKH | 12. |
| | factor 9 [Source:HGNC | | | AGKQDFREDQDAAFFKAWAIFKGKYKEGDTGGPAVWKTRLRCAL | |
| | Symbol; Acc:HGNC: | | | NKSSEFKEVPERGRMDVAEPYKVYQLLPPGIVSGQPGTQKVPSKRQ | |
| | 6131] | | | HSSVSSERKEEEDAMQNCTLSPSVLQDSLNNEEEGASGGAVHSDIG | |
| | | | | SSSSSSSPEPQEVTDTTEAPFQGDQRSLEFLLPPEPAPGRAVLSPDYS | |
| | | | | LLLTFIYNGRVVGEAQVQSLDCRLVAEPSGSESSMEQVL | |
| IRF9 | interferon regulatory | 10379 | 179 | MASGRARCTRKLRNWVVEQVESGQFPGVCWDDTAKTMFRIPWKH | 13. |
| | factor 9 [Source:HGNC | | | AGKQDFREDQDAAFFKAWAIFKGKYKEGDTGGPAVWKTRLRCAL | |
| | Symbol; Acc:HGNC: | | | NKSSEFKEVPERGRMDVAEPYKVYQLLPPGIVSGQPGTQKVPSKRQ | |
| | 6131] | | | HSSVSSERKEEEDAMQNCTLSPSVLQDSLNNLQTQLRPPFKGIRG | |
| RFXAP | regulatory factor X | 5994 | 273 | MEAQGVAEGAGPGAASGVPHPAALAPAAAPTLAPASVAAAASQFT | 14. |
| | associated protein | | | LLVMQPCAGQDEAAAPGGSVGAGKPVRYLCEGAGDGEEEAGEDE | |
| | [Source:HGNC | | | ADLLDTSDPPGGGESAASLEDLEDEETHSGGEGSSGGARRRGSGGG | |
| | Symbol; Acc:HGNC: | | | SMSKTCTYEGCSETTSQVAKQRKPWMCKKHRNKMYKDKYKKKK | |
| | 9988] | | | SDQALNCGGTASTGSAGNVKLEESADNILSIVKQRTGSFGDRPARP | |
| | | | | TLLEQVLNQKRLSLLRSPEVVQFLQKQQQLLNQQVLEQRQQQFPGT | |
| | | | | SM* | |
| IFNAR1 | interferon alpha and | 3454 | 558 | MMVVLLGATTLVLVAVAPWVLSAAAGGKNLKSPQKVEVDIIDDN | 15. |
| | beta receptor subunit 1 | | | FILRWNRSDESVGNVTFSFDYQKTGMDNWIKLSGCQNITSTKCNFS | |
| | [Source:HGNC | | | SLKLNVYEEIKLRIRAEKENTSSWYEVDSFTPFRKAQIGPPEVHLEA | |
| | Symbol; Acc:HGNC: | | | EDKAIVIHISPGTKDSVMWALDGLSFTYSLVIWKNSSGVEERIENIYS | |
| | 5432] | | | RHKIYKLSPETTYCLKVKAALLTSWKIGVYSPVHCIKTTVENELPPP | |
| | | | | ENIEVSVQNQNYVLKWDYTYANMTFQVQWLHAFLKRNPGNHLYK | |
| | | | | WKQIPDCENVKTTQCVFPQNVFQKGIYLLRVQASDGNNTSFWSEEI | |
| | | | | KFDTEIQAFLLPPVFNIRSLSDSFHIYIGAPKQSGNTPVIQDYPLIYEII | |
| | | | | FWENTSNAERKIIEKKTDVTVPNLKPLTVYCVKARAHTMDEKLNK | |
| | | | | SSVFSDAVCEKTKPGNTSKIWLIVGICIALFALPFVIYAAKVFLRCIN | |
| | | | | YVFFPSLKPSSSIDEYFSEQPLKNLLLSTSEEQIEKCFIIENISTIATVEE | |
| | | | | TNQTDEDHKKYSSQTSQDSGNYSNEDESESKTSEELQQDFV* | |
| IFNAR1 | interferon alpha and | 3454 | 189 | MMVVLLGATTLVLVAVAPWVLSAAAGGKNLKSPQKVEVDIIDDN | 16. |
| | beta receptor subunit 1 | | | FILRWNRSDESVGNVTFSFDYQKTGMDNWIKLSGCQNITSTKCNFS | |
| | [Source:HGNC | | | SLKLNVYEEIKLRIRAEKENTSSWYEVDSFTPFRKVENELPPPENIEV | |
| | Symbol; Acc:HGNC: | | | SVQNQNYVLKWDYTYANMTFQVQWLHAFLKRNPGNHLYKWKQI | |
| | 5432] | | | PDCENVKT | |
| IFNAR1 | interferon alpha and | 3454 | 68 | MMVVLLGATTLVLVAVAPWVLSAAAGGKNLKSPQKVEVDIIDDN | 17. |
| | beta receptor subunit 1 | | | FILRWNRSDESVGNVTFSFDYQN* | |
| | [Source:HGNC | | | | |
| | Symbol; Acc:HGNC: | | | | |
| | 5432] | | | | |
| IFNAR1 | interferon alpha and | 3454 | 237 | MMVVLLGATTLVLVAVAPWVLSAAAGGKNLKSPQKVEVDIIDDN | 18. |
| | beta receptor subunit 1 | | | FILRWNRSDESVGNVTFSFDYQKTGMDNWIKLSGCQNITSTKCNFS | |
| | [Source:HGNC | | | SLKLNVYEEIKLRIRAEKENTSSWYEVDSFTPFRKAQIGPPEVHLEA | |
| | Symbol; Acc:HGNC: | | | EDKAIVIHISPGTKDSVMWALDGLSFTYSLVIWKNSSGVEERIENIYS | |
| | 5432] | | | RHKIYKLSPETTYCLKVKAALLTSWKIGVYSPVHCIKTTVCILGGRG | |
| | | | | RWIT* | |
| IFNAR1 | interferon alpha and | 3454 | 129 | MMVVLLGATTLVLVAVAPWVLSAAAGGKNLKSPQKVEVDIIDDN | 19. |
| | beta receptor subunit 1 | | | FILRWNRSDESVGNVTFSFDYQKTGMDNWIKLSGCQNITSTKCNFS | |
| | [Source:HGNC | | | SLKLNVYEEIKLRIRAEKENTSSWYEVDSFTPFRKGKD* | |
| | Symbol; Acc:HGNC: | | | | |
| | 5432] | | | | |
| IFNAR1 | interferon alpha and | 3454 | 271 | MMVVLLGATTLVLVAVAPWVLSAAAGGKNLKSPQKVEVDIIDDN | 20. |
| | beta receptor subunit 1 | | | FILRWNRSDESVGNVTFSFDYQKTGMDNWIKLSGCQNITSTKCNFS | |
| | [Source:HGNC | | | SLKLNVYEEIKLRIRAEKENTSSWYEVDSFTPFRKAQIGPPEVHLEA | |
| | Symbol; Acc:HGNC: | | | EDKAIVIHISPGTKDSVMWALDGLSFTYSLVIWKNSSGVEERIENIYS | |
| | 3542] | | | RHKIYKLSPETTYCLKVKAALLTSWKIGVYSPVHCIKTTVENELPPP | |
| | | | | ENIEVSVQNQNYVLKWDYTYANMTFQVQWLHLYFRRPR* | |
| IFNAR1 | interferon alpha and | 3454 | 489 | MDNWIKLSGCQNITSTKCNFSSLKLNVYEEIKLRIRAEKENTSSWYE | 21. |
| | beta receptor subunit 1 | | | VDSFTPFRKAQIGPPEVHLEAEDKAIVIHISPGTKDSVMWALDGLSF | |
| | [Source:HGNC | | | TYSLVIWKNSSGVEERIENIYSRHKIYKLSPETTYCLKVKAALLTSW | |
| | Symbol; Acc:HGNC: | | | KIGVYSPVHCIKTTVENELPPPENIEVSVQNQNYVLKWDYTYANMT | |
| | 5432] | | | FQVQWLHAFLKRNPGNHLYKWKQIPDCENVKTTQCVFPQNVFQK | |
| | | | | GIYLLRVQASDGNNTSFWSEEIKFDTEIQAFLLPPVFNIRSLSDSFHIY | |
| | | | | IGAPKQSGNTPVIQDYPLIYEIIFWENTSNAERKIIEKKTDVTVPNLKP | |
| | | | | LTVYCVKARAHTMDEKLNKSSVFSDAVCEKTKPGNTSKIWLIVGIC | |
| | | | | IALFALPFVIYAAKVFLRCINYVFFPSLKPSSSIDEYFSEQPLKNLLLS | |
| | | | | TSEEQIEKCFIIENISTIATVEETNQTDEDHKKYSSQTSQDSGNYSNE | |
| | | | | DESESKTSEELQQDFV* | |
| NAXD | NAD(P)HX | 55739 | 391 | MVTRAGAGTAVAGAVVVALLSAALALYGPPLDAVLERAFSLRKA | 22. |
| | dehydratase | | | HSIKDMENTLQLVRNIIPPLSSTKHKGQDGRIGVVGGCQEYTGAPYF | |
| | [Source:HGNC | | | AAISALKVGADLSHVFCASAAAPVIKAYSPELIVHPVLDSPNAVHEV | |
| | Symbol; Acc:HGNC: | | | EKWLPRLHALVVGPGLGRDDALLRNVQGILEVSKARDIPVVIDADG | |
| | 25576] (encoded by | | | LWLVAQQPALIHGYRKAVLTPNHVEFSRLYDAVLRGPMDSDDSHG | |
| | CARKD gene set forth | | | SVLRLSQALGNVTVVQKGERDILSNGQQVLVCSQEGSSRRCGGQG | |
| | in Table 1) | | | DLLSGSLGVLVHWALLAGPQKTNGGISDLKLTIWGPEIETGVKTRA | |
| | | | | QGSCGPRTTTPTSPHLLLSPSPQVQPSPGGRVWRLLSHQAVQPPSLP | |
| | | | | EARSLHHHLRHDRRGGGRLQQAL* | |
| NAXD | NAD(P)HX | 55739 | 238 | MLPDGPGSSLWGNPGLQTSDSPNAVHEVEKWLPRLHALVVGPGLG | 23. |
| | dehydratase | | | RDDALLRNVQGILEVSKARDIPVVIDADGLWLVAQQPALIHGYRKA | |
| | [Source:HGNC | | | VLTPNHVEFSRLYDAVLRGPMDSDDSHGSVLRLSQALGNVTVVQK | |
| | Symbol; Acc:HGNC: | | | GERDILSNGQQVLVCSQEGSSRRCGGQGDLLSGSLGVLVHWALLA | |
| | 25576] (encoded by | | | GPQKTNGSSPLLVAAFGACSLTRQCNHQAFQKHGRSTTTSDMIAEV | |
| | CARKD gene set forth | | | GAAFSKLFET* | |
| | in Table 1) | | | | |
| CSNK2A1 | casein kinase 2 alpha 1 | 1457 | 392 | MSGPVPSRARVYTDVNTHRPREYWDYESHVVEWGNQDDYQLVR | 24. |
| | [Source:HGNC | | | KLGRGKYSEVFEAINITNNEKVVVKILKPVKKKKIKREIKILENLRG | |
| | Symbol; Acc:HGNC: | | | GPNIITLADIVKDPVSRTPALVFEHVNNTDFKQLYQTLTDYDIRFYM | |
| | 2457] | | | YEILKALDYCHSMGIMHRDVKPHNVMIDHEHRKLRLIDWGLAEFY | |
| | | | | HPGQEYNVRVASRYFKGPELLVDYQMYDYSLDMWSLGCMLASMI | |
| | | | | FRKEPFFHGHDNYDQLVRIAKVLGTEDLYDYIDKYNIELDPRFNDIL | |
| | | | | GRHSRKRWERFVHSENQHLVSPEALDFLDKLLRYDHQSRLTAREA | |
| | | | | MEHPYFYTVVKDQARMGSSSMPGGSTPVSSANMMSGISSVPTPSPL | |
| | | | | GPLAGSPVIAAANPLGMPVPAAAGAQQ* | |
| CSNK2A1 | casein kinase 2 alpha 1 | 1457 | 256 | MYEILKALDYCHSMGIMHRDVKPHNVMIDHEHRKLRLIDWGLAEF | 25. |
| | [Source:HGNC | | | YHPGQEYNVRVASRYFKGPELLVDYQMYDYSLDMWSLGCMLAS | |
| | Symbol; Acc:HGNC: | | | MIFRKEPFFHGHDNYDQLVRIAKVLGTEDLYDYIDKYNIELDPRFN | |
| | 2457] | | | DILGRHSRKRWERFVHSENQHLVSPEALDFLDKLLRYDHQSRLTAR | |
| | | | | EAMEHPYFYTVVKDQARMGSSSMPGGSTPVSSANMMSGISSVPTPS | |
| | | | | PLGPLAGSPVIAAANPLGMPVPAAAGAQQ* | |
| CSNK2A1 | casein kinase 2 alpha 1 | 1457 | 386 | MSGPVPSRARVYTDVNTHRPREYWDYESHVVEWGNQDDYQLVR | 26. |
| | [Source:HGNC | | | KLGRGKYSEVFEAINITNNEKVVVKILKPVKKKKIKREIKILENLRG | |
| | Symbol; Acc:HGNC: | | | GPNIITLADIVKDPVSRTPALVFEHVNNTDFKQLYQTLTDYDIRFYM | |
| | 2457] | | | YEILKALDYCHSMGIMHRDVKPHNVMIDHEHRKLRLIDWGLAEFY | |
| | | | | HPGQEYNVRVASRYFKGPELLVDYQMYDYSLDMWSLGCMLASMI | |
| | | | | FRKEPFFHGHDNYDQLVRIAKVLGTEDLYDYIDKYNIELDPRFNDIL | |
| | | | | GRHSRKRWERFVHSENQHLVSPEALDFLDKLLRYDHQSRLTAREA | |
| | | | | MEHPYFYTVVKDQARMGSSSMPGGSTPVSSANMMSEMKFCRVAQ | |
| | | | | AGLELLSSTVYPPWPPKVLRLEA* | |
| CSNK2A1 | casein kinase 2 alpha 1 | 1457 | 43 | MYEILKALDYCHSMGIMHRDVKPHNVMIDHEHRKLRLIDWGLA | 27. |
| | [Source:HGNC | | | | |
| | Symbol; Acc:HGNC: | | | | |
| | 2457] | | | | |
| CSNK2A1 | casein kinase 2 alpha 1 | 1457 | 118 | MSGPVPSRARVYTDVNTHRPREYWDYESHVVEWGNQDDYQLVR | 28. |
| | [Source:HGNC | | | KLGRGKYSEVFEAINITNNEKVVVKILKPVKKKKIKREIKILENLRG | |
| | Symbol; Acc:HGNC: | | | GPNIITLADIVKDPVAKRLIALETEMC* | |
| | 2457] | | | | |
| CSNK2A1 | casein kinase 2 alpha 1 | 1457 | 133 | MSGPVPSRARVYTDVNTHRPREYWDYESHVVEWGNQDDYQLVR | 29. |
| | [Source:HGNC | | | KLGRGKYSEVFEAINITNNEKVVVKILKPVKKKKIKREIKILENLRG | |
| | Symbol; Acc:HGNC: | | | GPNIITLADIVKDPVSRTPALVFEHVNNTDFKQLYQTLTDYDI | |
| | 2457] | | | | |
| CSNK2A1 | casein kinase 2 alpha 1 | 1457 | 74 | MSGPVPSRARVYTDVNTHRPREYWDYESHVVEWGNQDDYQLVR | 30. |
| | [Source:HGNC | | | KLGRGKYSEVFEAINITNNEKVVVKILKPVK | |
| | Symbol; Acc:HGNC: | | | | |
| | 2457] | | | | |
| CSNK2A1 | casein kinase 2 alpha 1 | 1457 | 207 | MSGPVPSRARVYTDVNTHRPREYWDYESHVVEWGNQDDYQLVR | 31. |
| | [Source:HGNC | | | KLGRGKYSEVFEAINITNNEKVVVKILKPVKKKKIKREIKILENLRG | |
| | Symbol; Acc:HGNC: | | | GPNIITLADIVKDPVSRTPALVFEHVNNTDFKQLYQTLTDYDIRFYM | |
| | 2457] | | | YEILKALDYCHSMGIMHRDVKPHNVMIDHEHRKLRLIDWGLAEFY | |
| | | | | HPGQEYNVRVASRYFKGPELLVDYQ | |
| CSNK2A1 | casein kinase 2 alpha 1 | 1457 | 153 | MSGPVPSRARVYTDVNTHRPREYWDYESHVVEWGNQDDYQLVR | 32. |
| | [Source:HGNC | | | KLGRGKYSEVFEAINITNNEKVVVKILKYLLFTTATTANPASQKYAI | |
| | Symbol; Acc:HGNC: | | | ARSITQSITRSVTQSNVETTNHLKLAVHPVSGRFNDGLMCLPQKCK | |
| | 2457] | | | VFHSIPVHCWGASHAL* | |
| CSNK2A1 | casein kinase 2 alpha 1 | 1457 | 131 | MSGPVPSRARVYTDVNTHRPREYWDYESHVVEWGNQDDYQLVR | 33. |
| | [Source:HGNC | | | KLGRGKYSEVFEAINITNNEKVVVKILKSRTPALVFEHVNNTDFKQ | |
| | Symbol; Acc:HGNC: | | | LYQTLTDYDIRFYMYEILKALDYCHSMGIMHRDVKPHNVMID | |
| | 2457] | | | | |
| CSNK2A1 | casein kinase 2 alpha 1 | 1457 | 87 | MSGPVPSRARVYTDVNTHRPREYWDYESHVVEWGNQDDYQLVR | 34. |
| | [Source:HGNC | | | KLGRGKYSEVFEAINITNNEKVVVKILKVSTRDNFRGIWNIDF* | |
| | Symbol; Acc:HGNC: | | | | |
| | 2457] | | | | |
| CSNK2A1 | casein kinase 2 alpha 1 | 1457 | 364 | MSGPVPSRARVYTDVNTHRPREYWDYESHVVEWGNQDDYQLVR | 35. |
| | [Source:HGNC | | | KLGRGKYSEVFEAINITNNEKVVVKILKPVKKKKIKREIKILENLRG | |
| | Symbol; Acc:HGNC: | | | GPNIITLADIVKDPVSRTPALVFEHVNNTDFKQLYQTLTDYDIRFYM | |
| | 2457] | | | YEILKLRLIDWGLAEFYHPGQEYNVRVASRYFKGPELLVDYQMYD | |
| | | | | YSLDMWSLGCMLASMIFRKEPFFHGHDNYDQLVRIAKVLGTEDLY | |
| | | | | DYIDKYNIELDPRFNDILGRHSRKRWERFVHSENQHLVSPEALDFLD | |
| | | | | KLLRYDHQSRLTAREAMEHPYFYTVVKDQARMGSSSMPGGSTPVS | |
| | | | | SANMMSGISSVPTPSPLGPLAGSPVIAAANPLGMPVPAAAGAQQ* | |
| CSNK2A1 | casein kinase 2 alpha 1 | 1457 | 375 | MSGPVPSRARVYTDVNTHRPREYWDYESHVVEWGNQDDYQLVR | 36. |
| | [Source:HGNC | | | KLGRGKYSEVFEAINITNNEKVVVKILKPVKKKKIKREIKILENLRG | |
| | Symbol; Acc:HGNC: | | | GPNIITLADIVKDPVQLYQTLTDYDIRFYMYEILKALDYCHSMGIMH | |
| | 2457] | | | RDVKPHNVMIDHEHRKLRLIDWGLAEFYHPGQEYNVRVASRYFKG | |
| | | | | PELLVDYQMYDYSLDMWSLGCMLASMIFRKEPFFHGHDNYDQLV | |
| | | | | RIAKVLGTEDLYDYIDKYNIELDPRFNDILGRHSRKRWERFVHSEN | |
| | | | | QHLVSPEALDFLDKLLRYDHQSRLTAREAMEHPYFYTVVKDQARM | |
| | | | | GSSSMPGGSTPVSSANMMSGISSVPTPSPLGPLAGSPVIAAANPLGM | |
| | | | | PVPAAAGAQQ* | |
| CSNK2A1 | casein kinase 2 alpha 1 | 1457 | 89 | MSGPVPSRARVYTDVNTHRPREYWDYESHVVEWGNQDDYQLVR | 37. |
| | [Source:HGNC | | | KLGRGKYSEVFEAINITNNEKVVVKILKYLQNTWHSARRRIQRRM* | |
| | Symbol; Acc:HGNC: | | | | |
| | 2457] | | | | |
| CSNK2A1 | casein kinase 2 alpha 1 | 1457 | 34 | MSGPVPSRARVYTDVNTHRPREYWDYESHVVEWG | 38. |
| | [Source:HGNC | | | | |
| | Symbol; Acc:HGNC: | | | | |
| | 2457] | | | | |
| CSNK2A1 | casein kinase 2 alpha 1 | 1457 | 257 | MSGPVPSRARVYTDVNTHRPREYWDYESHVVEWGNQDDYQLVR | 39. |
| | [Source:HGNC | | | KLGRGKYSEVFEAINITNNEKVVVKILKPVKKKKIKREIKILENLRG | |
| | Symbol; Acc:HGNC: | | | GPNIITLADIVKDPVSRTPALVFEHVNNTDFKQLYQTLTDYDIRFYM | |
| | 2457] | | | YEILKALDYCHSMGIMHRDVKPHNVMIDHEHRKLRLIDWGLAEFY | |
| | | | | HPGQEYNVRVASRYFKGPELLVDYQMYDYSLDMWSLGCMLASMI | |
| | | | | FRKEPFFHGHDNYDQVIYVIPIEKCTGLMQ* | |
| CSNK2A1 | casein kinase 2 alpha 1 | 1457 | 119 | DYIDKYNIELDPRFNDILGRHSRKRWERFVHSENQHLVSPEALDFLD | 40. |
| | [Source:HGNC | | | KLLRYDHQSRLTAREAMEHPYFYTVVKDQARMGSSSMPGGSTPVS | |
| | Symbol; Acc:HGNC: | | | SANMMSEPQFEGQRDIHHAAVRLGQL* | |
| | 2457] | | | | |
| CSNK2A1 | casein kinase 2 alpha 1 | 1457 | 355 | MSGPVPSRARVYTDVNTHRPREYWDYESHVVEWGNQDDYQLVR | 41. |
| | [Source:HGNC | | | KLGRGKYSEVFEAINITNNEKVVVKILKPVKKKKIKREIKILENLRG | |
| | Symbol; Acc:HGNC: | | | GPNIITLADIVKDPVALDYCHSMGIMHRDVKPHNVMIDHEHRKLRL | |
| | 2457] | | | IDWGLAEFYHPGQEYNVRVASRYFKGPELLVDYQMYDYSLDMWS | |
| | | | | LGCMLASMIFRKEPFFHGHDNYDQLVRIAKVLGTEDLYDYIDKYNI | |
| | | | | ELDPRFNDILGRHSRKRWERFVHSENQHLVSPEALDFLDKLLRYDH | |
| | | | | QSRLTAREAMEHPYFYTVVKDQARMGSSSMPGGSTPVSSANMMS | |
| | | | | GISSVPTPSPLGPLAGSPVIAAANPLGMPVPAAAGAQQ* | |
| CSNK2A1 | casein kinase 2 alpha 1 | 1457 | 92 | MSGPVPSRARVYTDVNTHRPREYWDYESHVVEWGNQDDYQLVR | 42. |
| | [Source:HGNC | | | KLGRGKYSEVFEAINITNNEKVVVKILKPVKKKKIKREIKILENLRG | |
| | Symbol; Acc:HGNC: | | | GP | |
| | 2457] | | | | |
| CSNK2A1 | casein kinase 2 alpha 1 | 1457 | 95 | MSGPVPSRARVYTDVNTHRPREYWDYESHVVEWGNQDDYQLVR | 43. |
| | [Source:HGNC | | | KLGRGKYSEVFEAINITNNEKVVVKILKSQVLEGLKWEDRLSLEVE | |
| | Symbol; Acc:HGNC: | | | GCTDL* | |
| | 2457] | | | | |
| CSNK2A1 | casein kinase 2 alpha 1 | 1457 | 355 | MSGPVPSRARVYTDVNTHRPREYWDYESHVVEWGNQDDYQLVR | 44. |
| | [Source:HGNC | | | KLGRGKYSEVFEAINITNNEKVVVKILKPVKKKKIKREIKILENLRG | |
| | Symbol; Acc:HGNC: | | | GPNIITLADIVKDPVSRTPALVFEHVNNTDFKQLYQTLTDYDIRFYM | |
| | 2457] | | | YEILKALDYCHSMGIMHRDVKPHNVMIDHEHRKMYDYSLDMWSL | |
| | | | | GCMLASMIFRKEPFFHGHDNYDQLVRIAKVLGTEDLYDYIDKYNIE | |
| | | | | LDPRFNDILGRHSRKRWERFVHSENQHLVSPEALDFLDKLLRYDHQ | |
| | | | | SRLTAREAMEHPYFYTVVKDQARMGSSSMPGGSTPVSSANMMSGI | |
| | | | | SSVPTPSPLGPLAGSPVIAAANPLGMPVPAAAGAQQ* | |
| CSNK2A1 | casein kinase 2 alpha 1 | 1457 | 377 | MSGPVPSRARVYTDVNTHRPREYWDYESHVVEWGNQDDYQLVR | 45. |
| | [Source:HGNC | | | KLGRGKYSEVFEAINITNNEKVVVKILKPVKKKKIKREIKILENLRG | |
| | Symbol; Acc:HGNC: | | | GPNIITLADIVKDPVSRTPALVFEHVNNTDFKQLYQTLTDYDIRFYM | |
| | 2457] | | | YEILKALDYCHSMGIMHRDVKPHNVMIDHEHRKLRLIDWGLAEFY | |
| | | | | HPGQEYNVRVASRYFKGPELLVDYQMYDYSLDMWSLGCMLASMI | |
| | | | | FRKEPFFHGHDNYDQLVRIAKVLGTEDLYDYIDKYNIELDPRFNDIL | |
| | | | | GRHSRKRWERFVHSENQHLVSPEALDFLDKLLRYDHQSRLTAREA | |
| | | | | MEHPYFYTVVKDQARMGSSSMPGGSTPVSSANMMSALFFQGFLQC | |
| | | | | QPLHPLDLWQAHQ* | |
| CSNK2A1 | casein kinase 2 alpha 1 | 1457 | 144 | MSGPVPSRARVYTDVNTHRPREYWDYESHVVEWGNQDDYQLVR | 46. |
| | [Source:HGNC | | | KLGRGKYSEVFEAINITNNEKVVVKILKPVKKKKIKREIKILENLRG | |
| | Symbol; Acc:HGNC: | | | GPNIITLADIVKDPVSRTPALVFEHVNNTDFKQLYQTLTDYDIRFYM | |
| | 2457] | | | YEILKAL | |
| KCNG2 | potassium voltage- | 26251 | 467 | MEPWPCSPGGGGGTRARHVIINVGGCRVRLAWAALARCPLARLER | 47. |
| | gated channel modifier | | | LRACRGHDDLLRVCDDYDVSRDEFFFDRSPCAFRAIVALLRAGKLR | |
| | subfamily G member 2 | | | LLRGPCALAFRDELAYWGIDEARLERCCLRRLRRREEEAAEARAGP | |
| | [Source:HGNC | | | TERGAQGSPARALGPRGRLQRGRRRLRDVVDNPHSGLAGKLFACV | |
| | Symbol; Acc:HGNC: | | | SVSFVAVTAVGLCLSTMPDIRAEEERGECSPKCRSLFVLETVCVAW | |
| | 6249] | | | FSFEFLLRSLQAESKCAFLRAPLNIIDILALLPFYVSLLLGLAAGPGGT | |
| | | | | KLLERAGLVLRLLRALRVLYVMRLARHSLGLRSLGLTMRRCAREF | |
| | | | | GLLLLFLCVAMALFAPLVHLAERELGARRDFSSVPASYWWAVISM | |
| | | | | TTVGYGDMVPRSLPGQVVALSSILSGILLMAFPVTSIFHTFSRSYSEL | |
| | | | | KEQQQRAASPEPALQEDSTHSATATEDSSQGPDSAGLADDSADAL | |
| | | | | WVRAGR* | |
| FAM122A | family with sequence | 116224 | 288 | MAQEKMELDLELPPGTGGSPAEGGGSGGGGGLRRSNSAPLIHGLSD | 48. |
| | similarity 122 A | | | TSPVFQAEAPSARRNSTTFPSRHGLLLPASPVRMHSSRLHQIKQEEG | |
| | [Source:HGNC | | | MDLINRETVHEREVQTAMQISHSWEESFSLSDNDVEKSASPKRIDFI | |
| | Symbol; Acc:HGNC: | | | PVSPAPSPTRGIGKQCFSPSLQSFVSSNGLPPSPIPSPTTRFTTRRSQSP | |
| | 23490] | | | INCIRPSVLGPLKRKCEMETEYQPKRFFQGITNMLSSDVAQLSDPGV | |
| | | | | CVSSDTLDGNSSSAGSSCNSPAKVSTTTDSPVSPAQAASPFIPLDELS | |
| | | | | SK* | |
| MGA | MAX dimerization | 23269 | 3066 | MEEKQQIILANQDGGTVAGAAPTFFVILKQPGNGKTDQGILVTNQD | 49. |
| | protein MGA | | | ACALASSVSSPVKSKGKICLPADCTVGGITVTLDNNSMWNEFYHRS | |
| | [Source:HGNC | | | TEMILTKQGRRMFPYCRYWITGLDSNLKYILVMDISPVDNHRYKW | |
| | Symbol; Acc:HGNC: | | | NGRWWEPSGKAEPHVLGRVFIHPESPSTGHYWMHQPVSFYKLKLT | |
| | 14010] | | | NNTLDQEGHIILHSMHRYLPRLHLVPAEKAVEVIQLNGPGVHTFTFP | |
| | | | | QTEFFAVTAYQNIQITQLKIDYNPFAKGFRDDGLNNKPQRDGKQKN | |
| | | | | SSDQEGNNISSSSGHRVRLTEGQGSEIQPGDLDPLSRGHETSGKGLE | |
| | | | | KTSLNIKRDFLGFMDTDSALSEVPQLKQEISECLIASSFEDDSRVASP | |
| | | | | LDQNGSFNVVIKEEPLDDYDYELGECPEGVTVKQEETDEETDVYSN | |
| | | | | SDDDPILEKQLKRHNKVDNPEADHLSSKWLPSSPSGVAKAKMFKL | |
| | | | | DTGKMPVVYLEPCAVTRSTVKISELPDNMLSTSRKDKSSMLAELEY | |
| | | | | LPTYIENSNETAFCLGKESENGLRKHSPDLRVVQKYPLLKEPQWKY | |
| | | | | PDISDSISTERILDDSKDSVGDSLSGKEDLGRKRTTMLKIATAAKVV | |
| | | | | NANQNASPNVPGKRGRPRKLKLCKAGRPPKNTGKSLISTKNTPVSP | |
| | | | | GSTFPDVKPDLEDVDGVLFVSFESKEALDIHAVDGTTEESSSLQAST | |
| | | | | TNDSGYRARISQLEKELIEDLKTLRHKQVIHPGLQEVGLKLNSVDPT | |
| | | | | MSIDLKYLGVQLPLAPATSFPFWNLTGTNPASPDAGFPFVSRTGKT | |
| | | | | NDFTKIKGWRGKFHSASASRNEGGNSESSLKNRSAFCSDKLDEYLE | |
| | | | | NEGKLMETSMGFSSNAPTSPVVYQLPTKSTSYVRTLDSVLKKQSTIS | |
| | | | | PSTSYSLKPHSVPPVSRKAKSQNRQATFSGRTKSSYKSILPYPVSPKQ | |
| | | | | KYSHVILGDKVTKNSSGIISENQANNFVVPTLDENIFPKQISLRQAQQ | |
| | | | | QQQQQQGSRPPGLSKSQVKLMDLEDCALWEGKPRTYITEERADVS | |
| | | | | LTTLLTAQASLKTKPIHTIIRKRAPPCNNDFCRLGCVCSSLALEKRQP | |
| | | | | AHCRRPDCMFGCTCLKRKVVLVKGGSKTKHFQRKAAHRDPVFYD | |
| | | | | TLGEEAREEEEGIREEEEQLKEKKKRKKLEYTICETEPEQPVRHYPL | |
| | | | | WVKVEGEVDPEPVYIPTPSVIEPMKPLLLPQPEVLSPTVKGKLLTGI | |
| | | | | KSPRSYTPKPNPVIREEDKDPVYLYFESMMTCARVRVYERKKEDQR | |
| | | | | QPSSSSSPSPSFQQQTSCHSSPENHNNAKEPDSEQQPLKQLTCDLED | |
| | | | | DSDKLQEKSWKSSCNEGESSSTSYMHQRSPGGPTKLIEIISDCNWEE | |
| | | | | DRNKILSILSQHINSNMPQSLKVGSFIIELASQRKSRGEKNPPVYSSR | |
| | | | | VKISMPSCQDQDDMAEKSGSETPDGPLSPGKMEDISPVQTDALDSV | |
| | | | | RERLHGGKGLPFYAGLSPAGKLVAYKRKPSSSTSGLIQVASNAKVA | |
| | | | | ASRKPRTLLPSTSNSKMASSSGTATNRPGKNLKAFVPAKRPIAARPS | |
| | | | | PGGVFTQFVMSKVGALQQKIPGVSTPQTLAGTQKFSIRPSPVMVVT | |
| | | | | PVVSSEPVQVCSPVTAAVTTTTPQVFLENTTAVTPMTAISDVETKET | |
| | | | | TYSSGATTTGVVEVSETNTSTSVTSTQSTATVNLTKTTGITTPVASV | |
| | | | | AFPKSLVASPSTITLPVASTASTSLVVVTAAASSSMVTTPTSSLGSVP | |
| | | | | IILSGINGSPPVSQRPENAAQIPVATPQVSPNTVKRAGPRLLLIPVQQ | |
| | | | | GSPTLRPVSNTQLQGHRMVLQPVRSPSGMNLFRHPNGQIVQLLPLH | |
| | | | | QLRGSNTQPNLQPVMFRNPGSVMGIRLPAPSKPSETPPSSTSSSAFSV | |
| | | | | MNPVIQAVGSSSAVNVITQAPSLLSSGASFVSQAGTLTLRISPPEPQS | |
| | | | | FASKTGSETKITYSSGGQPVGTASLIPLQSGSFALLQLPGQKPVPSSIL | |
| | | | | QHVASLQMKRESQNPDQKDETNSIKREQETKKVLQSEGEAVDPEA | |
| | | | | NVIKQNSGAATSEETLNDSLEDRGDHLDEECLPEEGCATVKPSEHS | |
| | | | | CITGSHTDQDYKDVNEEYGARNRKSSKEKVAVLEVRTISEKASNKT | |
| | | | | VQNLSKVQHQKLGDVKVEQQKGFDNPEENSSEFPVTFKEESKFELS | |
| | | | | GSKVMEQQSNLQPEAKEKECGDSLEKDRERWRKHLKGPLTRKCV | |
| | | | | GASQECKKEADEQLIKETKTCQENSDVFQQEQGISDLLGKSGITEDA | |
| | | | | RVLKTECDSWSRISNPSAFSIVPRRAAKSSRGNGHFQGHLLLPGEQI | |
| | | | | QPKQEKKGGRSSADFTVLDLEEDDEDDNEKTDDSIDEIVDVVSDYQ | |
| | | | | SEEVDDVEKNNCVEYIEDDEEHVDIETVEELSEEINVAHLKTTAAHT | |
| | | | | QSFKQPSCTHISADEKAAERSRKAPPIPLKLKPDYWSDKLQKEAEAF | |
| | | | | AYYRRTHTANERRRRGEMRDLFEKLKITLGLLHSSKVSKSLILTRAF | |
| | | | | SEIQGLTDQADKLIGQKNLLTRKRNILIRKVSSLSGKTEEVVLKKLE | |
| | | | | YIYAKQQALEAQKRKKKMGSDEFDISPRISKQQEGSSASSVDLGQM | |
| | | | | FINNRRGKPLILSRKKDQATENTSPLNTPHTSANLVMTPQGQLLTLK | |
| | | | | GPLFSGPVVAVSPDLLESDLKPQVAGSAVALPENDDLFMMPRIVNV | |
| | | | | TSLATEGGLVDMGGSKYPHEVPDSKPSDHLKDTVRNEDNSLEDKG | |
| | | | | RISSRGNRDGRVTLGPTQVFLANKDSGYPQIVDVSNMQKAQEFLPK | |
| | | | | KISGDMRGIQYKWKESESRGERVKSKDSSFHKLKMKDLKDSSIEME | |
| | | | | LRKVTSAIEEAALDSSELLTNMEDEDDTDETLTSLLNEIAFLNQQLN | |
| | | | | DDSVGLAELPSSMDTEFPGDARRAFISKVPPGSRATFQVEHLGTGL | |
| | | | | KELPDVQGESDSISPLLLHLEDDDFSENEKQLAEPASEPDVLKIVIDS | |
| | | | | EIKDSLLSNKKAIDGGKNTSGLPAEPESVSSPPTLHMKTGLENSNST | |
| | | | | DTLWRPMPKLAPLGLKVANPSSDADGQSLKVMPCLAPIAAKVGSV | |
| | | | | GHKMNLTGNDQEGRESKVMPTLAPVVAKLGNSGASPSSAGK* | |
| MGA | MAX dimerization | 23269 | 1138 | MEEKQQIILANQDGGTVAGAAPTFFVILKQPGNGKTDQGILVTNQD | 50. |
| | protein MGA | | | ACALASSVSSPVKSKGKICLPADCTVGGITVTLDNNSMWNEFYHRS | |
| | [Source:HGNC | | | TEMILTKQGRRMFPYCRYWITGLDSNLKYILVMDISPVDNHRYKW | |
| | Symbol; Acc:HGNC: | | | NGRWWEPSGKAEPHVLGRVFIHPESPSTGHYWMHQPVSFYKLKLT | |
| | 14010] | | | NNTLDQEGHIILHSMHRYLPRLHLVPAEKAVEVIQLNGPGVHTFTFP | |
| | | | | QTEFFAVTAYQNIQITQLKIDYNPFAKGFRDDGLNNKPQRDGKQKN | |
| | | | | SSDQEGNNISSSSGHRVRLTEGQGSEIQPGDLDPLSRGHETSGKGLE | |
| | | | | KTSLNIKRDFLGFMDTDSALSEVPQLKQEISECLIASSFEDDSRVASP | |
| | | | | LDQNGSFNVVIKEEPLDDYDYELGECPEGVTVKQEETDEETDVYSN | |
| | | | | SDDDPILEKQLKRHNKVDNPEADHLSSKWLPSSPSGVAKAKMFKL | |
| | | | | DTGKMPVVYLEPCAVTRSTVKISELPDNMLSTSRKDKSSMLAELEY | |
| | | | | LPTYIENSNETAFCLGKESENGLRKHSPDLRVVQKYPLLKEPQWKY | |
| | | | | PDISDSISTERILDDSKDSVGDSLSGKEDLGRKRTTMLKIATAAKVV | |
| | | | | NANQNASPNVPGKRGRPRKLKLCKAGRPPKNTGKSLISTKNTPVSP | |
| | | | | GSTFPDVKPDLEDVDGVLFVSFESKEALDIHAVDGTTEESSSLQAST | |
| | | | | TNDSGYRARISQLEKELIEDLKTLRHKQVIHPGLQEVGLKLNSVDPT | |
| | | | | MSIDLKYLGVQLPLAPATSFPFWNLTGTNPASPDAGFPFVSRTGKT | |
| | | | | NDFTKIKGWRGKFHSASASRNEGGNSESSLKNRSAFCSDKLDEYLE | |
| | | | | NEGKLMETSMGFSSNAPTSPVVYQLPTKSTSYVRTLDSVLKKQSTIS | |
| | | | | PSTSYSLKPHSVPPVSRKAKSQNRQATFSGRTKSSYKSILPYPVSPKQ | |
| | | | | KYSHVILGDKVTKNSSGIISENQANNFVVPTLDENIFPKQISLRQAQQ | |
| | | | | QQQQQQGSRPPGLSKSQVKLMDLEDCALWEGKPRTYITEERADVS | |
| | | | | LTTLLTAQASLKTKPIHTIIRKRAPPCNNDFCRLGCVCSSLALEKRQP | |
| | | | | AHCRRPDCMFGCTCLKRKVVLVKGGSKTKHFQRKAAHRDPVFYD | |
| | | | | TLGEEAREEEEGIREEEEQLKEKKKR | |
| MGA | MAX dimerization | 23269 | 2857 | MEEKQQIILANQDGGTVAGAAPTFFVILKQPGNGKTDQGILVTNQD | 51. |
| | protein MGA | | | ACALASSVSSPVKSKGKICLPADCTVGGITVTLDNNSMWNEFYHRS | |
| | [Source:HGNC | | | TEMILTKQGRRMFPYCRYWITGLDSNLKYILVMDISPVDNHRYKW | |
| | Symbol; Acc:HGNC: | | | NGRWWEPSGKAEPHVLGRVFIHPESPSTGHYWMHQPVSFYKLKLT | |
| | 14010] | | | NNTLDQEGHIILHSMHRYLPRLHLVPAEKAVEVIQLNGPGVHTFTFP | |
| | | | | QTEFFAVTAYQNIQITQLKIDYNPFAKGFRDDGLNNKPQRDGKQKN | |
| | | | | SSDQEGNNISSSSGHRVRLTEGQGSEIQPGDLDPLSRGHETSGKGLE | |
| | | | | KTSLNIKRDFLGFMDTDSALSEVPQLKQEISECLIASSFEDDSRVASP | |
| | | | | LDQNGSFNVVIKEEPLDDYDYELGECPEGVTVKQEETDEETDVYSN | |
| | | | | SDDDPILEKQLKRHNKVDNPEADHLSSKWLPSSPSGVAKAKMFKL | |
| | | | | DTGKMPVVYLEPCAVTRSTVKISELPDNMLSTSRKDKSSMLAELEY | |
| | | | | LPTYIENSNETAFCLGKESENGLRKHSPDLRVVQKYPLLKEPQWKY | |
| | | | | PDISDSISTERILDDSKDSVGDSLSGKEDLGRKRTTMLKIATAAKVV | |
| | | | | NANQNASPNVPGKRGRPRKLKLCKAGRPPKNTGKSLISTKNTPVSP | |
| | | | | GSTFPDVKPDLEDVDGVLFVSFESKEALDIHAVDGTTEESSSLQAST | |
| | | | | TNDSGYRARISQLEKELIEDLKTLRHKQVIHPGLQEVGLKLNSVDPT | |
| | | | | MSIDLKYLGVQLPLAPATSFPFWNLTGTNPASPDAGFPFVSRTGKT | |
| | | | | NDFTKIKGWRGKFHSASASRNEGGNSESSLKNRSAFCSDKLDEYLE | |
| | | | | NEGKLMETSMGFSSNAPTSPVVYQLPTKSTSYVRTLDSVLKKQSTIS | |
| | | | | PSTSYSLKPHSVPPVSRKAKSQNRQATFSGRTKSSYKSILPYPVSPKQ | |
| | | | | KYSHVILGDKVTKNSSGIISENQANNFVVPTLDENIFPKQISLRQAQQ | |
| | | | | QQQQQQGSRPPGLSKSQVKLMDLEDCALWEGKPRTYITEERADVS | |
| | | | | LTTLLTAQASLKTKPIHTIIRKRAPPCNNDFCRLGCVCSSLALEKRQP | |
| | | | | AHCRRPDCMFGCTCLKRKVVLVKGGSKTKHFQRKAAHRDPVFYD | |
| | | | | TLGEEAREEEEGIREEEEQLKEKKKRKKLEYTICETEPEQPVRHYPL | |
| | | | | WVKVEGEVDPEPVYIPTPSVIEPMKPLLLPQPEVLSPTVKGKLLTGI | |
| | | | | KSPRSYTPKPNPVIREEDKDPVYLYFESMMTCARVRVYERKKEDQR | |
| | | | | QPSSSSSPSPSFQQQTSCHSSPENHNNAKEPDSEQQPLKQLTCDLED | |
| | | | | DSDKLQEKSWKSSCNEGESSSTSYMHQRSPGGPTKLIEIISDCNWEE | |
| | | | | DRNKILSILSQHINSNMPQSLKVGSFIIELASQRKSRGEKNPPVYSSR | |
| | | | | VKISMPSCQDQDDMAEKSGSETPDGPLSPGKMEDISPVQTDALDSV | |
| | | | | RERLHGGKGLPFYAGLSPAGKLVAYKRKPSSSTSGLIQVASNAKVA | |
| | | | | ASRKPRTLLPSTSNSKMASSSGTATNRPGKNLKAFVPAKRPIENAA | |
| | | | | QIPVATPQVSPNTVKRAGPRLLLIPVQQGSPTLRPVSNTQLQGHRM | |
| | | | | VLQPVRSPSGMNLFRHPNGQIVQLLPLHQLRGSNTQPNLQPVMFRN | |
| | | | | PGSVMGIRLPAPSKPSETPPSSTSSSAFSVMNPVIQAVGSSSAVNVIT | |
| | | | | QAPSLLSSGASFVSQAGTLTLRISPPEPQSFASKTGSETKITYSSGGQP | |
| | | | | VGTASLIPLQSGSFALLQLPGQKPVPSSILQHVASLQMKRESQNPDQ | |
| | | | | KDETNSIKREQETKKVLQSEGEAVDPEANVIKQNSGAATSEETLND | |
| | | | | SLEDRGDHLDEECLPEEGCATVKPSEHSCITGSHTDQDYKDVNEEY | |
| | | | | GARNRKSSKEKVAVLEVRTISEKASNKTVQNLSKVQHQKLGDVKV | |
| | | | | EQQKGFDNPEENSSEFPVTFKEESKFELSGSKVMEQQSNLQPEAKE | |
| | | | | KECGDSLEKDRERWRKHLKGPLTRKCVGASQECKKEADEQLIKET | |
| | | | | KTCQENSDVFQQEQGISDLLGKSGITEDARVLKTECDSWSRISNPSA | |
| | | | | FSIVPRRAAKSSRGNGHFQGHLLLPGEQIQPKQEKKGGRSSADFTVL | |
| | | | | DLEEDDEDDNEKTDDSIDEIVDVVSDYQSEEVDDVEKNNCVEYIED | |
| | | | | DEEHVDIETVEELSEEINVAHLKTTAAHTQSFKQPSCTHISADEKAA | |
| | | | | ERSRKAPPIPLKLKPDYWSDKLQKEAEAFAYYRRTHTANERRRRGE | |
| | | | | MRDLFEKLKITLGLLHSSKVSKSLILTRAFSEIQGLTDQADKLIGQK | |
| | | | | NLLTRKRNILIRKVSSLSGKTEEVVLKKLEYIYAKQQALEAQKRKK | |
| | | | | KMGSDEFDISPRISKQQEGSSASSVDLGQMFINNRRGKPLILSRKKD | |
| | | | | QATENTSPLNTPHTSANLVMTPQGQLLTLKGPLFSGPVVAVSPDLL | |
| | | | | ESDLKPQVAGSAVALPENDDLFMMPRIVNVTSLATEGGLVDMGGS | |
| | | | | KYPHEVPDSKPSDHLKDTVRNEDNSLEDKGRISSRGNRDGRVTLGP | |
| | | | | TQVFLANKDSGYPQIVDVSNMQKAQEFLPKKISGDMRGIQYKWKE | |
| | | | | SESRGERVKSKDSSFHKLKMKDLKDSSIEMELRKVTSAIEEAALDSS | |
| | | | | ELLTNMEDEDDTDETLTSLLNEIAFLNQQLNDDSVGLAELPSSMDT | |
| | | | | EFPGDARRAFISKVPPGSRATFQVEHLGTGLKELPDVQGESDSISPLL | |
| | | | | LHLEDDDFSENEKQLAEPASEPDVLKIVIDSEIKDSLLSNKKAIDGG | |
| | | | | KNTSGLPAEPESVSSPPTLHMKTGLENSNSTDTLWRPMPKLAPLGL | |
| | | | | KVANPSSDADGQSLKVMPCLAPIAAKVGSVGHKMNLTGNDQEGR | |
| | | | | ESKVMPTLAPVVAKLGNSGASPSSAGK* | |
| MGA | MAX dimerization | 23269 | 1174 | XLTCDLEDDSDKLQEKSWKSSCNEGESSSTSYMHQRSPGGPTKLIEI | 52. |
| | protein MGA | | | ISDCNWEEDRNKILSILSQHINSNMPQSLKVNGKSYPQAKLLLGQM | |
| | [Source:HGNC | | | GALHPANRLAAYITGRLRPSVLDLSTLSTVISKVASNAKVAASRKPR | |
| | Symbol; Acc:HGNC: | | | TLLPSTSNSKMASSSGTATNRPGKNLKAFVPAKRPIAARPSPGGVFT | |
| | 14010] | | | QFVMSKVGALQQKIPGVSTPQTLAGTQKFSIRPSPVMVVTPVVSSEP | |
| | | | | VQVCSPVTAAVTTTTPQVFLENTTAVTPMTAISDVETKETTYSSGA | |
| | | | | TTTGVVEVSETNTSTSVTSTQSTATVNLTKTTGITTPVASVAFPKSL | |
| | | | | VASPSTITLPVASTASTSLVVVTAAASSSMVTTPTSSLGSVPIILSGIN | |
| | | | | GSPPVSQRPENAAQIPVATPQVSPNTVKRAGPRLLLIPVQQGSPTLR | |
| | | | | PVSNTQLQGHRMVLQPVRSPSGMNLFRHPNGQIVQLLPLHQLRGSN | |
| | | | | TQPNLQPVMFRNPGSVMGIRLPAPSKPSETPPSSTSSSAFSVMNPVIQ | |
| | | | | AVGSSSAVNVITQAPSLLSSGASFVSQAGTLTLRISPPEPQSFASKTG | |
| | | | | SETKITYSSGGQPVGTASLIPLQSGSFALLQLPGQKPVPSSILQHVAS | |
| | | | | LQMKRESQNPDQKDETNSIKREQETKKVLQSEGEAVDPEANVIKQ | |
| | | | | NSGAATSEETLNDSLEDRGDHLDEECLPEEGCATVKPSEHSCITGSH | |
| | | | | TDQDYKDVNEEYGARNRKSSKEKVAVLEVRTISEKASNKTVQNLS | |
| | | | | KVQHQKLGDVKVEQQKGFDNPEENSSEFPVTFKEESKFELSGSKV | |
| | | | | MEQQSNLQPEAKEKECGDSLEKDRERWRKHLKGPLTRKCVGASQE | |
| | | | | CKKEADEQLIKETKTCQENSDVFQQEQGISDLLGKSGITEDARVLKT | |
| | | | | ECDSWSRISNPSAFSIVPRRAAKSSRGNGHFQGHLLLPGEQIQPKQE | |
| | | | | KKGGRSSADFTVLDLEEDDEDDNEKTDDSIDEIVDVVSDYQSEEVD | |
| | | | | DVEKNNCVEYIEDDEEHVDIETVEELSEEINVAHLKTTAAHTQSFK | |
| | | | | QPSCTHISADEKAAERSRKAPPIPLKLKPDYWSDKLQKEAEAFAYY | |
| | | | | RRTHTANERRRRGEMRDLFEKLKITLGLLHSSKVSKSLILTRAFSEIQ | |
| | | | | GLTDQADKLIGQKNLLTRKRNILIRKVSSLSGKTEEVVLKKLEYIYA | |
| | | | | KQQALEA | |
| MGA | MAX dimerization | 23269 | 62 | MKFLFLFFLRQSLTLSPRLECSGAVLAHCKLCLPGLRHCPAPATREA | 53. |
| | protein MGA | | | EAREWLETRSRRLQ* | |
| | [Source:HGNC | | | | |
| | Symbol; Acc:HGNC: | | | | |
| | 14010] | | | | |
| MGA | MAX dimerization | 23269 | 1086 | MEEKQQIILANQDGGTVAGAAPTFFVILKQPGNGKTDQGILVTNQD | 54. |
| | protein MGA | | | ACALASSVSSPVKSKGKICLPADCTVGGITVTLDNNSMWNEFYHRS | |
| | [Source:HGNC | | | TEMILTKQGRRMFPYCRYWITGLDSNLKYILVMDISPVDNHRYKW | |
| | Symbol; Acc:HGNC: | | | NGRWWEPSGKAEPHVLGRVFIHPESPSTGHYWMHQPVSFYKLKLT | |
| | 14010] | | | NNTLDQEGHIILHSMHRYLPRLHLVPAEKAVEVIQLNGPGVHTFTFP | |
| | | | | QTEFFAVTAYQNIQITQLKIDYNPFAKGFRDDGLNNKPQRDGKQKN | |
| | | | | SSDQEGNNISSSSGHRVRLTEGQGSEIQPGDLDPLSRGHETSGKGLE | |
| | | | | KTSLNIKRDFLGFMDTDSALSEVPQLKQEISECLIASSFEDDSRVASP | |
| | | | | LDQNGSFNVVIKEEPLDDYDYELGECPEGVTVKQEETDEETDVYSN | |
| | | | | SDDDPILEKQLKRHNKVDNPEADHLSSKWLPSSPSGVAKAKMFKL | |
| | | | | DTGKMPVVYLEPCAVTRSTVKISELPDNMLSTSRKDKSSMLAELEY | |
| | | | | LPTYIENSNETAFCLGKESENGLRKHSPDLRVVQKYPLLKEPQWKY | |
| | | | | PDISDSISTERILDDSKDSVGDSLSGKEDLGRKRTTMLKIATAAKVV | |
| | | | | NANQNASPNVPGKRGRPRKLKLCKAGRPPKNTGKSLISTKNTPVSP | |
| | | | | GSTFPDVKPDLEDVDGVLFVSFESKEALDIHAVDGTTEESSSLQAST | |
| | | | | TNDSGYRARISQLEKELIEDLKTLRHKQVIHPGLQEVGLKLNSVDPT | |
| | | | | MSIDLKYLGVQLPLAPATSFPFWNLTGTNPASPDAGFPFVSRTGKT | |
| | | | | NDFTKIKGWRGKFHSASASRNEGGNSESSLKNRSAFCSDKLDEYLE | |
| | | | | NEGKLMETSMGFSSNAPTSPVVYQLPTKSTSYVRTLDSVLKKQSTIS | |
| | | | | PSTSYSLKPHSVPPVSRKAKSQNRQATFSGRTKSSYKSILPYPVSPKQ | |
| | | | | KYSHVILGDKVTKNSSGIISENQANNFVVPTLDENIFPKQISLRQAQQ | |
| | | | | QQQQQQGSRPPGLSKSQVKLMDLEDCALWEGKPRTYITEERADVS | |
| | | | | LTTLLTAQASLKTKPIHTIIRKRAPPCNNDFCRLGCVCSSLALEKRQP | |
| | | | | AHCRRPDCMFGCTCLKRK | |
| MGA | MAX dimerization | 23269 | 84 | XDQEGNNISSSSGHRVRLTEGQGSEIQPGDLDPLSRGHETSGKGLEK | 55. |
| | protein MGA | | | TSLNIKRDFLGFMDTDSALSEVPQLKQEISEWYSFA* | |
| | [Source:HGNC | | | | |
| | Symbol; Acc:HGNC: | | | | |
| | 14010] | | | | |
| G2E3 | G2/M-phase specific E3 | 55632 | 707 | MNESKPGDSQNLACVFCRKHDDCPNKYGEKKTKEKWNLTVHYYC | 56. |
| | ubiquitin protein ligase | | | LLMSSGIWQRGKEEEGVYGFLIEDIRKEVNRASKLKCCVCKKNGAS | |
| | [Source:HGNC | | | IGCVAPRCKRSYHFPCGLQRECIFQFTGNFASFCWDHRPVQIITSNN | |
| | Symbol; Acc:HGNC: | | | YRESLPCTICLEFIEPIPSYNILRSPCCKNAWFHRDCLQVQAINAGVF | |
| | 20338] | | | FFRCTICNNSDIFQKEMLRMGIHIPEKDASWELEENAYQELLQHYER | |
| | | | | CDVRRCRCKEGRDYNAPDSKWEIKRCQCCGSSGTHLACSSLRSWE | |
| | | | | QNWECLECRGIIYNSGEFQKAKKHVLPNSNNVGITDCLLEESSPKLP | |
| | | | | RQSPGSQSKDLLRQGSKFRRNVSTLLIELGFQIKKKTKRLYINKANI | |
| | | | | WNSALDAFRNRNFNPSYAIEVAYVIENDNFGSEHPGSKQEFLSLLM | |
| | | | | QHLENSSLFEGSLSKNLSLNSQALKENLYYEAGKMLAISLVHGGPS | |
| | | | | PGFFSKTLFNCLVYGPENTQPILDDVSDFDVAQIIIRINTATTVADLK | |
| | | | | SIINECYNYLELIGCLRLITTLSDKYMLVKDILGYHVIQRVHTPFESF | |
| | | | | KQGLKTLGVLEKIQAYPEAFCSILCHKPESLSAKILSELFTVHTLPDV | |
| | | | | KALGFWNSYLQAVEDGKSTTTMEDILIFATGCSSIPPAGFKPTPSIEC | |
| | | | | LHVDFPVGNKCNNCLAIPITNTYKEFQENMDFTIRNTLRLEKEESSH | |
| | | | | YIGH* | |
| G2E3 | G2/M-phase specific E3 | 55632 | 661 | MSSGIWQRGKEEEGVYGFLIEDIRKEVNRASKLKCCVCKKNGASIG | 57. |
| | ubiquitin protein ligase | | | CVAPRCKRSYHFPCGLQRECIFQFTGNFASFCWDHRPVQIITSNNYR | |
| | [Source:HGNC | | | ESLPCTICLEFIEPIPSYNILRSPCCKNAWFHRDCLQVQAINAGVFFFR | |
| | Symbol; Acc:HGNC: | | | CTICNNSDIFQKEMLRMGIHIPEKDASWELEENAYQELLQHYERCD | |
| | 20338] | | | VRRCRCKEGRDYNAPDSKWEIKRCQCCGSSGTHLACSSLRSWEQN | |
| | | | | WECLECRGIIYNSGEFQKAKKHVLPNSNNVGITDCLLEESSPKLPRQ | |
| | | | | SPGSQSKDLLRQGSKFRRNVSTLLIELGFQIKKKTKRLYINKANIWN | |
| | | | | SALDAFRNRNFNPSYAIEVAYVIENDNFGSEHPGSKQEFLSLLMQHL | |
| | | | | ENSSLFEGSLSKNLSLNSQALKENLYYEAGKMLAISLVHGGPSPGFF | |
| | | | | SKTLFNCLVYGPENTQPILDDVSDFDVAQIIIRINTATTVADLKSIINE | |
| | | | | CYNYLELIGCLRLITTLSDKYMLVKDILGYHVIQRVHTPFESFKQGL | |
| | | | | KTLGVLEKIQAYPEAFCSILCHKPESLSAKILSELFTVHTLPDVKALG | |
| | | | | FWNSYLQAVEDGKSTTTMEDILIFATGCSSIPPAGFKPTPSIECLHVD | |
| | | | | FPVGNKCNNCLAIPITNTYKEFQENMDFTIRNTLRLEKEESSHYIGH* | |
| G2E3 | G2/M-phase specific E3 | 55632 | 176 | MNESKPGDSQNLACVFCRKHDDCPNKYGEKKTKEKWNLTVHYYC | 58. |
| | ubiquitin protein ligase | | | LLMSSGIWQRGKEEEGVYGFLIEDIRKEVNRASKLKCCVCKKNGAS | |
| | [Source:HGNC | | | IGCVAPRCKRSYHFPCGLQRECIFQFTGNFASFCWDHRPVQIITSNN | |
| | Symbol; Acc:HGNC: | | | YRESLPCTICLEFIEPIPSYNILRSPCCKNAWFHRDCLQ | |
| | 20338] | | | | |
| G2E3 | G2/M-phase specific E3 | 55632 | 102 | XCRCKEGRDYNAPDSKWEIKRCQCCGSSGTHLACSSLRSWEQNWE | 59. |
| | ubiquitin protein ligase | | | CLECRGIIYNSGEFQKAKKHVLPNSNNVGITDCLLEESSPKLPRQSP | |
| | [Source:HGNC | | | GSQSKDLLR* | |
| | Symbol; Acc:HGNC: | | | | |
| | 20338] | | | | |
| G2E3 | G2/M-phase specific E3 | 55632 | 352 | MNESKPGDSQNLACVFCRKHDDCPNKYGEKKTKEKWNLTVHYYC | 60. |
| | ubiquitin protein ligase | | | LLMSSGIWQRGKEEEGVYGFLIEDIRKEVNRASKLKCCVCKKNGAS | |
| | [Source:HGNC | | | IGCVAPRCKRSYHFPCGLQRECIFQFTGNFASFCWDHRPVQIITSNN | |
| | Symbol; Acc:HGNC: | | | YRESLPCTICLEFIEPIPSYNILRSPCCKNAWFHRDCLQVQAINAGVF | |
| | 20338] | | | FFRCTICNNSDIFQKEMLRMGIHIPEKDASWELEENAYQELLQHYER | |
| | | | | CDVRRCRCKEGRDYNAPDSKWEIKRCQCCGSSGTHLACSSLRSWE | |
| | | | | QNWECLECRGIIYNSGEFQKAKKHVLPNSNNVGITDCLLEESSPKLP | |
| | | | | RQSPGSQSKDLLRNKVSLCYPGWSTVA* | |
| G2E3 | G2/M-phase specific E3 | 55632 | 121 | MNESKPGDSQNLACVFCRKHDDCPNKYGEKKTKEKWNLTVHYYC | 61. |
| | ubiquitin protein ligase | | | LLMSSGIWQRGKEEEGVYGFLIEDIRKEVNRASKLKCCVCKKNGAS | |
| | [Source:HGNC | | | IGCVAPRCKRSYHFPCGLQRECIFQFTGNFA | |
| | Symbol; Acc:HGNC: | | | | |
| | 20338] | | | | |
| G2E3 | G2/M-phase specific E3 | 55632 | 737 | MLIPALQISDSESVGLEDGIEQSAYLTESKMNESKPGDSQNLACVFC | 62. |
| | ubiquitin protein ligase | | | RKHDDCPNKYGEKKTKEKWNLTVHYYCLLMSSGIWQRGKEEEGV | |
| | [Source:HGNC | | | YGFLIEDIRKEVNRASKLKCCVCKKNGASIGCVAPRCKRSYHFPCG | |
| | Symbol; Acc:HGNC: | | | LQRECIFQFTGNFASFCWDHRPVQIITSNNYRESLPCTICLEFIEPIPSY | |
| | 20338] | | | NILRSPCCKNAWFHRDCLQVQAINAGVFFFRCTICNNSDIFQKEMLR | |
| | | | | MGIHIPEKDASWELEENAYQELLQHYERCDVRRCRCKEGRDYNAP | |
| | | | | DSKWEIKRCQCCGSSGTHLACSSLRSWEQNWECLECRGIIYNSGEF | |
| | | | | QKAKKHVLPNSNNVGITDCLLEESSPKLPRQSPGSQSKDLLRQGSKF | |
| | | | | RRNVSTLLIELGFQIKKKTKRLYINKANIWNSALDAFRNRNFNPSYA | |
| | | | | IEVAYVIENDNFGSEHPGSKQEFLSLLMQHLENSSLFEGSLSKNLSL | |
| | | | | NSQALKENLYYEAGKMLAISLVHGGPSPGFFSKTLFNCLVYGPENT | |
| | | | | QPILDDVSDFDVAQIIIRINTATTVADLKSIINECYNYLELIGCLRLITT | |
| | | | | LSDKYMLVKDILGYHVIQRVHTPFESFKQGLKTLGVLEKIQAYPEA | |
| | | | | FCSILCHKPESLSAKILSELFTVHTLPDVKALGFWNSYLQAVEDGKS | |
| | | | | TTTMEDILIFATGCSSIPPAGFKPTPSIECLHVDFPVGNKCNNCLAIPI | |
| | | | | TNTYKEFQENMDFTIRNTLRLEKEESSHYIGH* | |
| G2E3 | G2/M-phase specific E3 | 55632 | 131 | MNESKPGDSQNLACVFCRKHDDCPNKYGEKKTKEKWNLTVHYYC | 63. |
| | ubiquitin protein ligase | | | LLMSSGIWQRGKEEEGVYGFLIEDIRKEVNRASKLKCCVCKKNGAS | |
| | [Source:HGNC | | | IGCVAPRCKRSYHFPCGLQRECIFQFTGNFASFCWDHRPVQ | |
| | Symbol; Acc:HGNC: | | | | |
| | 20338] | | | | |
| G2E3 | G2/M-phase specific E3 | 55632 | 79 | MNESKPGDSQNLACVFCRKHDDCPNKYGEKKTKEKWNLTVHYYC | 64. |
| | ubiquitin protein ligase | | | LLMSSGIWQRGKEEEGVYGFLIEDIRKEVNRASKL | |
| | [Source:HGNC | | | | |
| | Symbol; Acc:HGNC: | | | | |
| | 20338] | | | | |
| G2E3 | G2/M-phase specific E3 | 55632 | 123 | XEVLVVRTLGFIETVYSKWEIKRCQCCGSSGTHLACSSLRSWEQNW | 65. |
| | ubiquitin protein ligase | | | ECLECRGIIYNSGEFQKAKKHVLPNSNNVGITDCLLEESSPKLPRQSP | |
| | [Source:HGNC | | | GSQSKDLLRQGSKFRRNVSTLLIELGFQI | |
| | Symbol; Acc:HGNC: | | | | |
| | 32038] | | | | |
| XK | X-linked Kx blood | 7504 | 445 | MKFPASVLASVFLFVAETTAALSLSSTYRSGGDRMWQALTLLFSLL | 66. |
| | group [Source:HGNC | | | PCALVQLTLLFVHRDLSRDRPLVLLLHLLQLGPLFRCFEVFCIYFQS | |
| | Symbol; Acc:HGNC: | | | GNNEEPYVSITKKRQMPKNGLSEEIEKEVGQAEGKLITHRSAFSRAS | |
| | 12811] | | | VIQAFLGSAPQLTLQLYISVMQQDVTVGRSLLMTISLLSIVYGALRC | |
| | | | | NILAIKIKYDEYEVKVKPLAYVCIFLWRSFEIATRVVVLVLFTSVLK | |
| | | | | TWVVVIILINFFSFFLYPWILFWCSGSPFPENIEKALSRVGTTIVLCFL | |
| | | | | TLLYTGINMFCWSAVQLKIDSPDLISKSHNWYQLLVYYMIRFIENAI | |
| | | | | LLLLWYLFKTDIYMYVCAPLLVLQLLIGYCTAILFMLVFYQFFHPC | |
| | | | | KKLFSSSVSEGFQRWLRCFCWACRQQKPCEPIGKEDLQSSRDRDET | |
| | | | | PSSSKTSPEPGQFLNAEDLCSA* | |
| MYLIP | myosin regulatory light | 29116 | 446 | MLCYVTRPDAVLMEVEVEAKANGEDCLNQVCRRLGIIEVDYFGLQ | 67. |
| | chain interacting | | | FTGSKGESLWLNLRNRISQQMDGLAPYRLKLRVKFFVEPHLILQEQ | |
| | protein [Source:HGNC | | | TRHIFFLHIKEALLAGHLLCSPEQAVELSALLAQTKFGDYNQNTAK | |
| | Symbol; Acc:HGNC: | | | YNYEELCAKELSSATLNSIVAKHKELEGTSQASAEYQVLQIVSAME | |
| | 21155] | | | NYGIEWHSVRDSEGQKLLIGVGPEGISICKDDFSPINRIAYPVVQMA | |
| | | | | TQSGKNVYLTVTKESGNSIVLLFKMISTRAASGLYRAITETHAFYRC | |
| | | | | DTVTSAVMMQYSRDLKGHLASLFLNENINLGKKYVFDIKRTSKEV | |
| | | | | YDHARRALYNAGVVDLVSRNNQSPSHSPLKSSESSMNCSSCEGLSC | |
| | | | | QQTRVLQEKLRKLKEAMLCMVCCEEEINSTFCPCGHTVCCESCAA | |
| | | | | QLQSCPVCRSRVEHVQHVYLPTHTSLLNLTVI* | |
| MYLIP | myosin regulatory light | 29116 | 265 | MENYGIEWHSVRDSEGQKLLIGVGPEGISICKDDFSPINRIAYPVVQ | 68. |
| | chain interacting | | | MATQSGKNVYLTVTKESGNSIVLLFKMISTRAASGLYRAITETHAF | |
| | protein [Source:HGNC | | | YRCDTVTSAVMMQYSRDLKGHLASLFLNENINLGKKYVFDIKRTS | |
| | Symbol; Acc:HGNC: | | | KEVYDHARRALYNAGVVDLVSRNNQSPSHSPLKSSESSMNCSSCE | |
| | 21155] | | | GLSCQQTRVLQEKLRKLKEAMLCMVCCEEEINSTFCPCGHTVCCES | |
| | | | | CAAQLQSCPVCRSRVEHVQHVYLPTHTSLLNLTVI* | |
| EFHC2 | EF-hand domain | 80258 | 750 | MALPLLPGNSFNRNVGKEKFHKSQHWGFCNNVMMLVSDEKPGIG | 69. |
| | containing 2 | | | GEPLLGQKIKPKCSIYPKGDGSDVPSWVAFDKQVLSFDAYLEEEVL | |
| | [Source:HGNC | | | DKSQTNYRIRYYKIYFYPEDDTIQVNEPEVKNSGLLQGTSIRRHRITL | |
| | Symbol; Acc:HGNC: | | | PPPDEDQFYTVYHFNVGTEVVFYGRTFKIYDCDAFTRNFLRKIGVK | |
| | 26233] | | | VNPPVQCPEDPYMKIRREVVEHVEPLRPYESLDTLKQFLQYHGKIL | |
| | | | | CFFCLWDDSVSMFGDRRELILHYFLCDDTIEIKELLPHSSGRDALKM | |
| | | | | FLRRSKLPKNCPPRVYQPGQITDRAVLNSYGDFIKNQADGYLFDRY | |
| | | | | KLGKVDQEFYKDSDLSLGVTINVWGRKVLLYDCDEFTKSYYKSKY | |
| | | | | GIENFTSVSCKPPSPPPKIERKFPPYNGFGSEEDSLRNCIDLKPTPHRR | |
| | | | | NFKKFMEKDSYGSKSNILRFFAKLVTDKCVDLDRMFVISYYLGDDT | |
| | | | | ISVFEPIERNSGIAGGMFLKRSRVKKPGQEVFKSELSEYIKAEELYIG | |
| | | | | VTVNVNGYLFRLLNADEYTLNYMEQNTDKYPFSNLKLALQKLKQE | |
| | | | | EGKSRELKQVFKAADSKHTNMVDYNTFRDILMSLTVGNLAEQEFV | |
| | | | | TIARHYRVPEGTCSDMDFLIALAHEKFKKNMFENFDTFIYSCVYED | |
| | | | | REKKNVLPTKDIKRLCKSSRLPLSDDLLESLLSRFEDSEKQIDYKSFF | |
| | | | | SALNWRKNPVPELQPASYLKERCEDVWLGMPSPIPAKYIDYWTFL | |
| | | | | KDAFGLEEE* | |
| FTSJ1 | FtsJ RNA 2′-O- | 24140 | 328 | MGRTSKDKRDVYYRLAKENGWRARSAFKLLQLDKEFQLFQGVTR | 70. |
| | methyltransferase 1 | | | AVDLCAAPGSWSQVLSQKIGGQGSGHVVAVDLQAMAPLPGVVQI | |
| | [Source:HGNC | | | QGDITQLSTAKEIIQHFKGCPADLVVCDGAPDVTGLHDVDEYMQA | |
| | Symbol; Acc:HGNC: | | | QLLLAALNIATHVLKPGGCFVAKIFRGRDVTLLYSQLQVFFSSVLCA | |
| | 13254] | | | KPRSSRNSSIEAFAVCQGYDPPEGFIPDLSKPLLDHSYDFNQLDGPTR | |
| | | | | IIVPFVTCGDLSSYDSDRSYPLDLEGGSEYKYTPPTQPPISPPYQEAC | |
| | | | | TLKRKGQLAKEIRPQDCPISRVDTFPQPLAAPQCHTLLAPEMEDNE | |
| | | | | MSCSP* | |
| FTSJ1 | FtsJ RNA 2′-O- | 24140 | 330 | MGRTSKDKRDVYYRLAKENGWRARSAFKLLQLDKEFQLFQGVTR | 71. |
| | methyltransferase 1 | | | AVDLCAAPGSWSQVLSQKIGGQGSGHVVAVDLQAMAPLPGVVQI | |
| | [Source:HGNC | | | QGDITQLSTAKEIIQHFKGCPADLVVCDGAPDVTGLHDVDEYMQA | |
| | Symbol; Acc:HGNC: | | | QLLLAALNIATHVLKPGGCFVAKIFRGRDVTLLYSQLQVFFSSVLCA | |
| | 13254] | | | KPRSSRNSSIEAFAVCQGYDPPEGFIPDLSKPLLDHSYDPDFNQLDGP | |
| | | | | TRIIVPFVTCGDLSSYDSDRSYPLDLEGGSEYKYTPPTQPPISPPYQE | |
| | | | | ACTLKRKGQLAKEIRPQDCPISRVDTFPQPLAAPQCHTLLAPEMED | |
| | | | | NEMSCSP* | |
| FTSJ1 | FtsJ RNA 2′-O- | 24140 | 193 | MALNIATHVLKPGGCFVAKIFRGRDVTLLYSQLQVFFSSVLCAKPR | 72. |
| | methyltransferase 1 | | | SSRNSSIEAFAVCQGYDPPEGFIPDLSKPLLDHSYDPDFNQLDGPTRII | |
| | [Source:HGNC | | | VPFVTCGDLSSYDSDRSYPLDLEGGSEYKYTPPTQPPISPPYQEACTL | |
| | Symbol; Acc:HGNC: | | | KRKGQLAKEIRPQDCPISRVDTFPQPLAAPQCHTLLAPEMEDNEMS | |
| | 21354] | | | CSP* | |
| BTN3A2 | butyrophilin subfamily | 11118 | 335 | MKMASSLAFLLLNFHVSLLLVQLLTPCSAQFSVLGPSGPILAMVGE | 73. |
| | 3 member A2 | | | DADLPCHLFPTMSAETMELKWVSSSLRQVVNVYADGKEVEDRQS | |
| | [Source:HGNC | | | APYRGRTSILRDGITAGKAALRIHNVTASDSGKYLCYFQDGDFYEK | |
| | Symbol; Acc:HGNC: | | | ALVELKVAALGSNLHVEVKGYEDGGIHLECRSTGWYPQPQIQWSN | |
| | 1139] | | | AKGENIPAVEAPVVADGVGLYEVAASVIMRGGSGEGVSCIIRNSLL | |
| | | | | GLEKTASISIADPFFRSAQPWIAALAGTLPILLLLLAGASYFLWRQQ | |
| | | | | KEITALSSEIESEQEMKEMGYAATEREISLRESLQEELKRKKIQYLTR | |
| | | | | GEESSSDTNKSA* | |
| BTN3A2 | butyrophilin subfamily | 11118 | 312 | MGIPRAQFSVLGPSGPILAMVGEDADLPCHLFPTMSAETMELKWVS | 74. |
| | 3 member A2 | | | SSLRQVVNVYADGKEVEDRQSAPYRGRTSILRDGITAGKAALRIHN | |
| | [Source:HGNC | | | VTASDSGKYLCYFQDGDFYEKALVELKVAALGSNLHVEVKGYED | |
| | Symbol; Acc:HGNC: | | | GGIHLECRSTGWYPQPQIQWSNAKGENIPAVEAPVVADGVGLYEV | |
| | 1139] | | | AASVIMRGGSGEGVSCIIRNSLLGLEKTASISIADPFFRSAQPWIAAL | |
| | | | | AGTLPILLLLLAGASYFLWRQQKEITALSSEIESEQEMKEMGYAATE | |
| | | | | REISLRESLQEELKRKKIQYLTRGEESSSDTNKSA* | |
| BTN3A2 | butyrophilin subfamily | 11118 | 196 | MVGEDADLPCHLFPTMSAETMELKWVSSSLRQVVNVYADGKEVE | 75. |
| | 3 member A2 | | | DRQSAPYRGRTSILRDGITAGKAALRIHNVTASDSGKYLCYFQDGD | |
| | [Source:HGNC | | | FYEKALVELKVAALGSNLHVEVKGYEDGGIHLECRSTGWYPQPQI | |
| | Symbol; Acc:HGNC: | | | QWSNAKGENIPAVEAPVVADGVGLYEVAASVIMRGGSGEGVSCIIR | |
| | 1139] | | | NSLLGLEKTASISIA | |
| BTN3A2 | butyrophilin subfamily | 11118 | 115 | MVGEDADLPCHLFPTMSAETMELKWVSSSLRQVVNVYADGKEVE | 76. |
| | 3 member A2 | | | DRQSAPYRGRTSILRDGITAGKAALRIHNVTASDSGKYLCYFQDGD | |
| | [Source:HGNC | | | FYEKALVELKVAALGSNLHVEVKGY | |
| | Symbol; Acc:HGNC: | | | | |
| | 1139] | | | | |
| BTN3A2 | butyrophilin subfamily | 11118 | 116 | RGGSGEGVSCIIRNSLLGLEKTASISIADPFFRSAQPWIAALAGTLPIL | 77. |
| | 3 member A2 | | | LLLLAGASYFLWRQQKEITALSSEIESEQEMKEMGYAATEREISLRE | |
| | [Source:HGNC | | | SLQEELKRKKIQYLTREWL* | |
| | Symbol; Acc:HGNC: | | | | |
| | 1139] | | | | |
| BTN3A2 | butyrophilin subfamily | 11118 | 160 | MKMASSLAFLLLNFHVSLLLVQLLTPCSAQFSVLGPSGPILAMVGE | 78. |
| | 3 member A2 | | | DADLPCHLFPTMSAETMELKWVSSSLRQVVNVYADGKEVEDRQS | |
| | [Source:HGNC | | | APYRGRTSILRDGITAGKAALRIHNVTASDSGKYLCYFQDGDFYEK | |
| | Symbol; Acc:HGNC: | | | ALVELKVADPFFRSAQPWIAALAG | |
| | 1139] | | | | |
| BTN3A2 | butyrophilin subfamily | 11118 | 44 | MKMASSLAFLLLNFHVSLLLVQLLTPCSGLCSPLGSLYLAFPV* | 79. |
| | 3 member A2 | | | | |
| | [Source:HGNC | | | | |
| | Symbol; Acc:HGNC: | | | | |
| | 1139] | | | | |
| BTN3A2 | butyrophilin subfamily | 11118 | 293 | MVGEDADLPCHLFPTMSAETMELKWVSSSLRQVVNVYADGKEVE | 80. |
| | 3 member A2 | | | DRQSAPYRGRTSILRDGITAGKAALRIHNVTASDSGKYLCYFQDGD | |
| | [Source:HGNC | | | FYEKALVELKVAALGSNLHVEVKGYEDGGIHLECRSTGWYPQPQI | |
| | Symbol; Acc:HGNC: | | | QWSNAKGENIPAVEAPVVADGVGLYEVAASVIMRGGSGEGVSCIIR | |
| | 1139] | | | NSLLGLEKTASISIADPFFRSAQPWIAALAGTLPILLLLLAGASYFLW | |
| | | | | RQQKEITALSSEIESEQEMKEMGYAATEREISLRESLQEELKRKKIQ | |
| | | | | YLTRGEESSSDTNKSA* | |
| BTN3A1 | butyrophilin subfamily | 11119 | 514 | MKMASFLAFLLLNFRVCLLLLQLLMPHSAQFSVLGPSGPILAMVGE | 81. |
| | 3 member A1 | | | DADLPCHLFPTMSAETMELKWVSSSLRQVVNVYADGKEVEDRQS | |
| | [Source:HGNC | | | APYRGRTSILRDGITAGKAALRIHNVTASDSGKYLCYFQDGDFYEK | |
| | Symbol; Acc:HGNC: | | | ALVELKVAALGSDLHVDVKGYKDGGIHLECRSTGWYPQPQIQWSN | |
| | 1138] | | | NKGENIPTVEAPVVADGVGLYAVAASVIMRGSSGEGVSCTIRSSLL | |
| | | | | GLEKTASISIADPFFRSAQRWIAALAGTLPVLLLLLGGAGYFLWQQ | |
| | | | | QEEKKTQFRKKKREQELREMAWSTMKQEQSTRVKLLEELRWRSIQ | |
| | | | | YASRGERHSAYNEWKKALFKPADVILDPKTANPILLVSEDQRSVQR | |
| | | | | AKEPQDLPDNPERFNWHYCVLGCESFISGRHYWEVEVGDRKEWHI | |
| | | | | GVCSKNVQRKGWVKMTPENGFWTMGLTDGNKYRTLTEPRTNLKL | |
| | | | | PKPPKKVGVFLDYETGDISFYNAVDGSHIHTFLDVSFSEALYPVFRIL | |
| | | | | TLEPTALTICPA* | |
| BTN3A1 | butyrophilin subfamily | 11119 | 43 | MKMASFLAFLLLNFRVCLLLLQLLMPHSAQFSVLGPSGPILAM | 82. |
| | 3 member A1 | | | | |
| | [Source:HGNC | | | | |
| | Symbol; Acc:HGNC: | | | | |
| | 1138] | | | | |
| BTN3A1 | butyrophilin subfamily | 11119 | 353 | MKMASFLAFLLLNFRVCLLLLQLLMPHSAQFSVLGPSGPILAMVGE | 83. |
| | 3 member A1 | | | DADLPCHLFPTMSAETMELKWVSSSLRQVVNVYADGKEVEDRQS | |
| | [Source:HGNC | | | APYRGRTSILRDGITAGKAALRIHNVTASDSGKYLCYFQDGDFYEK | |
| | Symbol; Acc:HGNC: | | | ALVELKVAALGSDLHVDVKGYKDGGIHLECRSTGWYPQPQIQWSN | |
| | 1138] | | | NKGENIPTVEAPVVADGVGLYAVAASVIMRGSSGEGVSCTIRSSLL | |
| | | | | GLEKTASISIADPFFRSAQRWIAALAGTLPVLLLLLGGAGYFLWQQ | |
| | | | | QEEKKTQFRKKKREQELREMAWSTMKQEQSTRVKLLEELRWRSIQ | |
| | | | | YASRGERHSAYNEWKKALFKPGEEMLQMRLHFVK* | |
| BTN3A1 | butyrophilin subfamily | 11119 | 119 | MKMASFLAFLLLNFRVCLLLLQLLMPHSAQFSVLGPSGPILAMVGE | 84. |
| | 3 member A1 | | | DADLPCHLFPTMSAETMELKWVSSSLRQVVNVYADGKEVEDRQS | |
| | [Source:HGNC | | | APYRGRTSILRDGITAGKAALRIHNVTAS | |
| | Symbol; Acc:HGNC: | | | | |
| | 1138] | | | | |
| BTN3A1 | butyrophilin subfamily | 11119 | 462 | MKMASFLAFLLLNFRVCLLLLQLLMPHSAQFSVLGPSGPILAMVGE | 85. |
| | 3 member A1 | | | DADLPCHLFPTMSAETMELKWVSSSLRQVVNVYADGKEVEDRQS | |
| | [Source:HGNC | | | APYRGRTSILRDGITAGKAALRIHNVTASDSGKYLCYFQDGDFYEK | |
| | Symbol; Acc:HGNC: | | | ALVELKVADGVGLYAVAASVIMRGSSGEGVSCTIRSSLLGLEKTASI | |
| | 1138] | | | SIADPFFRSAQRWIAALAGTLPVLLLLLGGAGYFLWQQQEEKKTQF | |
| | | | | RKKKREQELREMAWSTMKQEQSTRVKLLEELRWRSIQYASRGERH | |
| | | | | SAYNEWKKALFKPADVILDPKTANPILLVSEDQRSVQRAKEPQDLP | |
| | | | | DNPERFNWHYCVLGCESFISGRHYWEVEVGDRKEWHIGVCSKNVQ | |
| | | | | RKGWVKMTPENGFWTMGLTDGNKYRTLTEPRTNLKLPKPPKKVG | |
| | | | | VFLDYETGDISFYNAVDGSHIHTFLDVSFSEALYPVFRILTLEPTALTI | |
| | | | | CPA* | |
| BTN3A1 | butyrophilin subfamily | 11119 | 379 | MKMASFLAFLLLNFRVCLLLLQLLMPHSAQFSVLGPSGPILAMVGE | 86. |
| | 3 member A1 | | | DADLPCHLFPTMSAETMELKWVSSSLRQVVNVYADGKEVEDRQS | |
| | [Source:HGNC | | | APYRGRTSILRDGITAGKAALRIHNVTASDSGKYLCYFQDGDFYEK | |
| | Symbol; Acc:HGNC: | | | ALVELKVAALGSDLHVDVKGYKDGGIHLECRSTGWYPQPQIQWSN | |
| | 1138] | | | NKGENIPTVEAPVVADGVGLYAVAASVIMRGSSGEGVSCTIRSSLL | |
| | | | | GLEKTASISIADPFFRSAQRWIAALAGTLPVLLLLLGGAGYFLWQQ | |
| | | | | QEEKKTQFRKKKREQELREMAWSTMKQEQSTRVKLLEELRWRSIQ | |
| | | | | YASRGERHSAYNEWKKALFKPGPPIGQTQQQTRGQGSPVALSQES | |
| | | | | AQRTDSWGPEEGGES* | |
| BTN3A1 | butyrophilin subfamily | 11119 | 92 | MKMASFLAFLLLNFRVCLLLLQLLMPHSAQFSVLGPSGPILAMVGE | 87. |
| | 3 member A1 | | | DADLPCHLFPTMSAETMELKWVSSSLRQVVNVYADGKEVEDRQS | |
| | [Source:HGNC | | | AP | |
| | Symbol; Acc:HGNC: | | | | |
| | 1138] | | | | |
| BTN2A1 | butyrophilin subfamily | 11120 | 528 | MESAAALHFSRPASLLLLLLSLCALVSAQFIVVGPTDPILATVGENT | 88. |
| | 2 member A1 | | | TLRCHLSPEKNAEDMEVRWFRSQFSPAVFVYKGGRERTEEQMEEY | |
| | [Source:HGNC | | | RGRTTFVSKDISRGSVALVIHNITAQENGTYRCYFQEGRSYDEAILH | |
| | Symbol; Acc:HGNC: | | | LVVAGLGSKPLISMRGHEDGGIRLECISRGWYPKPLTVWRDPYGGV | |
| | 1136] | | | APALKEVSMPDADGLFMVTTAVIIRDKSVRNMSCSINNTLLGQKKE | |
| | | | | SVIFIPESFMPSVSPCAVALPIIVVILMIPIAVCIYWINKLQKEKKILSG | |
| | | | | EKEFERETREIALKELEKERVQKEEELQVKEKLQEELRWRRTFLHA | |
| | | | | VDVVLDPDTAHPDLFLSEDRRSVRRCPFRHLGESVPDNPERFDSQP | |
| | | | | CVLGRESFASGKHYWEVEVENVIEWTVGVCRDSVERKGEVLLIPQ | |
| | | | | NGFWTLEMHKGQYRAVSSPDRILPLKESLCRVGVFLDYEAGDVSF | |
| | | | | YNMRDRSHIYTCPRSAFSVPVRPFFRLGCEDSPIFICPALTGANGVTV | |
| | | | | PEEGLTLHRVGTHQSL* | |
| BTN2A1 | butyrophilin subfamily | 11120 | 152 | MESAAALHFSRPASLLLLLLSLCALVSAQFIVVGPTDPILATVGENT | 89. |
| | 2 member A1 | | | TLRCHLSPEKNAEDMEVRWFRSQFSPAVFVYKGGRERTEEQMEEY | |
| | [Source:HGNC | | | RGRTTFVSKDISRGSVALVIHNITAQENGTYRCYFQEGRSYDEAILH | |
| | Symbol; Acc:HGNC: | | | LVVAELLEAEVY* | |
| | 1136] | | | | |
| BTN2A1 | butyrophilin subfamily | 11120 | 331 | MESAAALHFSRPASLLLLLLSLCALVSAQFIVVGPTDPILATVGENT | 90. |
| | 2 member A1 | | | TLRCHLSPEKNAEDMEVRWFRSQFSPAVFVYKGGRERTEEQMEEY | |
| | [Source:HGNC | | | RGRTTFVSKDISRGSVALVIHNITAQENGTYRCYFQEGRSYDEAILH | |
| | Symbol; Acc:HGNC: | | | LVVAGLGSKPLISMRGHEDGGIRLECISRGWYPKPLTVWRDPYGGV | |
| | 1136] | | | APALKEVSMPDADGLFMVTTAVIIRDKSVRNMSCSINNTLLGQKKE | |
| | | | | SVIFIPESFMPSVSPCAVALPIIVVILMIPIAVCIYWINKLQKEKKILSG | |
| | | | | EKEFERETREIALKELEKERVQKEEELQVKEKLQEELRWRRTFLHA | |
| | | | | GPV* | |
| BTN2A1 | butyrophilin subfamily | 11120 | 79 | XIIVVILMIPIAVCIYWINKLQKEKKILSGEKEFERETREIALKELEKE | 91. |
| | 2 member A1 | | | RVQKEEELQVKEKLQEELRWRRTFLHAGW* | |
| | [Source:HGNC | | | | |
| | Symbol; Acc:HGNC: | | | | |
| | 1136] | | | | |
| BTN2A1 | butyrophilin subfamily | 11120 | 86 | MEVRWFRSQFSPAVFVYKGGRERTEEQMEEYRGRTTFVSKDISRGS | 92. |
| | 2 member A1 | | | VALVIHNITAQENGTYRCYFQEGRSYDEAILHLVVAGLGS | |
| | [Source:HGNC | | | | |
| | Symbol; Acc:HGNC: | | | | |
| | 1136] | | | | |
| BTN2A1 | butyrophilin subfamily | 11120 | 335 | MESAAALHFSRPASLLLLLLSLCALVSAQFIVVGPTDPILATVGENT | 93. |
| | 2 member A1 | | | TLRCHLSPEKNAEDMEVRWFRSQFSPAVFVYKGGRERTEEQMEEY | |
| | [Source:HGNC | | | RGRTTFVSKDISRGSVALVIHNITAQENGTYRCYFQEGRSYDEAILH | |
| | Symbol; Acc:HGNC: | | | LVVAGLGSKPLISMRGHEDGGIRLECISRGWYPKPLTVWRDPYGGV | |
| | 1136] | | | APALKEVSMPDADGLFMVTTAVIIRDKSVRNMSCSINNTLLGQKKE | |
| | | | | SVIFIPESFMPSVSPCAVALPIIVVILMIPIAVCIYWINKLQKEKKILSG | |
| | | | | EKEFERETREIALKELEKERVQKEEELQVKEKLQEELRWRRTFLHA | |
| | | | | ELQFFSN* | |
| BTN2A1 | butyrophilin subfamily | 11120 | 467 | MEVRWFRSQFSPAVFVYKGGRERTEEQMEEYRGRTTFVSKDISRGS | 94. |
| | 2 member A1 | | | VALVIHNITAQENGTYRCYFQEGRSYDEAILHLVVAGLGSKPLISMR | |
| | [Source:HGNC | | | GHEDGGIRLECISRGWYPKPLTVWRDPYGGVAPALKEVSMPDADG | |
| | Symbol; Acc:HGNC: | | | LFMVTTAVIIRDKSVRNMSCSINNTLLGQKKESVIFIPESFMPSVSPC | |
| | 1136] | | | AVALPIIVVILMIPIAVCIYWINKLQKEKKILSGEKEFERETREIALKE | |
| | | | | LEKERVQKEEELQVKEKLQEELRWRRTFLHAVDVVLDPDTAHPDL | |
| | | | | FLSEDRRSVRRCPFRHLGESVPDNPERFDSQPCVLGRESFASGKHY | |
| | | | | WEVEVENVIEWTVGVCRDSVERKGEVLLIPQNGFWTLEMHKGQY | |
| | | | | RAVSSPDRILPLKESLCRVGVFLDYEAGDVSFYNMRDRSHIYTCPRS | |
| | | | | AFSVPVRPFFRLGCEDSPIFICPALTGANGVTVPEEGLTLHRVGTHQS | |
| | | | | L* | |
| SGMS1 | sphingomyelin synthase | 259230 | 242 | MKEVVYWSPKKVADWLLENAMPEYCEPLEHFTGQDLINLTQEDFK | 95. |
| | 1 [Somce:HGNC | | | KPPLCRVSSDNGQRLLDMIETLKMEHHLEAHKNGHANGHLNIGVD | |
| | Symbol; Acc:HGNC: | | | IPTPDGSFSIKIKPNGMPNGYRKEMIKIPMPELERSQYPMEWGKTFL | |
| | 29799] | | | AFLYALSCFVLTTVMISVVHERVPPKEVQPPLPDTFFDHFNRVQWA | |
| | | | | FSICEINGMILVGLWLIQWLLLKYKDVGNESRVVMIFLSIVHPVLLL | |
| | | | | GSIAGFLSLSL* | |
| SGMS1 | sphingomyelin synthase | 259230 | 414 | MKEVVYWSPKKVADWLLENAMPEYCEPLEHFTGQDLINLTQEDFK | 96. |
| | 1 [Source:HGNC | | | KPPLCRVSSDNGQRLLDMIETLKMEHHLEAHKNGHANGHLNIGVD | |
| | Symbol; Ace:HGNC: | | | IPTPDGSFSIKIKPNGMPNGYRKEMIKIPMPELERSQYPMEWGKTFL | |
| | 29799] | | | AFLYALSCFVLTTVMISVVHERVPPKEVQPPLPDTFFDHFNRVQWA | |
| | | | | FSICEINGMILVGLWLIQWLLLKYKSIISRRFFCIVGTLYLYRCITMY | |
| | | | | VTTLPVPGMHFNCSPKLFGDWEAQLRRIMKLIAGGGLSITGSHNMC | |
| | | | | GDYLYSGHTVMLTLTYLFIKEYSPRRLWWYHWICWLLSVVGIFCIL | |
| | | | | LAHDHYTVDVVVAYYITTRLFWWYHTMANQQVLKEASQMNLLA | |
| | | | | RVWWYRPFQYFEKNVQGIVPRSYHWPFPWPVVHLSRQVKYSRLV | |
| | | | | NDT* | |
| SGMS1 | sphingomyelin synthase | 259230 | 220 | MKEVVYWSPKKVADWLLENAMPEYCEPLEHFTGQDLINLTQEDFK | 97. |
| | 1 [Source:HGNC | | | KPPLCRVSSDNGQRLLDMIETLKMEHHLEAHKNGHANGHLNIGVD | |
| | Symbol; Ace:HGNC: | | | IPTPDGSFSIKIKPNGMPNGYRKEMIKIPMPELERSQYPMEWGKTFL | |
| | 29799] | | | AFLYALSCFVLTTVMISVVHERVPPKEVQPPLPDTFFDHFNRVQWA | |
| | | | | FSICEINGMILVGLWLIQWLLLKYNFSETGKPNCEE* | |
| SGMS1 | sphingomyelin synthase | 259230 | 13 | LWLIQWLLLKYK* | 98. |
| | 1 [Source:HGNC | | | | |
| | Symbol; Ace:HGNC: | | | | |
| | 29799] | | | | |
| SGMS1 | sphingomyelin synthase | 259230 | 36 | MYVTTLPVPGMHFNCSPKLFGDWEAQLRRIMKLIAG | 99. |
| | 1 [Source:HGNC | | | | |
| | Symbol; Ace:HGNC: | | | | |
| | 29799] | | | | |
| GSPT2 | G1 to S phase transition | 23708 | 629 | MDSGSSSSDSAPDCWDQVDMESPGSAPSGDGVSSAVAEAQREPLSS | 100. |
| | 2 [Somce:HGNC | | | AFSRKLNVNAKPFVPNVHAAEFVPSFLRGPTQPPTLPAGSGSNDETC | |
| | Symbol; Acc:HGNC: | | | TGAGYPQGKRMGRGAPVEPSREEPLVSLEGSNSAVTMELSEPVVEN | |
| | 4622] | | | GEVEMALEESWEHSKEVSEAEPGGGSSGDSGPPEESGQEMMEEKE | |
| | | | | EIRKSKSVIVPSGAPKKEHVNVVFIGHVDAGKSTIGGQIMFLTGMVD | |
| | | | | KRTLEKYEREAKEKNRETWYLSWALDTNQEERDKGKTVEVGRAY | |
| | | | | FETERKHFTILDAPGHKSFVPNMIGGASQADLAVLVISARKGEFETG | |
| | | | | FEKGGQTREHAMLAKTAGVKHLIVLINKMDDPTVNWSIERYEECK | |
| | | | | EKLVPFLKKVGFSPKKDIHFMPCSGLTGANIKEQSDFCPWYTGLPFI | |
| | | | | PYLDNLPNFNRSIDGPIRLPIVDKYKDMGTVVLGKLESGSIFKGQQL | |
| | | | | VMMPNKHNVEVLGILSDDTETDFVAPGENLKIRLKGIEEEEILPGFIL | |
| | | | | CDPSNLCHSGRTFDVQIVIIEHKSIICPGYNAVLHIHTCIEEVEITALIS | |
| | | | | LVDKKSGEKSKTRPRFVKQDQVCIARLRTAGTICLETFKDFPQMGR | |
| | | | | FTLRDEGKTIAIGKVLKLVPEKD* | |
| CREBBP | CREB binding protein | 1387 | 2443 | MAENLLDGPPNPKRAKLSSPGFSANDSTDFGSLFDLENDLPDELIPN | 101. |
| | [Source:HGNC | | | GGELGLLNSGNLVPDAASKHKQLSELLRGGSGSSINPGIGNVSASSP | |
| | Symbol; Acc:HGNC: | | | VQQGLGGQAQGQPNSANMASLSAMGKSPLSQGDSSAPSLPKQAAS | |
| | 2348] | | | TSGPTPAASQALNPQAQKQVGLATSSPATSQTGPGICMNANFNQTH | |
| | | | | PGLLNSNSGHSLINQASQGQAQVMNGSLGAAGRGRGAGMPYPTPA | |
| | | | | MQGASSSVLAETLTQVSPQMTGHAGLNTAQAGGMAKMGITGNTS | |
| | | | | PFGQPFSQAGGQPMGATGVNPQLASKQSMVNSLPTFPTDIKNTSVT | |
| | | | | NVPNMSQMQTSVGIVPTQAIATGPTADPEKRKLIQQQLVLLLHAHK | |
| | | | | CQRREQANGEVRACSLPHCRTMKNVLNHMTHCQAGKACQVAHC | |
| | | | | ASSRQIISHWKNCTRHDCPVCLPLKNASDKRNQQTILGSPASGIQNT | |
| | | | | IGSVGTGQQNATSLSNPNPIDPSSMQRAYAALGLPYMNQPQTQLQP | |
| | | | | QVPGQQPAQPQTHQQMRTLNPLGNNPMNIPAGGITTDQQPPNLISE | |
| | | | | SALPTSLGATNPLMNDGSNSGNIGTLSTIPTAAPPSSTGVRKGWHEH | |
| | | | | VTQDLRSHLVHKLVQAIFPTPDPAALKDRRMENLVAYAKKVEGD | |
| | | | | MYESANSRDEYYHLLAEKIYKIQKELEEKRRSRLHKQGILGNQPAL | |
| | | | | PAPGAQPPVIPQAQPVRPPNGPLSLPVNRMQVSQGMNSFNPMSLGN | |
| | | | | VQLPQAPMGPRAASPMNHSVQMNSMGSVPGMAISPSRMPQPPNM | |
| | | | | MGAHTNNMMAQAPAQSQFLPQNQFPSSSGAMSVGMGQPPAQTGV | |
| | | | | SQGQVPGAALPNPLNMLGPQASQLPCPPVTQSPLHPTPPPASTAAG | |
| | | | | MPSLQHTTPPGMTPPQPAAPTQPSTPVSSSGQTPTPTPGSVPSATQT | |
| | | | | QSTPTVQAAAQAQVTPQPQTPVQPPSVATPQSSQQQPTPVHAQPPG | |
| | | | | TPLSQAAASIDNRVPTPSSVASAETNSQQPGPDVPVLEMKTETQAE | |
| | | | | DTEPDPGESKGEPRSEMMEEDLQGASQVKEETDIAEQKSEPMEVDE | |
| | | | | KKPEVKVEVKEEEESSSNGTASQSTSPSQPRKKIFKPEELRQALMPT | |
| | | | | LEALYRQDPESLPFRQPVDPQLLGIPDYFDIVKNPMDLSTIKRKLDT | |
| | | | | GQYQEPWQYVDDVWLMFNNAWLYNRKTSRVYKFCSKLAEVFEQ | |
| | | | | EIDPVMQSLGYCCGRKYEFSPQTLCCYGKQLCTIPRDAAYYSYQNR | |
| | | | | YHFCEKCFTEIQGENVTLGDDPSQPQTTISKDQFEKKKNDTLDPEPF | |
| | | | | VDCKECGRKMHQICVLHYDIIWPSGFVCDNCLKKTGRPRKENKFS | |
| | | | | AKRLQTTRLGNHLEDRVNKFLRRQNHPEAGEVFVRVVASSDKTVE | |
| | | | | VKPGMKSRFVDSGEMSESFPYRTKALFAFEEIDGVDVCFFGMHVQE | |
| | | | | YGSDCPPPNTRRVYISYLDSIHFFRPRCLRTAVYHEILIGYLEYVKKL | |
| | | | | GYVTGHIWACPPSEGDDYIFHCHPPDQKIPKPKRLQEWYKKMLDK | |
| | | | | AFAERIIHDYKDIFKQATEDRLTSAKELPYFEGDFWPNVLEESIKELE | |
| | | | | QEEEERKKEESTAASETTEGSQGDSKNAKKKNNKKTNKNKSSISRA | |
| | | | | NKKKPSMPNVSNDLSQKLYATMEKHKEVFFVIHLHAGPVINTLPPI | |
| | | | | VDPDPLLSCDLMDGRDAFLTLARDKHWEFSSLRRSKWSTLCMLVE | |
| | | | | LHTQGQDRFVYTCNECKHHVETRWHCTVCEDYDLCINCYNTKSH | |
| | | | | AHKMVKWGLGLDDEGSSQGEPQSKSPQESRRLSIQRCIQSLVHACQ | |
| | | | | CRNANCSLPSCQKMKRVVQHTKGCKRKTNGGCPVCKQLIALCCY | |
| | | | | HAKHCQENKCPVPFCLNIKHKLRQQQIQHRLQQAQLMRRRMATM | |
| | | | | NTRNVPQQSLPSPTSAPPGTPTQQPSTPQTPQPPAQPQPSPVSMSPAG | |
| | | | | FPSVARTQPPTTVSTGKPTSQVPAPPPPAQPPPAAVEAARQIEREAQ | |
| | | | | QQQHLYRVNINNSMPPGRTGMGTPGSQMAPVSLNVPRPNQVSGPV | |
| | | | | MPSMPPGQWQQAPLPQQQPMPGLPRPVISMQAQAAVAGPRMPSV | |
| | | | | QPPRSISPSALQDLLRTLKSPSSPQQQQQVLNILKSNPQLMAAFIKQR | |
| | | | | TAKYVANQPGMQPQPGLQSQPGMQPQPGMHQQPSLQNLNAMQA | |
| | | | | GVPRPGVPPQQQAMGGLNPQGQALNIMNPGHNPNMASMNPQYRE | |
| | | | | MLRRQLLQQQQQQQQQQQQQQQQQQGSAGMAGGMAGHGQFQQ | |
| | | | | PQGPGGYPPAMQQQQRMQQHLPLQGSSMGQMAAQMGQLGQMG | |
| | | | | QPGLGADSTPNIQQALQQRILQQQQMKQQIGSPGQPNPMSPQQHM | |
| | | | | LSGQPQASHLPGQQIATSLSNQVRSPAPVQSPRPQSQPPHSSPSPRIQ | |
| | | | | PQPSPHHVSPQTGSPHPGLAVTMASSIDQGHLGNPEQSAMLPQLNT | |
| | | | | PSRSALSSELSLVGDTTGDTLEKFVEGL* | |
| CREBBP | CREB binding protein | 1387 | 2405 | MAENLLDGPPNPKRAKLSSPGFSANDSTDFGSLFDLENDLPDELIPN | 102. |
| | [Source:HGNC | | | GGELGLLNSGNLVPDAASKHKQLSELLRGGSGSSINPGIGNVSASSP | |
| | Symbol; Acc:HGNC: | | | VQQGLGGQAQGQPNSANMASLSAMGKSPLSQGDSSAPSLPKQAAS | |
| | 2348] | | | TSGPTPAASQALNPQAQKQVGLATSSPATSQTGPGICMNANFNQTH | |
| | | | | PGLLNSNSGHSLINQASQGQAQVMNGSLGAAGRGRGAGMPYPTPA | |
| | | | | MQGASSSVLAETLTQVSPQMTGHAGLNTAQAGGMAKMGITGNTS | |
| | | | | PFGQPFSQAGGQPMGATGVNPQLASKQSMVNSLPTFPTDIKNTSVT | |
| | | | | NVPNMSQMQTSVGIVPTQAIATGPTADPEKRKLIQQQLVLLLHAHK | |
| | | | | CQRREQANGEVRACSLPHCRTMKNVLNHMTHCQAGKACQAILGS | |
| | | | | PASGIQNTIGSVGTGQQNATSLSNPNPIDPSSMQRAYAALGLPYMN | |
| | | | | QPQTQLQPQVPGQQPAQPQTHQQMRTLNPLGNNPMNIPAGGITTD | |
| | | | | QQPPNLISESALPTSLGATNPLMNDGSNSGNIGTLSTIPTAAPPSSTG | |
| | | | | VRKGWHEHVTQDLRSHLVHKLVQAIFPTPDPAALKDRRMENLVA | |
| | | | | YAKKVEGDMYESANSRDEYYHLLAEKIYKIQKELEEKRRSRLHKQ | |
| | | | | GILGNQPALPAPGAQPPVIPQAQPVRPPNGPLSLPVNRMQVSQGMN | |
| | | | | SFNPMSLGNVQLPQAPMGPRAASPMNHSVQMNSMGSVPGMAISPS | |
| | | | | RMPQPPNMMGAHTNNMMAQAPAQSQFLPQNQFPSSSGAMSVGM | |
| | | | | GQPPAQTGVSQGQVPGAALPNPLNMLGPQASQLPCPPVTQSPLHPT | |
| | | | | PPPASTAAGMPSLQHTTPPGMTPPQPAAPTQPSTPVSSSGQTPTPTPG | |
| | | | | SVPSATQTQSTPTVQAAAQAQVTPQPQTPVQPPSVATPQSSQQQPTP | |
| | | | | VHAQPPGTPLSQAAASIDNRVPTPSSVASAETNSQQPGPDVPVLEM | |
| | | | | KTETQAEDTEPDPGESKGEPRSEMMEEDLQGASQVKEETDIAEQKS | |
| | | | | EPMEVDEKKPEVKVEVKEEEESSSNGTASQSTSPSQPRKKIFKPEEL | |
| | | | | RQALMPTLEALYRQDPESLPFRQPVDPQLLGIPDYFDIVKNPMDLST | |
| | | | | IKRKLDTGQYQEPWQYVDDVWLMFNNAWLYNRKTSRVYKFCSKL | |
| | | | | AEVFEQEIDPVMQSLGYCCGRKYEFSPQTLCCYGKQLCTIPRDAAY | |
| | | | | YSYQNRYHFCEKCFTEIQGENVTLGDDPSQPQTTISKDQFEKKKND | |
| | | | | TLDPEPFVDCKECGRKMHQICVLHYDIIWPSGFVCDNCLKKTGRPR | |
| | | | | KENKFSAKRLQTTRLGNHLEDRVNKFLRRQNHPEAGEVFVRVVAS | |
| | | | | SDKTVEVKPGMKSRFVDSGEMSESFPYRTKALFAFEEIDGVDVCFF | |
| | | | | GMHVQEYGSDCPPPNTRRVYISYLDSIHFFRPRCLRTAVYHEILIGY | |
| | | | | LEYVKKLGYVTGHIWACPPSEGDDYIFHCHPPDQKIPKPKRLQEWY | |
| | | | | KKMLDKAFAERIIHDYKDIFKQATEDRLTSAKELPYFEGDFWPNVL | |
| | | | | EESIKELEQEEEERKKEESTAASETTEGSQGDSKNAKKKNNKKTNK | |
| | | | | NKSSISRANKKKPSMPNVSNDLSQKLYATMEKHKEVFFVIHLHAGP | |
| | | | | VINTLPPIVDPDPLLSCDLMDGRDAFLTLARDKHWEFSSLRRSKWS | |
| | | | | TLCMLVELHTQGQDRFVYTCNECKHHVETRWHCTVCEDYDLCINC | |
| | | | | YNTKSHAHKMVKWGLGLDDEGSSQGEPQSKSPQESRRLSIQRCIQS | |
| | | | | LVHACQCRNANCSLPSCQKMKRVVQHTKGCKRKTNGGCPVCKQL | |
| | | | | IALCCYHAKHCQENKCPVPFCLNIKHKLRQQQIQHRLQQAQLMRR | |
| | | | | RMATMNTRNVPQQSLPSPTSAPPGTPTQQPSTPQTPQPPAQPQPSPV | |
| | | | | SMSPAGFPSVARTQPPTTVSTGKPTSQVPAPPPPAQPPPAAVEAARQ | |
| | | | | IEREAQQQQHLYRVNINNSMPPGRTGMGTPGSQMAPVSLNVPRPN | |
| | | | | QVSGPVMPSMPPGQWQQAPLPQQQPMPGLPRPVISMQAQAAVAGP | |
| | | | | RMPSVQPPRSISPSALQDLLRTLKSPSSPQQQQQVLNILKSNPQLMA | |
| | | | | AFIKQRTAKYVANQPGMQPQPGLQSQPGMQPQPGMHQQPSLQNL | |
| | | | | NAMQAGVPRPGVPPQQQAMGGLNPQGQALNIMNPGHNPNMASM | |
| | | | | NPQYREMLRRQLLQQQQQQQQQQQQQQQQQQGSAGMAGGMAG | |
| | | | | HGQFQQPQGPGGYPPAMQQQQRMQQHLPLQGSSMGQMAAQMGQ | |
| | | | | LGQMGQPGLGADSTPNIQQALQQRILQQQQMKQQIGSPGQPNPMS | |
| | | | | PQQHMLSGQPQASHLPGQQIATSLSNQVRSPAPVQSPRPQSQPPHSS | |
| | | | | PSPRIQPQPSPHHVSPQTGSPHPGLAVTMASSIDQGHLGNPEQSAML | |
| | | | | PQLNTPSRSALSSELSLVGDTTGDTLEKFVEGL* | |
| CREBBP | CREB binding protein | 1387 | 255 | XDGSNSGNIGTLSTIPTAAPPSSTGVRKGWHEHVTQDLRSHLVHKL | 103. |
| | [Source:HGNC | | | VQAIFPTPDPAALKDRRMENLVAYAKKVEGDMYESANSRDEYYHL | |
| | Symbol; Acc:HGNC: | | | LAEKIYKIQKELEEKRRSRLHKQGILGNQPALPAPGAQPPVIPQAQP | |
| | 2348] | | | VRPPRMNSFNPMSLGNVQLPQAPMGPRAASPMNHSVQMNSMGSV | |
| | | | | PGMAISPSRMPQPPNMMGAHTNNMMAQAPAQSQFLPQNQFPSSSG | |
| | | | | AMSVGMGQPPAQTGVSQVLCPTGTVAP* | |
| CREBBP | CREB binding protein | 1387 | 1105 | NATSLSNPNPIDPSSMQRAYAALGLPYMNQPQTQLQPQVPGQQPA | 104. |
| | [Source:HGNC | | | QPQTHQQMRTLNPLGNNPMNIPAGGITTDQQPPNLISESALPTSLGA | |
| | Symbol; Acc:HGNC: | | | TNPLMNDGSNSGNIGTLSTIPTAAPPSSTGVRKGWHEHVTQDLRSH | |
| | 2348] | | | LVHKLVQAIFPTPDPAALKDRRMENLVAYAKKVEGDMYESANSRD | |
| | | | | EYYHLLAEKIYKIQKELEEKRRSRLHKQGILGNQPALPAPGAQPPVI | |
| | | | | PQAQPVRPPNGPLSLPVNRMQVSQGMNSFNPMSLGNVQLPQAPMG | |
| | | | | PRAASPMNHSVQMNSMGSVPGMAISPSRMPQPPNMMGAHTNNM | |
| | | | | MAQAPAQSQFLPQNQFPSSSGAMSVGMGQPPAQTGVSQGQVPGA | |
| | | | | ALPNPLNMLGPQASQLPCPPVTQSPLHPTPPPASTAAGMPSLQHTTP | |
| | | | | PGMTPPQPAAPTQPSTPVSSSGQTPTPTPGSVPSATQTQSTPTVQAA | |
| | | | | AQAQVTPQPQTPVQPPSVATPQSSQQQPTPVHAQPPGTPLSQAAASI | |
| | | | | DNRVPTPSSVASAETNSQQPGPDVPVLEMKTETQAEDTEPDPGESK | |
| | | | | GEPRSEMMEEDLQGASQVKEETDIAEQKSEPMEVDEKKPEVKVEV | |
| | | | | KEEEESSSNGTASQSTSPSQPRKKIFKPEELRQALMPTLEALYRQDP | |
| | | | | ESLPFRQPVDPQLLGIPDYFDIVKNPMDLSTIKRKLDTGQYQEPWQY | |
| | | | | VDDVWLMFNNAWLYNRKTSRVYKFCSKLAEVFEQEIDPVMQSLG | |
| | | | | YCCGRKYEFSPQTLCCYGKQLCTIPRDAAYYSYQNSSPKYGLLADR | |
| | | | | YHFCEKCFTEIQGENVTLGDDPSQPQTTISKDQFEKKKNDTLDPEPF | |
| | | | | VDCKECGRKMHQICVLHYDIIWPSGFVCDNCLKKTGRPRKENKFS | |
| | | | | AKRLQTTRLGNHLEDRVNKFLRRQNHPEAGEVFVRVVASSDKTVE | |
| | | | | VKPGMKSRFVDSGEMSESFPYRTKALFAFEEIDGVDVCFFGMHVQE | |
| | | | | YGSDCPPPNTRRVYISYLDSIHFFRPRCLRTAVYHEILIGYLEYVKKL | |
| | | | | GYVTGHIWACPPSEGDDYIFHCHPPDQKIPKPKRLQEWYKKMLDK | |
| | | | | AFAERIIHDYKDIFKQATEDRLTSAKELPYFEGDFWPNVLEESIKELE | |
| | | | | QEE | |
| CREBBP | CREB binding protein | 1387 | 33 | XTCNECKHHVETRWHCTVCEFLNAAVFISFCEH | 105. |
| | [Source:HGNC | | | | |
| | Symbol; Acc:HGNC: | | | | |
| | 2348] | | | | |
| CREBBP | CREB binding protein | 1387 | 197 | XRYHFCEKCFTEIQGENVTLGDDPSQPQTTISKDQFEKKKNDTLDPE | 106. |
| | [Source:HGNC | | | PFVDCKECGRKMHQICVLHYDIIWPSGFVCDNCLKKTGRPRKENKF | |
| | Symbol; Acc:HGNC: | | | SAKRLQTTRLGNHLEDRVNKFLRRQNHPEAGEVFVRVVASSDKTV | |
| | 2348] | | | EVKPGMKSRFVDSGEMSESFPYRTKALFAFEEIDGVDVCFFGMHVQ | |
| | | | | EYGSDCPPPNTR* | |
| CREBBP | CREB binding protein | 1387 | 182 | XHLLAEKIYKIQKELEEKRRSRLHKQGILGNQPALPAPGAQPPVIPQ | 107. |
| | [Source:HGNC | | | AQPVRPPNGPLSLPVNRMQVSQGMNSFNPMSLGNVQLPQAPMGPR | |
| | Symbol; Acc:HGNC: | | | AASPMNHSVQMNSMGSVPGMAISPSRMPQPPNMMGAHTNNMMA | |
| | 2348] | | | QAPAQSQFLPQNQFPSSSGAMSVGMGQPPAQTGVSQVLCPTGTVA | |
| | | | | P* | |
| TSPYL2 | TSPY like 2 | 64061 | 694 | MDRPDEGPPAKTRRLSSSESPQRDPPPPPPPPPLLRLPLPPPQQRPRL | 108. |
| | [Source:HGNC | | | QEETEAAQVLADMRGVGLGPALPPPPPYVILEEGGIRAYFTLGAECP | |
| | Symbol; Acc:HGNC: | | | GWDSTIESGYGEAPPPTESLEALPTPEASGGSLEIDFQVVQSSSFGGE | |
| | 24358] | | | GALETCSAVGWAPQRLVDPKSKEEAIIIVEDEDEDERESMRSSRRRR | |
| | | | | RRRRRKQRKVKRESRERNAERMESILQALEDIQLDLEAVNIKAGKA | |
| | | | | FLRLKRKFIQMRRPFLERRDLIIQHIPGFWVKAFLNHPRISILINRRDE | |
| | | | | DIFRYLTNLQVQDLRHISMGYKMKLYFQTNPYFTNMVIVKEFQRN | |
| | | | | RSGRLVSHSTPIRWHRGQEPQARRHGNQDASHSFFSWFSNHSLPEA | |
| | | | | DRIAEIIKNDLWVNPLRYYLRERGSRIKRKKQEMKKRKTRGRCEVV | |
| | | | | IMEDAPDYYAVEDIFSEISDIDETIHDIKISDFMETTDYFETTDNEITDI | |
| | | | | NENICDSENPDHNEVPNNETTDNNESADDHETTDNNESADDNNEN | |
| | | | | PEDNNKNTDDNEENPNNNENTYGNNFFKGGFWGSHGNNQDSSDS | |
| | | | | DNEADEASDDEDNDGNEGDNEGSDDDGNEGDNEGSDDDDRDIEY | |
| | | | | YEKVIEDFDKDQADYEDVIEIISDESVEEEGIEEGIQQDEDIYEEGNY | |
| | | | | EEEGSEDVWEEGEDSDDSDLEDVLQVPNGWANPGKRGKTG* | |
| TSPYL2 | TSPY like 2 | 64061 | 124 | XEEAIIIVEDEDEDERESMRSSRRRRRRRRRKQRKVKRESRERNAER | 109. |
| | [Source:HGNC | | | MESILQALEDIQLDLEAVNIKAGKAFLRLKRKFIQMRRPFLERRDLII | |
| | Symbol; Acc:HGNC: | | | QHIPGFWVKALHSSVPQPPQNFNFDQPT* | |
| | 24358] | | | | |
| TSPYL2 | TSPY like 2 | 64061 | 250 | MDRPDEGPPAKTRRLSSSESPQRDPPPPPPPPPLLRLPLPPPQQRPRL | 110. |
| | [Source:HGNC | | | QEETEAAQIIKNDLWVNPLRYYLRERGSRIKRKKQEMKKRKTRGR | |
| | Symbol; Acc:HGNC: | | | CEVVIMEDAPDYYAVEDIFSEISDIDETIHDIKISDFMETTDYFETTD | |
| | 24358] | | | NEITDINENICDSENPDHNEVPNNETTDNNESADDHETTDNNESAD | |
| | | | | DNNENPEDNNKNTDDNEENPNNNENTYGNNFFKGGFWGSHGNNQ | |
| | | | | DSSDSDNEADEASDDEDND | |
| ZBTB7A | zinc finger and BTB | 51341 | 585 | MAGGVDGPIGIPFPDHSSDILSGLNEQRTQGLLCDVVILVEGREFPT | 111. |
| | domain containing 7A | | | HRSVLAACSQYFKKLFTSGAVVDQQNVYEIDFVSAEALTALMDFA | |
| | [Source:HGNC | | | YTATLTVSTANVGDILSAARLLEIPAVSHVCADLLDRQILAADAGA | |
| | Symbol; Acc:HGNC: | | | DAGQLDLVDQIDQRNLLRAKEYLEFFQSNPMNSLPPAAAAAAASFP | |
| | 18078] | | | WSAFGASDDDLDATKEAVAAAVAAVAAGDCNGLDFYGPGPPAER | |
| | | | | PPTGDGDEGDSNPGLWPERDEDAPTGGLFPPPVAPPAATQNGHYG | |
| | | | | RGGEEEAASLSEAAPEPGDSPGFLSGAAEGEDGDGPDVDGLAASTL | |
| | | | | LQQMMSSVGRAGAAAGDSDEESRADDKGVMDYYLKYFSGAHDG | |
| | | | | DVYPAWSQKVEKKIRAKAFQKCPICEKVIQGAGKLPRHIRTHTGEK | |
| | | | | PYECNICKVRFTRQDKLKVHMRKHTGEKPYLCQQCGAAFAHNYD | |
| | | | | LKNHMRVHTGLRPYQCDSCCKTFVRSDHLHRHLKKDGCNGVPSR | |
| | | | | RGRKPRVRGGAPDPSPGATATPGAPAQPSSPDARRNGQEKHFKDE | |
| | | | | DEDEDVASPDGLGRLNVAGAGGGGDSGGGPGAATDGNFTAGLA* | |
| SIRT1 | sirtuin 1 | 23411 | 748 | MADEAALALQPGGSPSAAGADREAASSPAGEPLRKRPRRDGPGLE | 112. |
| | [Source:HGNC | | | RSPGEPGGAAPEREVPAAARGCPGAAAAALWREAEAEAAAAGGE | |
| | Symbol; Acc:HGNC: | | | QEAQATAAAGEGDNGPGLQGPSREPPLADNLYDEDDDDEGEEEEE | |
| | 14929] | | | AAAAAIGYRDNLLFGDEIITNGFHSCESDEEDRASHASSSDWTPRPR | |
| | | | | IGPYTFVQQHLMIGTDPRTILKDLLPETIPPPELDDMTLWQIVINILSE | |
| | | | | PPKRKKRKDINTIEDAVKLLQECKKIIVLTGAGVSVSCGIPDFRSRD | |
| | | | | GIYARLAVDFPDLPDPQAMFDIEYFRKDPRPFFKFAKEIYPGQFQPS | |
| | | | | LCHKFIALSDKEGKLLRNYTQNIDTLEQVAGIQRIIQCHGSFATASCL | |
| | | | | ICKYKVDCEAVRGDIFNQVVPRCPRCPADEPLAIMKPEIVFFGENLP | |
| | | | | EQFHRAMKYDKDEVDLLIVIGSSLKVRPVALIPSSIPHEVPQILINREP | |
| | | | | LPHLHFDVELLGDCDVIINELCHRLGGEYAKLCCNPVKLSEITEKPP | |
| | | | | RTQKELAYLSELPPTPLHVSEDSSSPERTSPPDSSVIVTLLDQAAKSN | |
| | | | | DDLDVSESKGCMEEKPQEVQTSRNVESIAEQMENPDLKNVGSSTGE | |
| | | | | KNERTSVAGTVRKCWPNRVAKEQISRRLDGNQYLFLPPNRYIFHGA | |
| | | | | EVYSDSEDDVLSSSSCGSNSDSGTCQSPSLEEPMEDESEIEEFYNGLE | |
| | | | | DEPDVPERAGGAGFGTDGDDQEAINEAISVKQEVTDMNYPSNKS* | |
| SIRT1 | sirtuin 1 | 23411 | 445 | MCLCSGRKTILEIYPGQFQPSLCHKFIALSDKEGKLLRNYTQNIDTLE | 113. |
| | [Source:HGNC | | | QVAGIQRIIQCHGSFATASCLICKYKVDCEAVRGDIFNQVVPRCPRC | |
| | Symbol; Acc:HGNC: | | | PADEPLAIMKPEIVFFGENLPEQFHRAMKYDKDEVDLLIVIGSSLKV | |
| | 14929] | | | RPVALIPSSIPHEVPQILINREPLPHLHFDVELLGDCDVIINELCHRLG | |
| | | | | GEYAKLCCNPVKLSEITEKPPRTQKELAYLSELPPTPLHVSEDSSSPE | |
| | | | | RTSPPDSSVIVTLLDQAAKSNDDLDVSESKGCMEEKPQEVQTSRNV | |
| | | | | ESIAEQMENPDLKNVGSSTGEKNERTSVAGTVRKCWPNRVAKEQIS | |
| | | | | RRLDGNQYLFLPPNRYIFHGAEVYSDSEDDVLSSSSCGSNSDSGTCQ | |
| | | | | SPSLEEPMEDESEIEEFYNGLEDEPDVPERAGGAGFGTDGDDQEAIN | |
| | | | | EAISVKQEVTDMNYPSNKS* | |
| SIRT1 | sirtuin 1 | 23411 | 453 | MFDIEYFRKDPRPFFKFAKEIYPGQFQPSLCHKFIALSDKEGKLLRN | 114. |
| | [Source HGNC | | | YTQNIDTLEQVAGIQRIIQCHGSFATASCLICKYKVDCEAVRGDIFN | |
| | Symbol; Acc:HGNC: | | | QVVPRCPRCPADEPLAIMKPEIVFFGENLPEQFHRAMKYDKDEVDL | |
| | 14929] | | | LIVIGSSLKVRPVALIPSSIPHEVPQILINREPLPHLHFDVELLGDCDVI | |
| | | | | INELCHRLGGEYAKLCCNPVKLSEITEKPPRTQKELAYLSELPPTPLH | |
| | | | | VSEDSSSPERTSPPDSSVIVTLLDQAAKSNDDLDVSESKGCMEEKPQ | |
| | | | | EVQTSRNVESIAEQMENPDLKNVGSSTGEKNERTSVAGTVRKCWP | |
| | | | | NRVAKEQISRRLDGNQYLFLPPNRYIFHGAEVYSDSEDDVLSSSSCG | |
| | | | | SNSDSGTCQSPSLEEPMEDESEIEEFYNGLEDEPDVPERAGGAGFGT | |
| | | | | DGDDQEAINEAISVKQEVTDMNYPSNKS* | |
| MTMR8 | myotubularin related | 55613 | 705 | MDHITVPKVENVKLVDRYVSKKPANGILYLTATHLIYVEASGAAR | 115. |
| | protein 8 | | | KETWIALHHIATVEKLPITSLGCPLTLRCKNFRVAHFVLDSDLVCHE | |
| | [Source:HGNC | | | VYISLLKLSQPALPEDLYAFSYNPKSSKEMRESGWKLIDPISDFGRM | |
| | Symbol; Acc:HGNC:16 | | | GIPNRNWTITDANRNYEICSTYPPEIVVPKSVTLGTVVGSSKFRSKER | |
| | 825] | | | VPVLSYLYKENNAAICRCSQPLSGFYTRCVDDELLLEAISQTNPGSQ | |
| | | | | FMYVVDTRPKLNAMANRAAGKGYENEDNYANIRFRFMGIENIHV | |
| | | | | MRSSLQKLLEVCELKTPTMSEFLSGLESSGWLRHIKAIMDAGIFITK | |
| | | | | AVKVEKASVLVHCSDGWDRTAQVCSVASILLDPFYRTFKGLMILIE | |
| | | | | KEWISMGHKFSQRCGHLDGDSKEVSPIFTQFLDCIWQLMEQFPCAF | |
| | | | | EFNENFLLEIHDHVFSCQFGNFLGNCQKDREDLRVYEKTHSVWPFL | |
| | | | | VQRKPDFRNPLYKGFTMYGVLNPSTVPYNIQFWCGMYNRFDKGLQ | |
| | | | | PKQSMLESLLEIKKQRAMLETDVHELEKKLKVRDEPPEEICTCSQLG | |
| | | | | NILSQHLGSPLTNPLGFMGINGDLNTLMENGTLSREGGLRAQMDQV | |
| | | | | KSQGADLHHNCCEIVGSLRAINISGDVGISEAMGISGDMCTFEATGF | |
| | | | | SKDLGICGAMDISEATGISGNLGISEARGFSGDMGILGDTGISKASTK | |
| | | | | EADYSKHQ* | |
| DDIT4 | DNA damage inducible | 54541 | 233 | MPSLWDRFSSSSTSSSPSSLPRTPTPDRPPRSAWGSATREEGFDRSTS | 116. |
| | transcript 4 | | | LESSDCESLDSSNSGFGPEEDTAYLDGVSLPDFELLSDPEDEHLCAN | |
| | [Source:HGNC | | | LMQLLQESLAQARLGSRRPARLLMPSQLVSQVGKELLRLAYSEPCG | |
| | Symbol; Acc:HGNC: | | | LRGALLDVCVEQGKSCHSVGQLALDPSLVPTFQLTLVLRLDSRLWP | |
| | 24944] | | | KIQGLFSSANSPFLPGFSQSLTLSTGFRVIKKKLYSSEQLLIEEC* | |
| ZNF699 | zinc finger protein 699 | 374879 | 643 | MEEERKTAELQKNRIQDSVVFEDVAVDFTQEEWALLDLAQRNLYR | 117. |
| | [Source:HGNC | | | DVMLENFQNLASLGYPLHTPHLISQWEQEEDLQTVKRELIQGIFMG | |
| | Symbol; Acc:HGNC: | | | EHREGFETQLKTNESVASQDICGEKISNEQKIVRFKRNDSWFSSLHE | |
| | 24750] | | | NQESCGIDYQNKSHERHLRNHMVENIYECYEENQDGQTFSQVPNL | |
| | | | | DSLKRNTEVKSCECHECGKAFVDHSSLKSHIRSHTGSKPYQCKECG | |
| | | | | KAFHFLACFKKHMKTPTEEKPYECKECTKAFSCSSFFRAHMKIHIG | |
| | | | | KTNYECKECGKGFSCSSSLTEHKRIHSGDKPYECKECGKAFSCSSSL | |
| | | | | SKHKRIHSGDKPYECKECGKAFSSSSHLIIHIRIHTGEKPYECKECGK | |
| | | | | AFSESSKLTVHGRTHTGEKPYKCKECGKAYNCPSSLSIHMRKHTGE | |
| | | | | KPYECLECGKAFYLPTSLNTHVKNQSREKPYECKECGKAFSCPSSF | |
| | | | | RAHVRDHTGKIQYECKECGKTFSRSSSLTEHLRTHSGEKPYECKEC | |
| | | | | GKAFISSSHLTVHIRTHTGEKPYECKKCGKAFIYPSALRIHMRTHTG | |
| | | | | EKPYECKECGKAFRHSSYLTVHARMHTGEKPFECLECGKAFSCPSS | |
| | | | | FRRHVRSHTGEKPYECKECGKAFVCPAYFRRHVKTHTRENI* | |
| GLA | galactosidase alpha | 2717 | 430 | MQLRNPELHLGCALALRFLALVSWDIPGARALDNGLARTPTMGWL | 118. |
| | [Source:HGNC | | | HWERFMCNLDCQEEPDSCISEKLFMEMAELMVSEGWKDAGYEYL | |
| | Symbol; Acc:HGNC: | | | CIDDCWMAPQRDSEGRLQADPQRFPHGIRQLANYVHSKGLKLGIY | |
| | 4296] | | | ADVGNKTCAGFPGSFGYYDIDAQTFADWGVDLLKFDGCYCDSLEN | |
| | | | | LADGYKHMSLALNRTGRSIVYSCEWPLYMWPFQKPNYTEIRQYCN | |
| | | | | HWRNFADIDDSWKSIKSILDWTSFNQERIVDVAGPGGWNDPDMLV | |
| | | | | IGNFGLSWNQQVTQMALWAIMAAPLFMSNDLRHISPQAKALLQDK | |
| | | | | DVIAINQDPLGKQGYQLRQGDNFEVWERPLSGLAWAVAMINRQEI | |
| | | | | GGPRSYTIAVASLGKGVACNPACFITQLLPVKRKLGFYEWTSRLRS | |
| | | | | HINPTGTVLLQLENTMQMSLKDLL* | |
| GLA | galactosidase alpha | 2717 | 200 | XDIPGARALDNGLARTPTMGWLHWERFMCNLDCQEEPDSCISEKL | 119. |
| | [Source:HGNC | | | FMEMAELMVSEGWKDAGYEYLCIDDCWMAPQRDSEGRLQADPQ | |
| | Symbol; Acc:HGNC: | | | RFPHGIRQLANYVHSKGLKLGIYADVGNKTCAGFPGSFGYYDIDAQ | |
| | 4296] | | | TFADWGVDLLKFDGCYCDSLENLADGESRLSIGHRERSIFIPAFPQY | |
| | | | | THHLCTTSSEQSQQSGRL* | |
| GLA | galactosidase alpha | 2717 | 223 | MQLRNPELHLGCALALRFLALVSWDIPGARALDNGLARTPTMGWL | 120. |
| | [Source:HGNC | | | HWERFMCNLDCQEEPDSCISEKLFMEMAELMVSEGWKDAGYEYL | |
| | Symbol; Acc:HGNC: | | | CIDDCWMAPQRDSEGRLQADPQRFPHGIRQLANYVHSKGLKLGIY | |
| | 4296] | | | ADVGNKTCAGFPGSFGYYDIDAQTFADWGVDLLKFDGCYCDSLEN | |
| | | | | LADGYKHMSLALNRTGRSIVYSCEWPLYMWPFQKSSTLFGSIC* | |
| GLA | galactosidase alpha | 2717 | 200 | MQLRNPELHLGCALALRFLALVSWDIPGARALDNGLARTPTMGWL | 121. |
| | [Source:HGNC | | | HWERFMCNLDCQEEPDSCISEKLFMEMAELMVSEGWKDAGYEYL | |
| | Symbol; Acc:HGNC: | | | CIDDCWMAPQRDSEGRLQADPQRFPHGIRQLANYVHSKGLKLGIY | |
| | 4296] | | | ADVGNKTCAGFPGSFGYYDIDAQTFADWGVDLLKFDGCYCDSLEN | |
| | | | | LADAQLYRNPTVLQSLAKFC* | |
| GLA | galactosidase alpha | 2717 | 471 | MQLRNPELHLGCALALRFLALVSWDIPGARALDNGLARTPTMGWL | 122. |
| | [Source:HGNC | | | HWERFMCNLDCQEEPDSCISWSFALVAQAGVQCHDLGSPQPPPPRF | |
| | Symbol; Acc:HGNC: | | | KQFSCLILASSWNYSEKLFMEMAELMVSEGWKDAGYEYLCIDDC | |
| | 4296] | | | WMAPQRDSEGRLQADPQRFPHGIRQLANYVHSKGLKLGIYADVGN | |
| | | | | KTCAGFPGSFGYYDIDAQTFADWGVDLLKFDGCYCDSLENLADGY | |
| | | | | KHMSLALNRTGRSIVYSCEWPLYMWPFQKPNYTEIRQYCNHWRNF | |
| | | | | ADIDDSWKSIKSILDWTSFNQERIVDVAGPGGWNDPDMLVIGNFGL | |
| | | | | SWNQQVTQMALWAIMAAPLFMSNDLRHISPQAKALLQDKDVIAIN | |
| | | | | QDPLGKQGYQLRQGDNFEVWERPLSGLAWAVAMINRQEIGGPRSY | |
| | | | | TIAVASLGKGVACNPACFITQLLPVKRKLGFYEWTSRLRSHINPTGT | |
| | | | | VLLQLENTMQMSLKDLL* | |
| GLA | galactosidase alpha | 2717 | 199 | DIPGARALDNGLARTPTMGWLHWERFMCNLDCQEEPDSCISEKLF | 123. |
| | [Source:HGNC | | | MEMAELMVSEGWKDAGYEYLCIDDCWMAPQRDSEGRLQADPQR | |
| | Symbol; Acc:HGNC: | | | FPHGIRQLANYVHSKGLKLGIYADVGNKTCAGFPGSFGYYDIDAQT | |
| | 4296] | | | FADWGVDLLKFDGCYCDSLENLADGESRLSIGHRERSIFIPAFPQYT | |
| | | | | HHLCTTSSEQSQQSGRL* | |
| GLA | galactosidase alpha | 2717 | 363 | MQLRNPELHLGCALALRFLALVSWDIPGARALDNGLARTPTMGWL | 124. |
| | [Source:HGNC | | | HWERFMCNLDCQEEPDSCISEKLFMEMAELMVSEGWKDAGYEYL | |
| | Symbol; Acc:HGNC: | | | CIDDCWMAPQRDSEGRLQADPQRFPHGIRQLANYVHSKGLKLGIY | |
| | 4296] | | | ADVGNKTCAGFPGSFGYYDIDAQTFADWGVDLLKFDGCYCDSLEN | |
| | | | | LADGYKHMSLALNRTGRSIVYSCEWPLYMWPFQKPNYTEIRQYCN | |
| | | | | HWRNFADIDDSWKSIKSILDWTSFNQERIVDVAGPGGWNDPDMLV | |
| | | | | IGNFGLSWNQQVTQMALWAIMAAPLFMSNDLRHISPQAKALLQDK | |
| | | | | DVIAINQDPLGKQGYQLRQVNKSIYFKMALYTQYQLCLGPKSIFFPC | |
| | | | | S* | |
| GLA | galactosidase alpha | 2717 | 364 | MQLRNPELHLGCALALRFLALVSWDIPGARALDNGLARTPTMGWL | 125. |
| | [Source:HGNC | | | HWERFMCNLDCQEEPDSCISEKLFMEMAELMVSEGWKDAGYEYL | |
| | Symbol; Acc:HGNC: | | | CIDDCWMAPQRDSEGRLQADPQRFPHGIRQLANYVHSKGLKLGIY | |
| | 4296] | | | ADVGNKTCAGFPGSFGYYDIDAQTFADWGVDLLKFDGCYCDSLEN | |
| | | | | LADGYKHMSLALNRTGRSIVYSCEWPLYMWPFQKPNYTEIRQYCN | |
| | | | | HWRNFADIDDSWKSIKSILDWTSFNQERIVDVAGPGGWNDPDMGD | |
| | | | | NFEVWERPLSGLAWAVAMINRQEIGGPRSYTIAVASLGKGVACNP | |
| | | | | ACFITQLLPVKRKLGFYEWTSRLRSHINPTGTVLLQLENTMQMSLK | |
| | | | | DLL* | |
| GLA | galactosidase alpha | 2717 | 418 | MQLRNPELHLGCALALRFLALVSWDIPGARALDNGLARTPTMGWL | 126. |
| | [Source:HGNC | | | HWERFMCNLDCQEEPDSCISEKLFMEMAELMVSEGWKDAGYEYL | |
| | Symbol; Acc:HGNC: | | | CIDDCWMAPQRDSEGRLQADPQRFPHGIRQLANYVHSKGLKLGIY | |
| | 4296] | | | ADVGNKTCAGFPGSFGYYDIDAQTFADWGVDLLKFDGYKHMSLA | |
| | | | | LNRTGRSIVYSCEWPLYMWPFQKPNYTEIRQYCNHWRNFADIDDS | |
| | | | | WKSIKSILDWTSFNQERIVDVAGPGGWNDPDMLVIGNFGLSWNQQ | |
| | | | | VTQMALWAIMAAPLFMSNDLRHISPQAKALLQDKDVIAINQDPLG | |
| | | | | KQGYQLRQGDNFEVWERPLSGLAWAVAMINRQEIGGPRSYTIAVA | |
| | | | | SLGKGVACNPACFITQLLPVKRKLGFYEWTSRLRSHINPTGTVLLQL | |
| | | | | ENTMQMSLKDLL* | |
| GLA | galactosidase alpha | 2717 | 271 | MQLRNPELHLGCALALRFLALVSWDIPGARALDNGLARTPTMGWL | 127. |
| | [Source:HGNC | | | HWERFMCNLDCQEEPDSCISEKLFMEMAELMVSEGWKDAGYEYL | |
| | Symbol; Acc:HGNC: | | | CIDDCWMAPQRDSEGRLQADPQRFPHGIRQLANYVHSKGLKLGIY | |
| | 4296] | | | ADVGNKTCAGFPGSFGYYDIDAQTFADWGVDLLKFDGCYCDSLEN | |
| | | | | LADGYKHMSLALNRTGRSIVYSCEWPLYMWPFQKPNYTEIRQYCN | |
| | | | | HWRNFADIDDSWKSIKSILDWTSFNQERIVDVAGPGGWNDPDMVK | |
| | | | | T* | |
| LRRC8A | leucine rich repeat | 56262 | 811 | MIPVTELRYFADTQPAYRILKPWWDVFTDYISIVMLMIAVFGGTLQ | 128. |
| | containing 8 VRAC | | | VTQDKMICLPCKWVTKDSCNDSFRGWAAPGPEPTYPNSTILPTPDT | |
| | subunit A | | | GPTGIKYDLDRHQYNYVDAVCYENRLHWFAKYFPYLVLLHTLIFL | |
| | [Source:HGNC | | | ACSNFWFKFPRTSSKLEHFVSILLKCFDSPWTTRALSETVVEESDPK | |
| | Symbol; Acc:HGNC:19 | | | PAFSKMNGSMDKKSSTVSEDVEATVPMLQRTKSRIEQGIVDRSETG | |
| | 027] | | | VLDKKEGEQAKALFEKVKKFRTHVEEGDIVYRLYMRQTIIKVIKFIL | |
| | | | | IICYTVYYVHNIKFDVDCTVDIESLTGYRTYRCAHPLATLFKILASFY | |
| | | | | ISLVIFYGLICMYTLWWMLRRSLKKYSFESIREESSYSDIPDVKNDF | |
| | | | | AFMLHLIDQYDPLYSKRFAVFLSEVSENKLRQLNLNNEWTLDKLR | |
| | | | | QRLTKNAQDKLELHLFMLSGIPDTVFDLVELEVLKLELIPDVTIPPSI | |
| | | | | AQLTGLKELWLYHTAAKIEAPALAFLRENLRALHIKFTDIKEIPLWI | |
| | | | | YSLKTLEELHLTGNLSAENNRYIVIDGLRELKRLKVLRLKSNLSKLP | |
| | | | | QVVTDVGVHLQKLSINNEGTKLIVLNSLKKMANLTELELIRCDLERI | |
| | | | | PHSIFSLHNLQEIDLKDNNLKTIEEIISFQHLHRLTCLKLWYNHIAYIP | |
| | | | | IQIGNLTNLERLYLNRNKIEKIPTQLFYCRKLRYLDLSHNNLTFLPAD | |
| | | | | IGLLQNLQNLAITANRIETLPPELFQCRKLRALHLGNNVLQSLPSRV | |
| | | | | GELTNLTQIELRGNRLECLPVELGECPLLKRSGLVVEEDLFNTLPPE | |
| | | | | VKERLWRADKEQA* | |
| S1PR2 | sphingosine-1- | 9294 | 354 | MGSLYSEYLNPNKVQEHYNYTKETLETQETTSRQVASAFIVILCCAI | 129. |
| | phosphate receptor 2 | | | VVENLLVLIAVARNSKFHSAMYLFLGNLAASDLLAGVAFVANTLL | |
| | [Source:HGNC | | | SGSVTLRLTPVQWFAREGSAFITLSASVFSLLAIAIERHVAIAKVKLY | |
| | Symbol; Acc:HGNC: | | | GSDKSCRMLLLIGASWLISLVLGGLPILGWNCLGHLEACSTVLPLY | |
| | 3169] | | | AKHYVLCVVTIFSIILLAIVALYVRIYCVVRSSHADMAAPQTLALLK | |
| | | | | TVTIVLGVFIVCWLPAFSILLLDYACPVHSCPILYKAHYFFAVSTLNS | |
| | | | | LLNPVIYTWRSRDLRREVLRPLQCWRPGVGVQGRRRGGTPGHHLL | |
| | | | | PLRSSSSLERGMHMPTSPTFLEGNTVV* | |
| ICAM1 | intercellular adhesion | 3383 | 533 | MAPSSPRPALPALLVLLGALFPGPGNAQTSVSPSKVILPRGGSVLVT | 130. |
| | molecule 1 | | | CSTSCDQPKLLGIETPLPKKELLLPGNNRKVYELSNVQEDSQPMCYS | |
| | [Source:HGNC | | | NCPDGQSTAKTFLTVYWTPERVELAPLPSWQPVGKNLTLRCQVEG | |
| | Symbol; Acc:HGNC: | | | GAPRANLTVVLLRGEKELKREPAVGEPAEVTTTVLVRRDHHGANF | |
| | 5344] | | | SCRTELDLRPQGLELFENTSAPYQLQTFVLPATPPQLVSPRVLEVDT | |
| | | | | QGTVVCSLDGLFPVSEAQVHLALGDQRLNPTVTYGNDSFSAKASV | |
| | | | | SVTAEDEGTQRLTCAVILGNQSQETLQTVTIYSFPAPNVILTKPEVSE | |
| | | | | GTEVTVKCEAHPRAKVTLNGVPAQPLGPRAQLLLKATPEDNGRSFS | |
| | | | | CSATLEVAGQLIHKNQTRELRVLYGPRLDERDCPGNWTWPENSQQ | |
| | | | | TPMCQAWGNPLPELKCLKDGTFPLPIGESVTVTRDLEGTYLCRARS | |
| | | | | TQGEVTRKVTVNVLSPRYEIVIITVVAAAVIMGTAGLSTYLYNRQR | |
| | | | | KIKKYRLQQAQKGTPMKPNTQATPP* | |
| ICAM1 | intercellular adhesion | 3383 | 311 | MAPSSPRPALPALLVLLGALFPVSEAQVHLALGDQRLNPTVTYGND | 131. |
| | molecule 1 | | | SFSAKASVSVTAEDEGTQRLTCAVILGNQSQETLQTVTIYSFPAPNVI | |
| | [Source:HGNC | | | LTKPEVSEGTEVTVKCEAHPRAKVTLNGVPAQPLGPRAQLLLKATP | |
| | Symbol; Acc:HGNC: | | | EDNGRSFSCSATLEVAGQLIHKNQTRELRVLYGPRLDERDCPGNWT | |
| | 5344] | | | WPENSQQTPMCQAWGNPLPELKCLKDGTFPLPIGESVTVTRDLEGT | |
| | | | | YLCRARSTQGEVTRKVTVNVLSPRYEIVIITVVAAAVIMGTAGLSTY | |
| | | | | LYNRQRKIKKYRLQQAQKGTPMKPNTQATPP* | |
| ICAM1 | intercellular adhesion | 3383 | 180 | MAPSSPRPALPALLVLLGALFPGPGNAQTSVSPSKVILPRGGSVLVT | 132. |
| | molecule 1 | | | CSTSCDQPKLLGIETPLPKKELLLPGNNRKVYELSNVQEDSQPMCYS | |
| | [Source:HGNC | | | NCPDGQSTAKTFLTVYWTPERVEGGAPRANLTVVLLRGEKELKRE | |
| | Symbol; Acc:HGNC: | | | PAVGEPAEVTTTVLVRRDHHGANFSCRTELDLRPQGLELFE | |
| | 5344] | | | | |
| AMMECR1 | AMMECR nuclear | 9949 | 334 | MAAGCCGVKKQKLSSSPPSGSGGGGGASSSSHCSGESQCRAGELGL | 133. |
| | protein 1 | | | GGAGTRLNGLGGLTGGGSGSGCTLSPPQGCGGGGGGIALSPPPSCG | |
| | [Source:HGNC | | | VGTLLSTPAAATSSSPSSSSAASSSSPGSRKMVVSAEMCCFCFDVLY | |
| | Symbol; Acc:HGNC: | | | CHLYGYQQPRTPRFTNEPYPLFVTWKIGRDKRLRGCIGTFSAMNLH | |
| | 467] | | | SGLREYTLTSALKDSRFPPMTRDELPRLFCSVSLLTNFEDVCDYLD | |
| | | | | WEVGVHGIRIEFINEKGSKRTATYLPEVAKEQGWDHIQTIDSLLRKG | |
| | | | | GYKAPITNEFRKTIKLTRYRSEKMTLSYAEYLAHRQHHHFQNGIGH | |
| | | | | PLPPYNHYS* | |
| AMMECR1 | AMMECR nuclear | 9949 | 211 | MVVSAEMCCFCFDVLYCHLYGYQQPRTPRFTNEPYPLFVTWKIGR | 134. |
| | protein 1 | | | DKRLRGCIGTFSAMNLHSGLREYTLTSALKDSRFPPMTRDELPRLFC | |
| | [Source:HGNC | | | SVSLLTNFEDVCDYLDWEVGVHGIRIEFINEKGSKRTATYLPEVAKE | |
| | Symbol; Acc:HGNC: | | | QGWDHIQTIDSLLRKGGYKAPITNEFRKTIKLTRYRSEKMTLSYAEY | |
| | 467] | | | LAHRQHHHFQNGIGHPLPPYNHYS* | |
| AMMECR1 | AMMECR nuclear | 9949 | 297 | MAAGCCGVKKQKLSSSPPSGSGGGGGASSSSHCSGESQCRAGELGL | 135. |
| | protein 1 | | | GGAGTRLNGLGGLTGGGSGSGCTLSPPQGCGGGGGGIALSPPPSCG | |
| | [Source:HGNC | | | VGTLLSTPAAATSSSPSSSSAASSSSPGSRKMVVSAEMCCFCFDVLY | |
| | Symbol; Acc:HGNC: | | | CHLYGYQQPRTPRFTNEPYALKDSRFPPMTRDELPRLFCSVSLLTNF | |
| | 467] | | | EDVCDYLDWEVGVHGIRIEFINEKGSKRTATYLPEVAKEQGWDHIQ | |
| | | | | TIDSLLRKGGYKAPITNEFRKTIKLTRYRSEKMTLSYAEYLAHRQHH | |
| | | | | HFQNGIGHPLPPYNHYS* | |
| HMGCR | 3-hydroxy-3- | 3156 | 889 | MLSRLFRMHGLFVASHPWEVIVGTVTLTICMMSMNMFTGNNKICG | 136. |
| | methylglutaryl-CoA | | | WNYECPKFEEDVLSSDIIILTITRCIAILYIYFQFQNLRQLGSKYILGIA | |
| | reductase | | | GLFTIFSSFVFSTVVIHFLDKELTGLNEALPFFLLLIDLSRASTLAKFA | |
| | [Source:HGNC | | | LSSNSQDEVRENIARGMAILGPTFTLDALVECLVIGVGTMSGVRQL | |
| | Symbol; Acc:HGNC: | | | EIMCCFGCMSVLANYFVFMTFFPACVSLVLELSRESREGRPIWQLSH | |
| | 5006] | | | FARVLEEEENKPNPVTQRVKMIMSLGLVLVHAHSRWIADPSPQNST | |
| | | | | ADTSKVSLGLDENVSKRIEPSVSLWQFYLSKMISMDIEQVITLSLAL | |
| | | | | LLAVKYIFFEQTETESTLSLKNPITSPVVTQKKVPDNCCRREPMLVR | |
| | | | | NNQKCDSVEEETGINRERKVEVIKPLVAETDTPNRATFVVGNSSLL | |
| | | | | DTSSVLVTQEPEIELPREPRPNEECLQILGNAEKGAKFLSDAEIIQLV | |
| | | | | NAKHIPAYKLETLMETHERGVSIRRQLLSKKLSEPSSLQYLPYRDYN | |
| | | | | YSLVMGACCENVIGYMPIPVGVAGPLCLDEKEFQVPMATTEGCLV | |
| | | | | ASTNRGCRAIGLGGGASSRVLADGMTRGPVVRLPRACDSAEVKAW | |
| | | | | LETSEGFAVIKEAFDSTSRFARLQKLHTSIAGRNLYIRFQSRSGDAM | |
| | | | | GMNMISKGTEKALSKLHEYFPEMQILAVSGNYCTDKKPAAINWIEG | |
| | | | | RGKSVVCEAVIPAKVVREVLKTTTEAMIEVNINKNLVGSAMAGSIG | |
| | | | | GYNAHAANIVTAIYIACGQDAAQNVGSSNCITLMEASGPTNEDLYI | |
| | | | | SCTMPSIEIGTVGGGTNLLPQQACLQMLGVQGACKDNPGENARQL | |
| | | | | ARIVCGTVMAGELSLMAALAAGHLVKSHMIHNRSKINLQDLQGAC | |
| | | | | TKKTA* | |
| HMGCR | 3-hydroxy-3- | 3156 | 836 | MLSRLFRMHGLFVASHPWEVIVGTVTLTICMMSMNMFTGNNKICG | 137. |
| | methylglutaryl-CoA | | | WNYECPKFEEDVLSSDIIILTITRCIAILYIYFQFQNLRQLGSKYILGIA | |
| | reductase | | | GLFTIFSSFVFSTVVIHFLDKELTGLNEALPFFLLLIDLSRASTLAKFA | |
| | [Source:HGNC | | | LSSNSQDEVRENIARGMAILGPTFTLDALVECLVIGVGTMSGVRQL | |
| | Symbol; Acc:HGNC: | | | EIMCCFGCMSVLANYFVFMTFFPACVSLVLELSRESREGRPIWQLSH | |
| | 5006] | | | FARVLEEEENKPNPVTQRVKMIMSLGLVLVHAHSRWIADPSPQNST | |
| | | | | ADTSKVSLGLDENVSKRIEPSVSLWQFYLSKMISMDIEQVITLSLAL | |
| | | | | LLAVKYIFFEQTETESTLSLKNPITSPVVTQKKVPDNCCRREPMLVR | |
| | | | | NNQKCDSVEEETGINRERKVEVIKPLVAETDTPNRATFVVGNSSLL | |
| | | | | DTSSVLVTQEPEIELPREPRPNEECLQILGNAEKGAKFLSDAEIIQLV | |
| | | | | NAKHIPAYKLETLMETHERGVSIRRQLLSKKLSEPSSLQYLPYRDYN | |
| | | | | YSLLGGGASSRVLADGMTRGPVVRLPRACDSAEVKAWLETSEGFA | |
| | | | | VIKEAFDSTSRFARLQKLHTSIAGRNLYIRFQSRSGDAMGMNMISK | |
| | | | | GTEKALSKLHEYFPEMQILAVSGNYCTDKKPAAINWIEGRGKSVVC | |
| | | | | EAVIPAKWREVLKTTTEAMIEVNINKNLVGSAMAGSIGGYNAHA | |
| | | | | ANIVTAIYIACGQDAAQNVGSSNCITLMEASGPTNEDLYISCTMPSIE | |
| | | | | IGTVGGGTNLLPQQACLQMLGVQGACKDNPGENARQLARIVCGTV | |
| | | | | MAGELSLMAALAAGHLVKSHMIHNRSKINLQDLQGACTKKTA* | |
| HMGCR | 3-hydroxy-3- | 3156 | 18 | MLSRLFRMHGLFVASHPW | 138. |
| | methylglutaryl-CoA | | | | |
| | reductase | | | | |
| | [Source:HGNC | | | | |
| | Symbol; Acc:HGNC: | | | | |
| | 5006] | | | | |
| HMGCR | 3-hydroxy-3- | 3156 | 116 | YFPEMQILAVSGNYCTDKKPAAINWIEGRGKSVVCEAVIPAKVVRE | 139. |
| | methylglutaryl-CoA | | | MLGVQGACKDNPGENARQLARIVCGTVMAGELSLMAALAAGHLV | |
| | reductase | | | KSHMIHNRSKINLQDLQGACTKKTA* | |
| | [Source:HGNC | | | | |
| | Symbol; Acc:HGNC: | | | | |
| | 5006] | | | | |
| HMGCR | 3-hydroxy-3- | 3156 | 166 | XHEYFPEMQILAVSGNYCTDKKPAAINWIEGRGKSVVCEAVIPAKV | 140. |
| | methylglutaryl-CoA | | | VREVLKTTTEAMIEVNINKNLVGSAMAGSIGGYNAHAANIVTAIYI | |
| | reductase | | | ACGQMLGVQGACKDNPGENARQLARIVCGTVMAGELSLMAALAA | |
| | [Source:HGNC | | | GHLVKSHMIHNRSKINLQDLQGACTKKTA* | |
| | Symbol; Acc:HGNC: | | | | |
| | 5006] | | | | |
| HMGCR | 3-hydroxy-3- | 3156 | 111 | MLSRLFRMHGLFVASHPWEVIVGTVTLTICMMSMNMFTGNNKICG | 141. |
| | methylglutaryl-CoA | | | WNYECPKFEEDVLSSDIIILTITRCIAILYIYFQFQNLRQLGSKYILGIA | |
| | reductase | | | GLFTIFSSFVFSTVVI | |
| | [Source:HGNC | | | | |
| | Symbol; Acc:HGNC: | | | | |
| | 5006] | | | | |
| MOSPD1 | motile sperm domain | 56180 | 214 | MHQQKRQPELVEGNLPVFVFPTELIFYADDQSTHKQVLTLYNPYEF | 142. |
| | containing 1 | | | ALKFKVLCTTPNKYVVVDAAGAVKPQCCVDIVIRHRDVRSCHYGV | |
| | [Source:HGNC | | | IDKFRLQVSEQSQRKALGRKEVVATLLPSAKEQQKEEEEKRLKEHL | |
| | Symbol; Acc:HGNC: | | | TESLFFEQSFQPENRAVSSGPSLLTVFLGVVCIAALMLPTLGDVESL | |
| | 25235] | | | VPLYLHLSVNQKLVAAYILGLITMAILRT* | |
| MOSPD1 | motile sperm domain | 56180 | 215 | MHQQKRQPELVEGNLPVFVFPTELIFYADDQSTHKQVLTLYNPYEF | 143. |
| | containing 1 | | | ALKFKVLCTTPNKYVVVDAAGAVKPQCCVDIVIRHRDVRSCHYGV | |
| | [Source:HGNC | | | IDKFRLQVSEQSQRKALGRKEVVATLLPSAKEQQKEEEEKRLKEHL | |
| | Symbol; Acc:HGNC: | | | TESLFFEQSFQPGKNRAVSSGPSLLTVFLGVVCIAALMLPTLGDVES | |
| | 25235] | | | LVPLYLHLSVNQKLVAAYILGLITMAILRT* | |
| MOSPD1 | motile sperm domain | 56180 | 160 | MHQQKRQPELVEGNLPVFVFPTELIFYADDQSTHKQVLTLYNPYEF | 144. |
| | containing 1 | | | ALKFKVLCTTPNKYVVVDAAGAVKPQCCVDIVIRHRDVRSCHYGV | |
| | [Source:HGNC | | | IDKFRLQVSEQSQRKALGRKEVVATLLPSAKEQQKEEEEKRLKEHL | |
| | Symbol; Acc:HGNC: | | | TESLFFEQSFQPGLITMAILRT* | |
| | 25235] | | | | |
| SULT1A2 | sulfotransferase family | 6799 | 296 | MELIQDISRPPLEYVKGVPLIKYFAEALGPLQSFQARPDDLLISTYPK | 145. |
| | 1A member 2 | | | SGTTWVSQILDMIYQGGDLEKCHRAPIFMRVPFLEFKVPGIPSGMET | |
| | [Source:HGNC | | | LKNTPAPRLLKTHLPLALLPQTLLDQKVKVVYVARNAKDVAVSYY | |
| | Symbol; Acc:HGNC: | | | HFYHMAKVYPHPGTWESFLEKFMAGEVSYGSWYQHVQEWWELS | |
| | 11454] | | | RTHPVLYLFYEDMKENPKREIQKILEFVGRSLPEETVDLMVEHTSFK | |
| | | | | EMKKNPMTNYTTVRREFMDHSISPFMRKGMAGDWKTTFTVAQNE | |
| | | | | RFDADYAKKMAGCSLSFRSEL* | |
| SULT1A2 | sulfotransferase family | 6799 | 222 | MELIQDISRPPLEYVKGVPLIKYFAEALGPLQSFQARPDDLLISTYPK | 146. |
| | 1A member 2 | | | SGTTWVSQILDMIYQGGDLEKCHRAPIFMRVPFLEFKVPGIPSGVCV | |
| | [Source:HGNC | | | LGARGVEEDRAGASAHQTFPDPLLRDGDSEKHTSPTTPEDTPAPGS | |
| | Symbol; Acc:HGNC: | | | APPDSVGSEGQGGLCCPQRKGCGGFLLPLLPHGQSVPSPWDLGKLP | |
| | 11454] | | | GEVHGWRSVLWVLVPARARVVGAEPHPPCSLPLL* | |
| SULT1A2 | sulfotransferase family | 6799 | 192 | MELIQDISRPPLEYVKGVPLIKYFAEALGPLQSFQARPDDLLISTYPK | 147. |
| | 1A member 2 | | | SGTTWVSQILDMIYQGGDLEKCHRAPIFMRVPFLEFKVPGIPSGMET | |
| | [Source:HGNC | | | LKNTPAPRLLKTHLPLALLPQTLLDQKVKVVYVARNAKDVAVSYY | |
| | Symbol; Acc:HGNC: | | | HFYHMAKVYPHPGTWESFLEKFMAGEVSYGSWYQHVQEWWELS | |
| | 11454] | | | RTHPVLYLF | |
| SULT1A2 | sulfotransferase family | 6799 | 263 | MELIQDISRPPLEYVKGVPLIKYFAEALGPLQSFQARPDDLLISTYPK | 148. |
| | 1A member 2 | | | SGTTWVSQILDMIYQGGDLEKCHRAPIFMRVPFLEFKVPGIPSGVCV | |
| | [Source:HGNC | | | LGARGVEEDRAGASAHQTFPDPLLRDGDSEKHTSPTTPEDTPAPGS | |
| | Symbol; Acc:HGNC: | | | APPDSVGSEGQAIQASTEEPSAAQNPKREIQKILEFVGRSLPEETVDL | |
| | 11454] | | | MVEHTSFKEMKKNPMTNYTTVRREFMDHSISPFMRKGMAGDWKT | |
| | | | | TFTVAQNERFDADYAKKMAGCSLSFRSEL* | |
| ZHX2 | zinc fingers and | 22882 | 838 | MASKRKSTTPCMVRTSQVVEQDVPEEVDRAKEKGIGTPQPDVAKD | 149. |
| | homeoboxes 2 | | | SWAAELENSSKENEVIEVKSMGESQSKKLQGGYECKYCPYSTQNL | |
| | [Source:HGNC | | | NEFTEHVDMQHPNVILNPLYVCAECNFTTKKYDSLSDHNSKFHPGE | |
| | Symbol; Acc:HGNC: | | | ANFKLKLIKRNNQTVLEQSIETTNHVVSITTSGPGTGDSDSGISVSKT | |
| | 18513] | | | PIMKPGKPKADAKKVPKKPEEITPENHVEGTARLVTDTAEILSRLGG | |
| | | | | VELLQDTLGHVMPSVQLPPNINLVPKVPVPLNTTKYNSALDTNATM | |
| | | | | INSFNKFPYPTQAELSWLTAASKHPEEHIRIWFATQRLKHGISWSPE | |
| | | | | EVEEARKKMFNGTIQSVPPTITVLPAQLAPTKVTQPILQTALPCQILG | |
| | | | | QTSLVLTQVTSGSTTVSCSPITLAVAGVTNHGQKRPLVTPQAAPEPK | |
| | | | | RPHIAQVPEPPPKVANPPLTPASDRKKTKEQIAHLKASFLQSQFPDD | |
| | | | | AEVYRLIEVTGLARSEIKKWFSDHRYRCQRGIVHITSESLAKDQLAI | |
| | | | | AASRHGRTYHAYPDFAPQKFKEKTQGQVKILEDSFLKSSFPTQAEL | |
| | | | | DRLRVETKLSRREIDSWFSERRKLRDSMEQAVLDSMGSGKKGQDV | |
| | | | | GAPNGALSRLDQLSGAQLTSSLPSPSPAIAKSQEQVHLLRSTFARTQ | |
| | | | | WPTPQEYDQLAAKTGLVRTEIVRWFKENRCLLKTGTVKWMEQYQ | |
| | | | | HQPMADDHGYDAVARKATKPMAESPKNGGDVVPQYYKDPKKLC | |
| | | | | EEDLEKLVTRVKVGSEPAKDCLPAKPSEATSDRSEGSSRDGQGSDE | |
| | | | | NEESSVVDYVEVTVGEEDAISDRSDSWSQAAAEGVSELAESDSDCV | |
| | | | | PAEAGQA* | |
| ZHX2 | zinc fingers and | 22882 | 166 | MASKRKSTTPCMVRTSQVVEQDVPEEVDRAKEKGIGTPQPDVAKD | 150. |
| | homeoboxes 2 | | | SWAAELENSSKENEVIEVKSMGESQSKKLQGGYECKYCPYSTQNL | |
| | [Source:HGNC | | | NEFTEHVDMQHPNVILNPLYVCAECNFTTKKYDSLSDHNSKFHPGE | |
| | Symbol; Acc:HGNC: | | | ANFKLKLIKRNNQTVLEQSIETTNHVVSIT | |
| | 18513] | | | | |
| SQLE | squalene epoxidase | 6713 | 575 | MWTFLGIATFTYFYKKFGDFITLANREVLLCVLVFLSLGLVLSYRCR | 151. |
| | [Source:HGNC | | | HRNGGLLGRQQSGSQFALFSDILSGLPFIGFFWAKSPPESENKEQLE | |
| | Symbol; Acc:HGNC: | | | ARRRRKGTNISETSLIGTAACTSTSSQNDPEVIIVGAGVLGSALAAV | |
| | 11279] | | | LSRDGRKVTVIERDLKEPDRIVGEFLQPGGYHVLKDLGLGDTVEGL | |
| | | | | DAQVVNGYMIHDQESKSEVQIPYPLSENNQVQSGRAFHHGRFIMSL | |
| | | | | RKAAMAEPNAKFIEGVVLQLLEEDDVVMGVQYKDKETGDIKELH | |
| | | | | APLTVVADGLFSKFRKSLVSNKVSVSSHFVGFLMKNAPQFKANHA | |
| | | | | ELILANPSPVLIYQISSSETRVLVDIRGEMPRNLREYMVEKIYPQIPD | |
| | | | | HLKEPFLEATDNSHLRSMPASFLPPSSVKKRGVLLLGDAYNMRHPL | |
| | | | | TGGGMTVAFKDIKLWRKLLKGIPDLYDDAAIFEAKKSFYWARKTS | |
| | | | | HSFVVNILAQALYELFSATDDSLHQLRKACFLYFKLGGECVAGPVG | |
| | | | | LLSVLSPNPLVLIGHFFAVAIYAVYFCFKSEPWITKPRALLSSGAVLY | |
| | | | | KACSVIFPLIYSEMKYMVH* | |
| SQLE | squalene epoxidase | 6713 | 184 | MLEFHFEGEEQPLSMMKKFLKSSWVRIGFVPEEERNVLRRRKGTNI | 152. |
| | [Source:HGNC | | | SETSLIGTAACTSTSSQNDPEVIIVGAGVLGSALAAVLSRDGRKVTVI | |
| | Symbol; Acc:HGNC: | | | ERDLKEPDRIVGEFLQPGGYHVLKDLGLGDTVEGLDAQVVNGYMI | |
| | 11279] | | | HDQESKSEVQIPYPLSENNQVQSGRAFHHGRFIMSLRKAAMAEPK | |
| SQLE | squalene epoxidase | 6713 | 480 | MLRRKGTNISETSLIGTAACTSTSSQNDPEVIIVGAGVLGSALAAVL | 153. |
| | [Source HGNC | | | SRDGRKVTVIERDLKEPDRIVGEFLQPGGYHVLKDLGLGDTVEGLD | |
| | Symbol; Acc:HGNC: | | | AQVVNGYMIHDQESKSEVQIPYPLSENNQVQSGRAFHHGRFIMSLR | |
| | 11279] | | | KAAMAEPNAKFIEGVVLQLLEEDDVVMGVQYKDKETGDIKELHAP | |
| | | | | LTVVADGLFSKFRKSLVSNKVSVSSHFVGFLMKNAPQFKANHAELI | |
| | | | | LANPSPVLIYQISSSETRVLVDIRGEMPRNLREYMVEKIYPQIPDHLK | |
| | | | | EPFLEATDNSHLRSMPASFLPPSSVKKRGVLLLGDAYNMRHPLTGG | |
| | | | | GMTVAFKDIKLWRKLLKGIPDLYDDAAIFEAKKSFYWARKTSHSF | |
| | | | | VVNILAQALYELFSATDDSLHQLRKACFLYFKLGGECVAGPVGLLS | |
| | | | | VLSPNPLVLIGHFFAVAIYAVYFCFKSEPWITKPRALLSSGAVLYKA | |
| | | | | CSVIFPLIYSEMKYMVH* | |
| SQLE | squalene epoxidase | 6713 | 164 | RGEMPRNLREYMVEKIYPQIPDHLKEPFLEATDNSHLRSMPASFLPP | 154. |
| | [Source:HGNC | | | SSVKKRGVLLLGDAYNMRHPLTGGGMTVAFKDIKLWRKLLKGIPD | |
| | Symbol; Acc:HGNC: | | | LYDDAAIFEAKKSFYWARKTSHSFVVNILAQALYELFSATDDSLHQ | |
| | 11279] | | | LRKACFLYFKLGGECVAGPVGLLSV* | |
| ADAM22 | ADAM | 53616 | 907 | MQAAVAVSVPFLLLCVLGTCPPARCGQAGDASLMELEKRKENRFV | 155. |
| | metallopeptidase | | | ERQSIVPLRLIYRSGGEDESRHDALDTRVRGDLGGPQLTHVDQASF | |
| | domain 22 | | | QVDAFGTSFILDVVLNHDLLSSEYIERHIEHGGKTVEVKGGEHCYY | |
| | [Source:HGNC | | | QGHIRGNPDSFVALSTCHGLHGMFYDGNHTYLIEPEENDTTQEDFH | |
| | Symbol; Acc:HGNC: | | | FHSVYKSRLFEFSLDDLPSEFQQVNITPSKFILKPRPKRSKRQLRRYP | |
| | 201] | | | RNVEEETKYIELMIVNDHLMFKKHRLSVVHTNTYAKSVVNMADLI | |
| | | | | YKDQLKTRIVLVAMETWATDNKFAISENPLITLREFMKYRRDFIKE | |
| | | | | KSDAVHLFSGSQFESSRSGAAYIGGICSLLKGGGVNEFGKTDLMAV | |
| | | | | TLAQSLAHNIGIISDKRKLASGECKCEDTWSGCIMGDTGYYLPKKF | |
| | | | | TQCNIEEYHDFLNSGGGACLFNKPSKLLDPPECGNGFIETGEECDCG | |
| | | | | TPAECVLEGAECCKKCTLTQDSQCSDGLCCKKCKFQPMGTVCREA | |
| | | | | VNDCDIRETCSGNSSQCAPNIHKMDGYSCDGVQGICFGGRCKTRDR | |
| | | | | QCKYIWGQKVTASDKYCYEKLNIEGTEKGNCGKDKDTWIQCNKR | |
| | | | | DVLCGYLLCTNIGNIPRLGELDGEITSTLVVQQGRTLNCSGGHVKLE | |
| | | | | EDVDLGYVEDGTPCGPQMMCLEHRCLPVASFNFSTCLSSKEGTICS | |
| | | | | GNGVCSNELKCVCNRHWIGSDCNTYFPHNDDAKTGITLSGNGVAG | |
| | | | | TNIIIGIIAGTILVLALILGITAWGYKNYREQRQLPQGDYVKKPGDGD | |
| | | | | SFYSDIPPGVSTNSASSSKKRSNGLSHSWSERIPDTKHISDICENGRPR | |
| | | | | SNSWQGNLGGNKKKIRGKRFRPRSNSTETLSPAKSPSSSTGSIASSR | |
| | | | | KYPYPMPPLPDEDKKVNRQSARLWETSI* | |
| ADAM22 | ADAM | 53616 | 196 | MQAAVAVSVPFLLLCVLGTCPPARCGQAGDASLMELEKRKENRFV | 156. |
| | metallopeptidase | | | ERQSIVPLRLIYRSGGEDESRHDALDTRVRGDLGGPQIQMFLKSESQ | |
| | domain 22 | | | KTIYQIQLTHVDQASFQVDAFGTSFILDVVLNHDLLSSEYIERHIEHG | |
| | [Source:HGNC | | | GKTVEVKGGEHCYYQGHIRGNPDSFVALSTCHGLHGMFYDGNHT | |
| | Symbol; Acc:HGNC: | | | YLIEPEENDTTQ | |
| | 201] | | | | |
| ADAM22 | ADAM | 53616 | 860 | MQAAVAVSVPFLLLCVLGTCPPARCGQAGDASLMELEKRKENRFV | 157. |
| | metallopeptidase | | | ERQSIVPLRLIYRSGGEDESRHDALDTRVRGDLGGPQLTHVDQASF | |
| | domain 22 | | | QVDAFGTSFILDVVLNHDLLSSEYIERHIEHGGKTVEVKGGEHCYY | |
| | [Source:HGNC | | | QGHIRGNPDSFVALSTCHGLHGMFYDGNHTYLIEPEENDTTQEDFH | |
| | Symbol; Acc:HGNC: | | | FHSVYKSRLFEFSLDDLPSEFQQVNITPSKFILKPRPKRSKRQLRRYP | |
| | 201] | | | RNVEEETKYIELMIVNDHLMFKKHRLSVVHTNTYAKSVVNMADLI | |
| | | | | YKDQLKTRIVLVAMETWATDNKFAISENPLITLREFMKYRRDFIKE | |
| | | | | KSDAVHLFSGSQFESSRSGAAYIGGICSLLKGGGVNEFGKTDLMAV | |
| | | | | TLAQSLAHNIGIISDKRKLASGECKCEDTWSGCIMGDTGYYLPKKF | |
| | | | | TQCNIEEYHDFLNSGGGACLFNKPSKLLDPPECGNGFIETGEECDCG | |
| | | | | TPAECVLEGAECCKKCTLTQDSQCSDGLCCKKCKFQPMGTVCREA | |
| | | | | VNDCDIRETCSGNSSQCAPNIHKMDGYSCDGVQGICFGGRCKTRDR | |
| | | | | QCKYIWGQKVTASDKYCYEKLNIEGTEKGNCGKDKDTWIQCNKR | |
| | | | | DVLCGYLLCTNIGNIPRLGELDGEITSTLVVQQGRTLNCSGGHVKLE | |
| | | | | EDVDLGYVEDGTPCGPQMMCLEHRCLPVASFNFSTCLSSKEGTICS | |
| | | | | GNGVCSNELKCVCNRHWIGSDCNTYFPHNDDAKTGITLSGNGVAG | |
| | | | | TNIIIGIIAGTILVLALILGITAWGYKNYREQRQLPQGDYVKKPGDGD | |
| | | | | SFYSDIPPGVSTNSASSSKKRSNGLSHSWSERIPDTKHISDICENGRPR | |
| | | | | SNSWQGNLGGNKKKIRGKRFRPRSNSTE* | |
| ADAM22 | ADAM | 53616 | 900 | MQAAVAVSVPFLLLCVLGTCPPARCGQAGDASLMELEKRKENRFV | 158. |
| | metallopeptidase | | | ERQSIVPLRLIYRSGGEDESRHDALDTRVRGDLGGPQLTHVDQASF | |
| | domain 22 | | | QVDAFGTSFILDVVLNHDLLSSEYIERHIEHGGKTVEVKGGEHCYY | |
| | [Source:HGNC | | | QGHIRGNPDSFVALSTCHGLHGMFYDGNHTYLIEPEENDTTQEDFH | |
| | Symbol; Acc:HGNC: | | | FHSVYKSRLFEFSLDDLPSEFQQVNITPSKFILKPRPKRSKRQLRRYP | |
| | 201] | | | RNVEEETKYIELMIVNDHLMFKKHRLSVVHTNTYAKSVVNMADLI | |
| | | | | YKDQLKTRIVLVAMETWATDNKFAISENPLITLREFMKYRRDFIKE | |
| | | | | KSDAVHLFSGSQFESSRSGAAYIGGICSLLKGGGVNEFGKTDLMAV | |
| | | | | TLAQSLAHNIGIISDKRKLASGECKCEDTWSGCIMGDTGYYLPKKF | |
| | | | | TQCNIEEYHDFLNSGGGACLFNKPSKLLDPPECGNGFIETGEECDCG | |
| | | | | TPAECVLEGAECCKKCTLTQDSQCSDGLCCKKCKFQPMGTVCREA | |
| | | | | VNDCDIRETCSGNSSQCAPNIHKMDGYSCDGVQGICFGGRCKTRDR | |
| | | | | QCKYIWGQKVTASDKYCYEKLNIEGTEKGNCGKDKDTWIQCNKR | |
| | | | | DVLCGYLLCTNIGNIPRLGELDGEITSTLVVQQGRTLNCSGGHVKLE | |
| | | | | EDVDLGYVEDGTPCGPQMMCLEHRCLPVASFNFSTCLSSKEGTICS | |
| | | | | GNGVCSNELKCVCNRHWIGSDCNTYFPHNDDAKTGITLSGNGVAG | |
| | | | | TNIIIGIIAGTILVLALILGITAWGYKNYREQRSNGLSHSWSERIPDTK | |
| | | | | HISDICENGRPRSNSWQGNLGGNKKKIRGKRFRPRSNSTEYLNPWF | |
| | | | | KRDYNVAKWVEDVNKNTEGPYFRTLSPAKSPSSSTGSIASSRKYPY | |
| | | | | PMPPLPDEDKKVNRQSARLWETSI* | |
| ADAM22 | ADAM | 53616 | 804 | MELEKRKENRFVERQSIVPLRLIYRSGGEDESRHDALDTRVRGDLG | 159. |
| | metallopeptidase | | | GPQLTHVDQASFQVDAFGTSFILDVVLNHDLLSSEYIERHIEHGGKT | |
| | domain 22 | | | VEVKGGEHCYYQGHIRGNPDSFVALSTCHGLHGMFYDGNHTYLIE | |
| | [Source HGNC | | | PEENDTTQEDFHFHSVYKSRLFEFSLDDLPSEFQQVNITPSKFILKPR | |
| | Symbol; Acc:HGNC: | | | PKRSKRQLRRYPRNVEEETKYIELMIVNDHLMFKKHRLSVVHTNTY | |
| | 201] | | | AKSVVNMADLIYKDQLKTRIVLVAMETWATDNKFAISENPLITLRE | |
| | | | | FMKYRRDFIKEKSDAVHLFSGSQFESSRSGAAYIGGICSLLKGGGVN | |
| | | | | EFGKTDLMAVTLAQSLAHNIGIISDKRKLASGECKCEDTWSGCIMG | |
| | | | | DTGYYLPKKFTQCNIEEYHDFLNSGGGACLFNKPSKLLDPPECGNG | |
| | | | | FIETGEECDCGTPAECVLEGAECCKKCTLTQDSQCSDGLCCKKCKF | |
| | | | | QPMGTVCREAVNDCDIRETCSGNSSQCAPNIHKMDGYSCDGVQGI | |
| | | | | CFGGRCKTRDRQCKYIWGQKVTASDKYCYEKLNIEGTEKGNCGKD | |
| | | | | KDTWIQCNKRDVLCGYLLCTNIGNIPRLGELDGEITSTLVVQQGRTL | |
| | | | | NCSGGHVKLEEDVDLGYVEDGTPCGPQMMCLEHRCLPVASFNFST | |
| | | | | CLSSKEGTICSGNGVCSNELKCVCNRHWIGSDCNTYFPHNDDAKTG | |
| | | | | ITLSGNGVAGTNIIIGIIAGTILVLALILGITAWGYKNYREQRQLPQG | |
| | | | | DYVKKPGDGDSFYSDIPPGVSTNSASSSKKRSNGLSHSWSERIPDTK | |
| | | | | HISDICENGRPRSNSWQG | |
| ADAM22 | ADAM | 53616 | 871 | MQAAVAVSVPFLLLCVLGTCPPARCGQAGDASLMELEKRKENRFV | 160. |
| | metallopeptidase | | | ERQSIVPLRLIYRSGGEDESRHDALDTRVRGDLGGPQLTHVDQASF | |
| | domain 22 | | | QVDAFGTSFILDVVLNHDLLSSEYIERHIEHGGKTVEVKGGEHCYY | |
| | [Source HGNC | | | QGHIRGNPDSFVALSTCHGLHGMFYDGNHTYLIEPEENDTTQEDFH | |
| | Symbol; Acc:HGNC: | | | FHSVYKSRLFEFSLDDLPSEFQQVNITPSKFILKPRPKRSKRQLRRYP | |
| | 201] | | | RNVEEETKYIELMIVNDHLMFKKHRLSVVHTNTYAKSVVNMADLI | |
| | | | | YKDQLKTRIVLVAMETWATDNKFAISENPLITLREFMKYRRDFIKE | |
| | | | | KSDAVHLFSGSQFESSRSGAAYIGGICSLLKGGGVNEFGKTDLMAV | |
| | | | | TLAQSLAHNIGIISDKRKLASGECKCEDTWSGCIMGDTGYYLPKKF | |
| | | | | TQCNIEEYHDFLNSGGGACLFNKPSKLLDPPECGNGFIETGEECDCG | |
| | | | | TPAECVLEGAECCKKCTLTQDSQCSDGLCCKKCKFQPMGTVCREA | |
| | | | | VNDCDIRETCSGNSSQCAPNIHKMDGYSCDGVQGICFGGRCKTRDR | |
| | | | | QCKYIWGQKVTASDKYCYEKLNIEGTEKGNCGKDKDTWIQCNKR | |
| | | | | DVLCGYLLCTNIGNIPRLGELDGEITSTLVVQQGRTLNCSGGHVKLE | |
| | | | | EDVDLGYVEDGTPCGPQMMCLEHRCLPVASFNFSTCLSSKEGTICS | |
| | | | | GNGVCSNELKCVCNRHWIGSDCNTYFPHNDDAKTGITLSGNGVAG | |
| | | | | TNIIIGIIAGTILVLALILGITAWGYKNYREQRSNGLSHSWSERIPDTK | |
| | | | | HISDICENGRPRSNSWQGNLGGNKKKIRGKRFRPRSNSTETLSPAKS | |
| | | | | PSSSTGSIASSRKYPYPMPPLPDEDKKVNRQSARLWETSI* | |
| ADAM22 | ADAM | 53616 | 343 | MQAAVAVSVPFLLLCVLGTCPPARCGQAGDASLMELEKRKENRFV | 161. |
| | metallopeptidase | | | ERQSIVPLRLIYRSGGEDESRHDALDTRVRGDLGGPQLTHVDQASF | |
| | domain 22 | | | QVDAFGTSFILDVVLNHDLLSSEYIERHIEHGGKTVEVKGGEHCYY | |
| | [Source HGNC | | | QGHIRGNPDSFVALSTCHGLHGMFYDGNHTYLIEPEENDTTQEDFH | |
| | Symbol; Acc:HGNC: | | | FHSVYKSRLFEFSLDDLPSEFQQVNITPSKFILKPRPKRSKRQLRRYP | |
| | 201] | | | RNVEEETKYIELMIVNDHLMFKKHRLSVVHTNTYAKSVVNMADLI | |
| | | | | YKDQLKTRIVLVAMETWATDNKFAISENPLITLREFMKYRRDFIKE | |
| | | | | KSDAVHLFSYVTSVMMYYFF* | |
| ADAM22 | ADAM | 53616 | 265 | XCNRHWIGSDCNTYFPHNDDAKTGITLSGNGVAGTNIIIGIIAGTILV | 162. |
| | metallopeptidase | | | LALILGITAWGYKNYREQRQLPQGDYVKKPGDGDSFYSDIPPGVST | |
| | domain 22 | | | NSASSSKKRSAFLSHFQISTCSITHYSISQNISLFCSRSNGLSHSWSERI | |
| | [Source HGNC | | | PDTKHISDICENGRPRSNSWQGNLGGNKKKIRGKRFRPRSNSTEYL | |
| | Symbol; Acc:HGNC: | | | NPWFKRDYNVAKWVEDVNKNTEGPYFRTLSPAKSPSSSTGSIASSR | |
| | 201] | | | KYPYPMPPLPDEDKKVNRQSARLWETSI* | |
| ADAM22 | ADAM | 53616 | 331 | EEDVDLGYVEDGTPCGPQMMCLEHRCLPVASFNFSTCLSSKEGTIC | 163. |
| | metallopeptidase | | | SGNGVCSNELKCVCNRHWIGSDCNTYFPHNDDAKTGITLSGNGVA | |
| | domain 22 | | | GTNIIIGIIAGTILVLALILGITAWGYKNYREQRQLPQGDYVKKPGDG | |
| | [Source HGNC | | | DSFYSDIPPGVSTNSASSSKKRSNGLSHSWSERIPDTKHISDICENGR | |
| | Symbol; Acc:HGNC: | | | PRSNSWQGNLGGNKKKIRGKRFRPRSNSTEREPQAPEPGHSLAQTV | |
| | 201] | | | PSQGISPGGSDSPQTGSLDHRYLNPWFKRDYNVAKWVEDVNKNTE | |
| | | | | GPYFRTLSPAKSPSSSTGSIASSRKYPYPMPPLPDEDKKVNRQSARL | |
| | | | | WETSI* | |
| NLRC5 | NLR family CARD | 84166 | 1867 | MDPVGLQLGNKNLWSCLVRLLTKDPEWLNAKMKFFLPNTDLDSR | 164. |
| | domain containing 5 | | | NETLDPEQRVILQLNKLHVQGSDTWQSFIHCVCMQLEVPLDLEVLL | |
| | [Source HGNC | | | LSTFGYDDGFTSQLGAEGKSQPESQLHHGLKRPHQSCGSSPRRKQC | |
| | Symbol; Acc:HGNC: | | | KKQQLELAKKYLQLLRTSAQQRYRSQIPGSGQPHAFHQVYVPPILR | |
| | 29933] | | | RATASLDTPEGAIMGDVKVEDGADVSISDLFNTRVNKGPRVTVLLG | |
| | | | | KAGMGKTTLAHRLCQKWAEGHLNCFQALFLFEFRQLNLITRFLTPS | |
| | | | | ELLFDLYLSPESDHDTVFQYLEKNADQVLLIFDGLDEALQPMGPDG | |
| | | | | PGPVLTLFSHLCNGTLLPGCRVMATSRPGKLPACLPAEAAMVHML | |
| | | | | GFDGPRVEEYVNHFFSAQPSREGALVELQTNGRLRSLCAVPALCQV | |
| | | | | ACLCLHHLLPDHAPGQSVALLPNMTQLYMQMVLALSPPGHLPTSS | |
| | | | | LLDLGEVALRGLETGKVIFYAKDIAPPLIAFGATHSLLTSFCVCTGP | |
| | | | | GHQQTGYAFTHLSLQEFLAALHLMASPKVNKDTLTQYVTLHSRWV | |
| | | | | QRTKARLGLSDHLPTFLAGLASCTCRPFLSHLAQGNEDCVGAKQA | |
| | | | | AVVQVLKKLATRKLTGPKVVELCHCVDETQEPELASLTAQSLPYQ | |
| | | | | LPFHNFPLTCTDLATLTNILEHREAPIHLDFDGCPLEPHCPEALVGCG | |
| | | | | QIENLSFKSRKCGDAFAEALSRSLPTMGRLQMLGLAGSKITARGISH | |
| | | | | LVKALPLCPQLKEVSFRDNQLSDQVVLNIVEVLPHLPRLRKLDLSSN | |
| | | | | SICVSTLLCLARVAVTCPTVRMLQAREADLIFLLSPPTETTAELQRA | |
| | | | | PDLQESDGQRKGAQSRSLTLRLQKCQLQVHDAEALIALLQEGPHLE | |
| | | | | EVDLSGNQLEDEGCRLMAEAASQLHIARKLDLSNNGLSVAGVHCV | |
| | | | | LRAVSACWTLAELHISLQHKTVIFMFAQEPEEQKGPQERAAFLDSL | |
| | | | | MLQMPSELPLSSRRMRLTHCGLQEKHLEQLCKALGGSCHLGHLHL | |
| | | | | DFSGNALGDEGAARLAQLLPGLGALQSLNLSENGLSLDAVLGLVR | |
| | | | | CFSTLQWLFRLDISFESQHILLRGDKTSRDMWATGSLPDFPAAAKFL | |
| | | | | GFRQRCIPRSLCLSECPLEPPSLTRLCATLKDCPGPLELQLSCEFLSD | |
| | | | | QSLETLLDCLPQLPQLSLLQLSQTGLSPKSPFLLANTLSLCPRVKKV | |
| | | | | DLRSLHHATLHFRSNEEEEGVCCGRFTGCSLSQEHVESLCWLLSKC | |
| | | | | KDLSQVDLSANLLGDSGLRCLLECLPQVPISGLLDLSHNSISQESAL | |
| | | | | YLLETLPSCPRVREASVNLGSEQSFRIHFSREDQAGKTLRLSECSFRP | |
| | | | | EHVSRLATGLSKSLQLTELTLTQCCLGQKQLAILLSLVGRPAGLFSL | |
| | | | | RVQEPWADRARVLSLLEVCAQASGSVTEISISETQQQLCVQLEFPRQ | |
| | | | | EENPEAVALRLAHCDLGAHHSLLVGQLMETCARLQQLSLSQVNLC | |
| | | | | EDDDASSLLLQSLLLSLSELKTFRLTSSCVSTEGLAHLASGLGHCHH | |
| | | | | LEELDLSNNQFDEEGTKALMRALEGKWMLKRLDLSHLLLNSSTLA | |
| | | | | LLTHRLSQMTCLQSLRLNRNSIGDVGCCHLSEALRAATSLEELDLS | |
| | | | | HNQIGDAGVQHLATILPGLPELRKIDLSGNSISSAGGVQLAESLVLC | |
| | | | | RRLEELMLGCNALGDPTALGLAQELPQHLRVLHLPFSHLGPGGALS | |
| | | | | LAQALDGSPHLEEISLAENNLAGGVLRFCMELPLLRQIDLVSCKIDN | |
| | | | | QTAKLLTSSFTSCPALEVILLSWNLLGDEAAAELAQVLPQMGRLKR | |
| | | | | VDLEKNQITALGAWLLAEGLAQGSSIQVIRLWNNPIPCDMAQHLKS | |
| | | | | QEPRLDFAFFDNQPQAPWGT* | |
| NLRC5 | NLR family CARD | 84166 | 158 | XLSANLLGDSGLRCLLECLPQVPISGLLDLSHNSISQESALYLLETLP | 165. |
| | domain containing 5 | | | SCPRVREASVNLGSEQSFRIHFSREDQAGKTLRLSECSFRPEHVSRL | |
| | [Source:HGNC | | | ATGLSKSLQLTELTLTQCCLGQKQLAILLSLVGRPAGLFSLRLHKAH | |
| | Symbol; Acc:HGNC: | | | RETVLPHTGGLCFGI* | |
| | 29933] | | | | |
| NLRC5 | NLR family CARD | 84166 | 631 | LTCTDLATLTNILEHREAPIHLDFDGCPLEPHCPEALVGCGQIENLSF | 166. |
| | domain containing 5 | | | KSRKCGDAFAEALSRSLPTMGRLQMLGLSSNSICVSTLLCLARVAV | |
| | [Source:HGNC | | | TCPTVRMLQAREADLIFLLSPPTETTAELQRAPDLQESDGQRKGAQ | |
| | Symbol; Acc:HGNC: | | | SRSLTLRLQKCQLQVHDAEALIALLQEGPHLEEVDLSGNQLEDEGC | |
| | 29933] | | | RLMAEAASQLHIARKLDLSNNGLSVAGVHCVLRAVSACWTLAELH | |
| | | | | ISLQHKTVIFMFAQEPEEQKGPQERAAFLDSLMLQMPSELPLSSRRM | |
| | | | | RLTHCGLQEKHLEQLCKALGGSCHLGHLHLDFSGNALGDEGAARL | |
| | | | | AQLLPGLGALQSLNLSENGLSLDAVLGLVRCFSTLQWLFRLDISFES | |
| | | | | QHILLRGDKTSSLSECPLEPPSLTRLCATLKDCPGPLELQLSCEFLSD | |
| | | | | QSLETLLDCLPQLPQLSLLQLSQTGLSPKSPFLLANTLSLCPRVKKV | |
| | | | | DLRSLHHATLHFRSNEEEEGVCCGRFTGCSLSQEHVESLCWLLSKC | |
| | | | | KDLSQVDLGSEQSFRIHFSREDQAGKTLRLSECSFRPEHVSRLATGL | |
| | | | | SKSLQLTELTLTQCCLGQKQLAILLSLVGRPAGLFSLRDSTRHTEKL | |
| | | | | SFPTLEACALGFKKKRKKERKKVSED | |
| NLRC5 | NLR family CARD | 84166 | 388 | XELPLSSRRMRLTHCGLQEKHLEQLCKALGGSCHLGHLHLDFSGN | 167. |
| | domain containing 5 | | | ALGDEGAARLAQLLPGLGALQSLNLSENGLSLDAVLGLVRCFSTLQ | |
| | [Source:HGNC | | | WLFRLDISFESQHILLRGDKTSRDMWATGSLPDFPAAAKFLGFRQR | |
| | Symbol; Acc:HGNC: | | | CIPRSLCLSECPLEPPSLTRLCATLKDCPGPLELQLSCEFLSDQSLETL | |
| | 92933] | | | LDCLPQLPQLSLLQLSQTGLSPKSPFLLANTLSLCPRVKKVDLRSLH | |
| | | | | HATLHFRSNEEEEGVCCGLSANLLGDSGLRCLLECLPQVPISGLLDL | |
| | | | | SHNSISQESALYLLETLPSCPRVREASVNLGSEQSFRIHFSREDQAGK | |
| | | | | TLRLSECSFRPEHVSRLATGLSKSLQLTELTLTQCCLGQKQLAILLSL | |
| | | | | VGRPAGLFSLS* | |
| NLRC5 | NLR family CARD | 84166 | 1838 | MDPVGLQLGNKNLWSCLVRLLTKDPEWLNAKMKFFLPNTDLDSR | 168. |
| | domain containing 5 | | | NETLDPEQRVILQLNKLHVQGSDTWQSFIHCVCMQLEVPLDLEVLL | |
| | [Source:HGNC | | | LSTFGYDDGFTSQLGAEGKSQPESQLHHGLKRPHQSCGSSPRRKQC | |
| | Symbol; Acc:HGNC: | | | KKQQLELAKKYLQLLRTSAQQRYRSQIPGSGQPHAFHQVYVPPILR | |
| | 29933] | | | RATASLDTPEGAIMGDVKVEDGADVSISDLFNTRVNKGPRVTVLLG | |
| | | | | KAGMGKTTLAHRLCQKWAEGHLNCFQALFLFEFRQLNLITRFLTPS | |
| | | | | ELLFDLYLSPESDHDTVFQYLEKNADQVLLIFDGLDEALQPMGPDG | |
| | | | | PGPVLTLFSHLCNGTLLPGCRVMATSRPGKLPACLPAEAAMVHML | |
| | | | | GFDGPRVEEYVNHFFSAQPSREGALVELQTNGRLRSLCAVPALCQV | |
| | | | | ACLCLHHLLPDHAPGQSVALLPNMTQLYMQMVLALSPPGHLPTSS | |
| | | | | LLDLGEVALRGLETGKVIFYAKDIAPPLIAFGATHSLLTSFCVCTGP | |
| | | | | GHQQTGYAFTHLSLQEFLAALHLMASPKVNKDTLTQYVTLHSRWV | |
| | | | | QRTKARLGLSDHLPTFLAGLASCTCRPFLSHLAQGNEDCVGAKQA | |
| | | | | AVVQVLKKLATRKLTGPKVVELCHCVDETQEPELASLTAQSLPYQ | |
| | | | | LPFHNFPLTCTDLATLTNILEHREAPIHLDFDGCPLEPHCPEALVGCG | |
| | | | | QIENLSFKSRKCGDAFAEALSRSLPTMGRLQMLGLAGSKITARGISH | |
| | | | | LVKALPLCPQLKEVSFRDNQLSDQVVLNIVEVLPHLPRLRKLDLSSN | |
| | | | | SICVSTLLCLARVAVTCPTVRMLQAREADLIFLLSPPTETTAELQRA | |
| | | | | PDLQESDGQRKGAQSRSLTLRLQKCQLQVHDAEALIALLQEGPHLE | |
| | | | | EVDLSGNQLEDEGCRLMAEAASQLHIARKLDLSNNGLSVAGVHCV | |
| | | | | LRAVSACWTLAELHISLQHKTVIFMFAQEPEEQKGPQERAAFLDSL | |
| | | | | MLQMPSELPLSSRRMRLTHCGLQEKHLEQLCKALGGSCHLGHLHL | |
| | | | | DFSGNALGDEGAARLAQLLPGLGALQSLNLSENGLSLDAVLGLVR | |
| | | | | CFSTLQWLFRLDISFESQHILLRGDKTSRDMWATGSLPDFPAAAKFL | |
| | | | | GFRQRCIPRSLCLSECPLEPPSLTRLCATLKDCPGPLELQLSCEFLSD | |
| | | | | QSLETLLDCLPQLPQLSLLQLSQTGLSPKSPFLLANTLSLCPRVKKV | |
| | | | | DLRSLHHATLHFRSNEEEEGVCCGLSANLLGDSGLRCLLECLPQVPI | |
| | | | | SGLLDLSHNSISQESALYLLETLPSCPRVREASVNLGSEQSFRIHFSR | |
| | | | | EDQAGKTLRLSECSFRPEHVSRLATGLSKSLQLTELTLTQCCLGQK | |
| | | | | QLAILLSLVGRPAGLFSLRVQEPWADRARVLSLLEVCAQASGSVTEI | |
| | | | | SISETQQQLCVQLEFPRQEENPEAVALRLAHCDLGAHHSLLVGQLM | |
| | | | | ETCARLQQLSLSQVNLCEDDDASSLLLQSLLLSLSELKTFRLTSSCV | |
| | | | | STEGLAHLASGLGHCHHLEELDLSNNQFDEEGTKALMRALEGKW | |
| | | | | MLKRLDLSHLLLNSSTLALLTHRLSQMTCLQSLRLNRNSIGDVGCC | |
| | | | | HLSEALRAATSLEELDLSHNQIGDAGVQHLATILPGLPELRKIDLSG | |
| | | | | NSISSAGGVQLAESLVLCRRLEELMLGCNALGDPTALGLAQELPQH | |
| | | | | LRVLHLPFSHLGPGGALSLAQALDGSPHLEEISLAENNLAGGVLRFC | |
| | | | | MELPLLRQIDLVSCKIDNQTAKLLTSSFTSCPALEVILLSWNLLGDE | |
| | | | | AAAELAQVLPQMGRLKRVDLEKNQITALGAWLLAEGLAQGSSIQV | |
| | | | | IRLWNNPIPCDMAQHLKSQEPRLDFAFFDNQPQAPWGT* | |
| NLRC5 | NLR family CARD | 84166 | 1058 | XSELLFDLYLSPESDHDTVFQYLEKNADQVLLIFDGLDEALQPMGP | 169. |
| | domain containing 5 | | | DGPGPVLTLFSHLCNGTLLPGCRVMATSRPGKLPACLPAEAAMVH | |
| | [Source:HGNC | | | MLGFDGPRVEEYVNHFFSAQPSREGALVELQTNGRLRSLCAVPALC | |
| | Symbol; Acc:HGNC: | | | QVACLCLHHLLPDHAPGQSVALLPNMTQLYMQMVLALSPPGHLPT | |
| | 29933] | | | SSLLDLGEVALRGLETGKVIFYAKDIAPPLIAFGATHSLLTSFCVCTG | |
| | | | | PGHQQTGYAFTHLSLQEFLAALHLMASPKVNKDTLTQYVTLHSRW | |
| | | | | VQRTKARLGLSDHLPTFLAGLASCTCRPFLSHLAQGNEDCVGAKQ | |
| | | | | AAVVQVLKKLATRKLTGPKVVELCHCVDETQEPELASLTAQSLPY | |
| | | | | QLPFHNFPLTCTDLATLTNILEHREAPIHLDFDGCPLEPHCPEALVGC | |
| | | | | GQIENLSFKSRKCGDAFAEALSRSLPTMGRLQMLGLAGSKITARGIS | |
| | | | | HLVKALPLCPQLKEVSFRDNQLSDQVVLNIVEVLPHLPRLRKLDLSS | |
| | | | | NSICVSTLLCLARVAVTCPTVRMLQAREADLIFLLSPPTETTAELQR | |
| | | | | APDLQESDGQRKGAQSRSLTLRLQKCQLQVHDAEALIALLQEGPHL | |
| | | | | EEVDLSGNQLEDEGCRLMAEAASQLHIARKLDLSNNGLSVAGVHC | |
| | | | | VLRAVSACWTLAELHISLQHKTVIFMFAQEPEEQKGPQERAAFLDS | |
| | | | | LMLQMPSELPLSSRRMRLTHCGLQEKHLEQLCKALGGSCHLGHLH | |
| | | | | LDFSGNALGDEGAARLAQLLPGLGALQSLNLSENGLSLDAVLGLV | |
| | | | | RCFSTLQWLFRLDISFESQHILLRGDKTSRDMWATGSLPDFPAAAKF | |
| | | | | LGFRQRCIPRSLCLSECPLEPPSLTRLCATLKDCPGPLELQLSCEFLSD | |
| | | | | QSLETLLDCLPQLPQLSLLQLSQTGLSPKSPFLLANTLSLCPRVKKV | |
| | | | | DLRSLHHATLHFRSNEEEEGVCCGRFTGCSLSQEHVESLCWLLSKC | |
| | | | | KDLSQVDLSANLLGDSGLRCLLECLPQVPISGLLDLSHNSISQESAL | |
| | | | | YLLETLPSCPRVREASVKCLGSWHVWAPCFPRPFSLQPGL* | |
| NLRC5 | NLR family CARD | 84166 | 74 | MDPVGLQLGNKNLWSCLVRLLTKDPEWLNAKMKFFLPNTDLDSR | 170. |
| | domain containing 5 | | | NETLDPEQRVILQLNKLHVQGSDTWQSFIH | |
| | [Source:HGNC | | | | |
| | Symbol; Acc:HGNC: | | | | |
| | 29933] | | | | |
| NLRC5 | NLR family CARD | 84166 | 111 | XASGLGHCHHLEELDLSNNQFDEEGTKALMRALEGKWMLKRLDL | 171. |
| | domain containing 5 | | | SHLLLNSSTLALLTHRLSQMTCLQSLSLTLSPRLECSGMISLHCNLCL | |
| | [Source:HGNC | | | LGSSDSGASAAQVAGITD* | |
| | Symbol; Acc:HGNC: | | | | |
| | 29933] | | | | |
| NLRC5 | NLR family CARD | 84166 | 703 | QKCQLQVHDAEALIALLQEGPHLEEVDLSGNQLEDEGCRLMAEAA | 172. |
| | domain containing 5 | | | SQLHIARKLDLSNNGLSVAGVHCVLRAVSACWTLAELHISLQHKTV | |
| | [Source:HGNC | | | IFMFAQEPEEQKGPQERAAFLDSLMLQMPSELPLSSRRMRLTHCGL | |
| | Symbol; Acc:HGNC: | | | QEKHLEQLCKALGGSCHLGHLHLDFSGNALGDEGAARLAQLLPGL | |
| | 29933] | | | GALQSLNLSENGLSLDAVLGLVRCFSTLQWLFRLDISFESQHILLRG | |
| | | | | DKTSRDMWATGSLPDFPAAAKFLGFRQRCIPRSLCLSECPLEPPSLT | |
| | | | | RLCATLKDCPGPLELQLSCEFLSDQSLETLLDCLPQLPQLSLLQLSQT | |
| | | | | GLSPKSPFLLANTLSLCPRVKKVDLRSLHHATLHFRSNEEEEGVCCG | |
| | | | | RFTGCSLSQEHVESLCWLLSKCKDLSQVDLSANLLGDSGLRCLLEC | |
| | | | | LPQVPISGLLDLSHNSISQESALYLLETLPSCPRVREASVNLGSEQSF | |
| | | | | RIHFSREDQAGKTLRLSECSFRPEHVSRLATGLSKSLQLTELTLTQC | |
| | | | | CLGQKQLAILLSLVGRPAGLFSLRVQEPWADRARVLSLLEVCAQAS | |
| | | | | GSVTEISISETQQQLCVQLEFPRQEENPEAVALRLAHCDLGAHHSLL | |
| | | | | VGQLMETCARLQQLSLSQVNLCEDDDASSLLLQSLLLSLSELKTFRI | |
| | | | | QSSQDPGRLLPDLLPFDPHQADLQLCEHRGPRPPGIWSGPLPPLGGA | |
| | | | | GLV* | |
| NLRC5 | NLR family CARD | 84166 | 1181 | XHLNCFQALFLFEFRQLNLITRFLTPSELLFDLYLSPESDHDTVFQYL | 173. |
| | domain containing 5 | | | EKNADQVLLIFDGLDEALQPMGPDGPGPVLTLFSHLCNGTLLPGCR | |
| | [Source:HGNC | | | VMATSRPGKLPACLPAEAAMVHMLGFDGPRVEEYVNHFFSAQPSR | |
| | Symbol; Acc:HGNC: | | | EGALVELQTNGRLRSLCAVPALCQVACLCLHHLLPDHAPGQSVAL | |
| | 29933] | | | LPNMTQLYMQMVLALSPPGHLPTSSLLDLGEVALRGLETGKVIFYA | |
| | | | | KDIAPPLIAFGATHSLLTSFCVCTGPGHQQTGYAFTHLSLQEFLAAL | |
| | | | | HLMASPKVNKDTLTQYVTLHSRWVQRTKARLGLSDHLPTFLAGLA | |
| | | | | SCTCRPFLSHLAQGNEDCVGAKQAAVVQVLKKLATRKLTGPKVVE | |
| | | | | LCHCVDETQEPELASLTAQSLPYQLPFHNFPLTCTDLATLTNILEHR | |
| | | | | EAPIHLDFDGCPLEPHCPEALVGCGQIENLSFKSRKCGDAFAEALSR | |
| | | | | SLPTMGRLQMLGLAGSKITARGISHLVKALPLCPQLKEVSFRDNQL | |
| | | | | SDQVVLNIVEVLPHLPRLRKLDLSSNSICVSTLLCLARVAVTCPTVR | |
| | | | | MLQAREADLIFLLSPPTETTAELQRAPDLQESDGQRKGAQSRSLTLR | |
| | | | | LQKCQLQVHDAEALIALLQEGPHLEEVDLSGNQLEDEGCRLMAEA | |
| | | | | ASQLHIARKLDLSNNGLSVAGVHCVLRAVSACWTLAELHISLQHKT | |
| | | | | VIFMFAQEPEEQKGPQERAAFLDSLMLQMPSELPLSSRRMRLTHCG | |
| | | | | LQEKHLEQLCKALGGSCHLGHLHLDFSGNALGDEGAARLAQLLPG | |
| | | | | LGALQSLNLSENGLSLDAVLGLVRCFSTLQWLFRLDISFESQHILLR | |
| | | | | GDKTSRDMWATGSLPDFPAAAKFLGFRQRCIPRSLCLSECPLEPPSL | |
| | | | | TRLCATLKDCPGPLELQLSCEFLSDQSLETLLDCLPQLPQLSLLQLSQ | |
| | | | | TGLSPKSPFLLANTLSLCPRVKKVDLRSLHHATLHFRSNEEEEGVCC | |
| | | | | GFTGCSLSQEHVESLCWLLSKCKDLSQVDLSANLLGDSGLRCLLEC | |
| | | | | LPQVPISGLLDLSHNSISQESALYLLETLPSCPRVREASVNLGSEQSF | |
| | | | | RIHFSREDQAGKTLRLSECSFRPEHVSRLATGLSKSLQLTELTLTQC | |
| | | | | CLGQKQLAILLSLVGRPAGLFSLRDSTRHTEKLSFPTLEACALGFKK | |
| | | | | KRKKERKKVSEDTELRTKIN* | |
| NLRC5 | NLR family CARD | 84166 | 885 | LTSFCVCTGPGHQQTGYAFTHLSLQEFLAALHLMASPKVNKDTLTQ | 174. |
| | domain containing 5 | | | YVTLHSRWVQRTKARLGLSDHLPTFLAGLASCTCRPFLSHLAQGNE | |
| | [Source:HGNC | | | DCVGAKQAAVVQVLKKLATRKLTGPKVVELCHCVDETQEPELASL | |
| | Symbol; Acc:HGNC: | | | TAQSLPYQLPFHNFPLTCTDLATLTNILEHREAPIHLDFDGCPLEPHC | |
| | 29933] | | | PEALVGCGQIENLSFKSRKCGDAFAEALSRSLPTMGRLQMLGLAGS | |
| | | | | KITARGISHLVKALPLCPQLKEVSFRDNQLSDQVVLNIVEVLPHLPR | |
| | | | | LRKLDLSSNSICVSTLLCLARVAVTCPTVRMLQAREADLIFLLSPPTE | |
| | | | | TTAELQRAPDLQESDGQRKGAQSRSLTLRLQKCQLQVHDAEALIAL | |
| | | | | LQEGPHLEEVDLSGNQLEDEGCRLMAEAASQLHIARKLDLSNNGLS | |
| | | | | VAGVHCVLRAVSACWTLAELHISLQHKTVIFMFAQEPEEQKGPQER | |
| | | | | AAFLDSLMLQMPSELPLSSRRMRLTHCGLQEKHLEQLCKALGGSC | |
| | | | | HLGHLHLDFSGNALGDEGAARLAQLLPGLGALQSLNLSENGLSLD | |
| | | | | AVLGLVRCFSTLQWLFRLDISFESQHILLRGDKTSSLSECPLEPPSLT | |
| | | | | RLCATLKDCPGPLELQLSCEFLSDQSLETLLDCLPQLPQLSLLQLSQT | |
| | | | | GLSPKSPFLLANTLSLCPRVKKVDLRSLHHATLHFRSNEEEEGVCCG | |
| | | | | FTGCSLSQEHVESLCWLLSKCKDLSQVDLSANLLGDSGLRCLLECL | |
| | | | | PQVPISGLLDLSHNSISQESALYLLETLPSCPRVREASVNLGSEQSFRI | |
| | | | | HFSREDQAGKTLRLSECSFRPEHVSRLATGLSKSLQLTELTLTQCCL | |
| | | | | GQKQLAILLSLVGRPAGLFSLRDSTRHTEKLSFPTLEACALGFKKK | |
| NLRC5 | NLR family CARD | 84166 | 326 | XWLFRLDISFESQHILLRGDKTSRDMWATGSLPDFPAAAKFLGFRQ | 175. |
| | domain containing 5 | | | RCIPRSLCLSECPLEPPSLTRLCATLKDCPGPLELQLSCEFLSDQSLET | |
| | [Source:HGNC | | | LLDCLPQLPQLSLLQLSQTGLSPKSPFLLANTLSLCPRVKKVDLRSL | |
| | Symbol; Acc:HGNC: | | | HHATLHFRSNEEEEGVCCGFTGCSLSQEHVESLCWLLSKCKDLSQV | |
| | 29933] | | | DLSANLLGDSGLRCLLECLPQVPISGLLDLSHNSISQESALYLLETLP | |
| | | | | SCPRVREASVNLGSEQSFRIHFSREDQAGKTLRLSECSFRPEHVSRL | |
| | | | | ATGLSKSLQLTELTLTQCCLGQKQLAILLSLVGRPAGLFSLS* | |
| NLRC5 | NLR family CARD | 84166 | 721 | MDPVGLQLGNKNLWSCLVRLLTKDPEWLNAKMKFFLPNTDLDSR | 176. |
| | domain containing 5 | | | NETLDPEQRVILQLNKLHVQGSDTWQSFIHCVCMQLEVPLDLEVLL | |
| | [Source:HGNC | | | LSTFGYDDGFTSQLGAEGKSQPESQLHHGLKRPHQSCGSSPRRKQC | |
| | Symbol; Acc:HGNC: | | | KKQQLELAKKYLQLLRTSAQQRYRSQIPGSGQPHAFHQVYVPPILR | |
| | 29933] | | | RATASLDTPEGAIMGDVKVEDGADVSISDLFNTRVNKGPRVTVLLG | |
| | | | | KAGMGKTTLAHRLCQKWAEGHLNCFQALFLFEFRQLNLITRFLTPS | |
| | | | | ELLFDLYLSPESDHDTVFQYLEKNADQVLLIFDGLDEALQPMGPDG | |
| | | | | PGPVLTLFSHLCNGTLLPGCRVMATSRPGKLPACLPAEAAMVHML | |
| | | | | GFDGPRVEEYVNHFFSAQPSREGALVELQTNGRLRSLCAVPALCQV | |
| | | | | ACLCLHHLLPDHAPGQSVALLPNMTQLYMQMVLALSPPGHLPTSS | |
| | | | | LLDLGEVALRGLETGKVIFYAKDIAPPLIAFGATHSLLTSFCVCTGP | |
| | | | | GHQQTGYAFTHLSLQEFLAALHLMASPKVNKDTLTQYVTLHSRWV | |
| | | | | QRTKARLGLSDHLPTFLAGLASCTCRPFLSHLAQGNEDCVGAKQA | |
| | | | | AVVQVLKKLATRKLTGPKVVELCHCVDETQEPELASLTAQSLPYQ | |
| | | | | LPFHNFPLTCTDLATLTNILEHREAPIHLDFDGCPLEPHCPEALVGCG | |
| | | | | QIENLSFKSRKCGDAFAEALSRSLPTMGRLQMLG* | |
| NLRC5 | NLR family CARD | 84166 | 19 | MDPVGLQLGNKNLWSCLVR | 177. |
| | domain containing 5 | | | | |
| | [Source:HGNC | | | | |
| | Symbol; Acc:HGNC: | | | | |
| | 29933] | | | | |
| IRF1 | interferon regulatory | 3659 | 326 | MPITRMRMRPWLEMQINSNQIPGLIWINKEEMIFQIPWKHAAKHGW | 178. |
| | factor 1 [Source:HGNC | | | DINKDACLFRSWAIHTGRYKAGEKEPDPKTWKANFRCAMNSLPDI | |
| | Symbol; Acc:HGNC: | | | EEVKDQSRNKGSSAVRVYRMLPPLTKNQRKERKSKSSRDAKSKAK | |
| | 6116] | | | RKSCGDSSPDTFSDGLSSSTLPDDHSSYTVPGYMQDLEVEQALTPA | |
| | | | | LSPCAVSSTLPDWHIPVEVVPDSTSDLYNFQVSPMPSTSEATTDEDE | |
| | | | | EGKLPEDIMKLLEQSEWQPTNVDGKGYLLNEPGVQPTSVYGDFSC | |
| | | | | KEEPEIDSPGGDIGLSLQRVFTDLKNMDATWLDSLLTPVRLPSIQAIP | |
| | | | | CAP* | |
| IRF1 | interferon regulatory | 3659 | 192 | MPITRMRMRPWLEMQINSNQIPGLIWINKEEMIFQIPWKHAAKHGW | 179. |
| | factor 1 [Source:HGNC | | | DINKDACLFRSWAIHTGRYKAGEKEPDPKTWKANFRCAMNSLPDI | |
| | Symbol; Acc:HGNC: | | | EEVKDQSRNKGSSAVRVYRMLPPLTKNQRKERKSKSSRDAKSKAK | |
| | 6116] | | | RKSCGDSSPDTFSDGLSSSTLPDDHSSYTVPGYMQDLEVEQALTPA | |
| | | | | LSPCAVSSTL | |
| IRF1 | interferon regulatory | 3659 | 198 | MPITRMRMRPWLEMQINSNQIPGLIWINKEEMIFQIPWKHAAKHGW | 180. |
| | factor 1 [Source:HGNC | | | DINKDACLFRSWAIHTGRYKAGEKEPDPKTWKANFRCAMNSLPDI | |
| | Symbol; Acc:HGNC: | | | EEVKDQSRNKGSSAVRVYRMLPPLTKNQRKERKSKSSRDAKSKAK | |
| | 1616] | | | RKSCGDSSPDTFSDGLSSSTLPDDHSSYTVPGYMQDLEVEQALTPA | |
| | | | | TTDEDEEGKLPEDIMK | |
| IRF1 | interferon regulatory | 3659 | 52 | MPITRMRMRPWLEMQINSNQIPGLIWINKADTKQGKRSQIPRRGRP | 181. |
| | factor 1 [Source:HGNC | | | TFAVP* | |
| | Symbol; Acc:HGNC: | | | | |
| | 6116] | | | | |
| IRF1 | interferon regulatory | 3659 | 118 | MPITRMRMRPWLEMQINSNQIPGLIWINKEEMIFQIPWKHAAKHGW | 182. |
| | factor 1 [Source:HGNC | | | DINKDACLFRSWAIHTGRYKAGEKEPDPKTWKANFRCAMNSLPDI | |
| | Symbol; Acc:HGNC: | | | EEVKDQSRNKGSSAVRVYRMLPPLTKN | |
| | 6116] | | | | |
| IRF1 | interferon regulatory | 3659 | 202 | MPITRMRMRPWLEMQINSNQIPGLIWINKEEMIFQIPWKHAAKHGW | 183. |
| | factor 1 [Source:HGNC | | | DINKDACLFRSWAIHTGRYKAGEKEPDPKTWKANFRCAMNSLPDI | |
| | Symbol; Acc:HGNC: | | | EEVKDQSRNKGSSAVRVYRMLPPLTKNQRKERKSKSSRDAKSKAK | |
| | 6116] | | | RKSCGDSSPDTFSDGLSSSTLPDDHSSYTVPGYMQDLEVEQALTPA | |
| | | | | LGAVGVAANKRGWEGVPTQ* | |
| LRBA | LPS responsive beige- | 987 | 2864 | MASEDNRVPSPPPTGDDGGGGGREETPTEGGALSLKPGLPIRGIRM | 184. |
| | like anchor protein | | | KFAVLTGLVEVGEVSNRDIVETVFNLLVGGQFDLEMNFIIQEGESIN | |
| | [Source:HGNC | | | CMVDLLEKCDITCQAEVWSMFTAILKKSIRNLQVCTEVGLVEKVLG | |
| | Symbol; Acc:HGNC: | | | KIEKVDNMIADLLVDMLGVLASYNLTVRELKLFFSKLQGDKGRWP | |
| | 1742] | | | PHAGKLLSVLKHMPQKYGPDAFFNFPGKSAAAIALPPIAKWPYQNG | |
| | | | | FTFHTWLRMDPVNNINVDKDKPYLYCFRTSKGLGYSAHFVGGCLI | |
| | | | | VTSIKSKGKGFQHCVKFDFKPQKWYMVTIVHIYNRWKNSELRCYV | |
| | | | | NGELASYGEITWFVNTSDTFDKCFLGSSETADANRVFCGQMTAVY | |
| | | | | LFSEALNAAQIFAIYQLGLGYKGTFKFKAESDLFLAEHHKLLLYDG | |
| | | | | KLSSAIAFTYNPRATDAQLCLESSPKDNPSIFVHSPHALMLQDVKAV | |
| | | | | LTHSIQSAMHSIGGVQVLFPLFAQLDYRQYLSDEIDLTICSTLLAFIM | |
| | | | | ELLKNSIAMQEQMLACKGFLVIGYSLEKSSKSHVSRAVLELCLAFS | |
| | | | | KYLSNLQNGMPLLKQLCDHVLLNPAIWIHTPAKVQLMLYTYLSTEF | |
| | | | | IGTVNIYNTIRRVGTVLLIMHTLKYYYWAVNPQDRSGITPKGLDGP | |
| | | | | RPNQKEMLSLRAFLLMFIKQLVMKDSGVKEDELQAILNYLLTMHE | |
| | | | | DDNLMDVLQLLVALMSEHPNSMIPAFDQRNGLRVIYKLLASKSEGI | |
| | | | | RVQALKAMGYFLKHLAPKRKAEVMLGHGLFSLLAERLMLQTNLIT | |
| | | | | MTTYNVLFEILIEQIGTQVIHKQHPDPDSSVKIQNPQILKVIATLLRNS | |
| | | | | PQCPESMEVRRAFLSDMIKLFNNSRENRRSLLQCSVWQEWMLSLC | |
| | | | | YFNPKNSDEQKITEMVYAIFRILLYHAVKYEWGGWRVWVDTLSIT | |
| | | | | HSKVTFEIHKENLANIFREQQGKVDEEIGLCSSTSVQAASGIRRDINV | |
| | | | | SVGSQQPDTKDSPVCPHFTTNGNENSSIEKTSSLESASNIELQTTNTS | |
| | | | | YEEMKAEQENQELPDEGTLEETLTNETRNADDLEVSSDIIEAVAISS | |
| | | | | NSFITTGKDSMTVSEVTASISSPSEEDASEMPEFLDKSIVEEEEDDDY | |
| | | | | VELKVEGSPTEEANLPTELQDNSLSPAASEAGEKLDMFGNDDKLIF | |
| | | | | QEGKPVTEKQTDTETQDSKDSGIQTMTASGSSAMSPETTVSQIAVES | |
| | | | | DLGQMLEEGKKATNLTRETKLINDCHGSVSEASSEQKIAKLDVSNV | |
| | | | | ATDTERLELKASPNVEAPQPHRHVLEISRQHEQPGQGIAPDAVNGQ | |
| | | | | RRDSRSTVFRIPEFNWSQMHQRLLTDLLFSIETDIQMWRSHSTKTV | |
| | | | | MDFVNSSDNVIFVHNTIHLISQVMDNMVMACGGILPLLSAATSATH | |
| | | | | ELENIEPTQGLSIEASVTFLQRLISLVDVLIFASSLGFTEIEAEKSMSSG | |
| | | | | GILRQCLRLVCAVAVRNCLECQQHSQLKTRGDKALKPMHSLIPLGK | |
| | | | | SAAKSPVDIVTGGISPVRDLDRLLQDMDINRLRAVVFRDIEDSKQA | |
| | | | | QFLALAVVYFISVLMVSKYRDILEPQNERHSQSCTETGSENENVSLS | |
| | | | | EITPAAFSTLTTASVEESESTSSARRRDSGIGEETATGLGSHVEVTPH | |
| | | | | TAPPGVSAGPDAISEVLSTLSLEVNKSPETKNDRGNDLDTKATPSVS | |
| | | | | VSKNVNVKDILRSLVNIPADGVTVDPALLPPACLGALGDLSVEQPV | |
| | | | | QFRSFDRSVIVAAKKSAVSPSTFNTSIPTNAVSVVSSVDSAQASDMG | |
| | | | | GESPGSRSSNAKLPSVPTVDSVSQDPVSNMSITERLEHALEKAAPLL | |
| | | | | REIFVDFAPFLSRTLLGSHGQELLIEGTSLVCMKSSSSVVELVMLLCS | |
| | | | | QEWQNSIQKNAGLAFIELVNEGRLLSQTMKDHLVRVANEAEFILSR | |
| | | | | QRAEDIHRHAEFESLCAQYSADKREDEKMCDHLIRAAKYRDHVTA | |
| | | | | TQLIQKIINILTDKHGAWGNSAVSRPLEFWRLDYWEDDLRRRRRFV | |
| | | | | RNPLGSTHPEATLKTAVEHVCIFKLRENSKATDEDILAKGKQSIRSQ | |
| | | | | ALGNQNSENEILLEGDDDTLSSVDEKDLENLAGPVSLSTPAQLVAPS | |
| | | | | VVVKGTLSVTSSELYFEVDEEDPNFKKIDPKILAYTEGLHGKWLFTE | |
| | | | | IRSIFSRRYLLQNTALEIFMANRVAVMFNFPDPATVKKVVNYLPRV | |
| | | | | GVGTSFGLPQTRRISLASPRQLFKASNMTQRWQHREISNFEYLMFL | |
| | | | | NTIAGRSYNDLNQYPVFPWVITNYESEELDLTLPTNFRDLSKPIGAL | |
| | | | | NPKRAAFFAERYESWEDDQVPKFHYGTHYSTASFVLAWLLRIEPFT | |
| | | | | TYFLNLQGGKFDHADRTFSSISRAWRNSQRDTSDIKELIPEFYYLPE | |
| | | | | MFVNFNNYNLGVMDDGTVVSDVELPPWAKTSEEFVHINRLALESE | |
| | | | | FVSCQLHQWIDLIFGYKQQGPEAVRALNVFYYLTYEGAVNLNSITD | |
| | | | | PVLREAVEAQIRSFGQTPSQLLIEPHPPRGSAMQVSPLMFTDKAQQD | |
| | | | | VIMVLKFPSNSPVTHVAANTQPGLATPAVITVTANRLFAVNKWHN | |
| | | | | LPAHQGAVQDQPYQLPVEIDPLIASNTGMHRRQITDLLDQSIQVHSQ | |
| | | | | CFVITSDNRYILVCGFWDKSFRVYSTDTGRLIQVVFGHWDVVTCLA | |
| | | | | RSESYIGGNCYILSGSRDATLLLWYWNGKCSGIGDNPGSETAAPRAI | |
| | | | | LTGHDYEVTCAAVCAELGLVLSGSQEGPCLIHSMNGDLLRTLEGPE | |
| | | | | NCLKPKLIQASREGHCVIFYENGLFCTFSVNGKLQATMETDDNIRAI | |
| | | | | QLSRDGQYLLTGGDRGVVVVRQVSDLKQLFAYPGCDAGIRAMALS | |
| | | | | YDQRCIISGMASGSIVLFYNDFNRWHHEYQTRY* | |
| LRBA | LPS responsive beige- | 987 | 135 | GFTEIEAEKSMSSGGILRQCLRLVCAVAVRNCLECQQHSQLKTRGD | 185. |
| | like anchor protein | | | KALKPMHSLIPLGKSAAKSPVDIVTGGISPVRDLDRLLQDMDINRLR | |
| | [Source:HGNC | | | AVVFRDITKKSLHMNLQNIIDFVRSRITNMSKNCIKWICSN* | |
| | Symbol; Acc:HGNC: | | | | |
| | 4172] | | | | |
| LRBA | LPS responsive beige- | 987 | 2576 | MASEDNRVPSPPPTGDDGGGGGREETPTEGGALSLKPGLPIRGIRM | 186. |
| | like anchor protein | | | KFAVLTGLVEVGEVSNRDIVETVFNLLVGGQFDLEMNFIIQEGESIN | |
| | [Source:HGNC | | | CMVDLLEKCDITCQAEVWSMFTAILKKSIRNLQVCTEVGLVEKVLG | |
| | Symbol; Acc:HGNC: | | | KIEKVDNMIADLLVDMLGVLASYNLTVRELKLFFSKLQGDKGRWP | |
| | 1742] | | | PHAGKLLSVLKHMPQKYGPDAFFNFPGKSAAAIALPPIAKWPYQNG | |
| | | | | FTFHTWLRMDPVNNINVDKDKPYLYCFRTSKGLGYSAHFVGGCLI | |
| | | | | VTSIKSKGKGFQHCVKFDFKPQKWYMVTIVHIYNRWKNSELRCYV | |
| | | | | NGELASYGEITWFVNTSDTFDKCFLGSSETADANRVFCGQMTAVY | |
| | | | | LFSEALNAAQIFAIYQLGLGYKGTFKFKAESDLFLAEHHKLLLYDG | |
| | | | | KLSSAIAFTYNPRATDAQLCLESSPKDNPSIFVHSPHALMLQDVKAV | |
| | | | | LTHSIQSAMHSIGGVQVLFPLFAQLDYRQYLSDEIDLTICSTLLAFIM | |
| | | | | ELLKNSIAMQEQMLACKGFLVIGYSLEKSSKSHVSRAVLELCLAFS | |
| | | | | KYLSNLQNGMPLLKQLCDHVLLNPAIWIHTPAKVQLMLYTYLSTEF | |
| | | | | IGTVNIYNTIRRVGTVLLIMHTLKYYYWAVNPQDRSGITPKGLDGP | |
| | | | | RPNQKEMLSLRAFLLMFIKQLVMKDSGVKEDELQAILNYLLTMHE | |
| | | | | DDNLMDVLQLLVALMSEHPNSMIPAFDQRNGLRVIYKLLASKSEGI | |
| | | | | RVQALKAMGYFLKHLAPKRKAEVMLGHGLFSLLAERLMLQTNLIT | |
| | | | | MTTYNVLFEILIEQIGTQVIHKQHPDPDSSVKIQNPQILKVIATLLRNS | |
| | | | | PQCPESMEVRRAFLSDMIKLFNNSRENRRSLLQCSVWQEWMLSLC | |
| | | | | YFNPKNSDEQKITEMVYAIFRILLYHAVKYEWGGWRVWVDTLSIT | |
| | | | | HSKVTFEIHKENLANIFREQQGKVDEEIGLCSSTSVQAASGIRRDINV | |
| | | | | SVGSQQPDTKDSPVCPHFTTNGNENSSIEKTSSLESASNIELQTTNTS | |
| | | | | YEEMKAEQENQELPDEGTLEETLTNETRNADDLEVSSDIIEAVAISS | |
| | | | | NSFITTGKDSMTVSEVTASISSPSEEDASEMPEFLDKSIVEEEEDDDY | |
| | | | | VELKVEGSPTEEANLPTELQDNSLSPAASEAGEKLDMFGNDDKLIF | |
| | | | | QEGKPVTEKQTDTETQDSKDSGIQTMTASGSSAMSPETTVSQIAVES | |
| | | | | DLGQMLEEGKKATNLTRETKLINDCHGSVSEASSEQKIAKLDVSNV | |
| | | | | ATDTERLELKASPNVEAPQPHRHVLEISRQHEQPGQGIAPDAVNGQ | |
| | | | | RRDSRSTVFRIPEFNWSQMHQRLLTDLLFSIETDIQMWRSHSTKTV | |
| | | | | MDFVNSSDNVIFVHNTIHLISQVMDNMVMACGGILPLLSAATSATH | |
| | | | | ELENIEPTQGLSIEASVTFLQRLISLVDVLIFASSLGFTEIEAEKSMSSG | |
| | | | | GILRQCLRLVCAVAVRNCLECQQHSQLKTRGDKALKPMHSLIPLGK | |
| | | | | SAAKSPVDIVTGGISPVRDLDRLLQDMDINRLRAVVFRDIEDSKQA | |
| | | | | QFLALAVVYFISVLMVSKYRDILEPQNERHSQSCTETGSENENVSLS | |
| | | | | EITPAAFSTLTTASVEESESTSSARRRDSGIGEETATGLGSHVEVTPH | |
| | | | | TAPPGVSAGPDAISEVLSTLSLEVNKSPETKNDRGNDLDTKATPSVS | |
| | | | | VSKNVNVKDILRSLVNIPADGVTVDPALLPPACLGALGDLSVEQPV | |
| | | | | QFRSFDRSVIVAAKKSAVSPSTFNTSIPTNAVSVVSSVDSAQASDMG | |
| | | | | GESPGSRSSNAKLPSVPTVDSVSQDPVSNMSITERLEHALEKAAPLL | |
| | | | | REIFVDFAPFLSRTLLGSHGQELLIEGTSLVCMKSSSSVVELVMLLCS | |
| | | | | QEWQNSIQKNAGLAFIELVNEGRLLSQTMKDHLVRVANEAEFILSR | |
| | | | | QRAEDIHRHAEFESLCAQYSADKREDEKMCDHLIRAAKYRDHVTA | |
| | | | | TQLIQKIINILTDKHGAWGNSAVSRPLEFWRLDYWEDDLRRRRRFV | |
| | | | | RNPLGSTHPEATLKTAVEHATDEDILAKGKQSIRSQALGNQNSENEI | |
| | | | | LLEGDDDTLSSVDEKDLENLAGPVSLSTPAQLVAPSVVVKGTLSVT | |
| | | | | SSELYFEVDEEDPNFKKIDPKILAYTEGLHGKWLFTEIRSIFSRRYLL | |
| | | | | QNTALEIFMANRVAVMFNFPDPATVKKVVNYLPRVGVGTSFGLPQ | |
| | | | | TRRISLASPRQLFKASNMTQRWQHREISNFEYLMFLNTIAGRSYNDL | |
| | | | | NQYPVFPWVITNYESEELDLTLPTNFRDLSKPIGALNPKRAAFFAER | |
| | | | | YESWEDDQVPKFHYGTHYSTASFVLAWLLRIEPFTTYFLNLQGGKF | |
| | | | | DHADRTFSSISRAWRNSQRDTSDIKELIPEFYYLPEMFVNFNNYNLG | |
| | | | | VMDDGTVVSDVELPPWAKTSEEFVHINRLALESEFVSCQLHQWIDL | |
| | | | | IFGYKQQGPEAVRALNVFYYLTYEGAVNLNSITDPVLREAVEAQIR | |
| | | | | SFGQTPSQLLIEPHPPRGSAMQVSPLMFTDKAQQDVIMVLKFPSNSP | |
| | | | | VTHVAANTQPGLATPAVITVTANRLFAVNKWHNLPAHQGAVQDQ | |
| | | | | PYQLPVEIDPLIGLSLPSLFAIH* | |
| LRBA | LPS responsive beige- | 987 | 148 | XYILSGSRDATLLLWYWNGKCSGIGDNPGSETAAPRAILTGHDYEV | 187. |
| | like anchor protein | | | TCAAVCAELGLVLSGSQEGPCLIHSMNGDLLRTLEGPENCLKPKLIQ | |
| | [Source:HGNC | | | ASREGHCVIFYENGLFCTFSVNGKLQATMETDDNIRVHHFWHGFR | |
| | Symbol; Acc:HGNC: | | | KHCAILQRL* | |
| | 1742] | | | | |
| LRBA | LPS responsive beige- | 987 | 1506 | XDNVIFVHNTIHLISQVMDNMVMACGGILPLLSAATSATHELENIEP | 188. |
| | like anchor protein | | | TQGLSIEASVTFLQRLISLVDVLIFASSLGFTEIEAEKSMSSGGILRQC | |
| | [Source:HGNC | | | LRLVCAVAVRNCLECQQHSQLKTRGDKALKPMHSLIPLGKSAAKS | |
| | Symbol; Acc:HGNC: | | | PVDIVTGGISPVRDLDRLLQDMDINRLRAVVFRDIEDSKQAQFLALA | |
| | 1742] | | | VVYFISVLMVSKYRDILEPQNERHSQSCTETGSENENVSLSEITPAAF | |
| | | | | STLTTASVEESESTSSARRRDSGIGEETATGLGSHVEVTPHTAPPGVS | |
| | | | | AGPDAISEVLSTLSLEVNKSPETKNDRGNDLDTKATPSVSVSKNVN | |
| | | | | VKDILRSLVNIPADGVTVDPALLPPACLGALGDLSVEQPVQFRSFDR | |
| | | | | SVIVAAKKSAVSPSTFNTSIPTNAVSVVSSVDSAQASDMGGESPGSR | |
| | | | | SSNAKLPSVPTVDSVSQDPVSNMSITERLEHALEKAAPLLREIFVDF | |
| | | | | APFLSRTLLGSHGQELLIEGTSLVCMKSSSSVVELVMLLCSQEWQN | |
| | | | | SIQKNAGLAFIELVNEGRLLSQTMKDHLVRVANEAEFILSRQRAEDI | |
| | | | | HRHAEFESLCAQYSADKREDEKMCDHLIRAAKYRDHVTATQLIQKI | |
| | | | | INILTDKHGAWGNSAVSRPLEFWRLDYWEDDLRRRRRFVRNPLGS | |
| | | | | THPEATLKTAVEHATDEDILAKGKQSIRSQALGNQNSENEILLEGDD | |
| | | | | DTLSSVDEKDLENLAGPVSLSTPAQLVAPSVVVKGTLSVTSSELYFE | |
| | | | | VDEEDPNFKKIDPKILAYTEGLHGKWLFTEIRSIFSRRYLLQNTALEI | |
| | | | | FMANRVAVMFNFPDPATVKKVVNYLPRVGVGTSFGLPQTRRISLA | |
| | | | | SPRQLFKASNMTQRWQHREISNFEYLMFLNTIAGRSYNDLNQYPVF | |
| | | | | PWVITNYESEELDLTLPTNFRDLSKPIGALNPKRAAFFAERYESWED | |
| | | | | DQVPKFHYGTHYSTASFVLAWLLRIEPFTTYFLNLQGGKFDHADRT | |
| | | | | FSSISRAWRNSQRDTSDIKELIPEFYYLPEMFVNFNNYNLGVMDDG | |
| | | | | TVVSDVELPPWAKTSEEFVHINRLALESEFVSCQLHQWIDLIFGYKQ | |
| | | | | QGPEAVRALNVFYYLTYEGAVNLNSITDPVLREAVEAQIRSFGQTP | |
| | | | | SQLLIEPHPPRGSAMQVSPLMFTDKAQQDVIMVLKFPSNSPVTHVA | |
| | | | | ANTQPGLATPAVITVTANRLFAVNKWHNLPAHQGAVQDQPYQLPV | |
| | | | | EIDPLIASNTGMHRRQITDLLDQSIQVHSQCFVITSDNRYILVCGFWD | |
| | | | | KSFRVYSTDTGRLIQVVFGHWDVVTCLARSESYIGGNCYILSGSRD | |
| | | | | ATLLLWYWNGKCSGIGDNPGSETAAPRAILTGHDYEVTCAAVCAE | |
| | | | | LGLVLSGSQEGPCLIHSMNGDLLRTLEGPENCLKPKLIQASREGHCV | |
| | | | | IFYENGLFCTFSVNGKLQATMETDDNIRAIQLSRDGQYLLTGGDRG | |
| | | | | VVVVRQVSDLKQLFAYPGCDAGIRAMALSYDQRCIISGMASGSIVL | |
| | | | | FYNDFNRWHHEYQTRY* | |
| LRBA | LPS responsive beige- | 987 | 1881 | MASEDNRVPSPPPTGDDGGGGGREETPTEGGALSLKPGLPIRGIRM | 189. |
| | like anchor protein | | | KFAVLTGLVEVGEVSNRDIVETVFNLLVGGQFDLEMNFIIQEGESIN | |
| | [Source:HGNC | | | CMVDLLEKCDITCQAEVWSMFTAILKKSIRNLQVCTEVGLVEKVLG | |
| | Symbol; Acc:HGNC: | | | KIEKVDNMIADLLVDMLGVLASYNLTVRELKLFFSKLQGDKGRWP | |
| | 1742] | | | PHAGKLLSVLKHMPQKYGPDAFFNFPGKSAAAIALPPIAKWPYQNG | |
| | | | | FTFHTWLRMDPVNNINVDKDKPYLYCFRTSKGLGYSAHFVGGCLI | |
| | | | | VTSIKSKGKGFQHCVKFDFKPQKWYMVTIVHIYNRWKNSELRCYV | |
| | | | | NGELASYGEITWFVNTSDTFDKCFLGSSETADANRVFCGQMTAVY | |
| | | | | LFSEALNAAQIFAIYQLGLGYKGTFKFKAESDLFLAEHHKLLLYDG | |
| | | | | KLSSAIAFTYNPRATDAQLCLESSPKDNPSIFVHSPHALMLQDVKAV | |
| | | | | LTHSIQSAMHSIGGVQVLFPLFAQLDYRQYLSDEIDLTICSTLLAFIM | |
| | | | | ELLKNSIAMQEQMLACKGFLVIGYSLEKSSKSHVSRAVLELCLAFS | |
| | | | | KYLSNLQNGMPLLKQLCDHVLLNPAIWIHTPAKVQLMLYTYLSTEF | |
| | | | | IGTVNIYNTIRRVGTVLLIMHTLKYYYWAVNPQDRSGITPKGLDGP | |
| | | | | RPNQKEMLSLRAFLLMFIKQLVMKDSGVKEDELQAILNYLLTMHE | |
| | | | | DDNLMDVLQLLVALMSEHPNSMIPAFDQRNGLRVIYKLLASKSEGI | |
| | | | | RVQALKAMGYFLKHLAPKRKAEVMLGHGLFSLLAERLMLQTNLIT | |
| | | | | MTTYNVLFEILIEQIGTQVIHKQHPDPDSSVKIQNPQILKVIATLLRNS | |
| | | | | PQCPESMEVRRAFLSDMIKLFNNSRENRRSLLQCSVWQEWMLSLC | |
| | | | | YFNPKNSDEQKITEMVYAIFRILLYHAVKYEWGGWRVWVDTLSIT | |
| | | | | HSKVTFEIHKENLANIFREQQGKVDEEIGLCSSTSVQAASGIRRDINV | |
| | | | | SVGSQQPDTKDSPVCPHFTTNGNENSSIEKTSSLESASNIELQTTNTS | |
| | | | | YEEMKAEQENQELPDEGTLEETLTNETRNADDLEVSSDIIEAVAISS | |
| | | | | NSFITTGKDSMTVSEVTASISSPSEEDASEMPEFLDKSIVEEEEDDDY | |
| | | | | VELKVEGSPTEEANLPTELQDNSLSPAASEAGEKLDMFGNDDKLIF | |
| | | | | QEGKPVTEKQTDTETQDSKDSGIQTMTASGSSAMSPETTVSQIAVES | |
| | | | | DLGQMLEEGKKATNLTRETKLINDCHGSVSEASSEQKIAKLDVSNV | |
| | | | | ATDTERLELKASPNVEAPQPHRHVLEISRQHEQPGQGIAPDAVNGQ | |
| | | | | RRDSRSTVFRIPEFNWSQMHQRLLTDLLFSIETDIQMWRSHSTKTV | |
| | | | | MDFVNSSDNVIFVHNTIHLISQVMDNMVMACGGILPLLSAATSATH | |
| | | | | ELENIEPTQGLSIEASVTFLQRLISLVDVLIFASSLGFTEIEAEKSMSSG | |
| | | | | GILRQCLRLVCAVAVRNCLECQQHSQLKTRGDKALKPMHSLIPLGK | |
| | | | | SAAKSPVDIVTGGISPVRDLDRLLQDMDINRLRAVVFRDIEDSKQA | |
| | | | | QFLALAVVYFISVLMVSKYRDILEPQNERHSQSCTETGSENENVSLS | |
| | | | | EITPAAFSTLTTASVEESESTSSARRRDSGIGEETATGLGSHVEVTPH | |
| | | | | TAPPGVSAGPDAISEVLSTLSLEVNKSPETKNDRGNDLDTKATPSVS | |
| | | | | VSKNVNVKDILRSLVNIPADGVTVDPALLPPACLGALGDLSVEQPV | |
| | | | | QFRSFDRSVIVAAKKSAVSPSTFNTSIPTNAVSVVSSVDSAQASDMG | |
| | | | | GESPGSRSSNAKLPSVPTVDSVSQDPVSNMSITERLEHALEKAAPLL | |
| | | | | REIFVDFAPFLSRTLLGSHGQELLIEGTSLVCMKSSSSVVELVMLLCS | |
| | | | | QVRVAKFYSEECRPCFYRTCQ* | |
| LRBA | LPS responsive beige- | 987 | 687 | GTSFGLPQTRRISLASPRQLFKASNMTQRWQHREISNFEYLMFLNTI | 190. |
| | like anchor protein | | | AGRSYNDLNQYPVFPWVITNYESEELDLTLPTNFRDLSKPIGALNPK | |
| | [Source:HGNC | | | RAAFFAERYESWEDDQVPKFHYGTHYSTASFVLAWLLRIEPFTTYF | |
| | Symbol; Acc:HGNC: | | | LNLQGGKFDHADRTFSSISRAWRNSQRDTSDIKELIPEFYYLPEMFV | |
| | 1742] | | | NFNNYNLGVMDDGTVVSDVELPPWAKTSEEFVHINRLALESEFVSC | |
| | | | | QLHQWIDLIFGYKQQGPEAVRALNVFYYLTYEGAVNLNSITDPVLR | |
| | | | | EAVEAQIRSFGQTPSQLLIEPHPPRGSAMQVYLLLQSPLMFTDKAQQ | |
| | | | | DVIMVLKFPSNSPVTHVAANTQPGLATPAVITVTANRLFAVNKWH | |
| | | | | NLPAHQGAVQDQPYQLPVEIDPLIASNTGMHRRQITDLLDQSIQVHS | |
| | | | | QCFVITSDNRYILVCGFWDKSFRVYSTDTGRLIQVVFGHWDVVTCL | |
| | | | | ARSESYIGGNCYILSGSRDATLLLWYWNGKCSGIGDNPGSETAAPR | |
| | | | | AILTGHDYEVTCAAVCAELGLVLSGSQEGPCLIHSMNGDLLRTLEG | |
| | | | | PENCLKPKLIQASREGHCVIFYENGLFCTFSVNGKLQATMETDDNIR | |
| | | | | AIQLSRDGQYLLTGGDRGVVVVRQVSDLKQLFAYPGCDAGIRAMA | |
| | | | | LSYDQRRPRSLQKFLSSVISVHHFWHGFRKHCAILQRL* | |
| LRBA | LPS responsive beige- | 987 | 2096 | GLFSLLAERLMLQTNLITMTTYNVLFEILIEQIGTQVIHKQHPDPDSS | 191. |
| | like anchor protein | | | VKIQNPQILKVIATLLRNSPQCPESMEVRRAFLSDMIKLFNNSRENR | |
| | [Source:HGNC | | | RSLLQCSVWQEWMLSLCYFNPKNSDEQKITEMVYAIFRILLYHAVK | |
| | Symbol; Acc:HGNC: | | | YEWGGWRVWVDTLSITHSKVTFEIHKENLANIFREQQGKVDEEIGL | |
| | 1742] | | | CSSTSVQAASGIRRDINVSVGSQQPDTKDSPVCPHFTTNGNENSSIE | |
| | | | | KTSSLESASNIELQTTNTSYEEMKAEQENQELPDEGTLEETLTNETR | |
| | | | | NADDLEVSSDIIEAVAISSNSFITTGKDSMTVSEVTASISSPSEEDASE | |
| | | | | MPEFLDKSIVEEEEDDDYVELKVEGSPTEEANLPTELQDNSLSPAAS | |
| | | | | EAGEKLDMFGNDDKLIFQEGKPVTEKQTDTETQDSKDSGIQTMTAS | |
| | | | | GSSAMSPETTVSQIAVESDLGQMLEEGKKATNLTRETKLINDCHGS | |
| | | | | VSEASSEQKIAKLDVSNVATDTERLELKASPNVEAPQPHRHVLEISR | |
| | | | | QHEQPGQGIAPDAVNGQRRDSRSTVFRIPEFNWSQMHQRLLTDLLF | |
| | | | | SIETDIQMWRSHSTKTVMDFVNSSDNVIFVHNTIHLISQVMDNMVM | |
| | | | | ACGGILPLLSAATSATHELENIEPTQGLSIEASVTFLQRLISLVDVLIF | |
| | | | | ASSLGFTEIEAEKSMSSGGILRQCLRLVCAVAVRNCLECQQHSQLK | |
| | | | | TRGDKALKPMHSLIPLGKSAAKSPVDIVTGGISPVRDLDRLLQDMDI | |
| | | | | NRLRAVVFRDIEDSKQAQFLALAVVYFISVLMVSKYRDILEPQNER | |
| | | | | HSQSCTETGSENENVSLSEITPAAFSTLTTASVEESESTSSARRRDSGI | |
| | | | | GEETATGLGSHVEVTPHTAPPGVSAGPDAISEVLSTLSLEVNKSPET | |
| | | | | KNDRGNDLDTKATPSVSVSKNVNVKDILRSLVNIPADGVTVDPALL | |
| | | | | PPACLGALGDLSVEQPVQFRSFDRSVIVAAKKSAVSPSTFNTSIPTN | |
| | | | | AVSVVSSVDSAQASDMGGESPGSRSSNAKLPSVPTVDSVSQDPVSN | |
| | | | | MSITERLEHALEKAAPLLREIFVDFAPFLSRTLLGSHGQELLIEGTSL | |
| | | | | VCMKSSSSVVELVMLLCSQEWQNSIQKNAGLAFIELVNEGRLLSQT | |
| | | | | MKDHLVRVANEAEFILSRQRAEDIHRHAEFESLCAQYSADKREDEK | |
| | | | | MCDHLIRAAKYRDHVTATQLIQKIINILTDKHGAWGNSAVSRPLEF | |
| | | | | WRLDYWEDDLRRRRRFVRNPLGSTHPEATLKTAVEHATDEDILAK | |
| | | | | GKQSIRSQALGNQNSENEILLEGDDDTLSSVDEKDLENLAGPVSLST | |
| | | | | PAQLVAPSVVVKGTLSVTSSELYFEVDEEDPNFKKIDPKILAYTEGL | |
| | | | | HGKWLFTEIRSIFSRRYLLQNTALEIFMANRVAVMFNFPDPATVKK | |
| | | | | VVNYLPRVGVGTSFGLPQTRRISLASPRQLFKASNMTQRWQHREIS | |
| | | | | NFEYLMFLNTIAGRSYNDLNQYPVFPWVITNYESEELDLTLPTNFRD | |
| | | | | LSKPIGALNPKRAAFFAERYESWEDDQVPKFHYGTHYSTASFVLA | |
| | | | | WLLRIEPFTTYFLNLQGGKFDHADRTFSSISRAWRNSQRDTSDIKELI | |
| | | | | PEFYYLPEMFVNFNNYNLGVMDDGTVVSDVELPPWAKTSEEFVHI | |
| | | | | NRLALESEFVSCQLHQWIDLIFGYKQQGPEAVRALNVFYYLTYEGA | |
| | | | | VNLNSITDPVLREAVEAQIRSFGQTPSQLLIEPHPPRGSAMQVYLLL | |
| | | | | QSPLMFTDKAQQDVIMVLKFPSNSPVTHVAANTQPGLATPAVITVT | |
| | | | | ANRLFAVNKWHNLPAHQGAVQDQPYQLPVEIDPLIASNTGMHRRQ | |
| | | | | ITDLLDQSIQVHSQCFVITSDNRYILVCGFWDKSFRVYSTDTGRLIQ | |
| | | | | VVFGHWDVVTCLARSESYIGGNCYILSGSRDATLLLWYWNGKCSG | |
| | | | | IGDNPGSETAAPRAILTGHDYEVTCAAVCAELGLVLSGSQEGPCLIH | |
| | | | | SMNGDLLRTLEGPENCLKPKLIQASREGHCVIFYENGLFCTFSVNGK | |
| | | | | LQATMETDDNIRAIQLSRDGQYLLTGGDRGVVVVRQVSDLKQLFA | |
| | | | | YPGCDAGIRAMALSYDQRCIISGMASGSIVLFYNDFNRWHHEYQTR | |
| | | | | Y* | |
| LRBA | LPS responsive beige- | 987 | 238 | HYSTASFVLAWLLRIEPFTTYFLNLQGGKFDHADRTFSSISRAWRNS | 192. |
| | like anchor protein | | | QRDTSDIKELIPEFYYLPEMFVNFNNYNLGVMDDGTVVSDVELPPW | |
| | [Source:HGNC | | | AKTSEEFVHINRLQGPEAVRALNVFYYLTYEGAVNLNSITDPVLRE | |
| | Symbol; Acc:HGNC: | | | AVEAQIRSFGQTPSQLLIEPHPPRGSAMQVYLLLQSPLMFTDKAQQ | |
| | 1742] | | | DVIMVLKFPSNSPVTHVAANTQPGLATPAVITVTANRLFAVNKWH | |
| | | | | NLPAHQGA | |
| LRBA | LPS responsive beige- | 987 | 2852 | MASEDNRVPSPPPTGDDGGGGGREETPTEGGALSLKPGLPIRGIRM | 193. |
| | like anchor protein | | | KFAVLTGLVEVGEVSNRDIVETVFNLLVGGQFDLEMNFIIQEGESIN | |
| | [Source:HGNC | | | CMVDLLEKCDITCQAEVWSMFTAILKKSIRNLQVCTEVGLVEKVLG | |
| | Symbol; Acc:HGNC: | | | KIEKVDNMIADLLVDMLGVLASYNLTVRELKLFFSKLQGDKGRWP | |
| | 1742] | | | PHAGKLLSVLKHMPQKYGPDAFFNFPGKSAAAIALPPIAKWPYQNG | |
| | | | | FTFHTWLRMDPVNNINVDKDKPYLYCFRTSKGLGYSAHFVGGCLI | |
| | | | | VTSIKSKGKGFQHCVKFDFKPQKWYMVTIVHIYNRWKNSELRCYV | |
| | | | | NGELASYGEITWFVNTSDTFDKCFLGSSETADANRVFCGQMTAVY | |
| | | | | LFSEALNAAQIFAIYQLGLGYKGTFKFKAESDLFLAEHHKLLLYDG | |
| | | | | KLSSAIAFTYNPRATDAQLCLESSPKDNPSIFVHSPHALMLQDVKAV | |
| | | | | LTHSIQSAMHSIGGVQVLFPLFAQLDYRQYLSDEIDLTICSTLLAFIM | |
| | | | | ELLKNSIAMQEQMLACKGFLVIGYSLEKSSKSHVSRAVLELCLAFS | |
| | | | | KYLSNLQNGMPLLKQLCDHVLLNPAIWIHTPAKVQLMLYTYLSTEF | |
| | | | | IGTVNIYNTIRRVGTVLLIMHTLKYYYWAVNPQDRSGITPKGLDGP | |
| | | | | RPNQKEMLSLRAFLLMFIKQLVMKDSGVKEDELQAILNYLLTMHE | |
| | | | | DDNLMDVLQLLVALMSEHPNSMIPAFDQRNGLRVIYKLLASKSEGI | |
| | | | | RVQALKAMGYFLKHLAPKRKAEVMLGHGLFSLLAERLMLQTNLIT | |
| | | | | MTTYNVLFEILIEQIGTQVIHKQHPDPDSSVKIQNPQILKVIATLLRNS | |
| | | | | PQCPESMEVRRAFLSDMIKLFNNSRENRRSLLQCSVWQEWMLSLC | |
| | | | | YFNPKNSDEQKITEMVYAIFRILLYHAVKYEWGGWRVWVDTLSIT | |
| | | | | HSKVTFEIHKENLANIFREQQGKVDEEIGLCSSTSVQAASGIRRDINV | |
| | | | | SVGSQQPDTKDSPVCPHFTTNGNENSSIEKTSSLESASNIELQTTNTS | |
| | | | | YEEMKAEQENQELPDEGTLEETLTNETRNADDLEVSSDIIEAVAISS | |
| | | | | NSFITTGKDSMTVSEVTASISSPSEEDASEMPEFLDKSIVEEEEDDDY | |
| | | | | VELKVEGSPTEEANLPTELQDNSLSPAASEAGEKLDMFGNDDKLIF | |
| | | | | QEGKPVTEKQTDTETQDSKDSGIQTMTASGSSAMSPETTVSQIAVES | |
| | | | | DLGQMLEEGKKATNLTRETKLINDCHGSVSEASSEQKIAKLDVSNV | |
| | | | | ATDTERLELKASPNVEAPQPHRHVLEISRQHEQPGQGIAPDAVNGQ | |
| | | | | RRDSRSTVFRIPEFNWSQMHQRLLTDLLFSIETDIQMWRSHSTKTV | |
| | | | | MDFVNSSDNVIFVHNTIHLISQVMDNMVMACGGILPLLSAATSATH | |
| | | | | ELENIEPTQGLSIEASVTFLQRLISLVDVLIFASSLGFTEIEAEKSMSSG | |
| | | | | GILRQCLRLVCAVAVRNCLECQQHSQLKTRGDKALKPMHSLIPLGK | |
| | | | | SAAKSPVDIVTGGISPVRDLDRLLQDMDINRLRAVVFRDIEDSKQA | |
| | | | | QFLALAVVYFISVLMVSKYRDILEPQNERHSQSCTETGSENENVSLS | |
| | | | | EITPAAFSTLTTASVEESESTSSARRRDSGIGEETATGLGSHVEVTPH | |
| | | | | TAPPGVSAGPDAISEVLSTLSLEVNKSPETKNDRGNDLDTKATPSVS | |
| | | | | VSKNVNVKDILRSLVNIPADGVTVDPALLPPACLGALGDLSVEQPV | |
| | | | | QFRSFDRSVIVAAKKSAVSPSTFNTSIPTNAVSVVSSVDSAQASDMG | |
| | | | | GESPGSRSSNAKLPSVPTVDSVSQDPVSNMSITERLEHALEKAAPLL | |
| | | | | REIFVDFAPFLSRTLLGSHGQELLIEGTSLVCMKSSSSVVELVMLLCS | |
| | | | | QEWQNSIQKNAGLAFIELVNEGRLLSQTMKDHLVRVANEAEFILSR | |
| | | | | QRAEDIHRHAEFESLCAQYSADKREDEKMCDHLIRAAKYRDHVTA | |
| | | | | TQLIQKIINILTDKHGAWGNSAVSRPLEFWRLDYWEDDLRRRRRFV | |
| | | | | RNPLGSTHPEATLKTAVEHATDEDILAKGKQSIRSQALGNQNSENEI | |
| | | | | LLEGDDDTLSSVDEKDLENLAGPVSLSTPAQLVAPSVVVKGTLSVT | |
| | | | | SSELYFEVDEEDPNFKKIDPKILAYTEGLHGKWLFTEIRSIFSRRYLL | |
| | | | | QNTALEIFMANRVAVMFNFPDPATVKKVVNYLPRVGVGTSFGLPQ | |
| | | | | TRRISLASPRQLFKASNMTQRWQHREISNFEYLMFLNTIAGRSYNDL | |
| | | | | NQYPVFPWVITNYESEELDLTLPTNFRDLSKPIGALNPKRAAFFAER | |
| | | | | YESWEDDQVPKFHYGTHYSTASFVLAWLLRIEPFTTYFLNLQGGKF | |
| | | | | DHADRTFSSISRAWRNSQRDTSDIKELIPEFYYLPEMFVNFNNYNLG | |
| | | | | VMDDGTVVSDVELPPWAKTSEEFVHINRLALESEFVSCQLHQWIDL | |
| | | | | IFGYKQQGPEAVRALNVFYYLTYEGAVNLNSITDPVLREAVEAQIR | |
| | | | | SFGQTPSQLLIEPHPPRGSAMQVSPLMFTDKAQQDVIMVLKFPSNSP | |
| | | | | VTHVAANTQPGLATPAVITVTANRLFAVNKWHNLPAHQGAVQDQ | |
| | | | | PYQLPVEIDPLIASNTGMHRRQITDLLDQSIQVHSQCFVITSDNRYIL | |
| | | | | VCGFWDKSFRVYSTDTGRLIQVVFGHWDVVTCLARSESYIGGNCYI | |
| | | | | LSGSRDATLLLWYWNGKCSGIGDNPGETAAPRAILTGHDYEVTCA | |
| | | | | AVCAELGLVLSGSQEGPCLIHSMNGDLLRTLEGPENCLKPKLIQASR | |
| | | | | EGHCVIFYENGLFCTFSVNGKLQATMETDDNIRAIQLSRDGQYLLT | |
| | | | | GGDRGVVVVRQVSDLKQLFAYPGCDAGIRAMALSYDQRCIISGMA | |
| | | | | SGSIVLFYNDFNRWHHEYQTRY* | |
| LRBA | LPS responsive beige- | 987 | 243 | XTVKKVVNYLPRVGVGTSFGLPQTRRISLASPRQLFKASNMTQRW | 194. |
| | like anchor protein | | | QHREISNFEYLMFLNTIAGRSYNDLNQYPVFPWVITNYESEELDLTL | |
| | [Source:HGNC | | | PTNFRDLSKPIGALNPKRAAFFAERYESWEDDQVPKFHYGTHYSTA | |
| | Symbol; Acc:HGNC: | | | SFVLAWLLRIEPFTTYFLNLQGGKFDHADRTFSSISRAWRNSQRDTS | |
| | 1742] | | | DIKELIPEFYYLPEMFVNFNNYNLGVMDDGTVVSDVELPPWAKTSE | |
| | | | | EFVHINRLVPF* | |
| LRBA | LPS responsive beige- | 987 | 2853 | MASEDNRVPSPPPTGDDGGGGGREETPTEGGALSLKPGLPIRGIRM | 195. |
| | like anchor protein | | | KFAVLTGLVEVGEVSNRDIVETVFNLLVGGQFDLEMNFIIQEGESIN | |
| | [Source:HGNC | | | CMVDLLEKCDITCQAEVWSMFTAILKKSIRNLQVCTEVGLVEKVLG | |
| | Symbol; Acc:HGNC: | | | KIEKVDNMIADLLVDMLGVLASYNLTVRELKLFFSKLQGDKGRWP | |
| | 1742] | | | PHAGKLLSVLKHMPQKYGPDAFFNFPGKSAAAIALPPIAKWPYQNG | |
| | | | | FTFHTWLRMDPVNNINVDKDKPYLYCFRTSKGLGYSAHFVGGCLI | |
| | | | | VTSIKSKGKGFQHCVKFDFKPQKWYMVTIVHIYNRWKNSELRCYV | |
| | | | | NGELASYGEITWFVNTSDTFDKCFLGSSETADANRVFCGQMTAVY | |
| | | | | LFSEALNAAQIFAIYQLGLGYKGTFKFKAESDLFLAEHHKLLLYDG | |
| | | | | KLSSAIAFTYNPRATDAQLCLESSPKDNPSIFVHSPHALMLQDVKAV | |
| | | | | LTHSIQSAMHSIGGVQVLFPLFAQLDYRQYLSDEIDLTICSTLLAFIM | |
| | | | | ELLKNSIAMQEQMLACKGFLVIGYSLEKSSKSHVSRAVLELCLAFS | |
| | | | | KYLSNLQNGMPLLKQLCDHVLLNPAIWIHTPAKVQLMLYTYLSTEF | |
| | | | | IGTVNIYNTIRRVGTVLLIMHTLKYYYWAVNPQDRSGITPKGLDGP | |
| | | | | RPNQKEMLSLRAFLLMFIKQLVMKDSGVKEDELQAILNYLLTMHE | |
| | | | | DDNLMDVLQLLVALMSEHPNSMIPAFDQRNGLRVIYKLLASKSEGI | |
| | | | | RVQALKAMGYFLKHLAPKRKAEVMLGHGLFSLLAERLMLQTNLIT | |
| | | | | MTTYNVLFEILIEQIGTQVIHKQHPDPDSSVKIQNPQILKVIATLLRNS | |
| | | | | PQCPESMEVRRAFLSDMIKLFNNSRENRRSLLQCSVWQEWMLSLC | |
| | | | | YFNPKNSDEQKITEMVYAIFRILLYHAVKYEWGGWRVWVDTLSIT | |
| | | | | HSKVTFEIHKENLANIFREQQGKVDEEIGLCSSTSVQAASGIRRDINV | |
| | | | | SVGSQQPDTKDSPVCPHFTTNGNENSSIEKTSSLESASNIELQTTNTS | |
| | | | | YEEMKAEQENQELPDEGTLEETLTNETRNADDLEVSSDIIEAVAISS | |
| | | | | NSFITTGKDSMTVSEVTASISSPSEEDASEMPEFLDKSIVEEEEDDDY | |
| | | | | VELKVEGSPTEEANLPTELQDNSLSPAASEAGEKLDMFGNDDKLIF | |
| | | | | QEGKPVTEKQTDTETQDSKDSGIQTMTASGSSAMSPETTVSQIAVES | |
| | | | | DLGQMLEEGKKATNLTRETKLINDCHGSVSEASSEQKIAKLDVSNV | |
| | | | | ATDTERLELKASPNVEAPQPHRHVLEISRQHEQPGQGIAPDAVNGQ | |
| | | | | RRDSRSTVFRIPEFNWSQMHQRLLTDLLFSIETDIQMWRSHSTKTV | |
| | | | | MDFVNSSDNVIFVHNTIHLISQVMDNMVMACGGILPLLSAATSATH | |
| | | | | ELENIEPTQGLSIEASVTFLQRLISLVDVLIFASSLGFTEIEAEKSMSSG | |
| | | | | GILRQCLRLVCAVAVRNCLECQQHSQLKTRGDKALKPMHSLIPLGK | |
| | | | | SAAKSPVDIVTGGISPVRDLDRLLQDMDINRLRAVVFRDIEDSKQA | |
| | | | | QFLALAVVYFISVLMVSKYRDILEPQNERHSQSCTETGSENENVSLS | |
| | | | | EITPAAFSTLTTASVEESESTSSARRRDSGIGEETATGLGSHVEVTPH | |
| | | | | TAPPGVSAGPDAISEVLSTLSLEVNKSPETKNDRGNDLDTKATPSVS | |
| | | | | VSKNVNVKDILRSLVNIPADGVTVDPALLPPACLGALGDLSVEQPV | |
| | | | | QFRSFDRSVIVAAKKSAVSPSTFNTSIPTNAVSVVSSVDSAQASDMG | |
| | | | | GESPGSRSSNAKLPSVPTVDSVSQDPVSNMSITERLEHALEKAAPLL | |
| | | | | REIFVDFAPFLSRTLLGSHGQELLIEGTSLVCMKSSSSVVELVMLLCS | |
| | | | | QEWQNSIQKNAGLAFIELVNEGRLLSQTMKDHLVRVANEAEFILSR | |
| | | | | QRAEDIHRHAEFESLCAQYSADKREDEKMCDHLIRAAKYRDHVTA | |
| | | | | TQLIQKIINILTDKHGAWGNSAVSRPLEFWRLDYWEDDLRRRRRFV | |
| | | | | RNPLGSTHPEATLKTAVEHATDEDILAKGKQSIRSQALGNQNSENEI | |
| | | | | LLEGDDDTLSSVDEKDLENLAGPVSLSTPAQLVAPSVVVKGTLSVT | |
| | | | | SSELYFEVDEEDPNFKKIDPKILAYTEGLHGKWLFTEIRSIFSRRYLL | |
| | | | | QNTALEIFMANRVAVMFNFPDPATVKKVVNYLPRVGVGTSFGLPQ | |
| | | | | TRRISLASPRQLFKASNMTQRWQHREISNFEYLMFLNTIAGRSYNDL | |
| | | | | NQYPVFPWVITNYESEELDLTLPTNFRDLSKPIGALNPKRAAFFAER | |
| | | | | YESWEDDQVPKFHYGTHYSTASFVLAWLLRIEPFTTYFLNLQGGKF | |
| | | | | DHADRTFSSISRAWRNSQRDTSDIKELIPEFYYLPEMFVNFNNYNLG | |
| | | | | VMDDGTVVSDVELPPWAKTSEEFVHINRLALESEFVSCQLHQWIDL | |
| | | | | IFGYKQQGPEAVRALNVFYYLTYEGAVNLNSITDPVLREAVEAQIR | |
| | | | | SFGQTPSQLLIEPHPPRGSAMQVSPLMFTDKAQQDVIMVLKFPSNSP | |
| | | | | VTHVAANTQPGLATPAVITVTANRLFAVNKWHNLPAHQGAVQDQ | |
| | | | | PYQLPVEIDPLIASNTGMHRRQITDLLDQSIQVHSQCFVITSDNRYIL | |
| | | | | VCGFWDKSFRVYSTDTGRLIQVVFGHWDVVTCLARSESYIGGNCYI | |
| | | | | LSGSRDATLLLWYWNGKCSGIGDNPGSETAAPRAILTGHDYEVTC | |
| | | | | AAVCAELGLVLSGSQEGPCLIHSMNGDLLRTLEGPENCLKPKLIQAS | |
| | | | | REGHCVIFYENGLFCTFSVNGKLQATMETDDNIRAIQLSRDGQYLL | |
| | | | | TGGDRGVVVVRQVSDLKQLFAYPGCDAGIRAMALSYDQRCIISGM | |
| | | | | ASGSIVLFYNDFNRWHHEYQTRY* | |
| FBXW7 | F-box and WD repeat | 55294 | 708 | MNQELLSVGSKRRRTGGSLRGNPSSSQVDEEQMNRVVEEEQQQQL | 196. |
| | domain containing 7 | | | RQQEEEHTARNGEVVGVEPRPGGQNDSQQGQLEENNNRFISVDED | |
| | [Source:HGNC | | | SSGNQEEQEEDEEHAGEQDEEDEEEEEMDQESDDFDQSDDSSREDE | |
| | Symbol; Acc:HGNC: | | | HTHTNSVTNSSSIVDLPVHQLSSPFYTKTTKMKRKLDHGSEVRSFSL | |
| | 16712] | | | GKKPCKVSEYTSTTGLVPCSATPTTFGDLRAANGQGQQRRRITSVQ | |
| | | | | PPTGLQEWLKMFQSWSGPEKLLALDELIDSCEPTQVKHMMQVIEPQ | |
| | | | | FQRDFISLLPKELALYVLSFLEPKDLLQAAQTCRYWRILAEDNLLW | |
| | | | | REKCKEEGIDEPLHIKRRKVIKPGFIHSPWKSAYIRQHRIDTNWRRG | |
| | | | | ELKSPKVLKGHDDHVITCLQFCGNRIVSGSDDNTLKVWSAVTGKC | |
| | | | | LRTLVGHTGGVWSSQMRDNIIISGSTDRTLKVWNAETGECIHTLYG | |
| | | | | HTSTVRCMHLHEKRVVSGSRDATLRVWDIETGQCLHVLMGHVAA | |
| | | | | VRCVQYDGRRVVSGAYDFMVKVWDPETETCLHTLQGHTNRVYSL | |
| | | | | QFDGIHVVSGSLDTSIRVWDVETGNCIHTLTGHQSLTSGMELKDNIL | |
| | | | | VSGNADSTVKIWDIKTGQCLQTLQGPNKHQSAVTCLQFNKNFVITS | |
| | | | | SDDGTVKLWDLKTGEFIRNLVTLESGGSGGVVWRIRASNTKLVCA | |
| | | | | VGSRNGTEETKLLVLDFDVDMK* | |
| FBXW7 | F-box and WD repeat | 55294 | 590 | MSKPGKPTLNHGLVPVDLKSAKEPLPHQTVMKIFSISIIAQGLPFCRR | 197. |
| | domain containing 7 | | | RMKRKLDHGSEVRSFSLGKKPCKVSEYTSTTGLVPCSATPTTFGDL | |
| | [Source:HGNC | | | RAANGQGQQRRRITSVQPPTGLQEWLKMFQSWSGPEKLLALDELI | |
| | Symbol; Acc:HGNC: | | | DSCEPTQVKHMMQVIEPQFQRDFISLLPKELALYVLSFLEPKDLLQA | |
| | 16712] | | | AQTCRYWRILAEDNLLWREKCKEEGIDEPLHIKRRKVIKPGFIHSPW | |
| | | | | KSAYIRQHRIDTNWRRGELKSPKVLKGHDDHVITCLQFCGNRIVSG | |
| | | | | SDDNTLKVWSAVTGKCLRTLVGHTGGVWSSQMRDNIIISGSTDRTL | |
| | | | | KVWNAETGECIHTLYGHTSTVRCMHLHEKRVVSGSRDATLRVWDI | |
| | | | | ETGQCLHVLMGHVAAVRCVQYDGRRVVSGAYDFMVKVWDPETE | |
| | | | | TCLHTLQGHTNRVYSLQFDGIHVVSGSLDTSIRVWDVETGNCIHTL | |
| | | | | TGHQSLTSGMELKDNILVSGNADSTVKIWDIKTGQCLQTLQGPNKH | |
| | | | | QSAVTCLQFNKNFVITSSDDGTVKLWDLKTGEFIRNLVTLESGGSG | |
| | | | | GVVWRIRASNTKLVCAVGSRNGTEETKLLVLDFDVDMK* | |
| FBXW7 | F-box and WD repeat | 55294 | 628 | MCVPRSGLILSCICLYCGVLLPVLLPNLPFLTCLSMSTLESVTYLPEK | 198. |
| | domain containing 7 | | | GLYCQRLPSSRTHGGTESLKGKNTENMGFYGTLKMIFYKMKRKLD | |
| | [Source:HGNC | | | HGSEVRSFSLGKKPCKVSEYTSTTGLVPCSATPTTFGDLRAANGQG | |
| | Symbol; Acc:HGNC: | | | QQRRRITSVQPPTGLQEWLKMFQSWSGPEKLLALDELIDSCEPTQV | |
| | 16712] | | | KHMMQVIEPQFQRDFISLLPKELALYVLSFLEPKDLLQAAQTCRYW | |
| | | | | RILAEDNLLWREKCKEEGIDEPLHIKRRKVIKPGFIHSPWKSAYIRQ | |
| | | | | HRIDTNWRRGELKSPKVLKGHDDHVITCLQFCGNRIVSGSDDNTLK | |
| | | | | VWSAVTGKCLRTLVGHTGGVWSSQMRDNIIISGSTDRTLKVWNAE | |
| | | | | TGECIHTLYGHTSTVRCMHLHEKRVVSGSRDATLRVWDIETGQCL | |
| | | | | HVLMGHVAAVRCVQYDGRRVVSGAYDFMVKVWDPETETCLHTL | |
| | | | | QGHTNRVYSLQFDGIHVVSGSLDTSIRVWDVETGNCIHTLTGHQSL | |
| | | | | TSGMELKDNILVSGNADSTVKIWDIKTGQCLQTLQGPNKHQSAVTC | |
| | | | | LQFNKNFVITSSDDGTVKLWDLKTGEFIRNLVTLESGGSGGVVWRI | |
| | | | | RASNTKLVCAVGSRNGTEETKLLVLDFDVDMK* | |
| FBXW7 | F-box and WD repeat | 55294 | 70 | MNQELLSVGSKRRRTGGSLRGNPSSSQVDEEQMNRVVEEEQQQQL | 199. |
| | domain containing 7 | | | RQQEEEHTARNGEVVGVEPRPGGQN | |
| | [Source:HGNC | | | | |
| | Symbol; Acc:HGNC: | | | | |
| | 16712] | | | | |
| FBXW7 | F-box and WD repeat | 55294 | 158 | MNQELLSVGSKRRRTGGSLRGNPSSSQVDEEQMNRVVEEEQQQQL | 200. |
| | domain containing 7 | | | RQQEEEHTARNGEVVGVEPRPGGQNDSQQGQLEENNNRFISVDED | |
| | [Source:HGNC | | | | |
| | Symbol; Acc:HGNC: | | | SSGNQEEQEEDEEHAGEQDEEDEEEEEMDQESDDFDQSDDSSREDE | |
| | 16712] | | | HTHTNSVTNSSSIVDLPVHQLS | |
| FBXW7 | F-box and WD repeat | 55294 | 177 | MNQELLSVGSKRRRTGGSLRGNPSSSQVDEEQMNRVVEEEQQQQL | 201. |
| | domain containing 7 | | | RQQEEEHTARNGEVVGVEPRPGGQNDSQQGQLEENNNRFISVDED | |
| | [Source:HGNC | | | SSGNQEEQEEDEEHAGEQDEEDEEEEEMDQESDDFDQSDDSSREDE | |
| | Symbol; Acc:HGNC: | | | HTHTNSVTNSSSIVDLPVHQLSSPFYTKTTKVSIFNILLT* | |
| | 16712] | | | | |
| PRKD3 | protein kinase D3 | 23683 | 891 | MSANNSPPSAQKSVLPTAIPAVLPAASPCSSPKTGLSARLSNGSFSAP | 202. |
| | [Source:HGNC | | | SLTNSRGSVHTVSFLLQIGLTRESVTIEAQELSLSAVKDLVCSIVYQK | |
| | Symbol; Acc:HGNC: | | | FPECGFFGMYDKILLFRHDMNSENILQLITSADEIHEGDLVEVVLSA | |
| | 9408] | | | LATVEDFQIRPHTLYVHSYKAPTFCDYCGEMLWGLVRQGLKCEGC | |
| | | | | GLNYHKRCAFKIPNNCSGVRKRRLSNVSLPGPGLSVPRPLQPEYVA | |
| | | | | LPSEESHVHQEPSKRIPSWSGRPIWMEKMVMCRVKVPHTFAVHSYT | |
| | | | | RPTICQYCKRLLKGLFRQGMQCKDCKFNCHKRCASKVPRDCLGEV | |
| | | | | TFNGEPSSLGTDTDIPMDIDNNDINSDSSRGLDDTEEPSPPEDKMFFL | |
| | | | | DPSDLDVERDEEAVKTISPSTSNNIPLMRVVQSIKHTKRKSSTMVKE | |
| | | | | GWMVHYTSRDNLRKRHYWRLDSKCLTLFQNESGSKYYKEIPLSEIL | |
| | | | | RISSPRDFTNISQGSNPHCFEIITDTMVYFVGENNGDSSHNPVLAATG | |
| | | | | VGLDVAQSWEKAIRQALMPVTPQASVCTSPGQGKDHKDLSTSISVS | |
| | | | | NCQIQENVDISTVYQIFADEVLGSGQFGIVYGGKHRKTGRDVAIKVI | |
| | | | | DKMRFPTKQESQLRNEVAILQNLHHPGIVNLECMFETPERVFVVME | |
| | | | | KLHGDMLEMILSSEKSRLPERITKFMVTQILVALRNLHFKNIVHCDL | |
| | | | | KPENVLLASAEPFPQVKLCDFGFARIIGEKSFRRSVVGTPAYLAPEV | |
| | | | | LRSKGYNRSLDMWSVGVIIYVSLSGTFPFNEDEDINDQIQNAAFMY | |
| | | | | PPNPWREISGEAIDLINNLLQVKMRKRYSVDKSLSHPWLQDYQTWL | |
| | | | | DLREFETRIGERYITHESDDARWEIHAYTHNLVYPKHFIMAPNPDD | |
| | | | | MEEDP* | |
| PRKD3 | protein kinase D3 | 23683 | 185 | XTSISVSNCQIQENVDISTVYQIFADEVLGSGQFGIVYGGKHRKTGR | 203. |
| | [Source:HGNC | | | DVAIKVIDKMRFPTKQESQLRNEVAILQILVALRNLHFKNIVHCDLK | |
| | Symbol; Acc:HGNC: | | | PENVLLASAEPFPQVKLCDFGFARIIGEKSFRRSVVGTPAYLAPEVL | |
| | 9408] | | | RSKGYNRSLDMWSVGVIIYVSLSGTFPFNEDEDINDQIQNAAFM | |
| PRKD3 | protein kinase D3 | 23683 | 173 | TDTMVYFVGENNGDSSHNPVLAATGVGLDVAQSWEKAIRQALMP | 204. |
| | [Source:HGNC | | | VTPQASVCTSPGQGKDHKDLSTSISVSNCQIQENVDISTVYQIFADE | |
| | Symbol; Acc:HGNC: | | | VLGSGQFGIVYGDLSVLRVVTSLEDFLSSEEQRTVRGKHRKTGRDV | |
| | 9408] | | | AIKVIDKMRFPTKQESQLRNEVAILQNLHHPGIVNL | |
| PRKD3 | protein kinase D3 | 23683 | 181 | MYDKILLFRHDMNSENILQLITSADEIHEGDLVEVVLSALATVEDFQ | 205. |
| | [Source:HGNC | | | IRPHTLYVHSYKAPTFCDYCGEMLWGLVRQGLKCEGCGLNYHKRC | |
| | Symbol; Acc:HGNC: | | | AFKIPNNCSGVRKRRLSNVSLPGPGLSVPRPLQPEYVALPSEESHVH | |
| | 9408] | | | QEPSKRIPSWSGRPIWMEKMVMCRVKVPHTFAVHSYTRPTIC | |
| XPNPEP1 | X-prolyl | 7511 | 643 | MAASRKPPRVRVNHQDFQLRNLRIIEPNEVTHSGDTGVETDGRMPP | 206. |
| | aminopeptidase 1 | | | KVTSELLRQLRQAMRNSEYVTEPIQAYIIPSGDAHQSEYIAPCDCRR | |
| | [Source:HGNC | | | AFVSGFDGSAGTAIITEEHAAMWTDGRYFLQAAKQMDSNWTLMK | |
| | Symbol; Acc:HGNC: | | | MGLKDTPTQEDWLVSVLPEGSRVGVDPLIIPTDYWKKMAKVLRSA | |
| | 12822] | | | GHHLIPVKENLVDKIWTDRPERPCKPLLTLGLDYTGISWKDKVADL | |
| | | | | RLKMAERNVMWFVVTALDEIAWLFNLRGSDVEHNPVFFSYAIIGLE | |
| | | | | TIMLFIDGDRIDAPSVKEHLLLDLGLEAEYRIQVHPYKSILSELKALC | |
| | | | | ADLSPREKVWVSDKASYAVSETIPKDHRCCMPYTPICIAKAVKNSA | |
| | | | | ESEGMRRAHIKDAVALCELFNWLEKEVPKGGVTEISAADKAEEFRR | |
| | | | | QQADFVDLSFPTISSTGPNGAIIHYADGTTDVTRTMHFGTPTAYEKE | |
| | | | | CFTYVLKGHIAVSAAVFPTGTKGHLLDSFARSALWDSGLDYLHGT | |
| | | | | GHGVGSFLNVHEGPCGISYKTFSDEPLEAGMIVTDEPGYYEDGAFGI | |
| | | | | RIENVVLVVPVKTKYNFNNRGSLTFEPLTLVPIQTKMIDVDSLTDKE | |
| | | | | CDWLNNYHLTCRDVIGKELQKQGRQEALEWLIRETQPISKQH* | |
| XPNPEP1 | X-prolyl | 7511 | 303 | MPPKVTSELLRQLRQAMRNSEYVTEPIQAYIIPSGDAHQSEYIAPCD | 207. |
| | aminopeptidase 1 | | | CRRAFVSGFDGSAGTAIITEEHAAMWTDGRYFLQAAKQMDSNWTL | |
| | [Source:HGNC | | | MKMGLKDTPTQEDWLVSVLPEGSRVGVDPLIIPTDYWKKMAKVL | |
| | Symbol; Acc:HGNC: | | | RSAGHHLIPVKENLVDKIWTDRPERPCKPLLTLGLDYTGISWKDKV | |
| | 12822] | | | ADLRLKMAERNVMWFVVTALDEIAWLFNLRGSDVEHNPVFFSYAI | |
| | | | | IGLETIMLFIDGDRIDAPSVKEHLLLDLGLEAEYRIQVHPYKSILSEL | |
| | | | | KALCADLSPREKVWVSDKASYAVSETIP | |
| XPNPEP1 | X-prolyl | 7511 | 553 | MWTDGRYFLQAAKQMDSNWTLMKMGLKDTPTQEDWLVSVLPEG | 208. |
| | aminopeptidase 1 | | | SRVGVDPLIIPTDYWKKMAKVLRSAGHHLIPVKENLVDKIWTDRPE | |
| | [Source:HGNC | | | RPCKPLLTLGLDYTGISWKDKVADLRLKMAERNVMWFVVTALDEI | |
| | Symbol; Acc:HGNC: | | | AWLFNLRGSDVEHNPVFFSYAIIGLETIMLFIDGDRIDAPSVKEHLLL | |
| | 12822] | | | DLGLEAEYRIQVHPYKSILSELKALCADLSPREKVWVSDKASYAVS | |
| | | | | ETIPKDHRCCMPYTPICIAKAVKNSAESEGMRRAHIKDAVALCELF | |
| | | | | NWLEKEVPKGGVTEISAADKAEEFRRQQADFVDLSFPTISSTGPNG | |
| | | | | AIIHYAPVPETNRTLSLDEVYLIDSGAQYKDGTTDVTRTMHFGTPTA | |
| | | | | YEKECFTYVLKGHIAVSAAVFPTGTKGHLLDSFARSALWDSGLDYL | |
| | | | | HGTGHGVGSFLNVHEGPCGISYKTFSDEPLEAGMIVTDEPGYYEDG | |
| | | | | AFGIRIENVVLVVPVKTKYNFNNRGSLTFEPLTLVPIQTKMIDVDSL | |
| | | | | TDKECDWLNNYHLTCRDVIGKELQKQGRQEALEWLIRETQPISKQ | |
| | | | | H* | |
| XPNPEP1 | X-prolyl | 7511 | 667 | MAASRKPPRVRVNHQDFQLRNLRIIEPNEVTHSGDTGVETDGRMPP | 209. |
| | aminopeptidase 1 | | | KVTSELLRQLRQAMRNSEYVTEPIQAYIIPSGDAHQSEYIAPCDCRR | |
| | [Source:HGNC | | | AFVSGFDGSAGTAIITEEHAAMWTDGRYFLQAAKQMDSNWTLMK | |
| | Symbol; Acc:HGNC: | | | MGLKDTPTQEDWLVSVLPEGSRVGVDPLIIPTDYWKKMAKVLRSA | |
| | 12822] | | | GHHLIPVKENLVDKIWTDRPERPCKPLLTLGLDYTGISWKDKVADL | |
| | | | | RLKMAERNVMWFVVTALDEIAWLFNLRGSDVEHNPVFFSYAIIGLE | |
| | | | | TIMLFIDGDRIDAPSVKEHLLLDLGLEAEYRIQVHPYKSILSELKALC | |
| | | | | ADLSPREKVWVSDKASYAVSETIPKDHRCCMPYTPICIAKAVKNSA | |
| | | | | ESEGMRRAHIKDAVALCELFNWLEKEVPKGGVTEISAADKAEEFRR | |
| | | | | QQADFVDLSFPTISSTGPNGAIIHYAPVPETNRTLSLDEVYLIDSGAQ | |
| | | | | YKDGTTDVTRTMHFGTPTAYEKECFTYVLKGHIAVSAAVFPTGTK | |
| | | | | GHLLDSFARSALWDSGLDYLHGTGHGVGSFLNVHEGPCGISYKTFS | |
| | | | | DEPLEAGMIVTDEPGYYEDGAFGIRIENVVLVVPVKTKYNFNNRGS | |
| | | | | LTFEPLTLVPIQTKMIDVDSLTDKECDWLNNYHLTCRDVIGKELQK | |
| | | | | QGRQEALEWLIRETQPISKQH* | |
| XPNPEP1 | X-prolyl | 7511 | 233 | MPPKVTSELLRQLRQAMRNSEYVTEPIQAYIIPSGDAHQSEYIAPCD | 210. |
| | aminopeptidase 1 | | | CRRAFVSGFDGSAGTAIITEEHAAMWTDGRYFLQAAKQMDSNWTL | |
| | [Source:HGNC | | | MKMGLKDTPTQEDWLVSVLPEGSRVGVDPLIIPTDYWKKMAKVL | |
| | Symbol; Acc:HGNC: | | | RSAGHHLIPVKENLVDKIWTDRPERPCKPLLTLGLDYTGLFNLRGS | |
| | 12822] | | | DVEHNPVFFSYAIIGLETIMLFIDGDRIDAPSVKEHLLLDLGLEAEYR | |
| | | | | IQV | |
| PRKCE | protein kinase C epsilon | 5581 | 738 | MVVFNGLLKIKICEAVSLKPTAWSLRHAVGPRPQTFLLDPYIALNV | 211. |
| | [Source:HGNC | | | DDSRIGQTATKQKTNSPAWHDEFVTDVCNGRKIELAVFHDAPIGYD | |
| | Symbol; Acc:HGNC: | | | DFVANCTIQFEELLQNGSRHFEDWIDLEPEGRVYVIIDLSGSSGEAP | |
| | 9401] | | | KDNEERVFRERMRPRKRQGAVRRRVHQVNGHKFMATYLRQPTYC | |
| | | | | SHCRDFIWGVIGKQGYQCQVCTCVVHKRCHELIITKCAGLKKQETP | |
| | | | | DQVGSQRFSVNMPHKFGIHNYKVPTFCDHCGSLLWGLLRQGLQCK | |
| | | | | VCKMNVHRRCETNVAPNCGVDARGIAKVLADLGVTPDKITNSGQR | |
| | | | | RKKLIAGAESPQPASGSSPSEEDRSKSAPTSPCDQEIKELENNIRKAL | |
| | | | | SFDNRGEEHRAASSPDGQLMSPGENGEVRQGQAKRLGLDEFNFIKV | |
| | | | | LGKGSFGKVMLAELKGKDEVYAVKVLKKDVILQDDDVDCTMTEK | |
| | | | | RILALARKHPYLTQLYCCFQTKDRLFFVMEYVNGGDLMFQIQRSRK | |
| | | | | FDEPRSRFYAAEVTSALMFLHQHGVIYRDLKLDNILLDAEGHCKLA | |
| | | | | DFGMCKEGILNGVTTTTFCGTPDYIAPEILQELEYGPSVDWWALGV | |
| | | | | LMYEMMAGQPPFEADNEDDLFESILHDDVLYPVWLSKEAVSILKA | |
| | | | | FMTKNPHKRLGCVASQNGEDAIKQHPFFKEIDWVLLEQKKIKPPFK | |
| | | | | PRIKTKRDVNNFDQDFTREEPVLTLVDEAIVKQINQEEFKGFSYFGE | |
| | | | | DLMP* | |
| PRKCE | protein kinase C epsilon | 5581 | 220 | MNVHRRCETNVAPNCGVDARGIAKVLADLGVTPDKITNSGQRRKK | 212. |
| | [Source:HGNC | | | LIAGAESPQPASGSSPSEEDRSKSAPTSPCDQEIKELENNIRKALSFD | |
| | Symbol; Acc:HGNC: | | | NRGEEHRAASSPDGQLMSPGENGEVRQGQAKRLGLDEFNFIKVLG | |
| | 9401] | | | KGSFGKVMLAELKGKDEVYAVKVLKKDVILQDDDVDCTMTEKRI | |
| | | | | LALARKHPYLTQLYCCFQTKGTGFSIQGCACEGMREL* | |
| PRKCE | protein kinase C epsilon | 5581 | 127 | MVVFNGLLKIKICEAVSLKPTAWSLRHAVGPRPQTFLLDPYIALNV | 213. |
| | [Source:HGNC | | | DDSRIGQTATKQKTNSPAWHDEFVTDVCNGRKIELAVFHDAPIGYD | |
| | Symbol; Acc:HGNC: | | | DFVANCTIQFEELLQNGSRHFEDWIDLEPEGRVYV | |
| | 9401] | | | | |
| STAT2 | signal transducer and | 6773 | 852 | MAQWEMLQNLDSPFQDQLHQLYSHSLLPVDIRQYLAVWIEDQNW | 214. |
| | activator of | | | QEAALGSDDSKATMLFFHFLDQLNYECGRCSQDPESLLLQHNLRKF | |
| | transcription 2 | | | CRDIQPFSQDPTQLAEMIFNLLLEEKRILIQAQRAQLEQGEPVLETPV | |
| | [Source:HGNC | | | ESQQHEIESRILDLRAMMEKLVKSISQLKDQQDVFCFRYKIQAKGK | |
| | Symbol; Acc:HGNC: | | | TPSLDPHQTKEQKILQETLNELDKRRKEVLDASKALLGRLTTLIELL | |
| | 11363] | | | LPKLEEWKAQQQKACIRAPIDHGLEQLETWFTAGAKLLFHLRQLLK | |
| | | | | ELKGLSCLVSYQDDPLTKGVDLRNAQVTELLQRLLHRAFVVETQP | |
| | | | | CMPQTPHRPLILKTGSKFTVRTRLLVRLQEGNESLTVEVSIDRNPPQ | |
| | | | | LQGFRKFNILTSNQKTLTPEKGQSQGLIWDFGYLTLVEQRSGGSGK | |
| | | | | GSNKGPLGVTEELHIISFTVKYTYQGLKQELKTDTLPVVIISNMNQL | |
| | | | | SIAWASVLWFNLLSPNLQNQQFFSNPPKAPWSLLGPALSWQFSSYV | |
| | | | | GRGLNSDQLSMLRNKLFGQNCRTEDPLLSWADFTKRESPPGKLPF | |
| | | | | WTWLDKILELVHDHLKDLWNDGRIMGFVSRSQERRLLKKTMSGTF | |
| | | | | LLRFSESSEGGITCSWVEHQDDDKVLIYSVQPYTKEVLQSLPLTEIIR | |
| | | | | HYQLLTEENIPENPLRFLYPRIPRDEAFGCYYQEKVNLQERRKYLKH | |
| | | | | RLIVVSNRQVDELQQPLELKPEPELESLELELGLVPEPELSLDLEPLL | |
| | | | | KAGLDLGPELESVLESTLEPVIEPTLCMVSQTVPEPDQGPVSQPVPE | |
| | | | | PDLPCDLRHLNTEPMEIFRNCVKIEEIMPNGDPLLAGQNTVDEVYVS | |
| | | | | RPSHFYTDGPLMPSDF* | |
| STAT2 | signal transducer and | 6773 | 461 | MAQWEMLQNLDSPFQDQLHQLYSHSLLPVDIRQYLAVWIEDQNW | 215. |
| | activator of | | | QEAALGSDDSKATMLFFHFLDQLNYECGRCSQDPESLLLQHNLRKF | |
| | transcription 2 | | | CRDIQDPTQLAEMIFNLLLEEKRILIQAQRAQLEQGEPVLETPVESQ | |
| | [Source:HGNC | | | QHEIESRILDLRAMMEKLVKSISQLKDQQDVFCFRYKIQAKGKTPSL | |
| | Symbol; Acc:HGNC: | | | DPHQTKEQKILQETLNELDKRRKEVLDASKALLGRLTTLIELLLPKL | |
| | 11363] | | | EEWKAQQQKACIRAPIDHGLEQLETWFTAGAKLLFHLRQLLKELK | |
| | | | | GLSCLVSYQDDPLTKGVDLRNAQVTELLQRLLHRAFVVETQPCMP | |
| | | | | QTPHRPLILKTGSKFTVRTRLLVRLQEGNESLTVEVSIDRNPPQLQG | |
| | | | | FRKFNILTSNQKTLTPEKGQSQGLIWDFGYLTLVEQRSGGSGKGSN | |
| | | | | KGPLGVTEELHIISFTVKYTYQGLKQELKVSENGGQGERKQLWKK | |
| | | | | A* | |
| STAT2 | signal transducer and | 6773 | 204 | MAQWEMLQNLDSPFQDQLHQLYSHSLLPVDIRQYLAVWIEDQNW | 216. |
| | activator of | | | QEAALGSDDSKATMLFFHFLDQLNYECGRCSQDPESLLLQHNLRKF | |
| | transcription 2 | | | CRDIQPFSQDPTQLAEMIFNLLLEEKRILIQAQRAQLEQGEPVLETPV | |
| | [Source:HGNC | | | ESQQHEIESRILDLRAMMEKLVKSISQLKDQQDVFCFRYKIQAKGR | |
| | Symbol; Acc:HGNC: | | | KHIEGLEKDKCLLRSRSWK* | |
| | 11363] | | | | |
| STAT2 | signal transducer and | 6773 | 200 | MAQWEMLQNLDSPFQDQLHQLYSHSLLPVDIRQYLAVWIEDQNW | 217. |
| | activator of | | | QEAALGSDDSKATMLFFHFLDQLNYECGRCSQDPESLLLQHNLRKF | |
| | transcription 2 | | | CRDIQDPTQLAEMIFNLLLEEKRILIQAQRAQLEQGEPVLETPVESQ | |
| | [Source:HGNC | | | QHEIESRILDLRAMMEKLVKSISQLKDQQDVFCFRYKIQAKGRKHIE | |
| | Symbol; Acc:HGNC: | | | GLEKDKCLLRSRSWK* | |
| | 11363] | | | | |
| STAT2 | signal transducer and | 6773 | 66 | MAQWEMLQNLDSPFQDQLHQLYSHSLLPVDIRQYLAVWIEDQNW | 218. |
| | activator of | | | KLHLGVMIPRLPCYSSTSWIS* | |
| | transcription 2 | | | | |
| | [Source:HGNC | | | | |
| | Symbol; Acc:HGNC: | | | | |
| | 11363] | | | | |
| STAT2 | signal transducer and | 6773 | 819 | MAQWEMLQNLDSPFQDQLHQLYSHSLLPVDIRQYLAVWIEDQNW | 219. |
| | activator of | | | QEAALGSDDSKATMLFFHFLDQLNYECGRCSQDPESLLLQHNLRKF | |
| | transcription 2 | | | CRDIQPFSQDPTQLAEMIFNLLLEEKRILIQAQRAQLEQGEPVLETPV | |
| | [Source:HGNC | | | ESQQHEIESRILDLRAMMEKLVKSISQLKDQQDVFCFRYKIQAKGK | |
| | Symbol; Acc:HGNC: | | | TPSLDPHQTKEQKILQETLNELDKRRKEVLDASKALLGRLTTLIELL | |
| | 11363] | | | LPKLEEWKAQQQKACIRAPIDHGLEQLETWFTAGAKLLFHLRQLLK | |
| | | | | ELKGLSCLVSYQDDPLTKGVDLRNAQVTELLQRLLHRAFVVETQP | |
| | | | | CMPQTPHRPLILKTGSKFTVRTRLLVRLQEGNESLTVEVSIDRNPPQ | |
| | | | | LQGFRKFNILTSNQKTLTPEKGQSQGLIWDFGYLTLVEQRSGGSGK | |
| | | | | GSNKGPLGVTEELHIISFTVKYTYQGLKQELKNQQFFSNPPKAPWSL | |
| | | | | LGPALSWQFSSYVGRGLNSDQLSMLRNKLFGQNCRTEDPLLSWAD | |
| | | | | FTKRESPPGKLPFWTWLDKILELVHDHLKDLWNDGRIMGFVSRSQE | |
| | | | | RRLLKKTMSGTFLLRFSESSEGGITCSWVEHQDDDKVLIYSVQPYT | |
| | | | | KEVLQSLPLTEIIRHYQLLTEENIPENPLRFLYPRIPRDEAFGCYYQE | |
| | | | | KVNLQERRKYLKHRLIVVSNRQVDELQQPLELKPEPELESLELELGL | |
| | | | | VPEPELSLDLEPLLKAGLDLGPELESVLESTLEPVIEPTLCMVSQTVP | |
| | | | | EPDQGPVSQPVPEPDLPCDLRHLNTEPMEIFRNCVKIEEIMPNGDPL | |
| | | | | LAGQNTVDEVYVSRPSHFYTDGPLMPSDF* | |
| STAT2 | signal transducer and | 6773 | 848 | MAQWEMLQNLDSPFQDQLHQLYSHSLLPVDIRQYLAVWIEDQNW | 220. |
| | activator of | | | QEAALGSDDSKATMLFFHFLDQLNYECGRCSQDPESLLLQHNLRKF | |
| | transcription 2 | | | CRDIQDPTQLAEMIFNLLLEEKRILIQAQRAQLEQGEPVLETPVESQ | |
| | [Source:HGNC | | | QHEIESRILDLRAMMEKLVKSISQLKDQQDVFCFRYKIQAKGKTPSL | |
| | Symbol; Acc:HGNC:11 | | | DPHQTKEQKILQETLNELDKRRKEVLDASKALLGRLTTLIELLLPKL | |
| | 363] | | | EEWKAQQQKACIRAPIDHGLEQLETWFTAGAKLLFHLRQLLKELK | |
| | | | | GLSCLVSYQDDPLTKGVDLRNAQVTELLQRLLHRAFVVETQPCMP | |
| | | | | QTPHRPLILKTGSKFTVRTRLLVRLQEGNESLTVEVSIDRNPPQLQG | |
| | | | | FRKFNILTSNQKTLTPEKGQSQGLIWDFGYLTLVEQRSGGSGKGSN | |
| | | | | KGPLGVTEELHIISFTVKYTYQGLKQELKTDTLPVVIISNMNQLSIA | |
| | | | | WASVLWFNLLSPNLQNQQFFSNPPKAPWSLLGPALSWQFSSYVGR | |
| | | | | GLNSDQLSMLRNKLFGQNCRTEDPLLSWADFTKRESPPGKLPFWT | |
| | | | | WLDKILELVHDHLKDLWNDGRIMGFVSRSQERRLLKKTMSGTFLL | |
| | | | | RFSESSEGGITCSWVEHQDDDKVLIYSVQPYTKEVLQSLPLTEIIRHY | |
| | | | | QLLTEENIPENPLRFLYPRIPRDEAFGCYYQEKVNLQERRKYLKHRL | |
| | | | | IVVSNRQVDELQQPLELKPEPELESLELELGLVPEPELSLDLEPLLKA | |
| | | | | GLDLGPELESVLESTLEPVIEPTLCMVSQTVPEPDQGPVSQPVPEPDL | |
| | | | | PCDLRHLNTEPMEIFRNCVKIEEIMPNGDPLLAGQNTVDEVYVSRPS | |
| | | | | HFYTDGPLMPSDF* | |
| STAT2 | signal transducer and | 6773 | 262 | MAQWEMLQNLDSPFQDQLHQLYSHSLLPVDIRQYLAVWIEDQNW | 221. |
| | activator of | | | QEAALGSDDSKATMLFFHFLDQLNYECGRCSQDPESLLLQHNLRKF | |
| | transcription 2 | | | CRDIQPFSQDPTQLAEMIFNLLLEEKRILIQAQRAQLEQGEPVLETPV | |
| | [Source:HGNC | | | ESQQHEIESRILDLRAMMEKLVKSISQLKDQQDVFCFRYKIQAKGK | |
| | Symbol; Acc:HGNC: | | | TPSLDPHQTKEQKILQETLNELDKRRKEVLDASKALLGRLTTLIELL | |
| | 11363] | | | LPKLEEWKAQQQKACIRAPIDHGLEQLETW* | |
| SPI1 | Spi-1 proto-oncogene | 6688 | 272 | MLQACKMEGFPLVPPQPSEDLVPYDTDLYQRQTHEYYPYLSSDGES | 222. |
| | [Source:HGNC | | | HSDHYWDFHPHHVHSEFESFAENNFTELQSVQPPQLQQLYRHMEL | |
| | Symbol; Acc:HGNC: | | | EQMHVLDTPMVPPHPSLGHQVSYLPRMCLQYPSLSPAQPSSDEEEG | |
| | 11241] | | | ERQSPPLEVSDGEADGLEPGPGLLPGETGSKKKIRLYQFLLDLLRSG | |
| | | | | DMKDSIWWVDKDKGTFQFSSKHKEALAHRWGIQKGNRKKMTYQ | |
| | | | | KMARALRNYGKTGEVKKVKKKLTYQFSGEVLGRGGLAERRHPPH | |
| | | | | * | |
| SPI1 | Spi-1 proto-oncogene | 6688 | 271 | MLQACKMEGFPLVPPPSEDLVPYDTDLYQRQTHEYYPYLSSDGESH | 223. |
| | [Source HGNC | | | SDHYWDFHPHHVHSEFESFAENNFTELQSVQPPQLQQLYRHMELE | |
| | Symbol; Acc:HGNC: | | | QMHVLDTPMVPPHPSLGHQVSYLPRMCLQYPSLSPAQPSSDEEEGE | |
| | 11241] | | | RQSPPLEVSDGEADGLEPGPGLLPGETGSKKKIRLYQFLLDLLRSGD | |
| | | | | MKDSIWWVDKDKGTFQFSSKHKEALAHRWGIQKGNRKKMTYQK | |
| | | | | MARALRNYGKTGEVKKVKKKLTYQFSGEVLGRGGLAERRHPPH* | |
| SPI1 | Spi-1 proto-oncogene | 6688 | 36 | MLQACKMEGFPLVPPAARRRSACTSSCWTCSAAAT* | 224. |
| | [Source:HGNC | | | | |
| | Symbol; Acc:HGNC: | | | | |
| | 11241] | | | | |
| SPI1 | Spi-1 proto-oncogene | 6688 | 246 | MLQACKMEGFPLVPPPSEDLVPYDTDLYQRQTHEYYPYLSSDGESH | 225. |
| | [Source:HGNC | | | SDHYWDFHPHHVHSEFESFAENNFTELQSVQPPQLQQLYRHMELE | |
| | Symbol; Acc:HGNC: | | | QMHVLDTPMVPPHPSLGHQVSYLPRMCLQYPSLSPAQPSSDEEEGE | |
| | 11241] | | | RQSPPLEVSDGEADGLEPGPGLLPGETGGAPGPPPWPRPWPCLCPAP | |
| | | | | TQPGQLTQQPRHRVACVCVCMFASVCVLWGCLWLSLCVCICCVV | |
| | | | | HTGISMKCARCVCLSVP* | |
| SLC37A3 | solute carrier family 37 | 84255 | 495 | MAWPNVFQRGSLLSQFSHHHVVVFLLTFFSYSLLHASRKTFSNVKV | 226. |
| | member 3 | | | SISEQWTPSAFNTSVELPVEIWSSNHLFPSAEKATLFLGTLDTIFLFSY | |
| | [Source:HGNC | | | AVGLFISGIVGDRLNLRWVLSFGMCSSALVVFVFGALTEWLRFYNK | |
| | Symbol; Acc:HGNC: | | | WLYCCLWIVNGLLQSTGWPCVVAVMGNWFGKAGRGVVFGLWSA | |
| | 20651] | | | CASVGNILGACLASSVLQYGYEYAFLVTASVQFAGGIVIFFGLLVSP | |
| | | | | EEIGLSGIEAEENFEEDSHRPLINGGENEDEYEPNYSIQDDSSVAQVK | |
| | | | | AISFYQACCLPGVIPYSLAYACLKLVNYSFFFWLPFYLSNNFGWKE | |
| | | | | AEADKLSIWYDVGGIIGGTLQGFISDVLQKRAPVLALSLLLAVGSLI | |
| | | | | GYSRSPNDKSINALLMTVTGFFIGGPSNMISSAISADLGRQELIQRSS | |
| | | | | EALATVTGIVDGSGSIGAAVGQYLVSLIRDKLGWMWVFYFFILMTS | |
| | | | | CTIVFISPLIVREIFSLVLRRQAHILRE* | |
| SLC37A3 | solute carrier family 37 | 84255 | 444 | MAWPNVFQRGSLLSQFSHHHVVVFLLTFFSYSLLHASRKTFSNVKV | 227. |
| | member 3 | | | SISEQWTPSAFNTSVELPVEIWSSNHLFPSAEKATLFLGTLDTIFLFSY | |
| | [Source:HGNC | | | AVGLFISGIVGDRLNLRWVLSFGMCSSALVVFVFGALTEWLRFYNK | |
| | Symbol; Acc:HGNC: | | | WLYCCLWIVNGLLQSTGWPCVVAVMGNWFGKAGRGVVFGLWSA | |
| | 20651] | | | CASVGNILGACLASSVLQYGYEYAFLVTASVQFAGGIVIFFGLLVSP | |
| | | | | EEIGLSGIEAEENFEEDSHRPLINGGENEDEYEPNYSIQDDSSVAQVK | |
| | | | | AISFYQACCLPGVIPYSLAYACLKLVNYSFFFWLPFYLSNNFGWKE | |
| | | | | AEADKLSIWYDVGGIIVFSVSDPGQARMDVGFLLFHSHDKLYNCV | |
| | | | | YLAINSEGNILSRAKETGSHIEGVTGARETERTMSATSGPLGLRVCP | |
| | | | | NLGLSRSSSLILDCQASLNTASHLRC* | |
| SLC37A3 | solute carrier family 37 | 84255 | 126 | GLFISGIVGDRLNLRWVLSFGMCSSALVVFVFGALTEWLRFYNKW | 228. |
| | member 3 | | | LYCCLWIVNGLLQSTGWPCVVAVMGNWFGKAGRGVVFGLWSAC | |
| | [Source:HGNC | | | ASVGNILGACLASSVLQYGYEICKDHLSPLSSSMPFW* | |
| | Symbol; Acc:HGNC: | | | | |
| | 20651] | | | | |
| SLC37A3 | solute carrier family 37 | 84255 | 67 | MAWPNVFQRGSLLSQFSHHHVVVFLLTFFSYSLLHASRKTFSNVKV | 229. |
| | member 3 | | | SISEQWTPSAFNTSVELPVE* | |
| | [Source:HGNC | | | | |
| | Symbol; Acc:HGNC: | | | | |
| | 20651] | | | | |
| SLC37A3 | solute carrier family 37 | 84255 | 57 | QRSSEALATVTGIVDGSGSIGAAVGQTSCTIVFISPLIVREIFSLVLRR | 230. |
| | member 3 | | | QAHILRE* | |
| | [Source:HGNC | | | | |
| | Symbol; Acc:HGNC: | | | | |
| | 20651] | | | | |
| SLC37A3 | solute carrier family 37 | 84255 | 151 | XLLLAVGSLIGYSRSPNDKSINALLMTVTGFFIGGPSNMISSAISADL | 231. |
| | member 3 | | | GRQELIQRSSEALATVTGIVDGSGSIGAAVGQYLVSLIRDKLGWMW | |
| | [Source:HGNC | | | VFYFFILMTGSHIEGVTGARETERTMSATSGPLGLRVCPNLGLSRSS | |
| | Symbol; Acc:HGNC: | | | SLILDCQASL | |
| | 20651] | | | | |
| SLC37A3 | solute carrier family 37 | 84255 | 178 | XAEKATLFLGTLDTIFLFSYAVGLFISGIVGDRLNLRWVLSFGMCSS | 232. |
| | member 3 | | | ALVVFVFGALTEWLRFYNKWLYCCLWIVNGLLQSTGWPCVVAVM | |
| | [Source:HGNC | | | GNWFGKAGRGVVFGLWSACASVGNILGACLASSVLQYGYEYSLA | |
| | Symbol; Acc:HGNC: | | | YACLKLVNYSFFFWLPFYLSNNFGWKEAEADKLSIWYDVGGII | |
| | 20651] | | | | |
| SLC37A3 | solute carrier family 37 | 84255 | 99 | XSAISADLGRQELIQRSSEALATVTGIVDGSGSIGAAVGQETGSHIEG | 233. |
| | member 3 | | | VTGARETERTMSATSGPLGLRVCPNLGLSRSSSLILDCQASLNTASH | |
| | [Source:HGNC | | | LRC* | |
| | Symbol; Acc:HGNC: | | | | |
| | 20651] | | | | |
| SLC37A3 | solute carrier family 37 | 84255 | 162 | LSLLLAVGSLIGYSRSPNDKSINALLMTVTGFFIGGPSNMISSAISAD | 234. |
| | member 3 | | | LGRQELIQRSSEALATVTGIVDGSGSIGAAVGQYLVSLIRDKLGWM | |
| | [Source:HGNC | | | WVFYFFILMETGSHIEGVTGARETERTMSATSGPLGLRVCPNLGLSR | |
| | Symbol; Acc:HGNC: | | | SSSLILDCQASLNTASHLRC* | |
| | 20651] | | | | |
| SLC37A3 | solute carrier family 37 | 84255 | 225 | MAWPNVFQRGSLLSQFSHHHVVVFLLTFFSYSLLHASRKTFSNVKV | 235. |
| | member 3 | | | SISEQWTPSAFNTSVELPVEIWSSNHLFPSAEKATLFLGTLDTIFLFSY | |
| | [Source:HGNC | | | AVGLFISGIVGDRLNLRWVLSFGMCSSALVVFVFGALTEWLRFYNK | |
| | Symbol; Acc:HGNC: | | | WLYCCLWIVNGLLQSTGWPCVVAVMGNWFGKAGRGVVFGLWSA | |
| | 20651] | | | CASVGNILGACLASSVLQYGYEVSRVLRQKKTLKKTHTGH* | |
| SLC37A3 | solute carrier family 37 | 84255 | 193 | XIFFGLLVSPEEIGLSGIEAEENFEEDSHRPLINGGENEDEYEPNYSIQ | 236. |
| | member 3 | | | DDSSVAQVKAISFYQACCLPGVIPYSLAYACLKLVNYSFFFWLPFYL | |
| | [Source:HGNC | | | SNNFGWKEAEADKLSIWYDVGGIIGGTLQGFISDVLQKRAPVLALS | |
| | Symbol; Acc:HGNC: | | | LLLAVGSLIGYSLFSVSDPGQARMDVGFLLFHSHDKLYNCVYLAIN | |
| | 20651] | | | SEGNI | |
| SLC37A3 | solute carrier family 37 | 84255 | 55 | MAWPNVFQRGSLLSQFSHHHVVVFLLTFFRCLSLVRSQNGCVSTTN | 237. |
| | member 3 | | | GCTAACGL* | |
| | [Source:HGNC | | | | |
| | Symbol; Acc:HGNC: | | | | |
| | 20651] | | | | |
| SLC37A3 | solute carrier family 37 | 84255 | 123 | GWKEAEADKLSIWYDVGGIIVFSVSDPGQARMDVGFLLFHSHDKL | 238. |
| | member 3 | | | YNCVYLAINSEGNILSRAKETGSHIEGVTGARETERTMSATSGPLGL | |
| | [Source:HGNC | | | RVCPNLGLSRSSSLILDCQASLNTASHLRC* | |
| | Symbol; Acc:HGNC: | | | | |
| | 20651] | | | | |
| SLC37A3 | solute carrier family 37 | 84255 | 153 | MAWPNVFQRGSLLSQFSHHHVVVFLLTFFSYSLLHASRKTFSNVKV | 239. |
| | member 3 | | | SISEQWTPSAFNTSVELPVEIWSSNHLFPSAEKATLFLGTLDTIFLFSY | |
| | [Source:HGNC | | | AVGLFISGIVGDRLNLRWVLSFGMCSSALVYAFLVTASVQFAGGIVI | |
| | Symbol; Acc:HGNC: | | | FFGLLVSPEEI | |
| | 20651] | | | | |
| SLC37A3 | solute carrier family 37 | 84255 | 82 | XYSIQDDSSVAQVKAISFYQACCLPGVIPYLVSLIRDKLGWMWVFY | 240. |
| | member 3 | | | FFILMTSCTIVFISPLIVREIFSLVLRRQAHILRE* | |
| | [Source:HGNC | | | | |
| | Symbol; Acc:HGNC: | | | | |
| | 20651] | | | | |
| SLC37A3 | solute carrier family 37 | 84255 | 151 | XFPSAEKATLFLGTLDTIFLFSYAVGLFISGIVGDRLNLRWVLSFGM | 241. |
| | member 3 | | | CSSALVVFVFGALTEWLRFYNKWLYCCLWIVNGLLQSTGWPCVV | |
| | [Source:HGNC | | | AVMGNWFGKAGRGVVFGLWSACASVGNILGACLASSVLQYGYEI | |
| | Symbol; Acc:HGNC: | | | CKDHLSPLSSSMPFW* | |
| | 20651] | | | | |
| SLC37A3 | solute carrier family 37 | 84255 | 479 | MAWPNVFQRGSLLSQFSHHHVVVFLLTFFSYSLLHASRKTFSNVKV | 242. |
| | member 3 | | | SISEQWTPSAFNTSVELPVEIWSSNHLFPSAEKATLFLGTLDTIFLFSY | |
| | [Source:HGNC | | | AVGLFISGIVGDRLNLRWVLSFGMCSSALVVFVFGALTEWLRFYNK | |
| | Symbol; Acc:HGNC:20 | | | WLYCCLWIVNGLLQSTGWPCVVAVMGNWFGKAGRGVVFGLWSA | |
| | 651] | | | CASVGNILGACLASSVLQYGYEYAFLVTASVQFAGGIVIFFGLLVSP | |
| | | | | EEIGLSGIEAEENFEEDSHRPLINGGENEDEYEPNYSIQDDSSVAQVK | |
| | | | | AISFYQACCLPGVIPYSLAYACLKLVNYSFFFWLPFYLSNNFGWKE | |
| | | | | AEADKLSIWYDVGGIIGGTLQGFISDVLQKRAPVLALSLLLAVGSLI | |
| | | | | GYSRFFIGGPSNMISSAISADLGRQELIQRSSEALATVTGIVDGSGSIG | |
| | | | | AAVGQYLVSLIRDKLGWMWVFYFFILMTSCTIVFISPLIVREIFSLVL | |
| | | | | RRQAHILRE* | |
| SLC37A3 | solute carrier family 37 | 84255 | 14 | MAWPNVFQRGSLLS | 243. |
| | member 3 | | | | |
| | [Source:HGNC | | | | |
| | Symbol; Acc:HGNC: | | | | |
| | 20651] | | | | |
| IRF8 | interferon regulatory | 3394 | 427 | MCDRNGGRRLRQWLIEQIDSSMYPGLIWENEEKSMFRIPWKHAGK | 244. |
| | factors [Source:HGNC | | | QDYNQEVDASIFKAWAVFKGKFKEGDKAEPATWKTRLRCALNKS | |
| | Symbol; Acc:HGNC: | | | PDFEEVTDRSQLDISEPYKVYRIVPEEEQKCKLGVATAGCVNEVTE | |
| | 5358] | | | MECGRSEIDELIKEPSVDDYMGMIKRSPSPPEACRSQLLPDWWAQQ | |
| | | | | PSTGVPLVTGYTTYDAHHSAFSQMVISFYYGGKLVGQATTTCPEGC | |
| | | | | RLSLSQPGLPGTKLYGPEGLELVRFPPADAIPSERQRQVTRKLFGHL | |
| | | | | ERGVLLHSSRQGVFVKRLCQGRVFCSGNAVVCKGRPNKLERDEVV | |
| | | | | QVFDTSQFFRELQQFYNSQGRLPDGRVVLCFGEEFPDMAPLRSKLIL | |
| | | | | VQIEQLYVRQLAEEAGKSCGAGSVMQAPEEPPPDQVFRMFPDICAS | |
| | | | | HQRSFFRENQQITV* | |
| IRF8 | interferon regulatory | 3394 | 153 | XAWAVFKGKFKEGDKAEPATWKTRLRCALNKSPDFEEVTDRSQL | 245. |
| | factors [Source:HGNC | | | DISEPYKVYRIVPEEEQKCKLGVATAGCVNEVTEMECGRSEIDELIK | |
| | Symbol; Acc:HGNC: | | | ESCSSSITARAGFLTAGWCCALGKSFRIWPPCAPNSFSCRLSSCMSG | |
| | 5358] | | | NWQKRLGRAVEPAL* | |
| IRF8 | interferon regulatory | 3394 | 328 | MCDRNGGRRLRQWLIEQIDSSMYPGLIWENEEKSMFRIPWKHAGK | 246. |
| | factors [Source:HGNC | | | QDYNQEVDASIFKAWAVFKGKFKEGDKAEPATWKTRLRCALNKS | |
| | Symbol; Acc:HGNC: | | | PDFEEVTDRSQLDISEPYKVYRIVPEEEQKCKLGVATAGCVNEVTE | |
| | 5358] | | | MECGRSEIDELIKEPSVDDYMGMIKRSPSPPEACRSQLLPDWWAQQ | |
| | | | | PSTGVPLVTGYTTYDAHHSAFSQMVISFYYGGKLVGQATTTCPEGC | |
| | | | | RLSLSQPGLPGTKLYGPEGLELVRFPPADAIPSERQRQVTRKLFGHL | |
| | | | | ERGVLLHSSRQGVFVKRLCQGRVFCSGNAVVCKGRPNKLERDEVV | |
| | | | | QVFDTSQFF | |
| IRF8 | interferon regulatory | 3394 | 14 | MGASWWARPPPPAP | 247. |
| | factors [Source:HGNC | | | | |
| | Symbol; Acc:HGNC: | | | | |
| | 5358] | | | | |
| IRF8 | interferon regulatory | 3394 | 223 | MVISFYYGGKLVGQATTTCPEGCRLSLSQPGLPGTKLYGPEGLELV | 248. |
| | factors [Source:HGNC | | | RFPPADAIPSERQRQVTRKLFGHLERGVLLHSSRQGVFVKRLCQGR | |
| | Symbol; Acc:HGNC: | | | VFCSGNAVVCKGRPNKLERDEVVQVFDTSQFFRELQQFYNSQGRL | |
| | 5358] | | | PDGRVVLCFGEEFPDMAPLRSKLILVQIEQLYVRQLAEEAGKSCGA | |
| | | | | GSVMQAPEEPPPDQVFRMFPDICASHQRSFFRENQQITV* | |
| IRF8 | interferon regulatory | 3394 | 159 | MCDRNGGRRLRQWLIEQIDSSMYPGLIWENEEKSMFRIPWKHAGK | 249. |
| | factors [Source:HGNC | | | QDYNQEVDASIFKAWAVFKGKFKEGDKAEPATWKTRLRCALNKS | |
| | Symbol; Acc:HGNC: | | | PDFEEVTDRSQLDISEPYKVYRIVPEEEQKCKLGVATAGCVNEVTE | |
| | 5358] | | | MECGRSEIDELIKEPSVDDYMGMI | |
| IRF8 | interferon regulatory | 3394 | 220 | MVISFYYGGKLVGQATTTCPEGCRLSLSQPGLPGTKLYGPEGLELV | 250. |
| | factors [Source:HGNC | | | RFPPADAIPSERQRQVTRKLFGHLERGVLLHSSRQGVFVKRLCQGR | |
| | Symbol; Acc:HGNC: | | | VFCSGNAVVCKGRPNKLERDEVVQVFDTSQFFRELQQFYNSQGRL | |
| | 5358] | | | PDGRVVLCFGEEFPDMAPLRSKLILVQIEQLYVRQLAEEAGKSCGA | |
| | | | | GSVMQAPEEPPPDQVFRMFPDICASHQRSFFRENQQI | |
| IRF8 | interferon regulatory | 3394 | 47 | MCDRNGGRRLRQWLIEQIDSSMYPGLIWENEEKSMFRIPWKHAGK | 251. |
| | factors [Source:HGNC | | | QD | |
| | Symbol; Acc:HGNC: | | | | |
| | 5358] | | | | |
| IRF8 | interferon regulatory | 3394 | 127 | MCDRNGGRRLRQWLIEQIDSSMYPGLIWENEEKSMFRIPWKHAGK | 252. |
| | factors [Source:HGNC | | | QDYNQEVDASIFKAWAVFKGKFKEGDKAEPATWKTRLRCALNKS | |
| | Symbol; Acc:HGNC: | | | PDFEEVTDRSQLDISEPYKVYRIVPEEEQKCNYPLWA* | |
| | 5358] | | | | |
| NPRL2 | NPR2 like, GATOR1 | 10641 | 381 | MGSGCRIECIFFSEFHPTLGPKITYQVPEDFISRELFDTVQVYIITKPEL | 253. |
| | complex subunit | | | QNKLITVTAMEKKLIGCPVCIEHKKYSRNALLFNLGFVCDAQAKTC | |
| | [Source:HGNC | | | ALEPIVKKLAGYLTTLELESSFVSMEESKQKLVPIMTILLEELNASGR | |
| | Symbol; Acc:HGNC: | | | CTLPIDESNTIHLKVIEQRPDPPVAQEYDVPVFTKDKEDFFNSQWDL | |
| | 24969] | | | TTQQILPYIDGFRHIQKISAEADVELNLVRIAIQNLLYYGVVTLVSIL | |
| | | | | QYSNVYCPTPKVQDLVDDKSLQEACLSYVTKQGHKRASLRDVFQL | |
| | | | | YCSLSPGTTVRDLIGRHPQQLQHVDERKLIQFGLMKNLIRRLQKYP | |
| | | | | VRVTREEQSHPARLYTGCHSYDEICCKTGMSYHELDERLENDPNIII | |
| | | | | CWK* | |
| NPRL2 | NPR2 like, GATOR1 | 10641 | 58 | MGSGCRIECIFFSEFHPTLGPKITYQVPEDFISRELFDTVQVYIITKPEL | 254. |
| | complex subunit | | | QNKLITV* | |
| | [Source HGNC | | | | |
| | Symbol; Acc:HGNC: | | | | |
| | 24969] | | | | |
| NPRL2 | NPR2 like, GATOR1 | 10641 | 88 | MGSGCRIECIFFSEFHPTLGPKITYQVPEDFISRELFDTVQVYIITKPEL | 255. |
| | complex subunit | | | QNKLITVYGKEADRLSCVHRTQEVQPQCSPLQPGLRV* | |
| | [Source:HGNC | | | | |
| | Symbol; Acc:HGNC: | | | | |
| | 24969] | | | | |
| NPRL2 | NPR2 like, GATOR1 | 10641 | 131 | MGSGCRIECIFFSEFHPTLGPKITYQVPEDFISRELFDTVQVYIITKPEL | 256. |
| | complex subunit | | | QNKLITVTAMEKKLIGCPVCIEHKKYSRNALLFNLGFVCDAQAKTC | |
| | [Source:HGNC | | | ALEPIVKKLAGYLTTLERAASCPWRRASRSWCPS* | |
| | Symbol; Acc:HGNC: | | | | |
| | 24969] | | | | |
| CDK5 | cyclin dependent kinase | 1020 | 261 | MQKYEKLEKIGEGTYGTVFKAKNRETHEIVALKRVRLDDDDEGVP | 257. |
| | 5 [Source:HGNC | | | SSALREICLLKELKHKNIVRLHDVLHSDKKLTLVFEFCDQDLKKYF | |
| | Symbol; Acc:HGNC: | | | DSCNGDLDPEIVKNGELKLADFGLARAFGIPVRCYSAEVVTLWYRP | |
| | 1774] | | | PDVLFGAKLYSTSIDMWSAGCIFAELANAGRPLFPGNDVDDQLKRI | |
| | | | | FRLLGTPTEEQWPSMTKLPDYKPYPMYPATTSLVNVVPKLNATGR | |
| | | | | DLLQNLLKCNPVQRISAEEALQHPYFSDFCPP* | |
| CDK5 | cyclin dependent kinase | 1020 | 293 | MQKYEKLEKIGEGTYGTVFKAKNRETHEIVALKRVRLDDDDEGVP | 258. |
| | 5 [Somce:HGNC | | | SSALREICLLKELKHKNIVRLHDVLHSDKKLTLVFEFCDQDLKKYF | |
| | Symbol; Acc:HGNC: | | | DSCNGDLDPEIVKSFLFQLLKGLGFCHSRNVLHRDLKPQNLLINRN | |
| | 1774] | | | GELKLADFGLARAFGIPVRCYSAEVVTLWYRPPDVLFGAKLYSTSI | |
| | | | | DMWSAGCIFAELANAGRPLFPGNDVDDQLKRIFRLLGTPTEEQWPS | |
| | | | | MTKLPDYKPYPMYPATTSLVNVVPKLNATGRDLLQNLLKCNPVQR | |
| | | | | ISAEEALQHPYFSDFCPP* | |
| ZC3H12A | zinc finger CCCH-type | 80149 | 600 | MSGPCGEKPVLEASPTMSLWEFEDSHSRQGTPRPGQELAAEEASAL | 259. |
| | containing 12A | | | ELQMKVDFFRKLGYSSTEIHSVLQKLGVQADTNTVLGELVKHGTA | |
| | [Source:HGNC | | | TERERQTSPDPCPQLPLVPRGGGTPKAPNLEPPLPEEEKEGSDLRPV | |
| | Symbol; Acc:HGNC: | | | VIDGSNVAMSHGNKEVFSCRGILLAVNWFLERGHTDITVFVPSWRK | |
| | 26259] | | | EQPRPDVPITDQHILRELEKKKILVFTPSRRVGGKRVVCYDDRFIVK | |
| | | | | LAYESDGIVVSNDTYRDLQGERQEWKRFIEERLLMYSFVNDKFMPP | |
| | | | | DDPLGRHGPSLDNFLRKKPLTLEHRKQPCPYGRKCTYGIKCRFFHP | |
| | | | | ERPSCPQRSVADELRANALLSPPRAPSKDKNGRRPSPSSQSSSLLTES | |
| | | | | EQCSLDGKKLGAQASPGSRQEGLTQTYAPSGRSLAPSGGSGSSFGP | |
| | | | | TDWLPQTLDSLPYVSQDCLDSGIGSLESQMSELWGVRGGGPGEPGP | |
| | | | | PRAPYTGYSPYGSELPATAAFSAFGRAMGAGHFSVPADYPPAPPAF | |
| | | | | PPREYWSEPYPLPPPTSVLQEPPVQSPGAGRSPWGRAGSLAKEQAS | |
| | | | | VYTKLCGVFPPHLVEAVMGRFPQLLDPQQLAAEILSYKSQHPSE* | |
| ZC3H12A | zinc finger CCCH-type | 80149 | 131 | DDRFIVKLAYESDGIVVSNDTYRDLQGERQEWKRFIEERLLMYSFV | 260. |
| | containing 12A | | | NDKLAIGPFSAIRSPCMAPTSREFFAPSAGLCPLMTHWAGTGPAWT | |
| | [Source:HGNC | | | TSCVRSHSLWSTGSSRVPMVWNPACPPRPSSLLPFLSPV | |
| | Symbol; Acc:HGNC: | | | | |
| | 26259] | | | | |
| ZC3H12A | zinc finger CCCH-type | 80149 | 284 | MSGPCGEKPVLEASPTMSLWEFEDSHSRQGTPRPGQELAAEEASAL | 261. |
| | containing 12A | | | ELQMKVDFFRKLGYSSTEIHSVLQKLGVQADTNTVLGELVKHGTA | |
| | [Source:HGNC | | | TERERQTSPDPCPQLPLVPRGGGTPKAPNLEPPLPEEEKEGSDLRPV | |
| | Symbol; Acc:HGNC: | | | VIDGSNVAMSHGNKEVFSCRGILLAVNWFLERGHTDITVFVPSWRK | |
| | 26259] | | | EQPRPDVPITDQHILRELEKKKILVFTPSRRVGGKRVVCYDDRFIVK | |
| | | | | LAYESDGIVVSNDTYRDLQGERQEWKRFIEERLLMYSFVNDNKAC | |
| | | | | HRPLLSN* | |
| BICRA | BRD4 interacting | 29998 | 1561 | MDDEDGRCLLDVICDPQALNDFLHGSEKLDSDDLLDNPGEAQSAF | 262. |
| | chromatin remodeling | | | YEGPGLHVQEASGNHLNPEPNQPAPSVDLDFLEDDILGSPATGGGG | |
| | complex associated | | | GGSGGADQPCDILQQSLQEANITEQTLEAEAELDLGPFQLPTLQPAD | |
| | protein [Source:HGNC | | | GGAGPTGAGGAAAVAAGPQALFPGSTDLLGLQGPPTVLTHQALVP | |
| | Symbol; Acc:HGNC: | | | PQDVVNKALSVQPFLQPVGLGNVTLQPIPGLQGLPNGSPGGATAAT | |
| | 4332] (encoded by | | | LGLAPIQVVGQPVMALNTPTSQLLAKQVPVSGYLASAAGPSEPVTL | |
| | GLTSCR1 gene set | | | ASAGVSPQGAGLVIQKNLSAAVATTLNGNSVFGGAGAASAPTGTPS | |
| | forth in Table 1) | | | GQPLAVAPGLGSSPLVPAPNVILHRTPTPIQPKPAGVLPPKLYQLTPK | |
| | | | | PFAPAGATLTIQGEPGALPQQPKAPQNLTFMAAGKAGQNVVLSGFP | |
| | | | | APALQANVFKQPPATTTGAAPPQPPGALSKPMSVHLLNQGSSIVIPA | |
| | | | | QHMLPGQNQFLLPGAPAVQLPQQLSALPANVGGQILAAAAPHTGG | |
| | | | | QLIANPILTNQNLAGPLSLGPVLAPHSGAHSAHILSAAPIQVGQPALF | |
| | | | | QMPVSLAAGSLPTQSQPAPAGPAATTVLQGVTLPPSAVAMLNTPD | |
| | | | | GLVQPATPAAATGEAAPVLTVQPAPQAPPAVSTPLPLGLQQPQAQQ | |
| | | | | PPQAPTPQAAAPPQATTPQPSPGLASSPEKIVLGQPPSATPTAILTQD | |
| | | | | SLQMFLPQERSQQPLSAEGPHLSVPASVIVSAPPPAQDPAPATPVAK | |
| | | | | GAGLGPQAPDSQASPAPAPQIPAAAPLKGPGPSSSPSLPHQAPLGDS | |
| | | | | PHLPSPHPTRPPSRPPSRPQSVSRPPSEPPLHPCPPPQAPPTLPGIFVIQ | |
| | | | | NQLGVPPPASNPAPTAPGPPQPPLRPQSQPPEGPLPPAPHLPPSSTSSA | |
| | | | | VASSSETSSRLPAPTPSDFQLQFPPSQGPHKSPTPPPTLHLVPEPAAPP | |
| | | | | PPPPRTFQMVTTPFPALPQPKALLERFHQVPSGIILQNKAGGAPAAP | |
| | | | | QTSTSLGPLTSPAASVLVSGQAPSGTPTAPSHAPAPAPMAATGLPPL | |
| | | | | LPAENKAFASNLPTLNVAKAASSGPGKPSGLQYESKLSGLKKPPTL | |
| | | | | QPSKEACFLEHLHKHQGSVLHPDYKTAFPSFEDALHRLLPYHVYQG | |
| | | | | ALPSPSDYHKVDEEFETVSTQLLKRTQAMLNKYRLLLLEESRRVSP | |
| | | | | SAEMVMIDRMFIQEEKTTLALDKQLAKEKPDEYVSSSRSLGLPIAAS | |
| | | | | SEGHRLPGHGPLSSSAPGASTQPPPHLPTKLVIRHGGAGGSPSVTWA | |
| | | | | RASSSLSSSSSSSSAASSLDADEDGPMPSRNRPPIKTYEARSRIGLKL | |
| | | | | KIKQEAGLSKVVHNTALDPVHQPPPPPATLKVAEPPPRPPPPPPPTG | |
| | | | | QMNGTVDHPPPAAPERKPLGTAPHCPRLPLRKTYRENVGGPGAPE | |
| | | | | GTPAGRARGGSPAPLPAKVDEATSGLIRELAAVEDELYQRMLKGPP | |
| | | | | PEPAASAAQGTGDPDWEAPGLPPAKRRKSESPDVDQASFSSDSPQD | |
| | | | | DTLTEHLQSAIDSILNLQQAPGRTPAPSYPHAASAGTPASPPPLHRPE | |
| | | | | AYPPSSHNGGLGARTLTR* | |
| BICRA | BRD4 interacting | 29998 | 126 | MDDEDGRCLLDVICDPQALNDFLHGSEKLDSDDLLDNPGEAQSAF | 263. |
| | chromatin remodeling | | | YEGPGLHVQEASGNHLNPEPNQPAPSVDLDFLEDDILGSPATGGGG | |
| | complex associated | | | GGSGGADQPCDILQQSLQEANITEQTLEAEAELDL | |
| | protein [Source:HGNC | | | | |
| | Symbol; Acc:HGNC: | | | | |
| | 4332] (encoded by | | | | |
| | GLTSCR1 gene set | | | | |
| | forth in Table 1) | | | | |
| BICRA | BRD4 interacting | 29998 | 1319 | MALNTPTSQLLAKQVPVSGYLASAAGPSEPVTLASAGVSPQGAGL | 264. |
| | chromatin remodeling | | | VIQKNLSAAVATTLNGNSVFGGAGAASAPTGTPSGQPLAVAPGLGS | |
| | complex associated | | | SPLVPAPNVILHRTPTPIQPKPAGVLPPKLYQLTPKPFAPAGATLTIQ | |
| | protein [Source:HGNC | | | GEPGALPQQPKAPQNLTFMAAGKAGQNVVLSGFPAPALQANVFKQ | |
| | Symbol; Acc:HGNC: | | | PPATTTGAAPPQPPGALSKPMSVHLLNQGSSIVIPAQHMLPGQNQFL | |
| | 4332] (encoded by | | | LPGAPAVQLPQQLSALPANVGGQILAAAAPHTGGQLIANPILTNQN | |
| | GLTSCR1 gene set | | | LAGPLSLGPVLAPHSGAHSAHILSAAPIQVGQPALFQMPVSLAAGSL | |
| | forth in Table 1) | | | PTQSQPAPAGPAATTVLQGVTLPPSAVAMLNTPDGLVQPATPAAAT | |
| | | | | GEAAPVLTVQPAPQAPPAVSTPLPLGLQQPQAQQPPQAPTPQAAAP | |
| | | | | PQATTPQPSPGLASSPEKIVLGQPPSATPTAILTQDSLQMFLPQERSQ | |
| | | | | QPLSAEGPHLSVPASVIVSAPPPAQDPAPATPVAKGAGLGPQAPDSQ | |
| | | | | ASPAPAPQIPAAAPLKGPGPSSSPSLPHQAPLGDSPHLPSPHPTRPPSR | |
| | | | | PPSRPQSVSRPPSEPPLHPCPPPQAPPTLPGIFVIQNQLGVPPPASNPA | |
| | | | | PTAPGPPQPPLRPQSQPPEGPLPPAPHLPPSSTSSAVASSSETSSRLPA | |
| | | | | PTPSDFQLQFPPSQGPHKSPTPPPTLHLVPEPAAPPPPPPRTFQMVTTP | |
| | | | | FPALPQPKALLERFHQVPSGIILQNKAGGAPAAPQTSTSLGPLTSPAA | |
| | | | | SVLVSGQAPSGTPTAPSHAPAPAPMAATGLPPLLPAENKAFASNLPT | |
| | | | | LNVAKAASSGPGKPSGLQYESKLSGLKKPPTLQPSKEACFLEHLHK | |
| | | | | HQGSVLHPDYKTAFPSFEDALHRLLPYHVYQGALPSPSDYHKVDEE | |
| | | | | FETVSTQLLKRTQAMLNKYRLLLLEESRRVSPSAEMVMIDRMFIQE | |
| | | | | EKTTLALDKQLAKEKPDEYVSSSRSLGLPIAASSEGHRLPGHGPLSS | |
| | | | | SAPGASTQPPPHLPTKLVIRHGGAGGSPSVTWARASSSLSSSSSSSSA | |
| | | | | ASSLDADEDGPMPSRNRPPIKTYEARSRIGLKLKIKQEAGLSKVVHN | |
| | | | | TALDPVHQPPPPPATLKVAEPPPRPPPPPPPTGQMNGTVDHPPPAAP | |
| | | | | ERKPLGTAPHCPRLPLRKTYRENVGGPGAPEGTPAGRARGGSPAPL | |
| | | | | PAKVDEATSGLIRELAAVEDELYQRMLKGPPPEPAASAAQGTGDPD | |
| | | | | WEAPGLPPAKRRKSESPDVDQASFSSDSPQDDTLTEHLQSAIDSILN | |
| | | | | LQQAPGRTPAPSYPHAASAGTPASPPPLHRPEAYPPSSHNGGLGART | |
| | | | | LTR* | |
| CPSF7 | cleavage and | 79869 | 515 | MGRPESAGGGSRGPFEGGGRARRAGGIFLTLSILRTRDLPSGAMSE | 265. |
| | polyadenylation | | | GVDLIDIYADEEFNQDPEFNNTDQIDLYDDVLTATSQPSDDRSSSTE | |
| | specific factor 7 | | | PPPPVRQEPSPKPNNKTPAILYTYSGLRNRRAAVYVGSFSWWTTDQ | |
| | [Source:HGNC | | | QLIQVIRSIGVYDVVELKFAENRANGQSKGYAEVVVASENSVHKLL | |
| | Symbol; Acc:HGNC: | | | ELLPGKVLNGEKVDVRPATRQNLSQFEAQARKRECVRVPRGGIPPR | |
| | 30098] | | | AHSRDSSDSADGRATPSENLVPSSARVDKPPSVLPYFNRPPSALPLM | |
| | | | | GLPPPPIPPPPPLSSSFGVPPPPPGIHYQHLMPPPPRLPPHLAVPPPGAI | |
| | | | | PPALHLNPAFFPPPNATVGPPPDTYMKASAPYNHHGSRDSGPPPSTV | |
| | | | | SEAEFEDIMKRNRAISSSAISKAVSGASAGDYSDAIETLLTAIAVIKQ | |
| | | | | SRVANDERCRVLISSLKDCLHGIEAKSYSVGASGSSSRKRHRSRERS | |
| | | | | PSRSRESSRRHRDLLHNEDRHDDYFQERNREHERHRDRERDRHH* | |
| CPSF7 | cleavage and | 79869 | 472 | MSEGVDLIDIYADEEFNQDPEFNNTDQIDLYDDVLTATSQPSDDRSS | 266. |
| | polyadenylation | | | STEPPPPVRQEPSPKPNNKTPAILYTYSGLRNRRAAVYVGSFSWWT | |
| | specific factor 7 | | | TDQQLIQVIRSIGVYDVVELKFAENRANGQSKGYAEVVVASENSVH | |
| | [Source:HGNC | | | KLLELLPGKVLNGEKVDVRPATRQNLSQFEAQARKRECVRVPRGGI | |
| | Symbol; Acc:HGNC: | | | PPRAHSRDSSDSADGRATPSENLVPSSARVDKPPSVLPYFNRPPSAL | |
| | 30098] | | | PLMGLPPPPIPPPPPLSSSFGVPPPPPGIHYQHLMPPPPRLPPHLAVPPP | |
| | | | | GAIPPALHLNPAFFPPPNATVGPPPDTYMKASAPYNHHGSRDSGPPP | |
| | | | | STVSEAEFEDIMKRNRAISSSAISKAVSGASAGDYSDAIETLLTAIAV | |
| | | | | IKQSRVANDERCRVLISSLKDCLHGIEAKSYSVGASGSSSRKRHRSR | |
| | | | | ERSPSRSRESSRRHRDLLHNEDRHDDYFQERNREHERHRDRERDRH | |
| | | | | H* | |
| CPSF7 | cleavage and | 79869 | 146 | MSEGVDLIDIYADEEFNQDPEFNNTDQIDLYDDVLTATSQPSDDRSS | 267. |
| | polyadenylation | | | STEPPPPVRQEPSPKPNNKTPAILYTYSGLRNRRAAVYVGSFSWWT | |
| | specific factor 7 | | | TDQQLIQVIRSIGVYDVVELKFAENRANGQSKGYAEVVVASENSVH | |
| | [Source:HGNC | | | KLLELLP | |
| | Symbol; Acc:HGNC: | | | | |
| | 30098] | | | | |
| CPSF7 | cleavage and | 79869 | 205 | MSEGVDLIDIYADEEFNQDPEFNNTDQIDLYDDVLTATSQPSDDRSS | 268. |
| | polyadenylation | | | STEPPPPVRQEPSPKPNNKTPAILYTYSGLRNRRAAVYVGSFSWWT | |
| | specific factor 7 | | | TDQQLIQVIRSIGVYDVVELKFAENRANGQSKGYAEVVVASENSVH | |
| | [Source:HGNC | | | KLLELLPGKVLNGEKVDVRPATRQNLSQFEAQARKRIPPRAHSRDS | |
| | Symbol; Acc:HGNC: | | | SDSADGRATPSENLVPSSAR | |
| | 30098] | | | | |
| CPSF7 | cleavage and | 79869 | 463 | MSEGVDLIDIYADEEFNQDPEFNNTDQIDLYDDVLTATSQPSDDRSS | 269. |
| | polyadenylation | | | STEPPPPVRQEPSPKPNNKTPAILYTYSGLRNRRAAVYVGSFSWWT | |
| | specific factor 7 | | | TDQQLIQVIRSIGVYDVVELKFAENRANGQSKGYAEVVVASENSVH | |
| | [Source:HGNC | | | KLLELLPGKVLNGEKVDVRPATRQNLSQFEAQARKRIPPRAHSRDS | |
| | Symbol; Acc:HGNC: | | | SDSADGRATPSENLVPSSARVDKPPSVLPYFNRPPSALPLMGLPPPPI | |
| | 30098] | | | PPPPPLSSSFGVPPPPPGIHYQHLMPPPPRLPPHLAVPPPGAIPPALHL | |
| | | | | NPAFFPPPNATVGPPPDTYMKASAPYNHHGSRDSGPPPSTVSEAEFE | |
| | | | | DIMKRNRAISSSAISKAVSGASAGDYSDAIETLLTAIAVIKQSRVAN | |
| | | | | DERCRVLISSLKDCLHGIEAKSYSVGASGSSSRKRHRSRERSPSRSRE | |
| | | | | SSRRHRDLLHNEDRHDDYFQERNREHERHRDRERDRHH* | |
| CPSF7 | cleavage and | 79869 | 374 | MSEGVDLIDIYADEEFNQDPEFNNTDQIDLYDDVLTATSQPSDDRSS | 270. |
| | polyadenylation | | | STEPPPPVRQEPSPKPNNKTPAILYTYSGLRNRRAAVYVGSFSWWT | |
| | specific factor 7 | | | TDQQLIQVIRSIGVYDVVELKFAENRANGQSKGYAEVVVASENSVH | |
| | [Source:HGNC | | | KLLELLPGKVLNGEKVDVRPATRQNLSQFEAQARKRIPPRAHSRDS | |
| | Symbol; Acc:HGNC: | | | SDSADGRATPSENLVPSSARVDKPPSVLPYFNRPPSALPLMVGPPPD | |
| | 30098] | | | TYMKASAPYNHHGSRDSGPPPSTVSEAEFEDIMKRNRAISSSAISKA | |
| | | | | VSGASAGDYSDAIETLLTAIAVIKQSRVANDERCRVLISSLKDCLHG | |
| | | | | IEAKSYSVGASGSSSRKRHRSRERSPSRSRESSRRHRDLLHNEDRHD | |
| | | | | D | |
| CPSF7 | cleavage and | 79869 | 144 | MSEGVDLIDIYADEEFNQDPEFNNTDQIDLYDDVLTATSQPSDDRSS | 271. |
| | polyadenylation | | | STEPPPPVRQEPSPKPNNKTPAILYTYSGLRNRRAAVYVGSFSWWT | |
| | specific factor 7 | | | TDQQLIQVIRSIGVYDVVELKFAENRANGQSKGYAEVVVASENSVH | |
| | [Source:HGNC | | | KLLEL | |
| | Symbol; Acc:HGNC: | | | | |
| | 30098] | | | | |
| CPSF7 | cleavage and | 79869 | 138 | MSEGVDLIDIYADEEFNQDPEFNNTDQIDLYDDVLTATSQPSDDRSS | 272. |
| | polyadenylation | | | STEPPPPVRQEPSPKPNNKTPAILYTYSGLRNRRAAVYVGSFSWWT | |
| | specific factor 7 | | | TDQQLIQVIRSIGVYDVVELKFAENRANGQSKGYAEVVVASENSV | |
| | [Source:HGNC | | | | |
| | Symbol; Acc:HGNC: | | | | |
| | 30098] | | | | |
| CPSF7 | cleavage and | 79869 | 102 | MSEGVDLIDIYADEEFNQDPEFNNTDQIDLYDDVLTATSQPSDDRSS | 273. |
| | polyadenylation | | | STEPPPPVRQEPSPKPNNKTPAILYTYSGLRNRRAAVYVGSFSWVGL | |
| | specific factor 7 | | | ISERELS* | |
| | [Source:HGNC | | | | |
| | Symbol; Acc:HGNC: | | | | |
| | 30098] | | | | |
| CPSF7 | cleavage and | 79869 | 101 | MSEGVDLIDIYADEEFNQDPEFNNTDQIDLYDDVLTATSQPSDDRSS | 274. |
| | polyadenylation | | | STEPPPPVRQEPSPKPNNKTPAILYTYSGLRNRRAAVYVGSFSWGHL | |
| | specific factor 7 | | | RDEISE* | |
| | [Source:HGNC | | | | |
| | Symbol; Acc:HGNC: | | | | |
| | 30098] | | | | |
| CPSF7 | cleavage and | 79869 | 99 | MSEGVDLIDIYADEEFNQDPEFNNTDQIDLYDDVLTATSQPSDDRSS | 275. |
| | polyadenylation | | | STEPPPPVRQEPSPKPNNKTPAILYTYSGLRNRRAAVYVGSFSWESM | |
| | specific factor 7 | | | MWWS* | |
| | [Source:HGNC | | | | |
| | Symbol; Acc:HGNC: | | | | |
| | 30098] | | | | |
| CPSF7 | cleavage and | 79869 | 103 | MSEGVDLIDIYADEEFNQDPEFNNTDQIDLYDDVLTATSQPSDDRSS | 276. |
| | polyadenylation | | | STEPPPPVRQEPSPKPNNKTPAILYTYSGLRNRRAAVYVGSFSWWT | |
| | specific factor 7 | | | TDQQLIQVC* | |
| | [Source:HGNC | | | | |
| | Symbol; Acc:HGNC: | | | | |
| | 30098] | | | | |
| CPSF7 | cleavage and | 79869 | 94 | MSEGVDLIDIYADEEFNQDPEFNNTDQIDLYDDVLTATSQPSDDRSS | 277. |
| | polyadenylation | | | STEPPPPVRQEPSPKPNNKTPAILYTYSGLRNRRAAVYVGSFSWVC* | |
| | specific factor 7 | | | | |
| | [Source:HGNC | | | | |
| | Symbol; Acc:HGNC: | | | | |
| | 30098] | | | | |
| CPSF7 | cleavage and | 79869 | 288 | MSEGVDLIDIYADEEFNQDPEFNNTDQIDLYDDVLTATSQPSDDRSS | 278. |
| | polyadenylation | | | STEPPPPVRQEPSPKPNNKTPAILYTYSGLRNRRAAVYVGSFSWWT | |
| | specific factor 7 | | | TDQQLIQVIRSIGVYDVVELKFAENRANGQSKGYAEVVVASENSVH | |
| | [Source:HGNC | | | KLLELLPGKVLNGEKVDVRPATRQNLSQFEAQARKRIPPRAHSRDS | |
| | Symbol; Acc:HGNC: | | | SDSADGRATPSENLVPSSARVDKPPSVLPYFNRPPSALPLMGLPPPPI | |
| | 30098] | | | PPPPPLSSSFGVPPPPPGIHYQHLMPPPPRLPPHLAVPPPGAIPPALHL | |
| | | | | NPAFFP | |
| CPSF7 | cleavage and | 79869 | 102 | MSEGVDLIDIYADEEFNQDPEFNNTDQIDLYDDVLTATSQPSDDRSS | 279. |
| | polyadenylation | | | STEPPPPVRQEPSPKPNNKTPAILYTYSGLRNRRAAVYVGSFSWQG | |
| | specific factor 7 | | | HLRDEISE* | |
| | [Source:HGNC | | | | |
| | Symbol; Acc:HGNC: | | | | |
| | 30098] | | | | |
| CPSF7 | cleavage and | 79869 | 219 | MSEGVDLIDIYADEEFNQDPEFNNTDQIDLYDDVLTATSQPSDDRSS | 280. |
| | polyadenylation | | | STEPPPPVRQEPSPKPNNKTPAILYTYSGLRNRRAAVYVGSFSWWT | |
| | specific factor 7 | | | TDQQLIQVIRSIGVYDVVELKFAENRANGQSKGYAEVVVASENSVH | |
| | [Source:HGNC | | | KLLELLPGKVLNGEKVDVRPATRQNLSQFEAQARKRIPPRAHSRDS | |
| | Symbol; Acc:HGNC: | | | SDSADGRATPSENLVPSSARVDKPPSVLPYFNRP | |
| | 30098] | | | | |
| MTA2 | metastasis associated 1 | 9219 | 669 | MAANMYRVGDYVYFENSSSNPYLVRRIEELNKTANGNVEAKVVC | 281. |
| | family member 2 | | | LFRRRDISSSLNSLADSNAREFEEESKQPGVSEQQRHQLKHRELFLS | |
| | [Source:HGNC | | | RQFESLPATHIRGKCSVTLLNETDILSQYLEKEDCFFYSLVFDPVQK | |
| | Symbol; Acc:HGNC: | | | TLLADQGEIRVGCKYQAEIPDRLVEGESDNRNQQKMEMKVWDPD | |
| | 74H] | | | NPLTDRQIDQFLVVARAVGTFARALDCSSSIRQPSLHMSAAAASRDI | |
| | | | | TLFHAMDTLQRNGYDLAKAMSTLVPQGGPVLCRDEMEEWSASEA | |
| | | | | MLFEEALEKYGKDFNDIRQDFLPWKSLASIVQFYYMWKTTDRYIQ | |
| | | | | QKRLKAAEADSKLKQVYIPTYTKPNPNQIISVGSKPGMNGAGFQKG | |
| | | | | LTCESCHTTQSAQWYAWGPPNMQCRLCASCWIYWKKYGGLKTPT | |
| | | | | QLEGATRGTTEPHSRGHLSRPEAQSLSPYTTSANRAKLLAKNRQTF | |
| | | | | LLQTTKLTRLARRMCRDLLQPRRAARRPYAPINANAIKAECSIRLPK | |
| | | | | AAKTPLKIHPLVRLPLATIVKDLVAQAPLKPKTPRGTKTPINRNQLS | |
| | | | | QNRGLGGIMVKRAYETMAGAGVPFSANGRPLASGIRSSSQPAAKR | |
| | | | | QKLNPADAPNPVVFVATKDTRALRKALTHLEMRRAARRPNLPLKV | |
| | | | | KPTLIAVRPPVPLPAPSHPASTNEPIVLED* | |
| MTA2 | metastasis associated 1 | 9219 | 496 | MEMKVWDPDNPLTDRQIDQFLVVARAVGTFARALDCSSSIRQPSL | 282. |
| | family member 2 | | | HMSAAAASRDITLFHAMDTLQRNGYDLAKAMSTLVPQGGPVLCR | |
| | [Source:HGNC | | | DEMEEWSASEAMLFEEALEKYGKDFNDIRQDFLPWKSLASIVQFY | |
| | Symbol; Acc:HGNC: | | | YMWKTTDRYIQQKRLKAAEADSKLKQVYIPTYTKPNPNQIISVGSK | |
| | 7411] | | | PGMNGAGFQKGLTCESCHTTQSAQWYAWGPPNMQCRLCASCWIY | |
| | | | | WKKYGGLKTPTQLEGATRGTTEPHSRGHLSRPEAQSLSPYTTSANR | |
| | | | | AKLLAKNRQTFLLQTTKLTRLARRMCRDLLQPRRAARRPYAPINA | |
| | | | | NAIKAECSIRLPKAAKTPLKIHPLVRLPLATIVKDLVAQAPLKPKTPR | |
| | | | | GTKTPINRNQLSQNRGLGGIMVKRAYETMAGAGVPFSANGRPLAS | |
| | | | | GIRSSSQPAAKRQKLNPADAPNPVVFVATKDTRALRKALTHLEMR | |
| | | | | RAARRPNLPLKVKPTLIAVRPPVPLPAPSHPASTNEPIVLED* | |
| ACAT2 | acetyl-CoA | 39 | 398 | MNAGSDPVVIVSAARTIIGSFNGALAAVPVQDLGSTVIKEVLKRAT | 283. |
| | acetyltransferase 2 | | | VAPEDVSEVIFGHVLAAGCGQNPVRQASVGAGIPYSVPAWSCQMIC | |
| | [Source:HGNC | | | GSGLKAVCLAVQSIGIGDSSIVVAGGMENMSKAPHLAYLRTGVKIG | |
| | Symbol; Acc:HGNC: | | | EMPLTDSILCDGLTDAFHNCHMGITAENVAKKWQVSREDQDKVA | |
| | 94] | | | VLSQNRTENAQKAGHFDKEIVPVLVSTRKGLIEVKTDEFPRHGSNIE | |
| | | | | AMSKLKPYFLTDGTGTVTPANASGINDGAAAVVLMKKSEADKRGL | |
| | | | | TPLARIVSWSQVGVEPSIMGIGPIPAIKQAVTKAGWSLEDVDIFEINE | |
| | | | | AFAAVSAAIVKELGLNPEKVNIEGGAIALGHPLGASGCRILVTLLHT | |
| | | | | LERMGRSRGVAALCIGGGMGIAMCVQRE* | |
| FOXP1 | forkhead box P1 | 27086 | 567 | MMQESGTETKSNGSAIQNGSGGSNHLLECGGLREGRSNGETPAVDI | 284. |
| | [Source:HGNC | | | GAADLAHAQQQQQQALQVARQLLLQQQQQQQVSGLKSPKRNDK | |
| | Symbol; Acc:HGNC: | | | QPALQVPVSVAMMTPQVITPQQMQQILQQQVLSPQQLQVLLQQQQ | |
| | 3823] | | | ALMLQQQQLQEFYKKQQEQLQLQLLQQQHAGKQPKEQQQVATQQ | |
| | | | | LAFQQQLLQMQQLQQQHLLSLQRQGLLTIQPGQPALPLQPLAQGMI | |
| | | | | PTELQQLWKEVTSAHTAEETTGNNHSSLDLTTTCVSSSAPSKTSLIM | |
| | | | | NPHASTNGQLSVHTPKRESLSHEEHPHSHPLYGHGVCKWPGCEAV | |
| | | | | CEDFQSFLKHLNSEHALDDRSTAQCRVQMQVVQQLELQLAKDKER | |
| | | | | LQAMMTHLHVKSTEPKAAPQPLNLVSSVTLSKSASEASPQSLPHTP | |
| | | | | TTPTAPLTPVTQGPSVITTTSMHTVGPIRRRYSDKYNVPISSADIAQN | |
| | | | | QEFYKNAEVRPPFTYASLIRQAILESPEKQLTLNEIYNWFTRMFAYF | |
| | | | | RRNAATWKNAVRHNLSLHKCFVRVENVKGAVWTVDEVEFQKRRP | |
| | | | | QKISGVPFAPTSHCISALSE* | |
| FOXP1 | forkhead box P1 | 27086 | 115 | MMQESGTETKSNGSAIQNGSGGSNHLLECGGLREGRSNGETPAVDI | 285. |
| | [Source:HGNC | | | GAADLAHAQQQQQQWHLINHQPSRSPSSWLKRLISSPWELEVLQV | |
| | Symbol; Acc:HGNC: | | | PLWGAVAETKMSGPVCQPNPSPF* | |
| | 3823] | | | | |
| FOXP1 | forkhead box P1 | 27086 | 678 | MMQESGTETKSNGSAIQNGSGGSNHLLECGGLREGRSNGETPAVDI | 286. |
| | [Source:HGNC | | | GAADLAHAQQQQQQALQVARQLLLQQQQQQQVSGLKSPKRNDK | |
| | Symbol; Acc:HGNC: | | | QPALQVPVSVAMMTPQVITPQQMQQILQQQVLSPQQLQVLLQQQQ | |
| | 3823] | | | ALMLQQQQLQEFYKKQQEQLQLQLLQQQHAGKQPKEQQQVATQQ | |
| | | | | LAFQQQLLQMQQLQQQHLLSLQRQGLLTIQPGQPALPLQPLAQGMI | |
| | | | | PTELQQLWKEVTSAHTAEETTGNNHSSLDLTTTCVSSSAPSKTSLIM | |
| | | | | NPHASTNGQLSVHTPKRESLSHEEHPHSHPLYGHGVCKWPGCEAV | |
| | | | | CEDFQSFLKHLNSEHALDDRSTAQCRVQMQVVQQLELQLAKDKER | |
| | | | | LQAMMTHLHVKSTEPKAAPQPLNLVSSVTLSKSASEASPQSLPHTP | |
| | | | | TTPTAPLTPVTQGPSVITTTSMHTVGPIRRRYSDKYNVPISSADIAQN | |
| | | | | QEFYKNAEVRPPFTYASLIRQAILESPEKQLTLNEIYNWFTRMFAYF | |
| | | | | RRNAATWKNAVRHNLSLHKCFVRVENVKGAVWTVDEVEFQKRRP | |
| | | | | QKISGNPSLIKNMQSSHAYCTPLNAALQASMAENSIPLYTTASMGN | |
| | | | | PTLGNLASAIREELNGAMEHTNSNESDSSPGRSPMQAVHPVHVKEE | |
| | | | | PLDPEEAEGPLSLVTTANHSPDFDHDRDYEDEPVNEDME* | |
| FOXP1 | forkhead box P1 | 27086 | 231 | MMTPQVITPQQMQQILQQQVLSPQQLQVLLQQQQALMLQQQQLQ | 287. |
| | [Source:HGNC | | | EFYKKQQEQLQLQLLQQQHAGKQPKEQVATQQLAFQQQLLQMQQ | |
| | Symbol; Acc:HGNC: | | | LQQQHLLSLQRQGLLTIQPGQPALPLQPLAQGMIPTELQQLWKEVT | |
| | 3823] | | | SAHTAEETTGNNHSSLDLTTTCVSSSAPSKTSLIMNPHASTNGQLSV | |
| | | | | HTPKRESLSHEEHPHSHPLYGHGVCKWPGCEAVCEDFQSFLKHLNS | |
| | | | | EHAL | |
| FOXP1 | forkhead box P1 | 27086 | 602 | MMQESGTETKSNGSAIQNGSGGSNHLLECGGLREGRSNGETPAVDI | 288. |
| | [Source:HGNC | | | GAADLAHAQQQQQQALQVARQLLLQQQQQQQVSGLKSPKRNDK | |
| | Symbol; Acc:HGNC: | | | QPALQQQQVATQQLAFQQQLLQMQQLQQQHLLSLQRQGLLTIQPG | |
| | 3823] | | | QPALPLQPLAQGMIPTELQQLWKEVTSAHTAEETTGNNHSSLDLTT | |
| | | | | TCVSSSAPSKTSLIMNPHASTNGQLSVHTPKRESLSHEEHPHSHPLY | |
| | | | | GHGVCKWPGCEAVCEDFQSFLKHLNSEHALDDRSTAQCRVQMQV | |
| | | | | VQQLELQLAKDKERLQAMMTHLHVKSTEPKAAPQPLNLVSSVTLS | |
| | | | | KSASEASPQSLPHTPTTPTAPLTPVTQGPSVITTTSMHTVGPIRRRYS | |
| | | | | DKYNVPISSADIAQNQEFYKNAEVRPPFTYASLIRQAILESPEKQLTL | |
| | | | | NEIYNWFTRMFAYFRRNAATWKNAVRHNLSLHKCFVRVENVKGA | |
| | | | | VWTVDEVEFQKRRPQKISGNPSLIKNMQSSHAYCTPLNAALQASM | |
| | | | | AENSIPLYTTASMGNPTLGNLASAIREELNGAMEHTNSNESDSSPGR | |
| | | | | SPMQAVHPVHVKEEPLDPEEAEGPLSLVTTANHSPDFDHDRDYEDE | |
| | | | | PVNEDME* | |
| FOXP1 | forkhead box P1 | 27086 | 677 | MMQESGTETKSNGSAIQNGSGGSNHLLECGGLREGRSNGETPAVDI | 289. |
| | [Source:HGNC | | | GAADLAHAQQQQQQALQVARQLLLQQQQQQQVSGLKSPKRNDK | |
| | Symbol; Acc:HGNC: | | | QPALQVPVSVAMMTPQVITPQQMQQILQQQVLSPQQLQVLLQQQQ | |
| | 3823] | | | ALMLQQQQLQEFYKKQQEQLQLQLLQQQHAGKQPKEQQQVATQQ | |
| | | | | LAFQQQLLQMQQLQQQHLLSLQRQGLLTIQPGQPALPLQPLAQGMI | |
| | | | | PTELQQLWKEVTSAHTAEETTGNNHSSLDLTTTCVSSSAPSKTSLIM | |
| | | | | NPHASTNGQLSVHTPKRESLSHEEHPHSHPLYGHGVCKWPGCEAV | |
| | | | | CEDFQSFLKHLNSEHALDDRSTAQCRVQMQVVQQLELQLAKDKER | |
| | | | | LQAMMTHLHVKSTEPKAAPQPLNLVSSVTLSKSASEASPQSLPHTP | |
| | | | | TTPTAPLTPVTQGPSVITTTSMHTVGPIRRRYSDKYNVPISSDIAQNQ | |
| | | | | EFYKNAEVRPPFTYASLIRQAILESPEKQLTLNEIYNWFTRMFAYFR | |
| | | | | RNAATWKNAVRHNLSLHKCFVRVENVKGAVWTVDEVEFQKRRP | |
| | | | | QKISGNPSLIKNMQSSHAYCTPLNAALQASMAENSIPLYTTASMGN | |
| | | | | PTLGNLASAIREELNGAMEHTNSNESDSSPGRSPMQAVHPVHVKEE | |
| | | | | PLDPEEAEGPLSLVTTANHSPDFDHDRDYEDEPVNEDME* | |
| FOXP1 | forkhead box P1 | 27086 | 577 | MMTPQVITPQQMQQILQQQVLSPQQLQVLLQQQQALMLQQQLQEF | 290. |
| | [Source:HGNC | | | YKKQQEQLQLQLLQQQHAGKQPKEQQQVATQQLAFQQQLLQMQ | |
| | Symbol; Acc:HGNC: | | | QLQQQHLLSLQRQGLLTIQPGQPALPLQPLAQGMIPTELQQLWKEV | |
| | 3823] | | | TSAHTAEETTGNNHSSLDLTTTCVSSSAPSKTSLIMNPHASTNGQLS | |
| | | | | VHTPKRESLSHEEHPHSHPLYGHGVCKWPGCEAVCEDFQSFLKHL | |
| | | | | NSEHALDDRSTAQCRVQMQVVQQLELQLAKDKERLQAMMTHLH | |
| | | | | VKSTEPKAAPQPLNLVSSVTLSKSASEASPQSLPHTPTTPTAPLTPVT | |
| | | | | QGPSVITTTSMHTVGPIRRRYSDKYNVPISSADIAQNQEFYKNAEVR | |
| | | | | PPFTYASLIRQAILESPEKQLTLNEIYNWFTRMFAYFRRNAATWKN | |
| | | | | AVRHNLSLHKCFVRVENVKGAVWTVDEVEFQKRRPQKISGNPSLI | |
| | | | | KNMQSSHAYCTPLNAALQASMAENSIPLYTTASMGNPTLGNLASAI | |
| | | | | REELNGAMEHTNSNESDSSPGRSPMQAVHPVHVKEEPLDPEEAEGP | |
| | | | | LSLVTTANHSPDFDHDRDYEDEPVNEDME* | |
| FOXP1 | forkhead box P1 | 27086 | 123 | MMTPQVITPQQMQQILQQQVLSPQQLQVLLQQQQALMLQQQQLQ | 291. |
| | [Source:HGNC | | | EFYKKQQEQLQLQLLQQQHAGKQPKEQQQVATQQLAFQQQLLQM | |
| | Symbol; Acc:HGNC: | | | QQLQQQHLLSLQRQGLLTIQPGQPALPLQPLAQGM | |
| | 3823] | | | | |
| FOXP1 | forkhead box P1 | 27086 | 36 | MMQESGTETKSNGSAIQNGSGGSNHLLECGGLREGR | 292. |
| | [Source:HGNC | | | | |
| | Symbol; Acc:HGNC: | | | | |
| | 3823] | | | | |
| FOXP1 | forkhead box P1 | 27086 | 208 | XALQVPVSVAMMTPQVITPQQMQQILQQQVLSPQQLQVLLQQQQA | 293. |
| | [Source:HGNC | | | LMLQQQQLQEFYKKQQEQLQLQLLQQQHAGKQPKEQQQVATQQL | |
| | Symbol; Acc:HGNC: | | | AFQQQLLQMQQLQQQHLLSLQRQGLLTIQPGQPALPLQPLAQGMIP | |
| | 3823] | | | TELQQLWKEVTSAHTAEETTGNNHSSLDLTTTCVSSSAPSKTSLIMN | |
| | | | | PHASTNGQLSVHTPKRERQSFHTQR* | |
| FOXP1 | forkhead box P1 | 27086 | 491 | MQQLQQQHLLSLQRQGLLTIQPGQPALPLQPLAQGMIPTELQQLWK | 294. |
| | [Source:HGNC | | | EVTSAHTAEETTGNNHSSLDLTTTCVSSSAPSKTSLIMNPHASTNGQ | |
| | Symbol; Acc:HGNC: | | | LSVHTPKRESLSHEEHPHSHPLYGHGVCKWPGCEAVCEDFQSFLKH | |
| | 3823] | | | LNSEHALDDRSTAQCRVQMQVVQQLELQLAKDKERLQAMMTHLH | |
| | | | | VKSTEPKAAPQPLNLVSSVTLSKSASEASPQSLPHTPTTPTAPLTPVT | |
| | | | | QGPSVITTTSMHTVGPIRRRYSDKYNVPISSADIAQNQEFYKNAEVR | |
| | | | | PPFTYASLIRQAILESPEKQLTLNEIYNWFTRMFAYFRRNAATWKN | |
| | | | | AVRHNLSLHKCFVRVENVKGAVWTVDEVEFQKRRPQKISGNPSLI | |
| | | | | KNMQSSHAYCTPLNAALQASMAENSIPLYTTASMGNPTLGNLASAI | |
| | | | | REELNGAMEHTNSNESDSSPGRSPMQAVHPVHVKEEPLDPEEAEGP | |
| | | | | LSLVTTANHSPDFDHDRDYEDEPVNEDME* | |
| FOXP1 | forkhead box P1 | 27086 | 613 | MMQESGTETKSNGSAIQNGSGGSNHLLECGGLREGRSNGETPAVDI | 295. |
| | [Source:HGNC | | | GAADLAHAQQQQQQALQVARQLLLQQQQQQQVSGLKSPKRNDK | |
| | Symbol; Acc:HGNC: | | | QPALQVPVSVAMMTPQVITPQQMQQILQQQVLSPQQLQVLLQQQQ | |
| | 3823] | | | ALMLQQQLQEFYKKQQEQLQLQLLQQQHAGKQPKEQQQVATQQL | |
| | | | | AFQQQLLQMQQLQQQHLLSLQRQGLLTIQPGQPALPLQPLAQGMIP | |
| | | | | TELQQLWKEVTSAHTAEETTGNNHSSLDLTTTCVSSSAPSKTSLIMN | |
| | | | | PHASTNGQLSVHTPKRESLSHEEHPHSHPLYGHGVCKWPGCEAVC | |
| | | | | EDFQSFLKHLNSEHALDDRSTAQCRVQMQVVQQLELQLAKDKERL | |
| | | | | QAMMTHLHVKSTEPKAAPQPLNLVSSVTLSKSASEASPQSLPHTPT | |
| | | | | TPTAPLTPVTQGPSVITTTSMHTVGPIRRRYSDKYNVPISSADIAQNQ | |
| | | | | EFYKNAEVRPPFTYASLIRQAILESPEKQLTLNEIYNWFTRMFAYFR | |
| | | | | RNAATWKASMAENSIPLYTTASMGNPTLGNLASAIREELNGAMEH | |
| | | | | TNSNESDSSPGRSPMQAVHPVHVKEEPLDPEEAEGPLSLVTTANHSP | |
| | | | | DFDHDRDYEDEPVNEDME* | |
| FOXP1 | forkhead box P1 | 27086 | 411 | MMTPQVITPQQMQQILQQQVLSPQQLQVLLQQQQALMLQQQQLQ | 296. |
| | [Source:HGNC | | | EFYKKQQEQLQLQLLQQQHAGKQPKEQQQVATQQLAFQQQLLQM | |
| | Symbol; Acc:HGNC: | | | QQLQQQHLLSLQRQGLLTIQPGQPALPLQPLAQGMIPTELQQLWKE | |
| | 2383] | | | VTSAHTAEETTGNNHSSLDLTTTCVSSSAPSKTSLIMNPHASTNGQL | |
| | | | | SVHTPKRESLSHEEHPHSHPLYGHGVCKWPGCEAVCEDFQSFLKHL | |
| | | | | NSEHALDDRSTAQCRVQMQVVQQLELQLAKDKERLQAMMTHLH | |
| | | | | VKSTEPKAAPQPLNLVSSVTLSKSASEASPQSLPHTPTTPTAPLTPVT | |
| | | | | QGPSVITTTSMHTVGPIRRRYSDKYNVPISSADIAQNQEFYKNAEVR | |
| | | | | PPFTYASLIRQAILESPEKQLTLNEIYNWFTRMFAYFRRNAATWK* | |
| FOXP1 | forkhead box P1 | 27086 | 335 | MMQESGTETKSNGSAIQNGSGGSNHLLECGGLREGRSNGETPAVDI | 297. |
| | [Source:HGNC | | | GAADLAHAQQQQQQALQVARQLLLQQQQQQQVSGLKSPKRNDK | |
| | Symbol; Acc:HGNC: | | | QPALQVPVSVAMMTPQVITPQQMQQILQQQVLSPQQLQVLLQQQQ | |
| | 3823] | | | ALMLQQQLQEFYKKQQEQLQLQLLQQQHAGKQPKEQQQVATQQL | |
| | | | | AFQQQLLQMQQLQQQHLLSLQRQGLLTIQPGQPALPLQPLAQGMIP | |
| | | | | TELQQLWKEVTSAHTAEETTGNNHSSLDLTTTCVSSSAPSKTSLIMN | |
| | | | | PHASTNGQLSVHTPKRESLSHEEHPHSHPLYGHGVCKWPGCEAVC | |
| | | | | EDFQSFLNLQKTKNACKP* | |
| FOXP1 | forkhead box P1 | 27086 | 578 | MMTPQVITPQQMQQILQQQVLSPQQLQVLLQQQQALMLQQQQLQ | 298. |
| | [Source:HGNC | | | EFYKKQQEQLQLQLLQQQHAGKQPKEQQQVATQQLAFQQQLLQM | |
| | Symbol; Acc:HGNC: | | | QQLQQQHLLSLQRQGLLTIQPGQPALPLQPLAQGMIPTELQQLWKE | |
| | 3823] | | | VTSAHTAEETTGNNHSSLDLTTTCVSSSAPSKTSLIMNPHASTNGQL | |
| | | | | SVHTPKRESLSHEEHPHSHPLYGHGVCKWPGCEAVCEDFQSFLKHL | |
| | | | | NSEHALDDRSTAQCRVQMQVVQQLELQLAKDKERLQAMMTHLH | |
| | | | | VKSTEPKAAPQPLNLVSSVTLSKSASEASPQSLPHTPTTPTAPLTPVT | |
| | | | | QGPSVITTTSMHTVGPIRRRYSDKYNVPISSADIAQNQEFYKNAEVR | |
| | | | | PPFTYASLIRQAILESPEKQLTLNEIYNWFTRMFAYFRRNAATWKN | |
| | | | | AVRHNLSLHKCFVRVENVKGAVWTVDEVEFQKRRPQKISGNPSLI | |
| | | | | KNMQSSHAYCTPLNAALQASMAENSIPLYTTASMGNPTLGNLASAI | |
| | | | | REELNGAMEHTNSNESDSSPGRSPMQAVHPVHVKEEPLDPEEAEGP | |
| | | | | LSLVTTANHSPDFDHDRDYEDEPVNEDME* | |
| FOXP1 | forkhead box P1 | 27086 | 17 | MMQESGTETKSNGSAIQ | 299. |
| | [Source:HGNC | | | | |
| | Symbol; Acc:HGNC: | | | | |
| | 3823] | | | | |
| FOXP1 | forkhead box P1 | 27086 | 677 | MMQESGTETKSNGSAIQNGSGGSNHLLECGGLREGRSNGETPAVDI | 300. |
| | [Source:HGNC | | | GAADLAHAQQQQQQALQVARQLLLQQQQQQQVSGLKSPKRNDK | |
| | Symbol; Acc:HGNC: | | | QPALQVPVSVAMMTPQVITPQQMQQILQQQVLSPQQLQVLLQQQQ | |
| | 3823] | | | ALMLQQQLQEFYKKQQEQLQLQLLQQQHAGKQPKEQQQVATQQL | |
| | | | | AFQQQLLQMQQLQQQHLLSLQRQGLLTIQPGQPALPLQPLAQGMIP | |
| | | | | TELQQLWKEVTSAHTAEETTGNNHSSLDLTTTCVSSSAPSKTSLIMN | |
| | | | | PHASTNGQLSVHTPKRESLSHEEHPHSHPLYGHGVCKWPGCEAVC | |
| | | | | EDFQSFLKHLNSEHALDDRSTAQCRVQMQVVQQLELQLAKDKERL | |
| | | | | QAMMTHLHVKSTEPKAAPQPLNLVSSVTLSKSASEASPQSLPHTPT | |
| | | | | TPTAPLTPVTQGPSVITTTSMHTVGPIRRRYSDKYNVPISSADIAQNQ | |
| | | | | EFYKNAEVRPPFTYASLIRQAILESPEKQLTLNEIYNWFTRMFAYFR | |
| | | | | RNAATWKNAVRHNLSLHKCFVRVENVKGAVWTVDEVEFQKRRP | |
| | | | | QKISGNPSLIKNMQSSHAYCTPLNAALQASMAENSIPLYTTASMGN | |
| | | | | PTLGNLASAIREELNGAMEHTNSNESDSSPGRSPMQAVHPVHVKEE | |
| | | | | PLDPEEAEGPLSLVTTANHSPDFDHDRDYEDEPVNEDME* | |
| FOXP1 | forkhead box P1 | 27086 | 673 | MMQESGTETKSNGSAIQNGSGGSNHLLECGGLREGRSNGETPAVDI | 301. |
| | [Source:HGNC | | | GAADLAHAQQQQQQVARQLLLQQQQQQQVSGLKSPKRNDKQPAL | |
| | Symbol; Acc:HGNC: | | | QVPVSVAMMTPQVITPQQMQQILQQQVLSPQQLQVLLQQQQALML | |
| | 3823] | | | QQQLQEFYKKQQEQLQLQLLQQQHAGKQPKEQQQVATQQLAFQQ | |
| | | | | QLLQMQQLQQQHLLSLQRQGLLTIQPGQPALPLQPLAQGMIPTELQ | |
| | | | | QLWKEVTSAHTAEETTGNNHSSLDLTTTCVSSSAPSKTSLIMNPHAS | |
| | | | | TNGQLSVHTPKRESLSHEEHPHSHPLYGHGVCKWPGCEAVCEDFQ | |
| | | | | SFLKHLNSEHALDDRSTAQCRVQMQVVQQLELQLAKDKERLQAM | |
| | | | | MTHLHVKSTEPKAAPQPLNLVSSVTLSKSASEASPQSLPHTPTTPTA | |
| | | | | PLTPVTQGPSVITTTSMHTVGPIRRRYSDKYNVPISSDIAQNQEFYKN | |
| | | | | AEVRPPFTYASLIRQAILESPEKQLTLNEIYNWFTRMFAYFRRNAAT | |
| | | | | WKNAVRHNLSLHKCFVRVENVKGAVWTVDEVEFQKRRPQKISGN | |
| | | | | PSLIKNMQSSHAYCTPLNAALQASMAENSIPLYTTASMGNPTLGNL | |
| | | | | ASAIREELNGAMEHTNSNESDSSPGRSPMQAVHPVHVKEEPLDPEE | |
| | | | | AEGPLSLVTTANHSPDFDHDRDYEDEPVNEDME* | |
| FOXP1 | forkhead box P1 | 27086 | 148 | MMQESGTETKSNGSAIQNGSGGSNHLLECGGLREGRSNGETPAVDI | 302. |
| | [Source:HGNC | | | GAADLAHAQQQQQQALQVARQLLLQQQQQQQVSGLKSPKRNDK | |
| | Symbol; Acc:HGNC: | | | QPALQVPVSVAMMTPQVITPQQMQQILQQQVLSPQQLQVLLQQQQ | |
| | 2383] | | | ALMLQQQQLQEFYK | |
| FOXP1 | forkhead box P1 | 27086 | 210 | MMTPQVITPQQMQQILQQQVLSPQQLQVLLQQQQALMLQQQLQEF | 303. |
| | [Source:HGNC | | | YKKQQEQLQLQLLQQQHAGKQPKEQQQVATQQLAFQQQLLQMQ | |
| | Symbol; Acc:HGNC: | | | QLQQQHLLSLQRQGLLTIQPGQPALPLQPLAQGMIPTELQQLWKEV | |
| | 3823] | | | TSAHTAEETTGNNHSSLDLTTTCVSSSAPSKTSLIMNPHASTNGQLS | |
| | | | | VHTPKRESLSHEEHPHSHPLYGHGVCKWP | |
| FOXP1 | forkhead box P1 | 27086 | 576 | MMTPQVITPQQMQQILQQQVLSPQQLQVLLQQQQALMLQQQLQEF | 304. |
| | [Source:HGNC | | | YKKQQEQLQLQLLQQQHAGKQPKEQQQVATQQLAFQQQLLQMQ | |
| | Symbol; Acc:HGNC: | | | QLQQQHLLSLQRQGLLTIQPGQPALPLQPLAQGMIPTELQQLWKEV | |
| | 3823] | | | TSAHTAEETTGNNHSSLDLTTTCVSSSAPSKTSLIMNPHASTNGQLS | |
| | | | | VHTPKRESLSHEEHPHSHPLYGHGVCKWPGCEAVCEDFQSFLKHL | |
| | | | | NSEHALDDRSTAQCRVQMQVVQQLELQLAKDKERLQAMMTHLH | |
| | | | | VKSTEPKAAPQPLNLVSSVTLSKSASEASPQSLPHTPTTPTAPLTPVT | |
| | | | | QGPSVITTTSMHTVGPIRRRYSDKYNVPISSADIAQNQEFYKNAEVR | |
| | | | | PPFTYASLIRQAILESPEKQLTLNEIYNWFTRMFAYFRRNAATWKN | |
| | | | | AVRHNLSLHKCFVRVENVKGAVWTVDEVEFQKRRPQKISGNPSLI | |
| | | | | KNMQSSHAYCTPLNAALQASMAENSIPLYTTASMGNPTLGNLASAI | |
| | | | | REELNGAMEHTNSNESDSSPGRSPMQAVHPVHVKEEPLDPEEAEGP | |
| | | | | LSLVTTANHSPDFDHDRDYEDEPVNEDME | |
| FOXP1 | forkhead box P1 | 27086 | 74 | MMTPQVITPQQMQQILQQQVLSPQQLQVLLQQQQALMLQQQQLQ | 305. |
| | [Source:HGNC | | | EFYKKQQEQLQLQLLQQQHAGKQPKEFVP* | |
| | Symbol; Acc:HGNC: | | | | |
| | 3823] | | | | |
| FOXP1 | forkhead box P1 | 27086 | 694 | MMQESGTETKSNGSAIQNGSGGSNHLLECGGLREGRSNGETPAVDI | 306. |
| | [Source:HGNC | | | GAADLAHAQQQQQQALQVARQLLLQQQQQQQVSGLKSPKRNDK | |
| | Symbol; Acc:HGNC: | | | QPALQVPVSVAMMTPQVITPQQMQQILQQQVLSPQQLQVLLQQQQ | |
| | 3823] | | | ALMLQQQQLQEFYKKQQEQLQLQLLQQQHAGKQPKEQQQVATQQ | |
| | | | | LAFQQQLLQMQQLQQQHLLSLQRQGLLTIQPGQPALPLQPLAQGMI | |
| | | | | PTELQQLWKEVTSAHTAEETTGNNHSSLDLTTTCVSSSAPSKTSLIM | |
| | | | | NPHASTNGQLSVHTPKRESLSHEEHPHSHPLYGHGVCKWPGCEAV | |
| | | | | CEDFQSFLKHLNSEHALDDRSTAQCRVQMQVVQQLELQLAKDKER | |
| | | | | LQAMMTHLHVKSTEPKAAPQPLNLVSSVTLSKSASEASPQSLPHTP | |
| | | | | TTPTAPLTPVTQGPSVITTTSMHTVGPIRRRYSDKYNVPISSADIAQN | |
| | | | | QEFYKNAEVRPPFTYASLIRQAILESPEKQLTLNEIYNWFTRMFAYF | |
| | | | | RRNAATWKGAIRTNLSLHKCFIRVEDEFGSFWTVDDEEFKRGRHIQ | |
| | | | | RGRPRKYCPDENFDELVAHNPSLIKNMQSSHAYCTPLNAALQASM | |
| | | | | AENSIPLYTTASMGNPTLGNLASAIREELNGAMEHTNSNESDSSPGR | |
| | | | | SPMQAVHPVHVKEEPLDPEEAEGPLSLVTTANHSPDFDHDRDYEDE | |
| | | | | PVNEDME* | |
| FOXP1 | forkhead box P1 | 27086 | 75 | MMQESGTETKSNGSAIQNGSGGSNHLLECGGLREGRSNGETPAVDI | 307. |
| | [Source:HGNC | | | GAADLAHAQQQQQQALQVARQLLLQQQQQ | |
| | Symbol; Acc:HGNC: | | | | |
| | 3823] | | | | |
| FOXP1 | forkhead box P1 | 27086 | 676 | MMQESGTETKSNGSAIQNGSGGSNHLLECGGLREGRSNGETPAVDI | 308. |
| | [Source:HGNC | | | GAADLAHAQQQQQQALQVARQLLLQQQQQQQVSGLKSPKRNDK | |
| | Symbol; Acc:HGNC: | | | QPALQVPVSVAMMTPQVITPQQMQQILQQQVLSPQQLQVLLQQQQ | |
| | 3823] | | | ALMLQQQLQEFYKKQQEQLQLQLLQQQHAGKQPKEQQQVATQQL | |
| | | | | AFQQQLLQMQQLQQQHLLSLQRQGLLTIQPGQPALPLQPLAQGMIP | |
| | | | | TELQQLWKEVTSAHTAEETTGNNHSSLDLTTTCVSSSAPSKTSLIMN | |
| | | | | PHASTNGQLSVHTPKRESLSHEEHPHSHPLYGHGVCKWPGCEAVC | |
| | | | | EDFQSFLKHLNSEHALDDRSTAQCRVQMQVVQQLELQLAKDKERL | |
| | | | | QAMMTHLHVKSTEPKAAPQPLNLVSSVTLSKSASEASPQSLPHTPT | |
| | | | | TPTAPLTPVTQGPSVITTTSMHTVGPIRRRYSDKYNVPISSDIAQNQE | |
| | | | | FYKNAEVRPPFTYASLIRQAILESPEKQLTLNEIYNWFTRMFAYFRR | |
| | | | | NAATWKNAVRHNLSLHKCFVRVENVKGAVWTVDEVEFQKRRPQ | |
| | | | | KISGNPSLIKNMQSSHAYCTPLNAALQASMAENSIPLYTTASMGNPT | |
| | | | | LGNLASAIREELNGAMEHTNSNESDSSPGRSPMQAVHPVHVKEEPL | |
| | | | | DPEEAEGPLSLVTTANHSPDFDHDRDYEDEPVNEDME* | |
| FOXP1 | forkhead box P1 | 27086 | 210 | MQQLQQQHLLSLQRQGLLTIQPGQPALPLQPLAQGMIPTELQQLWK | 309. |
| | [Source:HGNC | | | EVTSAHTAEETTGNNHSSLDLTTTCVSSSAPSKTSLIMNPHASTNGQ | |
| | Symbol; Acc:HGNC: | | | LSVHTPKRESLSHEEHPHSHPLYGHGVCKWPGCEAVCEDFQSFLKH | |
| | 3823] | | | LNSEHALDDRSTAQCRVQMQVVQQLELQLAKDKERLQAMMTHLH | |
| | | | | VKSTEPKAAPQPLNLVSSVTLSKSASE | |
| FOXP1 | forkhead box P1 | 27086 | 628 | MMQESGTETKSNGSAIQNGSGGSNHLLECGGLREGRSNGETPAVDI | 310. |
| | [Source:HGNC | | | GAADLAHAQQQQQQALQVARQLLLQQQQQQQVSGLKSPKRNDK | |
| | Symbol; Acc:HGNC: | | | QPALQVPVSVAMMTPQVITPQQMQQILQQQVLSPQQLQVLLQQQQ | |
| | 3823] | | | ALMLQQQQLQEFYKKQQEQLQLQLLQQQHAGKQPKEQQQVATQQ | |
| | | | | LAFQQQLLQMQQLQQQHLLSLQRQGLLTIQPGQPALPLQPLAQGMI | |
| | | | | PTELQQLWKEVTSAHTAEETTGNNHSSLDLTTTCVSSSAPSKTSLIM | |
| | | | | NPHASTNGQLSVHTPKRESLSHEEHPHSHPLYGHGVCKWPGCEAV | |
| | | | | CEDFQSFLKHLNSEHALDDRSTAQCRVQMQVVQQLELQLAKDKER | |
| | | | | LQAMMTHLHVKSTEPKAAPQPLNLVSSVTLSKSASEASPQSLPHTP | |
| | | | | TTPTAPLTPVTQGPSVITTTSMHTVGPIRRRYSDKYNVPISSADIAQN | |
| | | | | QEFYKNAEVRPPFTYASLIRQAILESPEKQLTLNEIYNWFTRMFAYF | |
| | | | | RRNAATWKNAVRHNLSLHKCFVRVENVKGAVWTVDEVEFQKRRP | |
| | | | | QKISGNPSLIKNMQSSHAYCTPLNAALQASMAENSIPLYTTASMGN | |
| | | | | PTLGNLASAIREELNGAMEHTNSNESDSSPGRSPMQ | |
| FOXP1 | forkhead box P1 | 27086 | 57 | MMQESGTETKSNGSAIQNGSGGSNHLLECGGLREGRSNGETPAVDI | 311. |
| | [Source:HGNC | | | GAADLAHAQQQ | |
| | Symbol; Acc:HGNC: | | | | |
| | 2383] | | | | |
| FOXP1 | forkhead box P1 | 27086 | 577 | MMTPQVITPQQMQQILQQQVLSPQQLQVLLQQQQALMLQQQQLQ | 312. |
| | [Source:HGNC | | | EFYKKQQEQLQLQLLQQQHAGKQPKEQQQVATQQLAFQQQLLQM | |
| | Symbol; Acc:HGNC: | | | QQLQQQHLLSLQRQGLLTIQPGQPALPLQPLAQGMIPTELQQLWKE | |
| | 3823] | | | VTSAHTAEETTGNNHSSLDLTTTCVSSSAPSKTSLIMNPHASTNGQL | |
| | | | | SVHTPKRESLSHEEHPHSHPLYGHGVCKWPGCEAVCEDFQSFLKHL | |
| | | | | NSEHALDDRSTAQCRVQMQVVQQLELQLAKDKERLQAMMTHLH | |
| | | | | VKSTEPKAAPQPLNLVSSVTLSKSASEASPQSLPHTPTTPTAPLTPVT | |
| | | | | QGPSVITTTSMHTVGPIRRRYSDKYNVPISSDIAQNQEFYKNAEVRP | |
| | | | | PFTYASLIRQAILESPEKQLTLNEIYNWFTRMFAYFRRNAATWKNA | |
| | | | | VRHNLSLHKCFVRVENVKGAVWTVDEVEFQKRRPQKISGNPSLIK | |
| | | | | NMQSSHAYCTPLNAALQASMAENSIPLYTTASMGNPTLGNLASAIR | |
| | | | | EELNGAMEHTNSNESDSSPGRSPMQAVHPVHVKEEPLDPEEAEGPL | |
| | | | | SLVTTANHSPDFDHDRDYEDEPVNEDME* | |
| FOXP1 | forkhead box P1 | 27086 | 89 | MMQESGTETKSNGSAIQNGSGGSNHLLECGGLREGRSNGETPAVDI | 313. |
| | [Source:HGNC | | | GAADLAHAQQQQQQALQVARQLLLQQQQQQQVSGLKSPKRNDK | |
| | Symbol; Acc:HGNC: | | | | |
| | 3823] | | | | |
| FOXP1 | forkhead box P1 | 27086 | 336 | MMQESGTETKSNGSAIQNGSGGSNHLLECGGLREGRSNGETPAVDI | 314. |
| | [Source:HGNC | | | GAADLAHAQQQQQQALQVARQLLLQQQQQQQVSGLKSPKRNDK | |
| | Symbol; Acc:HGNC: | | | QPALQVPVSVAMMTPQVITPQQMQQILQQQVLSPQQLQVLLQQQQ | |
| | 3823] | | | ALMLQQQQLQEFYKKQQEQLQLQLLQQQHAGKQPKEQQQVATQQ | |
| | | | | LAFQQQLLQMQQLQQQHLLSLQRQGLLTIQPGQPALPLQPLAQGMI | |
| | | | | PTELQQLWKEVTSAHTAEETTGNNHSSLDLTTTCVSSSAPSKTSLIM | |
| | | | | NPHASTNGQLSVHTPKRESLSHEEHPHSHPLYGHGVCKWPGCEAV | |
| | | | | CEDFQSFLNLQKTKNACKP* | |
| FOXP1 | forkhead box P1 | 27086 | 468 | MQQLQQQHLLSLQRQGLLTIQPGQPALPLQPLAQGMIPTELQQLWK | 315. |
| | [Source:HGNC | | | EVTSAHTAEETTGNNHSSLDLTTTCVSSSAPSKTSLIMNPHASTNGQ | |
| | Symbol; Acc:HGNC: | | | LSVHTPKRESLSHEEHPHSHPLYGHGVCKWPGCEAVCEDFQSFLKH | |
| | 3823] | | | LNSEHALDDRSTAQCRVQMQVVQQLELQLAKDKERLQAMMTHLH | |
| | | | | VKSTEPKAAPQPLNLVSSVTLSKSASEASPQSLPHTPTTPTAPLTPVT | |
| | | | | QGPSVITTTSMHTVGPIRRRYSDKYNVPISSADIAQNQEFYKNAEVR | |
| | | | | PPFTYASLIRQAILESPEKQLTLNEIYNWFTRMFAYFRRNAATWKN | |
| | | | | AVRHNLSLHKCFVRVENVKGAVWTVDEVEFQKRRPQKISGNPSLI | |
| | | | | KNMQSSHAYCTPLNAALQASMAENSIPLYTTASMGNPTLGNLASAI | |
| | | | | REELNGAMEHTNSNESDSSPGRSPMQAVHPVHVKEEPLDPEEAEGP | |
| | | | | LSLVTTA | |
| FOXP1 | forkhead box P1 | 27086 | 680 | MFQCVFSSSVLQPHSTSCLFKHLFYHSATPASQKQPEPIYSKKTEIQR | 316. |
| | [Source:HGNC | | | QTVRAPFAKLFIFSALQVARQLLLQQQQQQQVSGLKSPKRNDKQPA | |
| | Symbol; Acc:HGNC: | | | LQVPVSVAMMTPQVITPQQMQQILQQQVLSPQQLQVLLQQQQALM | |
| | 3823] | | | LQQQQLQEFYKKQQEQLQLQLLQQQHAGKQPKEQQQVATQQLAF | |
| | | | | QQQLLQMQQLQQQHLLSLQRQGLLTIQPGQPALPLQPLAQGMIPTE | |
| | | | | LQQLWKEVTSAHTAEETTGNNHSSLDLTTTCVSSSAPSKTSLIMNP | |
| | | | | HASTNGQLSVHTPKRESLSHEEHPHSHPLYGHGVCKWPGCEAVCE | |
| | | | | DFQSFLKHLNSEHALDDRSTAQCRVQMQVVQQLELQLAKDKERLQ | |
| | | | | AMMTHLHVKSTEPKAAPQPLNLVSSVTLSKSASEASPQSLPHTPTTP | |
| | | | | TAPLTPVTQGPSVITTTSMHTVGPIRRRYSDKYNVPISSADIAQNQEF | |
| | | | | YKNAEVRPPFTYASLIRQAILESPEKQLTLNEIYNWFTRMFAYFRRN | |
| | | | | AATWKNAVRHNLSLHKCFVRVENVKGAVWTVDEVEFQKRRPQKI | |
| | | | | SGNPSLIKNMQSSHAYCTPLNAALQASMAENSIPLYTTASMGNPTL | |
| | | | | GNLASAIREELNGAMEHTNSNESDSSPGRSPMQAVHPVHVKEEPLD | |
| | | | | PEEAEGPLSLVTTANHSPDFDHDRDYEDEPVNEDME* | |
| SPIB | Spi-B transcription | 6689 | 178 | MLALEAAQLDGPHFSCLYPDGVFYDLDSCKHSSYPDSEGAPDSLW | 317. |
| | factor [Source:HGNC | | | DWTVAPPVPATPYEAFDPAAAAFSHPQAAQLCYEPPTYSPAGNLEL | |
| | Symbol; Acc:HGNC: | | | APSLEAPGPGLPAYPTENFASQTLVPPAYAPYPSPVLSEEEDLPLDSP | |
| | 11242] | | | ALEVSDSESDEALVAGPEGKGSEGLARSCACTSSCWGY* | |
| SPIB | Spi-B transcription | 6689 | 172 | MASSMTWTAASIPATLIQRGLLTLVPPAYAPYPSPVLSEEEDLPLDS | 318. |
| | factor [Source:HGNC | | | PALEVSDSESDEALVAGPEGKGSEAGTRKKLRLYQFLLGLLTRGDM | |
| | Symbol; Acc:HGNC: | | | RECVWWVEPGAGVFQFSSKHKELLARRWGQQKGNRKRMTYQKL | |
| | 11242] | | | ARALRNYAKTGEIRKVKRKLTYQFDSALLPAVRRA* | |
| SPIB | Spi-B transcription | 6689 | 127 | MLALEAAQFRWLEAASACPCVSPPPGSTGHTSAVCTQMASSMTWT | 319. |
| | factor [Source:HGNC | | | AASIPATLIQRGLLTPCGTGLWPHLSQPPPMKPSTRQQPLLATPRLPS | |
| | Symbol; Acc:HGNC: | | | SATNPPPTALQGTSNWPPAWRPRGLASLHTPRRT | |
| | 11242] | | | | |
| SPIB | Spi-B transcription | 6689 | 78 | MLALEAAQLDGPHFSCLYPDGVFYDLDSCKHSSYPDSEGAPDPGSP | 320. |
| | factor [Source:HGNC | | | GICPVPQPCAIRGGRLTVGQPCPGGLGQRVG* | |
| | Symbol; Acc:HGNC: | | | | |
| | 11242] | | | | |
| SPIB | Spi-B transcription | 6689 | 263 | MLALEAAQLDGPHFSCLYPDGVFYDLDSCKHSSYPDSEGAPDSLW | 321. |
| | factor [Source:HGNC | | | DWTVAPPVPATPYEAFDPAAAAFSHPQAAQLCYEPPTYSPAGNLEL | |
| | Symbol; Acc:HGNC: | | | APSLEAPGPGLPAYPTENFASQTLVPPAYAPYPSPVLSEEEDLPLDSP | |
| | 11242] | | | ALEVSDSESDEALVAGPEGKGSEAGTRKKLRLYQFLLGLLTRGDM | |
| | | | | RECVWWVEPGAGVFQFSSKHKELLARRWGQQKGNRKRMTYQKL | |
| | | | | ARALRNYAKTGEIRKVKRKLTYQFDSALLPAVRRA* | |
| SPIB | Spi-B transcription | 6689 | 138 | MLALEAAQLDGPHFSCLYPDGVFYDLDSCKHSSYPDSEGAPDSLW | 322. |
| | factor [Source:HGNC | | | DWTVAPPVPATPYEAFDPAAAAFSHPQAAQLCYEPPTYSPAGNLEL | |
| | Symbol; Acc:HGNC: | | | APSLEAPGPGLPAYPTENFASQPCAIRGGRLTVGQPCPGGLGQRVG* | |
| | 11242] | | | | |
| AFF3 | AF4/FMR2 family | 3899 | 1227 | MDSFDLALLQEWDLESLCVYEPDRNALRRKERERRNQETQQDDGT | 323. |
| | member 3 | | | FNSSYSLFSEPYKTNKGDELSNRIQNTLGNYDEMKDFLTDRSNQSH | |
| | [Source:HGNC | | | LVGVPKPGVPQTPVNKIDEHFVADSRAQNQPSSICSTTTSTPAAVPV | |
| | Symbol; Acc:HGNC: | | | QQSKRGTMGWQKAGHPPSDGQQRATQQGSLRTLLGDGVGRQQPR | |
| | 6473] | | | AKQVCNVEVGLQTQERPPAMAAKHSSSGHCVQNFPPSLASKPSLV | |
| | | | | QQKPTAYVRPMDGQDQAPDESPKLKSSSETSVHCTSYRGVPASKPE | |
| | | | | PARAKAKLSKFSIPKQGEESRSGETNSCVEEIIREMTWLPPLSAIQAP | |
| | | | | GKVEPTKFPFPNKDSQLVSSGHNNPKKGDAEPESPDNGTSNTSMLE | |
| | | | | DDLKLSSDEEENEQQAAQRTALRALSDSAVVQQPNCRTSVPSSKGS | |
| | | | | SSSSSSGSSSSSSDSESSSGSDSETESSSSESEGSKPPHFSSPEAEPASS | |
| | | | | NKWQLDKWLNKVNPHKPPILIQNESHGSESNQYYNPVKEDVQDCG | |
| | | | | KVPDVCQPSLREKEIKSTCKEEQRPRTANKAPGSKGVKQKSPPAAV | |
| | | | | AVAVSAAAPPPAVPCAPAENAPAPARRSAGKKPTRRTERTSAGDG | |
| | | | | ANCHRPEEPAAADALGTSVVVPPEPTKTRPCGNNRASHRKELRSSV | |
| | | | | TCEKRRTRGLSRIVPKSKEFIETESSSSSSSSDSDLESEQEEYPLSKAQ | |
| | | | | TVAASASSGNDQRLKEAAANGGSGPRAPVGSINARTTSDIAKELEE | |
| | | | | QFYTLVPFGRNELLSPLKDSDEIRSLWVKIDLTLLSRIPEHLPQEPGV | |
| | | | | LSAPATKDSESAPPSHTSDTPAEKALPKSKRKRKCDNEDDYREIKKS | |
| | | | | QGEKDSSSRLATSTSNTLSANHCNMNINSVAIPINKNEKMLRSPISPL | |
| | | | | SDASKHKYTSEDLTSSSRPNGNSLFTSASSSKKPKADSQLQPHGGDL | |
| | | | | TKAAHNNSENIPLHKSRPQTKPWSPGSNGHRDCKRQKLVFDDMPR | |
| | | | | SADYFMQEAKRMKHKADAMVEKFGKALNYAEAALSFIECGNAME | |
| | | | | QGPMESKSPYTMYSETVELIRYAMRLKTHSGPNATPEDKQLAALC | |
| | | | | YRCLALLYWRMFRLKRDHAVKYSKALIDYFKNSSKAAQAPSPWG | |
| | | | | ASGKSTGTPSPMSPNPSPASSVGSQGSLSNASALSPSTIVSIPQRIHQ | |
| | | | | MAANHVSITNSILHSYDYWEMADNLAKENREFFNDLDLLMGPVTL | |
| | | | | HSSMEHLVQYSQQGLHWLRNSAHLS* | |
| AFF3 | AF4/FMR2 family | 3899 | 1252 | MDSFDLALLQEWDLESLWGEDILNQRNDSLVVEFQSSASRCRSVYE | 324. |
| | member 3 | | | PDRNALRRKERERRNQETQQDDGTFNSSYSLFSEPYKTNKGDELSN | |
| | [Source:HGNC | | | RIQNTLGNYDEMKDFLTDRSNQSHLVGVPKPGVPQTPVNKIDEHFV | |
| | Symbol; Acc:HGNC: | | | ADSRAQNQPSSICSTTTSTPAAVPVQQSKRGTMGWQKAGHPPSDG | |
| | 6473] | | | QQRATQQGSLRTLLGDGVGRQQPRAKQVCNVEVGLQTQERPPAM | |
| | | | | AAKHSSSGHCVQNFPPSLASKPSLVQQKPTAYVRPMDGQDQAPDE | |
| | | | | SPKLKSSSETSVHCTSYRGVPASKPEPARAKAKLSKFSIPKQGEESRS | |
| | | | | GETNSCVEEIIREMTWLPPLSAIQAPGKVEPTKFPFPNKDSQLVSSGH | |
| | | | | NNPKKGDAEPESPDNGTSNTSMLEDDLKLSSDEEENEQQAAQRTA | |
| | | | | LRALSDSAVVQQPNCRTSVPSSKGSSSSSSSGSSSSSSDSESSSGSDSE | |
| | | | | TESSSSESEGSKPPHFSSPEAEPASSNKWQLDKWLNKVNPHKPPILIQ | |
| | | | | NESHGSESNQYYNPVKEDVQDCGKVPDVCQPSLREKEIKSTCKEEQ | |
| | | | | RPRTANKAPGSKGVKQKSPPAAVAVAVSAAAPPPAVPCAPAENAP | |
| | | | | APARRSAGKKPTRRTERTSAGDGANCHRPEEPAAADALGTSVVVP | |
| | | | | PEPTKTRPCGNNRASHRKELRSSVTCEKRRTRGLSRIVPKSKEFIETE | |
| | | | | SSSSSSSSDSDLESEQEEYPLSKAQTVAASASSGNDQRLKEAAANGG | |
| | | | | SGPRAPVGSINARTTSDIAKELEEQFYTLVPFGRNELLSPLKDSDEIR | |
| | | | | SLWVKIDLTLLSRIPEHLPQEPGVLSAPATKDSESAPPSHTSDTPAEK | |
| | | | | ALPKSKRKRKCDNEDDYREIKKSQGEKDSSSRLATSTSNTLSANHC | |
| | | | | NMNINSVAIPINKNEKMLRSPISPLSDASKHKYTSEDLTSSSRPNGNS | |
| | | | | LFTSASSSKKPKADSQLQPHGGDLTKAAHNNSENIPLHKSRPQTKP | |
| | | | | WSPGSNGHRDCKRQKLVFDDMPRSADYFMQEAKRMKHKADAMV | |
| | | | | EKFGKALNYAEAALSFIECGNAMEQGPMESKSPYTMYSETVELIRY | |
| | | | | AMRLKTHSGPNATPEDKQLAALCYRCLALLYWRMFRLKRDHAVK | |
| | | | | YSKALIDYFKNSSKAAQAPSPWGASGKSTGTPSPMSPNPSPASSVGS | |
| | | | | QGSLSNASALSPSTIVSIPQRIHQMAANHVSITNSILHSYDYWEMAD | |
| | | | | NLAKENREFFNDLDLLMGPVTLHSSMEHLVQYSQQGLHWLRNSA | |
| | | | | HLS* | |
| AFF3 | AF4/FMR2 family | 3899 | 17 | MDSFDLALLQEWDLESL | 325. |
| | member 3 | | | | |
| | [Source:HGNC | | | | |
| | Symbol; Acc:HGNC: | | | | |
| | 6473] | | | | |
| AFF3 | AF4/FMR2 family | 3899 | 165 | MDSFDLALLQEWDLESLWGEDILNQRNDSLVVEFQSSASRCRSVYE | 326. |
| | member 3 | | | PDRNALRRKERERRNQETQQDDGTFNSSYSLFSEPYKTNKGDELSN | |
| | [Source:HGNC | | | RIQNTLGNYDEMKDFLTDRSNQSHLVGVPKPGVPQTPVNKIDEHFV | |
| | Symbol; Acc:HGNC: | | | ADSRAQNQPSSICSTTTSTPAAVPVQQ | |
| | 6473] | | | | |
| AFF3 | AF4/FMR2 family | 3899 | 129 | MDSFDLALLQEWDLESLCVYEPDRNALRRKERERRNQETQQDDGT | 327. |
| | member 3 | | | FNSSYSLFSEPYKTNKGDELSNRIQNTLGNYDEMKDFLTDRSNQSH | |
| | [Source:HGNC | | | LVGVPKPGVPQTPVNKIDEHFVADSRAQNQPSSICSTT | |
| | Symbol; Acc:HGNC: | | | | |
| | 6473] | | | | |
| AFF3 | AF4/FMR2 family | 3899 | 99 | MDSFDLALLQEWDLESLCVYEPDRNALRRKERERRNQETQQDDGT | 328. |
| | member 3 | | | FNSSYSLFSEPYKTNKGDELSNRIQNTLGNYDEMKDFLTDRSNQSH | |
| | [Source:HGNC | | | LVGVPKPG | |
| | Symbol; Acc:HGNC: | | | | |
| | 6473] | | | | |
| AFF3 | AF4/FMR2 family | 3899 | 581 | GNNRASHRKELRSSVTCEKRRTRGLSRIVPKSKEFIETESSSSSSSSDS | 329. |
| | member 3 | | | DLESEQEEYPLSKAQTVAASASSGNDQRLKEAAANGGSGPRAPVG | |
| | [Source:HGNC | | | SINARTTSDIAKELEEQFYTLVPFGRNELLSPLKDSDEIRSLWVKIDL | |
| | Symbol; Acc:HGNC: | | | TLLSRIPEHLPQEPGVLSAPATKDSESAPPSHTSDTPAEKALPKSKRK | |
| | 6473] | | | RKCDNEDDYREIKKSQGEKDSSSRLATSTSNTLSANHCNMNINSVA | |
| | | | | IPINKNEKMLRSPISPLSDASKHKYTSEDLTSSSRPNGNSLFTSASSSK | |
| | | | | KPKADSQLQPHGGDLTKAAHNNSENIPLHKSRPQTKPWSPGSNGH | |
| | | | | RDCKRQKLVFDDMPRSADYFMQEAKRMKHKADAMVEKFGKALN | |
| | | | | YAEAALSFIECGNAMEQGPMESKSPYTMYSETVELIRYAMRLKTHS | |
| | | | | GPNATPEDKQLAALCYRCLALLYWRMFRLKRDHAVKYSKALIDYF | |
| | | | | KNSSKAAQAPSPWGASGNSVGSQGSLSNASALSPSTIVSIPQRIHQM | |
| | | | | AANHVSITNSILHSYDYWEMADNLAKENREFFNDLDLLMGPVTLH | |
| | | | | SSMEHLVQYSQQGLHWLRNSAHLS* | |
| AFF3 | AF4/FMR2 family | 3899 | 180 | XASKPSLVQQKPTAYVRPMDGQDQAPDESPKLKSSSETSVHCTSYR | 330. |
| | member 3 | | | GVPASKPEPARAKAKLSKFSIPKQGEESRSGETNSCVEEIIREMTWL | |
| | [Source:HGNC | | | PPLSAIQAPGKVEPTKFPFPNKDSQLVSSGHNNPKKGDAEPESPDNG | |
| | Symbol; Acc:HGNC: | | | TSNTSMLEDDLKLSSDEEENEQQAAQRTALRALSDSLSW* | |
| | 6473] | | | | |
| AFF3 | AF4/FMR2 family | 3899 | 161 | MDSFDLALLQEWDLESLCVYEPDRNALRRKERERRNQETQQDDGT | 331. |
| | member 3 | | | FNSSYSLFSEPYKTNKGDELSNRIQNTLGNYDEMKDFLTDRSNQSH | |
| | [Source:HGNC | | | LVGVPKPGVPQTPVNKIDEHFVADSRAQNQPSSICSTTTSTPAAVPV | |
| | Symbol; Acc:HGNC: | | | QQSKRGTMGWQKAGHPPSDGQQR | |
| | 6473] | | | | |
| AFF3 | AF4/FMR2 family | 3899 | 54 | MDSFDLALLQEWDLESLWGEDILNQRNDSLVVEFQSSASRCRSVYE | 332. |
| | member 3 | | | PDRNALRR | |
| | [Source:HGNC | | | | |
| | Symbol; Acc:HGNC: | | | | |
| | 6473] | | | | |
| AFF3 | AF4/FMR2 family | 3899 | 103 | MDSFDLALLQEWDLESLWGEDILNQRNDSLVVEFQSSASRCRSVYE | 333. |
| | member 3 | | | PDRNALRRKERERRNQETQQDDGTFNSSYSLFSEPYKTNKGDELSN | |
| | [Source:HGNC | | | RIQNTLGNYDE | |
| | Symbol; Acc:HGNC: | | | | |
| | 6473] | | | | |
| AFF3 | AF4/FMR2 family | 3899 | 114 | MDSFDLALLQEWDLESLCVYEPDRNALRRKERERRNQETQQDDGT | 334. |
| | member 3 | | | FNSSYSLFSEPYKTNKGDELSNRIQNTLGNYDEMKDFLTDRSNQSH | |
| | [Source:HGNC | | | LVGVPKPGVPQTPVNKIDEHFVA | |
| | Symbol; Acc:HGNC: | | | | |
| | 6473] | | | | |
| AFF3 | AF4/FMR2 family | 3899 | 380 | MDSFDLALLQEWDLESLWGEDILNQRNDSLVVEFQSSASRCRSVYE | 335. |
| | member 3 | | | PDRNALRRKERERRNQETQQDDGTFNSSYSLFSEPYKTNKGDELSN | |
| | [Source:HGNC | | | RIQNTLGNYDEMKDFLTDRSNQSHLVGVPKPGVPQTPVNKIDEHFV | |
| | Symbol; Acc:HGNC: | | | ADSRAQNQPSSICSTTTSTPAAVPVQQSKRGTMGWQKAGHPPSDG | |
| | 6473] | | | QQRATQQGSLRTLLGDGVGRQQPRAKQVCNVEVGLQTQERPPAM | |
| | | | | AAKHSSSGHCVQNFPPSLASKPSLVQQKPTAYVRPMDGQDQAPDE | |
| | | | | SPKLKSSSETSVHCTSYRGVPASKPEPARAKAKLSKFSIPKQGEESRS | |
| | | | | GETNSCVEEIIRYPRVCFSNAVQTEPALPGPGCTGHSLWRICSVGPP | |
| | | | | GAGRRQPTCLCA* | |
| AFF3 | AF4/FMR2 family | 3899 | 115 | MDSFDLALLQEWDLESLWGEDILNQRNDSLVVEFQSSASRCRSVYE | 336. |
| | member 3 | | | PDRNALRRKERERRNQETQQDDGTFNSSYSLFSEPYKTNKGDELSN | |
| | [Source:HGNC | | | RIQNTLGNYDEMKDFLTDRSNQS | |
| | Symbol; Acc:HGNC: | | | | |
| | 6473] | | | | |
| AFF3 | AF4/FMR2 family | 3899 | 608 | MDSFDLALLQEWDLESLCVYEPDRNALRRKERERRNQETQQDDGT | 337. |
| | member 3 | | | FNSSYSLFSEPYKTNKGDELSNRIQNTLGNYDEMKDFLTDRSNQSH | |
| | [Source:HGNC | | | LVGVPKPGVPQTPVNKIDEHFVADSRAQNQPSSICSTTTSTPAAVPV | |
| | Symbol; Acc:HGNC: | | | QQSKRGTMGWQKAGHPPSDGQQRATQQGSLRTLLGDGVGRQQPR | |
| | 6473] | | | AKQVCNVEVGLQTQERPPAMAAKHSSSGHCVQNFPPSLASKPSLV | |
| | | | | QQKPTAYVRPMDGQDQAPDESPKLKSSSETSVHCTSYRGVPASKPE | |
| | | | | PARAKAKLSKFSIPKQGEESRSGETNSCVEEIIREMTWLPPLSAIQAP | |
| | | | | GKVEPTKFPFPNKDSQLVSSGHNNPKKGDAEPESPDNGTSNTSMLE | |
| | | | | DDLKLSSDEEENEQQAAQRTALRALSDSAVVQQPNCRTSVPSSKGS | |
| | | | | SSSSSSGSSSSSSDSESSSGSDSETESSSSESEGSKPPHFSSPEAEPASS | |
| | | | | NKWQLDKWLNKVNPHKPPILIQNESHGSESNQYYNPVKEDVQDCG | |
| | | | | KVPDVCQPSLREKEIKSTCKEEQRPRTANKAPGSKGVKQKSPPAAV | |
| | | | | AVAVSAAAPPPAVPCAPAENAPAPARRSAGKKPTRRTERTSAGDG | |
| | | | | ANCHRPEEP | |
| AFF3 | AF4/FMR2 family | 3899 | 43 | MDSFDLALLQEWDLESLCVYEPDRNALRRKERERRNQETQQDD | 338. |
| | member 3 | | | | |
| | [Source:HGNC | | | | |
| | Symbol; Acc:HGNC: | | | | |
| | 6473] | | | | |
| AFF3 | AF4/FMR2 family | 3899 | 135 | XSAPPSHTSDTPAEKALPKSKRKRKCDNEDDYREIKKSQGEKDSSS | 339. |
| | member 3 | | | RLATSTSNTLSANHCNMNINSVAIPINKNEKMLRSPISPLSDASKHK | |
| | [Source:HGNC | | | YTSEDLTSSSRPNGNSLFTSASSSKKPKADSQLQPHGGDLT* | |
| | Symbol; Acc:HGNC: | | | | |
| | 6473] | | | | |
| AFF3 | AF4/FMR2 family | 3899 | 143 | MDSFDLALLQEWDLESLWGEDILNQRNDSLVVEFQSSASRCRSVYE | 340. |
| | member 3 | | | PDRNALRRKERERRNQETQQDDGTFNSSYSLFSEPYKTNKGDELSN | |
| | [Source:HGNC | | | RIQNTLGNYDEMKDFLTDRSNQSHLVGVPKPGVPQTPVNKIDEHFV | |
| | Symbol; Acc:HGNC: | | | ADSRA | |
| | 6473] | | | | |
| RCEI | Ras converting CAAX | 9986 | 330 | MAALGGDGLRLLSVSRPERPPESAALGGLGPGLCCWVSVFSCLSLA | 341. |
| | endopeptidase 1 | | | CSYVGSLYVWKSELPRDHPAVIKRRFTSVLVVSSLSPLCVLLWREL | |
| | [Source:HGNC | | | TGIQPGTSLLTLMGFRLEGIFPAALLPLLLTMILFLGPLMQLSMDCPC | |
| | Symbol; Acc:HGNC: | | | DLADGLKVVLAPRSWARCLTDMRWLRNQVIAPLTEELVFRACMLP | |
| | 13721] | | | MLAPCMGLGPAVFTCPLFFGVAHFHHIIEQLRFRQSSVGNIFLSAAF | |
| | | | | QFSYTAVFGAYTAFLFIRTGHLIGPVLCHSFCNYMGFPAVCAALEHP | |
| | | | | QRRPLLAGYALGVGLFLLLLQPLTDPKLYGSLPLCVLLERAGDSEA | |
| | | | | PLCS* | |
| RCE1 | Ras converting CAAX | 9986 | 103 | MAALGGDGLRLLSVSRPERPPESAALGGLGPGLCCWVSVFSCLSLA | 342. |
| | endopeptidase 1 | | | CSYVGSLYVWKSELPRDHPAVIKRRFTSVLVVSSLSPLCVLLWREL | |
| | [Source:HGNC | | | TGIQAHPCSP* | |
| | Symbol; Acc:HGNC: | | | | |
| | 13721] | | | | |
| RCE1 | Ras converting CAAX | 9986 | 309 | MAALGGDGLRLLSVSRPERPPESAALGGLGPGLCCWVSVFSCLSLA | 343. |
| | endopeptidase 1 | | | CSYVGSLYVWKSELPRDHPAVIKRRFTSVLVVSSLSPLCVLLWREL | |
| | [Source:HGNC | | | TGIQPGTSLLTLMGFRLEGIFPAALLPLLLTMILFLGPLMQLSMDCPC | |
| | Symbol; Acc:HGNC: | | | DLADGLKVVLAPRSWARCLTDMRWLRNQVIAPLTEELVFRACMLP | |
| | 13721] | | | MLAPCMGLGPAVFTCPLFFGVAHFHHIIEQLRFRQSSVGNIFLSAGH | |
| | | | | LIGPVLCHSFCNYMGFPAVCAALEHPQRRPLLAGYALGVGLFLLLL | |
| | | | | QPLTDPKLYGSLPLCVLLERAGDSEAPLCS* | |
| RCE1 | Ras converting CAAX | 9986 | 207 | MILFLGPLMQLSMDCPCDLADGLKVVLAPRSWARCLTDMRWLRN | 344. |
| | endopeptidase 1 | | | QVIAPLTEELVFRACMLPMLAPCMGLGPAVFTCPLFFGVAHFHHIIE | |
| | [Source:HGNC | | | QLRFRQSSVGNIFLSAAFQFSYTAVFGAYTAFLFIRTGHLIGPVLCHS | |
| | Symbol; Acc:HGNC: | | | FCNYMGFPAVCAALEHPQRRPLLAGYALGVGLFLLLLQPLTDPKLY | |
| | 13721] | | | GSLPLCVLLERAGDSEAPLCS* | |
| JAK1 | Janus kinase 1 | 3716 | 1155 | MQYLNIKEDCNAMAFCAKMRSSKKTEVNLEAPEPGVEVIFYLSDR | 345. |
| | [Source:HGNC | | | EPLRLGSGEYTAEELCIRAAQACRISPLCHNLFALYDENTKLWYAP | |
| | Symbol; Acc:HGNC: | | | NRTITVDDKMSLRLHYRMRFYFTNWHGTNDNEQSVWRHSPKKQK | |
| | 6190] | | | NGYEKKKIPDATPLLDASSLEYLFAQGQYDLVKCLAPIRDPKTEQD | |
| | | | | GHDIENECLGMAVLAISHYAMMKKMQLPELPKDISYKRYIPETLNK | |
| | | | | SIRQRNLLTRMRINNVFKDFLKEFNNKTICDSSVSTHDLKVKYLATL | |
| | | | | ETLTKHYGAEIFETSMLLISSENEMNWFHSNDGGNVLYYEVMVTG | |
| | | | | NLGIQWRHKPNVVSVEKEKNKLKRKKLENKHKKDEEKNKIREEW | |
| | | | | NNFSYFPEITHIVIKESVVSINKQDNKKMELKLSSHEEALSFVSLVDG | |
| | | | | YFRLTADAHHYLCTDVAPPLIVHNIQNGCHGPICTEYAINKLRQEGS | |
| | | | | EEGMYVLRWSCTDFDNILMTVTCFEKSEQVQGAQKQFKNFQIEVQ | |
| | | | | KGRYSLHGSDRSFPSLGDLMSHLKKQILRTDNISFMLKRCCQPKPRE | |
| | | | | ISNLLVATKKAQEWQPVYPMSQLSFDRILKKDLVQGEHLGRGTRT | |
| | | | | HIYSGTLMDYKDDEGTSEEKKIKVILKVLDPSHRDISLAFFEAASMM | |
| | | | | RQVSHKHIVYLYGVCVRDVENIMVEEFVEGGPLDLFMHRKSDVLT | |
| | | | | TPWKFKVAKQLASALSYLEDKDLVHGNVCTKNLLLAREGIDSECG | |
| | | | | PFIKLSDPGIPITVLSRQECIERIPWIAPECVEDSKNLSVAADKWSFGT | |
| | | | | TLWEICYNGEIPLKDKTLIEKERFYESRCRPVTPSCKELADLMTRCM | |
| | | | | NYDPNQRPFFRAIMRDINKLEEQNPDIVSEKKPATEVDPTHFEKRFL | |
| | | | | KRIRDLGEGHFGKVELCRYDPEGDNTGEQVAVKSLKPESGGNHIAD | |
| | | | | LKKEIEILRNLYHENIVKYKGICTEDGGNGIKLIMEFLPSGSLKEYLP | |
| | | | | KNKNKINLKQQLKYAVQICKGMDYLGSRQYVHRDLAARNVLVES | |
| | | | | EHQVKIGDFGLTKAIETDKEYYTVKDDRDSPVFWYAPECLMQSKF | |
| | | | | YIASDVWSFGVTLHELLTYCDSDSSPMALFLKMIGPTHGQMTVTRL | |
| | | | | VNTLKEGKRLPCPPNCPDEVYQLMRKCWEFQPSNRTSFQNLIEGFE | |
| | | | | ALLK* | |
| JAK1 | Janus kinase 1 | 3716 | 709 | MQYLNIKEDCNAMAFCAKMRSSKKTEVNLEAPEPGVEVIFYLSDR | 346. |
| | [Source HGNC | | | EPLRLGSGEYTAEELCIRAAQACRISPLCHNLFALYDENTKLWYAP | |
| | Symbol; Acc:HGNC: | | | NRTITVDDKMSLRLHYRMRFYFTNWHGTNDNEQSVWRHSPKKQK | |
| | 6190] | | | NGYEKKKIPDATPLLDASSLEYLFAQGQYDLVKCLAPIRDPKTEQD | |
| | | | | GHDIENECLGMAVLAISHYAMMKKMQLPELPKDISYKRYIPETLNK | |
| | | | | SIRQRNLLTRMRINNVFKDFLKEFNNKTICDSSVSTHDLKVKYLATL | |
| | | | | ETLTKHYGAEIFETSMLLISSENEMNWFHSNDGGNVLYYEVMVTG | |
| | | | | NLGIQWRHKPNVVSVEKEKNKLKRKKLENKHKKDEEKNKIREEW | |
| | | | | NNFSYFPEITHIVIKESVVSINKQDNKKMELKLSSHEEALSFVSLVDG | |
| | | | | YFRLTADAHHYLCTDVAPPLIVHNIQNGCHGPICTEYAINKLRQEGS | |
| | | | | EEGMYVLRWSCTDFDNILMTVTCFEKSEQVQGAQKQFKNFQIEVQ | |
| | | | | KGRYSLHGSDRSFPSLGDLMSHLKKQILRTDNISFMLKRCCQPKPRE | |
| | | | | ISNLLVATKKAQEWQPVYPMSQLSFDRILKKDLVQGEHLGRGTRT | |
| | | | | HIYSGTLMDYKDDEGTSEEKKIKVILKVLDPSHRDISLAFFEAASMM | |
| | | | | RQVSHKHIVYLYGVCVRDVESKCVLFQRGGWPGWGAWVLWRW | |
| | | | | WRLFPPSRCPGFLPFFLQLRRLRD* | |
| JAK1 | Janus kinase 1 | 3716 | 1153 | MQYLNIKEDCNAMAFCAKMRSSKKTEVNLEAPEPGVEVIFYLSDR | 347. |
| | [Source HGNC | | | EPLRLGSGEYTAEELCIRAAQACRISPLCHNLFALYDENTKLWYAP | |
| | Symbol; Acc:HGNC: | | | NRTITVDDKMSLRLHYRMRFYFTNWHGTNDNEQSVWRHSPKKQK | |
| | 6190] | | | NGYEKKKIPDATPLLDASSLEYLFAQGQYDLVKCLAPIRDPKTEQD | |
| | | | | GHDIENECLGMAVLAISHYAMMKKMQLPELPKDISYKRYIPETLNK | |
| | | | | SIRQRNLLTRMRINNVFKDFLKEFNNKTICDSSVSTHDLKVKYLATL | |
| | | | | ETLTKHYGAEIFETSMLLISSENEMNWFHSNDGGNVLYYEVMVTG | |
| | | | | NLGIQWRHKPNVVSVEKEKNKLKRKKLENKHKKDEEKNKIREEW | |
| | | | | NNFSYFPEITHIVIKESVVSINKQDNKKMELKLSSHEEALSFVSLVDG | |
| | | | | YFRLTADAHHYLCTDVAPPLIVHNIQNGCHGPICTEYAINKLRQEGS | |
| | | | | EEGMYVLRWSCTDFDNILMTVTCFEKSEQVQGAQKQFKNFQIEVQ | |
| | | | | KGRYSLHGSDRSFPSLGDLMSHLKKQILRTDNISFMLKRCCQPKPRE | |
| | | | | ISNLLVATKKAQEWQPVYPMSQLSFDRILKKDLVQGEHLGRGTRT | |
| | | | | HIYSGTLMDYKDDEGTSEEKKIKVILKVLDPSHRDISLAFFEAASMM | |
| | | | | RQVSHKHIVYLYGVCVRDVENIMVEEFVEGGPLDLFMHRKSDVLT | |
| | | | | TPWKFKVAKQLASALSYLEDKDLVHGNVCTKNLLLAREGIDSECG | |
| | | | | PFIKLSDPGIPITVLSRQECIERIPWIAPECVEDSKNLSVAADKWSFGT | |
| | | | | TLWEICYNGEIPLKDKTLIEKERFYESRCRPVTPSCKELADLMTRCM | |
| | | | | NYDPNQRPFFRAIMRDINKLEEQNIVSEKKPATEVDPTHFEKRFLKR | |
| | | | | IRDLGEGHFGKVELCRYDPEGDNTGEQVAVKSLKPESGGNHIADLK | |
| | | | | KEIEILRNLYHENIVKYKGICTEDGGNGIKLIMEFLPSGSLKEYLPKN | |
| | | | | KNKINLKQQLKYAVQICKGMDYLGSRQYVHRDLAARNVLVESEH | |
| | | | | QVKIGDFGLTKAIETDKEYYTVKDDRDSPVFWYAPECLMQSKFYIA | |
| | | | | SDVWSFGVTLHELLTYCDSDSSPMALFLKMIGPTHGQMTVTRLVN | |
| | | | | TLKEGKRLPCPPNCPDEVYQLMRKCWEFQPSNRTSFQNLIEGFEAL | |
| | | | | LK* | |
| JAK1 | Janus kinase 1 | 3716 | 88 | MQYLNIKEDCNAMAFCAKMRSSKKTEVNLEAPEPGVEVIFYLSDR | 348. |
| | [Source HGNC | | | EPLRLGSGEYTAEELCIRAAQACQWLLSAPSLMNYKIPGDDA* | |
| | Symbol; Acc:HGNC: | | | | |
| | 6190] | | | | |
| JAK1 | Janus kinase 1 | 3716 | 469 | XLYGVCVRDVENIMVEEFVEGGPLDLFMHRKSDVLTTPWKFKVAK | 349. |
| | [Source HGNC | | | QLASALSYLEDKDLVHGNVCTKNLLLAREGIDSECGPFIKLSDPGIPI | |
| | Symbol; Acc:HGNC: | | | TVLSRQECIERIPWIAPECVEDSKNLSVAADKWSFGTTLWEICYNGE | |
| | 6190] | | | IPLKDKTLIEKERFYESRCRPVTPSYPDIVSEKKPATEVDPTHFEKRF | |
| | | | | LKRIRDLGEGHFGKVELCRYDPEGDNTGEQVAVKSLKPESGGNHIA | |
| | | | | DLKKEIEILRNLYHENIVKYKGICTEDGGNGIKLIMEFLPSGSLKEYL | |
| | | | | PKNKNKINLKQQLKYAVQICKGMDYLGSRQYVHRDLAARNVLVE | |
| | | | | SEHQVKIGDFGLTKAIETDKEYYTVKDDRDSPVFWYAPECLMQSKF | |
| | | | | YIASDVWSFGVTLHELLTYCDSDSSPMALFLKMIGPTHGQMTVTRL | |
| | | | | VNTLKEGKRLPCPPNCPDEVYQLMRKCWEFQPSNRTSFQNLIEGFE | |
| | | | | ALLK* | |
| JAK1 | Janus kinase 1 | 3716 | 1111 | MQYLNIKEDCNAMAFCAKMRSSKKTEVNLEAPEPGVEVIFYLSDR | 350. |
| | [Source HGNC | | | EPLRLGSGEYTAEELCIRAAQACRISPLCHNLFALYDENTKLWYAP | |
| | Symbol; Acc:HGNC: | | | NRTITVDDKMSLRLHYRMRFYFTNWHGTNDNEQSVWRHSPKKQK | |
| | 6190] | | | NGYEKKKIPDATPLLDASSLEYLFAQGQYDLVKCLAPIRDPKTEQD | |
| | | | | GHDIENECLGMAVLAISHYAMMKKMQLPELPKDISVSTHDLKVKY | |
| | | | | LATLETLTKHYGAEIFETSMLLISSENEMNWFHSNDGGNVLYYEVM | |
| | | | | VTGNLGIQWRHKPNVVSVEKEKNKLKRKKLENKHKKDEEKNKIRE | |
| | | | | EWNNFSYFPEITHIVIKESVVSINKQDNKKMELKLSSHEEALSFVSL | |
| | | | | VDGYFRLTADAHHYLCTDVAPPLIVHNIQNGCHGPICTEYAINKLR | |
| | | | | QEGSEEGMYVLRWSCTDFDNILMTVTCFEKSEQVQGAQKQFKNFQ | |
| | | | | IEVQKGRYSLHGSDRSFPSLGDLMSHLKKQILRTDNISFMLKRCCQP | |
| | | | | KPREISNLLVATKKAQEWQPVYPMSQLSFDRILKKDLVQGEHLGRG | |
| | | | | TRTHIYSGTLMDYKDDEGTSEEKKIKVILKVLDPSHRDISLAFFEAA | |
| | | | | SMMRQVSHKHIVYLYGVCVRDVENIMVEEFVEGGPLDLFMHRKSD | |
| | | | | VLTTPWKFKVAKQLASALSYLEDKDLVHGNVCTKNLLLAREGIDS | |
| | | | | ECGPFIKLSDPGIPITVLSRQECIERIPWIAPECVEDSKNLSVAADKWS | |
| | | | | FGTTLWEICYNGEIPLKDKTLIEKERFYESRCRPVTPSCKELADLMT | |
| | | | | RCMNYDPNQRPFFRAIMRDINKLEEQNPDIVSEKKPATEVDPTHFE | |
| | | | | KRFLKRIRDLGEGHFGKVELCRYDPEGDNTGEQVAVKSLKPESGG | |
| | | | | NHIADLKKEIEILRNLYHENIVKYKGICTEDGGNGIKLIMEFLPSGSL | |
| | | | | KEYLPKNKNKINLKQQLKYAVQICKGMDYLGSRQYVHRDLAARN | |
| | | | | VLVESEHQVKIGDFGLTKAIETDKEYYTVKDDRDSPVFWYAPECL | |
| | | | | MQSKFYIASDVWSFGVTLHELLTYCDSDSSPMALFLKMIGPTHGQ | |
| | | | | MTVTRLVNTLKEGKRLPCPPNCPDEVYQLMRKCWEFQPSNRTSFQ | |
| | | | | NLIEGFEALLK* | |
| JAK1 | Janus kinase 1 | 3716 | 131 | MQYLNIKEDCNAMAFCAKMRSSKKTEVNLEAPEPGVEVIFYLSDR | 351. |
| | [Source HGNC | | | EPLRLGSGEYTAEELCIRAAQACRISPLCHNLFALYDENTKLWYAP | |
| | Symbol; Acc:HGNC: | | | NRTITVDDKMSLRLHYRMRFYFTNWHGTNDNEQSVWRHSP | |
| | 6190] | | | | |
| JAK1 | Janus kinase 1 | 3716 | 345 | MQYLNIKEDCNAMAFCAKMRSSKKTEVNLEAPEPGVEVIFYLSDR | 352. |
| | [Source HGNC | | | EPLRLGSGEYTAEELCIRAAQACRISPLCHNLFALYDENTKLWYAP | |
| | Symbol; Acc:HGNC: | | | NRTITVDDKMSLRLHYRMRFYFTNWHGTNDNEQSVWRHSPKKQK | |
| | 6190] | | | NGYEKKKIPDATPLLDASSLEYLFAQGQYDLVKCLAPIRDPKTEQD | |
| | | | | GHDIENECLGMAVLAISHYAMMKKMQLPELPKDISYKRYIPETLNK | |
| | | | | SIRQRNLLTRMRINNVFKDFLKEFNNKTICDSSVSTHDLKVKYLATL | |
| | | | | ETLTKHYGAEIFETSMLLISSENEMNWFHSNDGGNVLYYEVMVTG | |
| | | | | NLGIQWRHKPNVVSVEKEKNKLKRKK | |
| JAK1 | Janus kinase 1 | 3716 | 130 | MQYLNIKEDCNAMAFCAKMRSSKKTEVNLEAPEPGVEVIFYLSDR | 353. |
| | [Source HGNC | | | EPLRLGSGEYTAEELCIRAAQACRISPLCHNLFALYDENTKLWYAP | |
| | Symbol; Acc:HGNC: | | | NRTITVDDKMSLRLHYRMSLCSSGEATHYVWSPQPDAV* | |
| | 6190] | | | | |
| JAK1 | Janus kinase 1 | 3716 | 994 | XNDNEQSVWRHSPKKQKNGYEKKKIPDATPLLDASSLEYLFAQGQ | 354. |
| | [Source HGNC | | | YDLVKCLAPIRDPKTEQDGHDIENECLGMAVLAISHYAMMKKMQL | |
| | Symbol; Acc:HGNC: | | | PELPKDISSVSTHDLKVKYLATLETLTKHYGAEIFETSMLLISSENEM | |
| | 6190] | | | NWFHSNDGGNVLYYEVMVTGNLGIQWRHKPNVVSVEKEKNKLK | |
| | | | | RKKLENKHKKDEEKNKIREEWNNFSYFPEITHIVIKESVVSINKQDN | |
| | | | | KKMELKLSSHEEALSFVSLVDGYFRLTADAHHYLCTDVAPPLIVHN | |
| | | | | IQNGCHGPICTEYAINKLRQEGSEEGMYVLRWSCTDFDNILMTVTC | |
| | | | | FEKSEQVQGAQKQFKNFQIEVQKGRYSLHGSDRSFPSLGDLMSHLK | |
| | | | | KQILRTDNISFMLKRCCQPKPREISNLLVATKKAQEWQPVYPMSQL | |
| | | | | SFDRILKKDLVQGEHLGRGTRTHIYSGTLMDYKDDEGTSEEKKIKVI | |
| | | | | LKVLDPSHRDISLAFFEAASMMRQVSHKHIVYLYGVCVRDVENIM | |
| | | | | VEEFVEGGPLDLFMHRKSDVLTTPWKFKVAKQLASALSYLEDKDL | |
| | | | | VHGNVCTKNLLLAREGIDSECGPFIKLSDPGIPITVLSRQECIERIPWI | |
| | | | | APECVEDSKNLSVAADKWSFGTTLWEICYNGEIPLKDKTLIEKERF | |
| | | | | YESRCRPVTPSCKELADLMTRCMNYDPNQRPFFRAIMRDINKLEEQ | |
| | | | | NPDIVSEKKPATEVDPTHFEKRFLKRIRDLGEGHFGKVELCRYDPEG | |
| | | | | DNTGEQVAVKSLKPESGGNHIADLKKEIEILRNLYHENIVKYKGICT | |
| | | | | EDGGNGIKLIMEFLPSGSLKEYLPKNKNKINLKQQLKYAVQICKGM | |
| | | | | DYLGSRQYVHRDLAARNVLVESEHQVKIGDFGLTKAIETDKEYYT | |
| | | | | VKDDRDSPVFWYAPECLMQSKFYIASDVWSFGVTLHELLTYCDSD | |
| | | | | SSPMALFLKMIGPTHGQMTVTRLVNTLKEGKRLPCPPNCPDEVYQL | |
| | | | | MRKCWEFQPSNRTSFQNLIEGFEALLK* | |
| LRRC8C | leucine rich repeat | 84230 | 804 | MIPVTEFRQFSEQQPAFRVLKPWWDVFTDYLSVAMLMIGVFGCTL | 355. |
| | containing 8 VRAC | | | QVMQDKIICLPKRVQPAQNHSSLSNVSQAVASTTPLPPPKPSPANPIT | |
| | subunit C | | | VEMKGLKTDLDLQQYSFINQMCYERALHWYAKYFPYLVLIHTLVF | |
| | [Source:HGNC | | | MLCSNFWFKFPGSSSKIEHFISILGKCFDSPWTTRALSEVSGEDSEEK | |
| | Symbol; Acc:HGNC: | | | DNRKNNMNRSNTIQSGPEDSLVNSQSLKSIPEKFVVDKSTAGALDK | |
| | 25075] | | | KEGEQAKALFEKVKKFRLHVEEGDILYAMYVRQTVLKVIKFLIIIAY | |
| | | | | NSALVSKVQFTVDCNVDIQDMTGYKNFSCNHTMAHLFSKLSFCYL | |
| | | | | CFVSIYGLTCLYTLYWLFYRSLREYSFEYVRQETGIDDIPDVKNDFA | |
| | | | | FMLHMIDQYDPLYSKRFAVFLSEVSENKLKQLNLNNEWTPDKLRQ | |
| | | | | KLQTNAHNRLELPLIMLSGLPDTVFEITELQSLKLEIIKNVMIPATIA | |
| | | | | QLDNLQELSLHQCSVKIHSAALSFLKENLKVLSVKFDDMRELPPW | |
| | | | | MYGLRNLEELYLVGSLSHDISRNVTLESLRDLKSLKILSIKSNVSKIP | |
| | | | | QAVVDVSSHLQKMCIHNDGTKLVMLNNLKKMTNLTELELVHCDL | |
| | | | | ERIPHAVFSLLSLQELDLKENNLKSIEEIVSFQHLRKLTVLKLWHNSI | |
| | | | | TYIPEHIKKLTSLERLSFSHNKIEVLPSHLFLCNKIRYLDLSYNDIRFIP | |
| | | | | PEIGVLQSLQYFSITCNKVESLPDELYFCKKLKTLKIGKNSLSVLSPK | |
| | | | | IGNLLFLSYLDVKGNHFEILPPELGDCRALKRAGLVVEDALFETLPS | |
| | | | | DVREQMKTE* | |
| LRRC8C | leucine rich repeat | 84230 | 59 | MIPVTEFRQFSEQQPAFRVLKPWWDVFTDYLSVAMLMIGVFGCTL | 356. |
| | containing 8 VRAC | | | QAVSLKLSGFILF* | |
| | subunit C | | | | |
| | [Source:HGNC | | | | |
| | Symbol; Acc:HGNC: | | | | |
| | 25075] | | | | |
| GFI1 | growth factor | 2672 | 423 | MPRSFLVKSKKAHSYHQPRSPGPDYSLRLENVPAPSRADSTSNAGG | 357. |
| | independent 1 | | | AKAEPRDRLSPESQLTEAPDRASASPDSCEGSVCERSSEFEDFWRPP | |
| | transcriptional | | | SPSASPASEKSMCPSLDEAQPFPLPFKPYSWSGLAGSDLRHLVQSYR | |
| | repressor | | | PCGALERGAGLGLFCEPAPEPGHPAALYGPKRAAGGAGAGAPGSC | |
| | [Source:HGNC | | | SAGAGATAGPGLGLYGDFGSAAAGLYERPTAAAGLLYPERGHGLH | |
| | Symbol; Acc:HGNC: | | | ADKGAGVKVESELLCTRLLLGGGSYKCIKCSKVFSTPHGLEVHVRR | |
| | 4237] | | | SHSGTRPFACEMCGKTFGHAVSLEQHKAVHSQERSFDCKICGKSFK | |
| | | | | RSSTLSTHLLIHSDTRPYPCQYCGKRFHQKSDMKKHTFIHTGEKPHK | |
| | | | | CQVCGKAFSQSSNLITHSRKHTGFKPFGCDLCGKGFQRKVDLRRHR | |
| | | | | ETQHGLK* | |
| HPS3 | HPS3 biogenesis of | 84343 | 1005 | MVQLYNLHPFGSQQVVPCKLEPDRFCGGGRDALFVAAGCKVEAFA | 358. |
| | lysosomal organelles | | | VAGQELCQPRCAFSTLGRVLRLAYSEAGDYLVAIEEKNKATFLRAY | |
| | complex 2 subunit 1 | | | VNWRNKRTENSRVCIRMIGHNVEGPFSKAFRDQMYIIEMPLSEAPL | |
| | [Source:HGNC | | | CISCCPVKGDLLVGCTNKLVLFSLKYQIINEEFSLLDFERSLIIHIDNIT | |
| | Symbol; Acc:HGNC: | | | PVEVSFCVGYVAVMSDLEVLIVKLESGPKNGERVHHHPHKTNNRIR | |
| | 15597] | | | RTEEGISNEISQLESDDFVICQKPLELLGEKSEQSGLSVTLESTGLAD | |
| | | | | EKRKYSHFQHLLYRRFAPDISSYVLSDDIKLHSLQLLPIYQTGSLTSD | |
| | | | | GKNLSQEKELLSLFCFFSLPHVGYLYMVVKSVELMSVYQYPEKSQ | |
| | | | | QAVLTPQFLHVITSNNLQCFTVRCSAAAAREEDPYMDTTLKACPPV | |
| | | | | SMDVCALRIQLFIGLKAICHFKNHIILLTKAEPEAIPERRQSPKRLLSR | |
| | | | | KDTSVKIKIPPVAEAGWNLYIVNTISPVQLYKEMVDYSNTYKTVKT | |
| | | | | QSCIHLLSEAHLLVRAALMDASQLEPGEKAELLEAFKESCGHLGDC | |
| | | | | YSRLDSQHSHLTLPYYKMSGLSMAEVLARTDWTVEDGLQKYERG | |
| | | | | LIFYINHSLYENLDEELNEELAAKVVQMFYVAEPKQVPHILCSPSMK | |
| | | | | NINPLTAMSYLRKLDTSGFSSILVTLTKAAVALKMGDLDMHRNEM | |
| | | | | KSHSEMKLVCGFILEPRLLIQQRKGQIVPTELALHLKETQPGLLVAS | |
| | | | | VLGLQKNNKIGIEEADSFFKVLCAKDEDTIPQLLVDFWEAQLVACL | |
| | | | | PDVVLQELFFKLTSQYIWRLSKRQPPDTTPLRTSEDLINACSHYGLIY | |
| | | | | PWVHVVISSDSLADKNYTEDLSKLQSLICGPSFDIASIIPFLEPLSEDTI | |
| | | | | AGLSVHVLCRTRLKEYEQCIDILLERCPEAVIPYANHELKEENRTLW | |
| | | | | WKKLLPELCQRIKCGGEKYQLYLSSLKETLSIVAVELELKDFMNVL | |
| | | | | PEDGTATFFLPYLLYCSRKKPLT* | |
| HPS3 | HPS3 biogenesis of | 84343 | 840 | MVQLYNLHPFGSQQVVPCKLEPDRFCGGGRDALFVAAGCKVEAFA | 359. |
| | lysosomal organelles | | | VAGQELCQPRCAFSTLGRVLRLAYSEAGISNEISQLESDDFVICQKP | |
| | complex 2 subunit 1 | | | LELLGEKSEQSGLSVTLESTGLADEKRKYSHFQHLLYRRFAPDISSY | |
| | [Source:HGNC | | | VLSDDIKLHSLQLLPIYQTGSLTSDGKNLSQEKELLSLFCFFSLPHVG | |
| | Symbol; Acc:HGNC: | | | YLYMVVKSVELMSVYQYPEKSQQAVLTPQFLHVITSNNLQCFTVR | |
| | 15597] | | | CSAAAAREEDPYMDTTLKACPPVSMDVCALRIQLFIGLKAICHFKN | |
| | | | | HIILLTKAEPEAIPERRQSPKRLLSRKDTSVKIKIPPVAEAGWNLYIV | |
| | | | | NTISPVQLYKEMVDYSNTYKTVKTQSCIHLLSEAHLLVRAALMDAS | |
| | | | | QLEPGEKAELLEAFKESCGHLGDCYSRLDSQHSHLTLPYYKMSGLS | |
| | | | | MAEVLARTDWTVEDGLQKYERGLIFYINHSLYENLDEELNEELAA | |
| | | | | KVVQMFYVAEPKQVPHILCSPSMKNINPLTAMSYLRKLDTSGFSSIL | |
| | | | | VTLTKAAVALKMGDLDMHRNEMKSHSEMKLVCGFILEPRLLIQQR | |
| | | | | KGQIVPTELALHLKETQPGLLVASVLGLQKNNKIGIEEADSFFKVLC | |
| | | | | AKDEDTIPQLLVDFWEAQLVACLPDVVLQELFFKLTSQYIWRLSKR | |
| | | | | QPPDTTPLRTSEDLINACSHYGLIYPWVHVVISSDSLADKNYTEDLS | |
| | | | | KLQSLICGPSFDIASIIPFLEPLSEDTIAGLSVHVLCRTRLKEYEQCIDI | |
| | | | | LLERCPEAVIPYANHELKEENRTLWWKKLLPELCQRIKCGGEKYQL | |
| | | | | YLSSLKETLSIVAVELELKDFMNVLPEDGTATFFLPYLLYCSRKKPL | |
| | | | | T* | |
| HPS3 | HPS3 biogenesis of | 84343 | 267 | ELAAKVVQMFYVAEPKQVPHILCSPSMKNINPLTAMSYLRKLDTSG | 360. |
| | lysosomal organelles | | | FSSILVTLTKAAVALKMGDLDMHRNEMKSHSEMKLVCGFILEPRLL | |
| | complex 2 subunit 1 | | | IQQRKGQIVPTELALHLKETQPGLLVASVLGLQKNNKIGIEEADSFF | |
| | [Source:HGNC | | | KVLCAKDEDTIPQLLVDFWEAQLVACLPDVVLQELFFKLTSQYIWR | |
| | Symbol; Acc:HGNC: | | | LSKRQPPDTTPLRTSEDLINACSHYGLIYPWVHVVISSDSLADKNYT | |
| | 15597] | | | EDLSKLQLPLFRSWSHFQKTLLPASVSMFCVVHA* | |
| ZNF687 | zinc finger protein 687 | 57592 | 1238 | MGDMKTPDFDDLLAAFDIPDIDANEAIHSGPEENEGPGGPGKPEPG | 361. |
| | [Source:HGNC | | | VGSESEDTAAASAGDGPGVPAQASDHGLPPPDISVVSVIVKNTVCP | |
| | Symbol; Acc:HGNC: | | | EQSEALAGGSAGDGAQAAGVTKEGPVGPHRMQNGFGSPEPSLPGT | |
| | 29277] | | | PHSPAPPSGGTWKEKGMEGKTPLDLFAHFGPEPGDHSDPLPPSAPSP | |
| | | | | TREGALTPPPFPSSFELAQENGPGMQPPVSSPPLGALKQESCSPHHP | |
| | | | | QVLAQQGSGSSPKATDIPASASPPPVAGVPFFKQSPGHQSPLASPKV | |
| | | | | PVCQPLKEEDDDEGPVDKSSPGSPQSPSSGAEAADEDSNDSPASSSS | |
| | | | | RPLKVRIKTIKTSCGNITRTVTQVPSDPDPPAPLAEGAFLAEASLLKL | |
| | | | | SPATPTSEGPKVVSVQLGDGTRLKGTVLPVATIQNASTAMLMAASV | |
| | | | | ARKAVVLPGGTATSPKMIAKNVLGLVPQALPKADGRAGLGTGGQ | |
| | | | | KVNGASVVMVQPSKTATGPSTGGGTVISRTQSSLVEAFNKILNSKN | |
| | | | | LLPAYRPNLSPPAEAGLALPPTGYRCLECGDAFSLEKSLARHYDRRS | |
| | | | | MRIEVTCNHCARRLVFFNKCSLLLHAREHKDKGLVMQCSHLVMRP | |
| | | | | VALDQMVGQPDITPLLPVAVPPVSGPLALPALGKGEGAITSSAITTV | |
| | | | | AAEAPVLPLSTEPPAAPATSAYTCFRCLECKEQCRDKAGMAAHFQ | |
| | | | | QLGPPAPGATSNVCPTCPMMLPNRCSFSAHQRMHKNRPPHVCPEC | |
| | | | | GGNFLQANFQTHLREACLHVSRRVGYRCPSCSVVFGGVNSIKSHIQ | |
| | | | | TSHCEVFHKCPICPMAFKSGPSAHAHLYSQHPSFQTQQAKLIYKCA | |
| | | | | MCDTVFTHKPLLSSHFDQHLLPQRVSVFKCPSCPLLFAQKRTMLEH | |
| | | | | LKNTHQSGRLEETAGKGAGGALLTPKTEPEELAVSQGGAAPATEES | |
| | | | | SSSSEEEEVPSSPEPPRPAKRPRRELGSKGLKGGGGGPGGWTCGLCH | |
| | | | | SWFPERDEYVAHMKKEHGKSVKKFPCRLCERSFCSAPSLRRHVRV | |
| | | | | NHEGIKRVYPCRYCTEGKRTFSSRLILEKHVQVRHGLQLGAQSPGR | |
| | | | | GTTLARGSSARAQGPGRKRRQSSDSCSEEPDSTTPPAKSPRGGPGSG | |
| | | | | GHGPLRYRSSSSTEQSLMMGLRVEDGAQQCLDCGLCFASPGSLSRH | |
| | | | | RFISHKKRRGVGKASALGLGDGEEEAPPSRSDPDGGDSPLPASGGPL | |
| | | | | TCKVCGKSCDSPLNLKTHFRTHGMAFIRARQGAVGDN* | |
| ZNF687 | zinc finger protein 687 | 57592 | 100 | RTFSSRLILEKHVQVRHGLQLGAQSPGRGTTLARGSSARAQFLPLFF | 362. |
| | [Source:HGNC | | | RGQVGNAASLLTLAVRSLTARHRQPSPPGADLDLEAMALCATGAA | |
| | Symbol; Acc:HGNC: | | | APQNRAS* | |
| | 29277] | | | | |
| ZNF687 | zinc finger protein 687 | 57592 | 707 | XTVLPVATIQNASTAMLMAASVARKAVVLPGGTATSPKMIAKNVL | 363. |
| | [Source:HGNC | | | GLVPQALPKADGRAGLGTGGQKVNGASVVMVQPSKTATGPSTGG | |
| | Symbol; Acc:HGNC: | | | GTVISRTQSSLVEAFNKILNSKNLLPAYRPNLSPPAEAGLALPPTGYR | |
| | 29277] | | | CLECGDAFSLEKSLARHYDRRSMRIEVTCNHCARRLVFFNKCSLLL | |
| | | | | HAREHKDKGLVMQCSHLVMRPVALDQMVGQPDITPLLPVAVPPVS | |
| | | | | GPLALPALGKGEGAITSSAITTVAAEAPVLPLSTEPPAAPATSAYTCF | |
| | | | | RCLECKEQCRDKAGMAAHFQQLGPPAPGATSNVCPTCPMMLPNRC | |
| | | | | SFSAHQRMHKNRPPHVCPECGGNFLQANFQTHLREACLHVSRRVG | |
| | | | | YRCPSCSVVFGGVNSIKSHIQTSHCEVFHKCPICPMAFKSGPSAHAH | |
| | | | | LYSQHPSFQTQQAKLIYKCAMCDTVFTHKPLLSSHFDQHLLPQRVS | |
| | | | | VFKCPSCPLLFAQKRTMLEHLKNTHQSGRLEETAGKGAGGALLTP | |
| | | | | KTEPEELAVSQGGAAPATEESSSSSEEEEVPSSPEPPRPAKRPRRELG | |
| | | | | SKGLKGGGGGPGGWTCGLCHSWFPERDEYVAHMKKEHGKSVKKF | |
| | | | | PCRLCERSFCSAPSLRRHVRVNHEGIKRVYPCRSKGPGLRAVPLLPS | |
| | | | | CPLPFQVLHRGKTHLQQPPDPRETCPGPARLAAWGPVPWPGDHLG | |
| | | | | SGFQCQSPGARSETPPVF* | |
| ZNF687 | zinc finger protein 687 | 57592 | 786 | MGDMKTPDFDDLLAAFDIPDIDANEAIHSGPEENEGPGGPGKPEPG | 364. |
| | [Source:HGNC | | | VGSESEDTAAASAGDGPGVPAQASDHGLPPPDISVVSVIVKNTVCP | |
| | Symbol; Acc:HGNC: | | | EQSEALAGGSAGDGAQAAGVTKEGPVGPHRMQNGFGSPEPSLPGT | |
| | 29277] | | | PHSPAPPSGGTWKEKGMEGKTPLDLFAHFGPEPGDHSDPLPPSAPSP | |
| | | | | TREGALTPPPFPSSFELAQENGPGMQPPVSSPPLGALKQESCSPHHP | |
| | | | | QVLAQQGSGSSPKATDIPASASPPPVAGVPFFKQSPGHQSPLASPKV | |
| | | | | PVCQPLKEEDDDEGPVDKSSPGSPQSPSSGAEAADEDSNDSPASSSS | |
| | | | | RPLKVRIKTIKTSCGNITRTVTQVPSDPDPPAPLAEGAFLAEASLLKL | |
| | | | | SPATPTSEGPKVVSVQLGDGTRLKGTVLPVATIQNASTAMLMAASV | |
| | | | | ARKAVVLPGGTATSPKMIAKNVLGLVPQALPKADGRAGLGTGGQ | |
| | | | | KVNGASVVMVQPSKTATGPSTGGGTVISRTQSSLVEAFNKILNSKN | |
| | | | | LLPAYRPNLSPPAEAGLALPPTGYRCLECGDAFSLEKSLARHYDRRS | |
| | | | | MRIEVTCNHCARRLVFFNKCSLLLHAREHKDKGLVMQCSHLVMRP | |
| | | | | VALDQMVGQPDITPLLPVAVPPVSGPLALPALGKGEGAITSSAITTV | |
| | | | | AAEAPVLPLSTEPPAAPATSAYTCFRCLECKEQCRDKAGMAAHFQ | |
| | | | | QLGPPAPGATSNVCPTCPMMLPNRCSFSAHQRMHKNRPPHVCPEC | |
| | | | | GGNFLQANFQTHLREACLHVSRRVGYRTPISLGAWRRLLGKGPGV | |
| | | | | PC* | |
| ZNF687 | zinc finger protein 687 | 57592 | 147 | MGRGLGSADMGDMKTPDFDDLLAAFDIPDIDANEAIHSGPEENEGP | 365. |
| | [Source:HGNC | | | GGPGKPEPGVGSESEDTAAASAGDGPGVPAQASDHGLPPPDISVVS | |
| | Symbol; Acc:HGNC: | | | VIVKNTVCPEQSEALAGGSAGDGAQAAGVTKEGPVGPHRMQNGF | |
| | 29277] | | | GSPEPSLPGTP | |
| THY1 | Thy-1 cell surface | 7070 | 162 | MNLAISIALLLTVLQVSRGQKVTSLTACLVDQSLRLDCRHENTSSSP | 366. |
| | antigen [Source:HGNC | | | IQYEFSLTRETKKHVLFGTVGVPEHTYRSRTNFTSKYNMKVLYLSA | |
| | Symbol; Acc:HGNC: | | | FTSKDEGTYTCALHHSGHSPPISSQNVTVLRDKLVKCEGISLLAQNT | |
| | 11801] | | | SWLLLLLLSLSLLQATDFMSL* | |
| THY1 | Thy-1 cell surface | 7070 | 145 | MNLAISIALLLTVDQSLRLDCRHENTSSSPIQYEFSLTRETKKHVLFG | 367. |
| | antigen [Somce:HGNC | | | TVGVPEHTYRSRTNFTSKYNMKVLYLSAFTSKDEGTYTCALHHSG | |
| | Symbol; Acc:HGNC: | | | HSPPISSQNVTVLRDKLVKCEGISLLAQNTSWLLLLLLSLSLLQATD | |
| | 11801] | | | FMSL* | |
| THY1 | Thy-1 cell surface | 7070 | 153 | MNLAISIALLLTVLQVSRGQKVTSLTACLVDQSLRLDCRHENTSSSP | 368. |
| | antigen [Somce:HGNC | | | IQYEFSLTRETKKHVLFGTVGVPEHTYRSRTNFTSKYNMKVLYLSA | |
| | Symbol; Acc:HGNC: | | | FTSKDEGTYTCALHHSGHSPPISSQNVTVLRDKLVKCEGISLLAQNT | |
| | 11801] | | | SWLLLLLLSLSLL | |
| THY1 | Thy-1 cell surface | 7070 | 165 | MNLAISIALLLTVLQVSRGQKVTSLTACLVDQSLRLDCRHENTSSSP | 369. |
| | antigen [Source:HGNC | | | IQYEFSLTRETKKHVLFGTVGVPEHTYRSRTNFTSKYNMKVLYLSA | |
| | Symbol; Acc:HGNC: | | | FTSKDEGTYTCALHHSGHSPPISSQNVTVLRGHGFHVPVTGGAHGG | |
| | 11801] | | | DRKPQVPVQRSYFSESADPLPPIPQT | |
| THY1 | Thy-1 cell surface | 7070 | 38 | MNLAISIALLLTGLPRAEGDQPNGLPSGPEPSSGLPP* | 370. |
| | antigen [Source:HGNC | | | | |
| | Symbol; Acc:HGNC: | | | | |
| | 11801] | | | | |
| STAT1 | signal transducer and | 6772 | 751 | MSQWYELQQLDSKFLEQVHQLYDDSFPMEIRQYLAQWLEKQDWE | 371. |
| | activator of | | | HAANDVSFATIRFHDLLSQLDDQYSRFSLENNFLLQHNIRKSKRNL | |
| | transcription 1 | | | QDNFQEDPIQMSMIIYSCLKEERKILENAQRFNQAQSGNIQSTVMLD | |
| | [Source:HGNC | | | KQKELDSKVRNVKDKVMCIEHEIKSLEDLQDEYDFKCKTLQNREH | |
| | Symbol; Acc:HGNC: | | | ETNGVAKSDQKQEQLLLKKMYLMLDNKRKEWHKIIELLNVTELT | |
| | 11362] | | | QNALINDELVEWKRRQQSACIGGPPNACLDQLQNWFTIVAESLQQ | |
| | | | | VRQQLKKLEELEQKYTYEHDPITKNKQVLWDRTFSLFQQLIQSSFV | |
| | | | | VERQPCMPTHPQRPLVLKTGVQFTVKLRLLVKLQELNYNLKVKVL | |
| | | | | FDKDVNERNTVKGFRKFNILGTHTKVMNMEESTNGSLAAEFRHLQ | |
| | | | | LKEQKNAGTRTNEGPLIVTEELHSLSFETQLCQPGLVIDLETTSLPVV | |
| | | | | VISNVSQLPSGWASILWYNMLVAEPRNLSFFLTPPCARWAQLSEVL | |
| | | | | SWQFSSVTKRGLNVDQLNMLGEKLLGPNASPDGLIPWTRFCKENIN | |
| | | | | DKNFPFWLWIESILELIKKHLLPLWNDGCIMGFISKERERALLKDQQ | |
| | | | | PGTFLLRFSESSREGAITFTWVERSQNGGEPDFHAVEPYTKKELSAV | |
| | | | | TFPDIIRNYKVMAAENIPENPLKYLYPNIDKDHAFGKYYSRPKEAPE | |
| | | | | PMELDGPKGTGYIKTELISVSEVHPSRLQTTDNLLPMSPEEFDEVSRI | |
| | | | | VGSVEFDSMMNTV* | |
| STAT1 | signal transducer and | 6772 | 713 | MSQWYELQQLDSKFLEQVHQLYDDSFPMEIRQYLAQWLEKQDWE | 372. |
| | activator of | | | HAANDVSFATIRFHDLLSQLDDQYSRFSLENNFLLQHNIRKSKRNL | |
| | transcription 1 | | | QDNFQEDPIQMSMIIYSCLKEERKILENAQRFNQAQSGNIQSTVMLD | |
| | [Source:HGNC | | | KQKELDSKVRNVKDKVMCIEHEIKSLEDLQDEYDFKCKTLQNREH | |
| | Symbol; Acc:HGNC: | | | ETNGVAKSDQKQEQLLLKKMYLMLDNKRKEWHKIIELLNVTELT | |
| | 11362] | | | QNALINDELVEWKRRQQSACIGGPPNACLDQLQNWFTIVAESLQQ | |
| | | | | VRQQLKKLEELEQKYTYEHDPITKNKQVLWDRTFSLFQQLIQSSFV | |
| | | | | VERQPCMPTHPQRPLVLKTGVQFTVKLRLLVKLQELNYNLKVKVL | |
| | | | | FDKDVNERNTVKGFRKFNILGTHTKVMNMEESTNGSLAAEFRHLQ | |
| | | | | LKEQKNAGTRTNEGPLIVTEELHSLSFETQLCQPGLVIDLETTSLPVV | |
| | | | | VISNVSQLPSGWASILWYNMLVAEPRNLSFFLTPPCARWAQLSEVL | |
| | | | | SWQFSSVTKRGLNVDQLNMLGEKLLGPNASPDGLIPWTRFCKENIN | |
| | | | | DKNFPFWLWIESILELIKKHLLPLWNDGCIMGFISKERERALLKDQQ | |
| | | | | PGTFLLRFSESSREGAITFTWVERSQNGGEPDFHAVEPYTKKELSAV | |
| | | | | TFPDIIRNYKVMAAENIPENPLKYLYPNIDKDHAFGKYYSRPKEAPE | |
| | | | | PMELDGPKGTGYIKTELISVSEV* | |
| STAT1 | signal transducer and | 6772 | 715 | MWMSQWYELQQLDSKFLEQVHQLYDDSFPMEIRQYLAQWLEKQD | 373. |
| | activator of | | | WEHAANDVSFATIRFHDLLSQLDDQYSRFSLENNFLLQHNIRKSKR | |
| | transcription 1 | | | NLQDNFQEDPIQMSMIIYSCLKEERKILENAQRFNQAQSGNIQSTVM | |
| | [Source:HGNC | | | LDKQKELDSKVRNVKDKVMCIEHEIKSLEDLQDEYDFKCKTLQNR | |
| | Symbol; Acc:HGNC: | | | EHETNGVAKSDQKQEQLLLKKMYLMLDNKRKEVVHKIIELLNVTE | |
| | 11362] | | | LTQNALINDELVEWKRRQQSACIGGPPNACLDQLQNWFTIVAESLQ | |
| | | | | QVRQQLKKLEELEQKYTYEHDPITKNKQVLWDRTFSLFQQLIQSSF | |
| | | | | VVERQPCMPTHPQRPLVLKTGVQFTVKLRLLVKLQELNYNLKVKV | |
| | | | | LFDKDVNERNTVKGFRKFNILGTHTKVMNMEESTNGSLAAEFRHL | |
| | | | | QLKEQKNAGTRTNEGPLIVTEELHSLSFETQLCQPGLVIDLETTSLPV | |
| | | | | VVISNVSQLPSGWASILWYNMLVAEPRNLSFFLTPPCARWAQLSEV | |
| | | | | LSWQFSSVTKRGLNVDQLNMLGEKLLGPNASPDGLIPWTRFCKENI | |
| | | | | NDKNFPFWLWIESILELIKKHLLPLWNDGCIMGFISKERERALLKDQ | |
| | | | | QPGTFLLRFSESSREGAITFTWVERSQNGGEPDFHAVEPYTKKELSA | |
| | | | | VTFPDIIRNYKVMAAENIPENPLKYLYPNIDKDHAFGKYYSRPKEAP | |
| | | | | EPMELDGPKGTGYIKTELISVSEV* | |
| STAT1 | signal transducer and | 6772 | 150 | MSQWYELQQLDSKFLEQVHQLYDDSFPMEIRQYLAQWLEKQDWE | 374. |
| | activator of | | | HAANDVSFATIRFHDLLSQLDDQYSRFSLENNFLLQHNIRKSKRNL | |
| | transcription 1 | | | QDNFQEDPIQMSMIIYSCLKEERKILENAQRFNQAQSGNIQSTVMLD | |
| | [Source:HGNC | | | KQKELDSKVRNVK | |
| | Symbol; Acc:HGNC: | | | | |
| | 11362] | | | | |
| STAT1 | signal transducer and | 6772 | 194 | MSQWYELQQLDSKFLEQVHQLYDDSFPMEIRQYLAQWLEKQDWE | 375. |
| | activator of | | | HAANDVSFATIRFHDLLSQLDDQYSRFSLENNFLLQHNIRKSKRNL | |
| | transcription 1 | | | QDNFQEDPIQMSMIIYSCLKEERKILENAQRFNQAQSGNIQSTVMLD | |
| | [Source:HGNC | | | KQKELDSKVRNVKDKVMCIEHEIKSLEDLQDEYDFKCKTLQNRGS | |
| | Symbol; Acc:HGNC: | | | SSQNNRVAECH* | |
| | 11362] | | | | |
| STAT1 | signal transducer and | 6772 | 70 | MSQWYELQQLDSKFLEQVHQLYDDSFPMEIRQYLAQWLEKQDWE | 376. |
| | activator of | | | HAANDVSFATIRFHDLLSQLDDQYSR | |
| | transcription 1 | | | | |
| | [Source HGNC | | | | |
| | Symbol; Acc:HGNC: | | | | |
| | 11362] | | | | |
| STAT1 | signal transducer and | 6772 | 43 | MSQWYELQQLDSKFLEQVHQLYDDSFPMEIRQYLAQWLEKQD* | 377. |
| | activator of | | | | |
| | transcription 1 | | | | |
| | [Source:HGNC | | | | |
| | Symbol; Acc:HGNC: | | | | |
| | 11362] | | | | |
| STAT1 | signal transducer and | 6772 | 377 | MSQWYELQQLDSKFLEQVHQLYDDSFPMEIRQYLAQWLEKQDWE | 378. |
| | activator of | | | HAANDVSFATIRFHDLLSQLDDQYSRFSLENNFLLQHNIRKSKRNL | |
| | transcription 1 | | | QDNFQEDPIQMSMIIYSCLKEERKILENAQRFNQCIEHEIKSLEDLQD | |
| | [Source:HGNC | | | EYDFKCKTLQNREHETNGVAKSDQKQEQLLLKKMYLMLDNKRKE | |
| | Symbol; Acc:HGNC: | | | VVHKIIELLNVTELTQNALINDELVEWKRRQQSACIGGPPNACLDQ | |
| | 11362] | | | LQNWFTIVAESLQQVRQQLKKLEELEQKYTYEHDPITKNKQVLWD | |
| | | | | RTFSLFQQLIQSSFVVERQPCMPTHPQRPLVLKTGVQFTVKLRLLVK | |
| | | | | LQELNYNLKVKVLFDKDVNERNTVKGFRKFNILGTHTKVMNMEES | |
| | | | | TNGSLAAEFRHL | |
| STAT1 | signal transducer and | 6772 | 573 | MSQWYELQQLDSKFLEQVHQLYDDSFPMEIRQYLAQWLEKQDWE | 379. |
| | activator of | | | HAANDVSFATIRFHDLLSQLDDQYSRFSLENNFLLQHNIRKSKRNL | |
| | transcription 1 | | | QDNFQEDPIQMSMIIYSCLKEERKILENAQRFNQAQSGNIQSTVMLD | |
| | [Source:HGNC | | | KQKELDSKVRNVKDKVMCIEHEIKSLEDLQDEYDFKCKTLQNREH | |
| | Symbol; Acc:HGNC: | | | ETNGVAKSDQKQEQLLLKKMYLMLDNKRKEWHKIIELLNVTELT | |
| | 11362] | | | QNALINDELVEWKRRQQSACIGGPPNACLDQLQNWFTIVAESLQQ | |
| | | | | VRQQLKKLEELEQKYTYEHDPITKNKQVLWDRTFSLFQQLIQSSFV | |
| | | | | VERQPCMPTHPQRPLVLKTGVQFTVKLRLLVKLQELNYNLKVKVL | |
| | | | | FDKDVNERNTVKGFRKFNILGTHTKVMNMEESTNGSLAAEFRHLQ | |
| | | | | LKEQKNAGTRTNEGPLIVTEELHSLSFETQLCQPGLVIDLETTSLPVV | |
| | | | | VISNVSQLPSGWASILWYNMLVAEPRNLSFFLTPPCARWAQLSEVL | |
| | | | | SWQFSSVTKRGLNVDQLNMLGEKLLGPNASPDGLIPWTRFCKVHH | |
| | | | | GLHQQGARACPVEGPAAGDLPAAVQ* | |
| STAT1 | signal transducer and | 6772 | 27 | RQYLAQWLEKQDWSLSSFRRPLCPLW* | 380. |
| | activator of | | | | |
| | transcription 1 | | | | |
| | [Source:HGNC | | | | |
| | Symbol; Acc:HGNC: | | | | |
| | 11362] | | | | |
| STAT1 | signal transducer and | 6772 | 749 | MSQWYELQQLDSKFLEQVHQLYDDSFPMEIRQYLAQWLEKQDWE | 381. |
| | activator of | | | HAANDVSFATIRFHDLLSQLDDQYSRFSLENNFLLQHNIRKSKRNL | |
| | transcription 1 | | | QDNFQEDPIQMSMIIYSCLKEERKILENAQRFNQAQSGNIQSTVMLD | |
| | [Source:HGNC | | | KQKELDSKVRNVKDKVMCIEHEIKSLEDLQDEYDFKCKTLQNREH | |
| | Symbol; Acc:HGNC: | | | ETNGVAKSDQKQEQLLLKKMYLMLDNKRKEVVHKIIELLNVTELT | |
| | 11362] | | | QNALINDELVEWKRRQQSACIGGPPNACLDQLQNWFTIVAESLQQ | |
| | | | | VRQQLKKLEELEQKYTYEHDPITKNKQVLWDRTFSLFQQLIQSSFV | |
| | | | | VERQPCMPTHPQRPLVLKTGVQFTVKLRLLVKLQELNYNLKVKVL | |
| | | | | FDKDVNERNTVKGFRKFNILGTHTKVMNMEESTNGSLAAEFRHLQ | |
| | | | | LKEQKNAGTRTNEGPLIVTEELHSLSFETQLCQPGLVIDLETTSLPVV | |
| | | | | VISNVSQLPSGWASILWYNMLVAEPRNLSFFLTPPCARWAQLSEVL | |
| | | | | SWQFSSVTKRGLNVDQLNMLGEKLLGPNASPDGLIPWTRFCKENIN | |
| | | | | DKNFPFWLWIESILELIKKHLLPLWNDGCIMGFISKERERALLKDQQ | |
| | | | | PGTFLLRFSESSREGAITFTWVERSQNGGEPDFHAVEPYTKKELSAV | |
| | | | | TFPDIIRNYKVMAAENIPENPLKYLYPNIDKDHAFGKYYSRPKEAPE | |
| | | | | PMELDGPKGTGYIKTELISVSEVHPSRLQTTDNLLPMSPEEFDEVSRI | |
| | | | | VGSVEFDSMSF* | |
| STAT1 | signal transducer and | 6772 | 792 | MSQWYELQQLDSKFLEQVHQLYDDSFPMEIRQYLAQWLEKQDWE | 382. |
| | activator of | | | HAANDVSFATIRFHDLLSQLDDQYSRFSLENNFLLQHNIRKSKRNL | |
| | transcription 1 | | | QDNFQEDPIQMSMIIYSCLKEERKILENAQRFNQAQSGNIQSTVMLD | |
| | [Source:HGNC | | | KQKELDSKVRNVKDKVMCIEHEIKSLEDLQDEYDFKCKTLQNREH | |
| | Symbol; Acc:HGNC: | | | ETNGVAKSDQKQEQLLLKKMYLMLDNKRKEWHKIIELLNVTELT | |
| | 11362] | | | QNALINDELVEWKRRQQSACIGGPPNACLDQLQNWFTIVAESLQQ | |
| | | | | VRQQLKKLEELEQKYTYEHDPITKNKQVLWDRTFSLFQQLIQSSFV | |
| | | | | VERQPCMPTHPQRPLVLKTGVQFTVKLRLLVKLQELNYNLKVKVL | |
| | | | | FDKDVNERNTVKGFRKFNILGTHTKVMNMEESTNGSLAAEFRHLQ | |
| | | | | LKEQKNAGTRTNEGPLIVTEELHSLSFETQLCQPGLVIDLETTSLPVV | |
| | | | | VISNVSQLPSGWASILWYNMLVAEPRNLSFFLTPPCARWAQLSEVL | |
| | | | | SWQFSSVTKRGLNVDQLNMLGEKLLGPNASPDGLIPWTRFCKENIN | |
| | | | | DKNFPFWLWIESILELIKKHLLPLWNDGCIMGFISKERERALLKDQQ | |
| | | | | PGTFLLRFSESSREGAITFTWVERSQNGGEPDFHAVEPYTKKELSAV | |
| | | | | TFPDIIRNYKVMAAENIPENPLKYLYPNIDKDHAFGKYYSRPKEAPE | |
| | | | | PMELDGPKGTGYIKTELISVSEVHPSRLQTTDNLLPMSPEEFDEVSRI | |
| | | | | VGSVEFDSMVSTTAGLCVALNKCVCSEAGSAPWQIPGLSAGGSPG | |
| | | | | GAKESAVLR* | |
| STAT1 | signal transducer and | 6772 | 749 | MSQWYELQQLDSKFLEQVHQLYDDSFPMEIRQYLAQWLEKQDWE | 383. |
| | activator of | | | HAANDVSFATIRFHDLLSQLDDQYSRFSLENNFLLQHNIRKSKRNL | |
| | transcription 1 | | | QDNFQEDPIQMSMIIYSCLKEERKILENAQRFNQAQSGNIQSTVMLD | |
| | [Source:HGNC | | | KQKELDSKVRNVKDKVMCIEHEIKSLEDLQDEYDFKCKTLQNREH | |
| | Symbol; Acc:HGNC: | | | ETNGVAKSDQKQEQLLLKKMYLMLDNKRKEVVHKIIELLNVTELT | |
| | 11362] | | | QNALINDELVEWKRRQQSACIGGPPNACLDQLQNWFTIVAESLQQ | |
| | | | | VRQQLKKLEELEQKYTYEHDPITKNKQVLWDRTFSLFQQLIQSSFV | |
| | | | | VERQPCMPTHPQRPLVLKTGVQFTVKLRLLVKLQELNYNLKVKVL | |
| | | | | FDKDVNERNTVKGKFNILGTHTKVMNMEESTNGSLAAEFRHLQLK | |
| | | | | EQKNAGTRTNEGPLIVTEELHSLSFETQLCQPGLVIDLETTSLPVVVI | |
| | | | | SNVSQLPSGWASILWYNMLVAEPRNLSFFLTPPCARWAQLSEVLS | |
| | | | | WQFSSVTKRGLNVDQLNMLGEKLLGPNASPDGLIPWTRFCKENIN | |
| | | | | DKNFPFWLWIESILELIKKHLLPLWNDGCIMGFISKERERALLKDQQ | |
| | | | | PGTFLLRFSESSREGAITFTWVERSQNGGEPDFHAVEPYTKKELSAV | |
| | | | | TFPDIIRNYKVMAAENIPENPLKYLYPNIDKDHAFGKYYSRPKEAPE | |
| | | | | PMELDGPKGTGYIKTELISVSEVHPSRLQTTDNLLPMSPEEFDEVSRI | |
| | | | | VGSVEFDSMMNTV* | |
| STAT1 | signal transducer and | 6772 | 274 | MSQWYELQQLDSKFLEQVHQLYDDSFPMEIRQYLAQWLEKQDWE | 384. |
| | activator of | | | HAANDVSFATIRFHDLLSQLDDQYSRFSLENNFLLQHNIRKSKRNL | |
| | transcription 1 | | | QDNFQEDPIQMSMIIYSCLKEERKILENAQRFNQAQSGNIQSTVMLD | |
| | [Source:HGNC | | | KQKELDSKVRNVKDKVMCIEHEIKSLEDLQDEYDFKCKTLQNREH | |
| | Symbol; Acc:HGNC: | | | ETNGVAKSDQKQEQLLLKKMYLMLDNKRKEVVHKIIELLNVTELT | |
| | 11362] | | | QNALINDELVEWKRRQQSACIGGPPNACLDQLQNCCGESAASSAA | |
| | | | | A* | |
| STAT1 | signal transducer and | 6772 | 717 | MSQWYELQQLDSKFLEQVHQLYDDSFPMEIRQYLAQWLEKQDWE | 385. |
| | activator of | | | HAANDVSFATIRFHDLLSQLDDQYSRFSLENNFLLQHNIRKSKRNL | |
| | transcription 1 | | | QDNFQEDPIQMSMIIYSCLKEERKILENAQRFNQAQSGNIQSTVMLD | |
| | [Source:HGNC | | | KQKELDSKVRNVKDKVMCIEHEIKSLEDLQDEYDFKCKTLQNREH | |
| | Symbol; Acc:HGNC: | | | ETNGVAKSDQKQEQLLLKKMYLMLDNKRKEVVHKIIELLNVTELT | |
| | 11362] | | | QNALINDELVEWKRRQQSACIGGPPNACLDQLQNWFTIVAESLQQ | |
| | | | | VRQQLKKLEELEQKYTYEHDPITKNKQVLWDRTFSLFQQLIQSSFV | |
| | | | | VERQPCMPTHPQRPLVLKTGVQFTVKLRLLVKLQELNYNLKVKVL | |
| | | | | FDKDVNERNTVKGFRKFNILGTHTKVMNMEESTNGSLAAEFRHLQ | |
| | | | | LKEQKNAGTRTNEGPLIVTEELHSLSFETQLCQPGLVIDLETTSLPVV | |
| | | | | VISNVSQLPSGWASILWYNMLVAEPRNLSFFLTPPCARWAQLSEVL | |
| | | | | SWQFSSVTKRGLNVDQLNMLGEKLLGPNASPDGLIPWTRFCKENIN | |
| | | | | DKNFPFWLWIESILELIKKHLLPLWNDGCIMGFISKERERALLKDQQ | |
| | | | | PGTFLLRFSESSREGAITFTWVERSQNGGEPDFHAVEPYTKKELSAV | |
| | | | | TFPDIIRNYKVMAAENIPENPLKYLYPNIDKDHAFGKYYSRPKEAPE | |
| | | | | PMELDGPKGTGYIKTELISVSEVTVLG* | |
| STAT1 | signal transducer and | 6772 | 637 | MSQWYELQQLDSKFLEQVHQLYDDSFPMEIRQYLAQWLEKQDWE | 386. |
| | activator of | | | HAANDVSFATIRFHDLLSQLDDQYSRFSLENNFLLQHNIRKSKRNL | |
| | transcription 1 | | | QDNFQEDPIQMSMIIYSCLKEERKILENAQRFNQAQSGNIQSTVMLD | |
| | [Source:HGNC | | | KQKELDSKVRNVKDKVMCIEHEIKSLEDLQDEYDFKCKTLQNREH | |
| | Symbol; Acc:HGNC: | | | ETNGVAKSDQKQEQLLLKKMYLMLDNKRKEVVHKIIELLNVTELT | |
| | 11362] | | | QNALINDELVEWKRRQQSACIGGPPNACLDQLQNWFTIVAESLQQ | |
| | | | | VRQQLKKLEELEQKYTYEHDPITKNKQVLWDRTFSLFQQLIQSSFV | |
| | | | | VERQPCMPTHPQRPLVLKTGVQFTVKLRLLVKLQELNYNLKVKVL | |
| | | | | FDKDVNERNTVKGFRKFNILGTHTKVMNMEESTNGSLAAEFRHLQ | |
| | | | | LKEQKNAGTRTNEGPLIVTEELHSLSFETQLCQPGLVIDLETTSLPVV | |
| | | | | VISNVSQLPSGWASILWYNMLVAEPRNLSFFLTPPCARWAQLSEVL | |
| | | | | SWQFSSVTKRGLNVDQLNMLGEKLLGPNASPDGLIPWTRFCKENIN | |
| | | | | DKNFPFWLWIESILELIKKHLLPLWNDGCIMGFISKERERALLKDQQ | |
| | | | | PGTFLLRFSESSREGAITFTWVERSQNGGGLLSADQGCESM* | |
| STAT1 | signal transducer and | 6772 | 780 | MSQWYELQQLDSKFLEQVHQLYDDSFPMEIRQYLAQWLEKQDWE | 387. |
| | activator of | | | HAANDVSFATIRFHDLLSQLDDQYSRFSLENNFLLQHNIRKSKRNL | |
| | transcription 1 | | | QDNFQEDPIQMSMIIYSCLKEERKILENAQRFNQAQSGNIQSTVMLD | |
| | [Source:HGNC | | | KQKELDSKVRNVKDKVMCIEHEIKSLEDLQDEYDFKCKTLQNREH | |
| | Symbol; Acc:HGNC: | | | ETNGVAKSDQKQEQLLLKKMYLMLDNKRKEVVHKIIELLNVTELT | |
| | 11362] | | | QNALINDELVEWKRRQQSACIGGPPNACLDQLQNWFTIVAESLQQ | |
| | | | | VRQQLKKLEELEQKYTYEHDPITKNKQVLWDRTFSLFQQLIQSSFV | |
| | | | | VERQPCMPTHPQRPLVLKTGVQFTVKLRLLVKLQELNYNLKVKVL | |
| | | | | FDKDVNERNTVKGFRKFNILGTHTKVMNMEESTNGSLAAEFRHLQ | |
| | | | | LKEQKNAGTRTNEGPLIVTEELHSLSFETQLCQPGLVIDLETTSLPVV | |
| | | | | VISNVSQLPSGWASILWYNMLVAEPRNLSFFLTPPCARWAQLSEVL | |
| | | | | SWQFSSVTKRGLNVDQLNMLGEKLLGPNASPDGLIPWTRFCKENIN | |
| | | | | DKNFPFWLWIESILELIKKHLLPLWNDGCIMGFISKERERALLKDQQ | |
| | | | | PGTFLLRFSESSREGAITFTWVERSQNGGEPDFHAVEPYTKKELSAV | |
| | | | | TFPDIIRNYKVMAAENIPENPLKYLYPNIDKDHAFGKYYSRPKEAPE | |
| | | | | PMELDGPKGTGYIKTELISVSEVHPSRLQTTDNLLPMSPEEFDEVSRI | |
| | | | | VGSVEFDSMALALLPRLECRAAIMAHCSLDLPSSTDPPNSAS* | |
| MPC2 | mitochondrial pyruvate | 25874 | 128 | MSAAGARGLRATYHRLLDKVELMLPEKLRPLYNHPAGPRTVFFWA | 388. |
| | carrier 2 | | | PIMKWGLVCAGLADMARPAEKLSTAQSAVLMATGFIWSRYSLVIIP | |
| | [Source:HGNC | | | KNWSLFAVNFFVGAAGASQLFRIWRYNQELKAKAHK* | |
| | Symbol; Acc:HGNC: | | | | |
| | 24515] | | | | |
| MPC2 | mitochondrial pyruvate | 25874 | 105 | MSAAGARGLRATYHRLLDKVELMLPEKLRPLYNHPAGPRTVFFWA | 389. |
| | carrier 2 | | | PIMKWGLVCAGLADMARPAEKLSTAQSAVLMATGFIWSRYSLVIIP | |
| | [Source:HGNC | | | KNWSLFAVNFFVGA | |
| | Symbol; Acc:HGNC: | | | | |
| | 24515] | | | | |
| PNKD | PNKD metallo-beta- | 25953 | 143 | MAAVVAATALKGRGARNARVLRGILAGATANKASHNRTRALQSH | 390. |
| | lactamase domain | | | SSPEGKEEPEPLSPELEYIPRKRGKNPMKAVGLAWAIGFPCGILLFIL | |
| | containing | | | TKREVDKDRVKQMKARQNMRLSNTGEYESQRFRASSQSAPSPDVG | |
| | [Source:HGNC | | | SGVQT* | |
| | Symbol; Acc:HGNC: | | | | |
| | 9153] | | | | |
| PNKD | PNKD metallo-beta- | 25953 | 362 | MAWQGWPAAWQWVAGCWLLLVLVLVLLVSPRGCRARRGLRGLL | 391. |
| | lactamase domain | | | MAHSQRLLFRIGYSLYTRTWLGYLFYRQQLRRARNRYPKGHSKTQ | |
| | containing | | | PRLFNGVKVLPIPVLSDNYSYLIIDTQAQLAVAVDPSDPRAVQASIE | |
| | [Source:HGNC | | | KEGVTLVAILCTHKHWDHSGGNRDLSRRHRDCRVYGSPQDGIPYL | |
| | Symbol; Acc:HGNC: | | | THPLCHQDVVSVGRLQIRALATPGHTQGHLVYLLDGEPYKGPSCLF | |
| | 9153] | | | SGDLLFLSGCGRTFEGNAETMLSSLDTVLGLGDDTLLWPGHEYAEE | |
| | | | | NLGFAGVVEPENLARERKMQWVQRQRLERKGTCPSTLGEERSYNP | |
| | | | | FLRTHCLALQEALGPGPGPTGDDDYSRAQLLEELRRLKDMHKSK* | |
| PNKD | PNKD metallo-beta- | 25953 | 386 | MAAVVAATALKGRGARNARVLRGILAGATANKASHNRTRALQSH | 392. |
| | lactamase domain | | | SSPEGKEEPEPLSPELEYIPRKRGKNPMKAVGLAWYSLYTRTWLGY | |
| | containing | | | LFYRQQLRRARNRYPKGHSKTQPRLFNGVKVLPIPVLSDNYSYLIID | |
| | [Source:HGNC | | | TQAQLAVAVDPSDPRAVQASIEKEGVTLVAILCTHKHWDHSGGNR | |
| | Symbol; Acc:HGNC: | | | DLSRRHRDCRVYGSPQDGIPYLTHPLCHQDVVSVGRLQIRALATPG | |
| | 9153] | | | HTQGHLVYLLDGEPYKGPSCLFSGDLLFLSGCGRTFEGNAETMLSS | |
| | | | | LDTVLGLGDDTLLWPGHEYAEENLGFAGVVEPENLARERKMQWV | |
| | | | | QRQRLERKGTCPSTLGEERSYNPFLRTHCLALQEALGPGPGPTGDD | |
| | | | | DYSRAQLLEELRRLKDMHKSK* | |
| PNKD | PNKD metallo-beta- | 25953 | 326 | MPSSVHHTKRQMMSIYCYWYSLYTRTWLGYLFYRQQLRRARNRY | 393. |
| | lactamase domain | | | PKGHSKTQPRLFNGVKVLPIPVLSDNYSYLIIDTQAQLAVAVDPSDP | |
| | containing | | | RAVQASIEKEGVTLVAILCTHKHWDHSGGNRDLSRRHRDCRVYGS | |
| | [Source:HGNC | | | PQDGIPYLTHPLCHQDVVSVGRLQIRALATPGHTQGHLVYLLDGEP | |
| | Symbol; Acc:HGNC: | | | YKGPSCLFSGDLLFLSGCGRTFEGNAETMLSSLDTVLGLGDDTLLW | |
| | 9153] | | | PGHEYAEENLGFAGVVEPENLARERKMQWVQRQRLERKGTCPSTL | |
| | | | | GEERSYNPFLRTHCLALQEALGPGPGPTGDDDYSRAQLLEELRRLK | |
| | | | | DMHKSK* | |
| ACSL3 | acyl-CoA synthetase | 2181 | 721 | MNNHVSSKPSTMKLKHTINPILLYFIHFLISLYTILTYIPFYFFSESRQE | 394. |
| | long chain family | | | KSNRIKAKPVNSKPDSAYRSVNSLDGLASVLYPGCDTLDKVFTYAK | |
| | member 3 | | | NKFKNKRLLGTREVLNEEDEVQPNGKIFKKVILGQYNWLSYEDVF | |
| | [Source:HGNC | | | VRAFNFGNGLQMLGQKPKTNIAIFCETRAEWMIAAQACFMYNFQL | |
| | Symbol; Acc:HGNC: | | | VTLYATLGGPAIVHALNETEVTNIITSKELLQTKLKDIVSLVPRLRHII | |
| | 3570] | | | TVDGKPPTWSEFPKGIIVHTMAAVEALGAKASMENQPHSKPLPSDI | |
| | | | | AVIMYTSGSTGLPKGVMISHSNIIAGITGMAERIPELGEEDVYIGYLP | |
| | | | | LAHVLELSAELVCLSHGCRIGYSSPQTLADQSSKIKKGSKGDTSML | |
| | | | | KPTLMAAVPEIMDRIYKNVMNKVSEMSSFQRNLFILAYNYKMEQIS | |
| | | | | KGRNTPLCDSFVFRKVRSLLGGNIRLLLCGGAPLSATTQRFMNICFC | |
| | | | | CPVGQGYGLTESAGAGTISEVWDYNTGRVGAPLVCCEIKLKNWEE | |
| | | | | GGYFNTDKPHPRGEILIGGQSVTMGYYKNEAKTKADFFEDENGQR | |
| | | | | WLCTGDIGEFEPDGCLKIIDRKKDLVKLQAGEYVSLGKVEAALKNL | |
| | | | | PLVDNICAYANSYHSYVIGFVVPNQKELTELARKKGLKGTWEELC | |
| | | | | NSCEMENEVLKVLSEAAISASLEKFEIPVKIRLSPEPWTPETGLVTDA | |
| | | | | FKLKRKELKTHYQADIERMYGRK* | |
| ACSL3 | acyl-CoA synthetase | 2181 | 19 | XEQISKGRNTPLCDRWIL* | 395. |
| | long chain family | | | | |
| | member 3 | | | | |
| | [Source:HGNC | | | | |
| | Symbol; Acc:HGNC: | | | | |
| | 3570] | | | | |
| ACSL3 | acyl-CoA synthetase | 2181 | 88 | MNNHVSSKPSTMKLKHTINPILLYFIHFLISLYTILTYIPFYFFSESRQE | 396. |
| | long chain family | | | KSNRIKAKPVNSKPDSAYRSVNSLDGLASVLYPGCDTL | |
| | member 3 | | | | |
| | [Source:HGNC | | | | |
| | Symbol; Acc:HGNC: | | | | |
| | 3570] | | | | |
| ACSL3 | acyl-CoA synthetase | 2181 | 95 | MNNHVSSKPSTMKLKHTINPILLYFIHFLISLYTILTYIPFYFFSESRQE | 397. |
| | long chain family | | | KSNRIKAKPVNSKPDSAYRSVNSLDGLASVLYPGCDTLDKVFTYA | |
| | member 3 | | | | |
| | [Source:HGNC | | | | |
| | Symbol; Acc:HGNC: | | | | |
| | 3570] | | | | |
| ACSL3 | acyl-CoA synthetase | 2181 | 141 | MLGQKPKTNIAIFCETRAEWMIAAQACFMYNFQLVTLYATLGGPAI | 398. |
| | long chain family | | | VHALNETEVTNIITSKELLQTKLKDIVSLVPRLRHIITVDGKPPTWSE | |
| | member 3 | | | FPKGIIVHTMAAVEALGAKASMENQPHSKPLPSDIAVIMYTSGSTGL | |
| | [Source:HGNC | | | | |
| | Symbol; Acc:HGNC: | | | | |
| | 3570] | | | | |
| HDAC4 | histone deacetylase 4 | 9759 | 1085 | MSSQSHPDGLSGRDQPVELLNPARVNHMPSTVDVATALPLQVAPS | 399. |
| | [Source:HGNC | | | AVPMDLRLDHQFSLPVAEPALREQQLQQELLALKQKQQIQRQILIA | |
| | Symbol; Acc:HGNC: | | | EFQRQHEQLSRQHEAQLHEHIKQQQEMLAMKHQQELLEHQRKLER | |
| | 14063] | | | HRQEQELEKQHREQKLQQLKNKEKGKESAVASTEVKMKLQEFVL | |
| | | | | NKKKALAHRNLNHCISSDPRYWYGKTQHSSLDQSSPPQSGVSTSYN | |
| | | | | HPVLGMYDAKDDFPLRKTASEPNLKLRSRLKQKVAERRSSPLLRRK | |
| | | | | DGPVVTALKKRPLDVTDSACSSAPGSGPSSPNNSSGSVSAENGIAPA | |
| | | | | VPSIPAETSLAHRLVAREGSAAPLPLYTSPSLPNITLGLPATGPSAGT | |
| | | | | AGQQDAERLTLPALQQRLSLFPGTHLTPYLSTSPLERDGGAAHSPLL | |
| | | | | QHMVLLEQPPAQAPLVTGLGALPLHAQSLVGADRVSPSIHKLRQHR | |
| | | | | PLGRTQSAPLPQNAQALQHLVIQQQHQQFLEKHKQQFQQQQLQMN | |
| | | | | KIIPKPSEPARQPESHPEETEEELREHQALLDEPYLDRLPGQKEAHA | |
| | | | | QAGVQVKQEPIESDEEEAEPPREVEPGQRQPSEQELLFRQQALLLEQ | |
| | | | | QRIHQLRNYQASMEAAGIPVSFGGHRPLSRAQSSPASATFPVSVQEP | |
| | | | | PTKPRFTTGLVYDTLMLKHQCTCGSSSSHPEHAGRIQSIWSRLQETG | |
| | | | | LRGKCECIRGRKATLEELQTVHSEAHTLLYGTNPLNRQKLDSKKLL | |
| | | | | GSLASVFVRLPCGGVGVDSDTIWNEVHSAGAARLAVGCVVELVFK | |
| | | | | VATGELKNGFAVVRPPGHHAEESTPMGFCYFNSVAVAAKLLQQRL | |
| | | | | SVSKILIVDWDVHHGNGTQQAFYSDPSVLYMSLHRYDDGNFFPGS | |
| | | | | GAPDEVGTGPGVGFNVNMAFTGGLDPPMGDAEYLAAFRTVVMPI | |
| | | | | ASEFAPDVVLVSSGFDAVEGHPTPLGGYNLSARCFGYLTKQLMGL | |
| | | | | AGGRIVLALEGGHDLTAICDASEACVSALLGNELDPLPEKVLQQRP | |
| | | | | NANAVRSMEKVMEIHSKYWRCLQRTTSTAGRSLIEAQTCENEEAE | |
| | | | | TVTAMASLSVGVKPAEKRPDEEPMEEEPPL* | |
| HDAC4 | histone deacetylase 4 | 9759 | 123 | MPSTVDVATALPLQVAPSAVPMDLRLDHQFSLPVAEPALREQQLQ | 400. |
| | [Source:HGNC | | | QELLALKQKQQIQRQILIAEFQRQHEQLSRQHEAQLHEHIKQQQEM | |
| | Symbol; Acc:HGNC: | | | LAMKHQQELLEHQRKLERHRQEQELEKQHREQ | |
| | 14063] | | | | |
| HDAC4 | histone deacetylase 4 | 9759 | 162 | MDVATALPLQVAPSAVPMDLRLDHQFSLPVAEPALREQQLQQELL | 401. |
| | [Source:HGNC | | | ALKQKQQIQRQILIAEFQRQHEQLSRQHEAQLHEHIKQQQEMLAMK | |
| | Symbol; Acc:HGNC: | | | HQQELLEHQRKLERHRQEQELEKQHREQKLQQLKNKEKGKESAV | |
| | 14063] | | | ASTEVKMKLQEFVLNKKKALAHRNLNH | |
| HDAC4 | histone deacetylase 4 | 9759 | 176 | XAWPCVSTPQPPVFLRCHETRFWAVLAAVLPRSHCSLPFPVAGFGY | 402. |
| | [Source:HGNC | | | LTKQLMGLAGGRIVLALEGGHDLTAICDASEACVSALLGNELDPLP | |
| | Symbol; Acc:HGNC: | | | EKVLQQRPNANAVRSMEKVMEIHSKYWRCLQRTTSTAGRSLIEAQ | |
| | 14063] | | | TCENEEAETVTAMASLSVGVKPAEKRPDEEPMEEEPPL* | |
| HDAC4 | histone deacetylase 4 | 9759 | 148 | XWLYLFPHPPHPPHCGSGLYLQSHHLQQAWIPHSACSSAPGSGPSSP | 403. |
| | [Source:HGNC | | | NNSSGSVSAENGIAPAVPSIPAETSLAHRLVAREGSAAPLPLYTSPSL | |
| | Symbol; Acc:HGNC: | | | PNITLGLPATGPSAGTAGQQDAERLTLPALQQRLSLFPGTHLTPYLS | |
| | 14063] | | | TSPLER | |
| HDAC4 | histone deacetylase 4 | 9759 | 1090 | MSSQSHPDGLSGRDQPVELLNPARVNHMPSTVDVATALPLQVAPS | 404. |
| | [Source:HGNC | | | AVPMDLRLDHQFSLPVAEPALREQQLQQELLALKQKQQIQRQILIA | |
| | Symbol; Acc:HGNC: | | | EFQRQHEQLSRQHEAQLHEHIKQQQEMLAMKHQQELLEHQRKLER | |
| | 14063] | | | HRQEQELEKQHREQKLQQLKNKEKGKESAVASTEVKMKLQEFVL | |
| | | | | NKKKALAHRNLNHCISSDPRYWYGKTQHSSLDQSSPPQSGVSTSYN | |
| | | | | HPVLGMYDAKDDFPLRKTASEPNLKLRSRLKQKVAERRSSPLLRRK | |
| | | | | DGPVVTALKKRPLDVTDSACSSAPGSGPSSPNNSSGSVSAENGIAPA | |
| | | | | VPSIPAETSLAHRLVAREGSAAPLPLYTSPSLPNITLGLPATGPSAGT | |
| | | | | AGQQDAERLTLPALQQRLSLFPGTHLTPYLSTSPLERDGGAAHSPLL | |
| | | | | QHMVLLEQPPAQAPLVTDWYLSGLGALPLHAQSLVGADRVSPSIH | |
| | | | | KLRQHRPLGRTQSAPLPQNAQALQHLVIQQQHQQFLEKHKQQFQQ | |
| | | | | QQLQMNKIIPKPSEPARQPESHPEETEEELREHQALLDEPYLDRLPG | |
| | | | | QKEAHAQAGVQVKQEPIESDEEEAEPPREVEPGQRQPSEQELLFRQ | |
| | | | | QALLLEQQRIHQLRNYQASMEAAGIPVSFGGHRPLSRAQSSPASATF | |
| | | | | PVSVQEPPTKPRFTTGLVYDTLMLKHQCTCGSSSSHPEHAGRIQSIW | |
| | | | | SRLQETGLRGKCECIRGRKATLEELQTVHSEAHTLLYGTNPLNRQK | |
| | | | | LDSKKLLGSLASVFVRLPCGGVGVDSDTIWNEVHSAGAARLAVGC | |
| | | | | VVELVFKVATGELKNGFAVVRPPGHHAEESTPMGFCYFNSVAVAA | |
| | | | | KLLQQRLSVSKILIVDWDVHHGNGTQQAFYSDPSVLYMSLHRYDD | |
| | | | | GNFFPGSGAPDEVGTGPGVGFNVNMAFTGGLDPPMGDAEYLAAFR | |
| | | | | TVVMPIASEFAPDVVLVSSGFDAVEGHPTPLGGYNLSARCFGYLTK | |
| | | | | QLMGLAGGRIVLALEGGHDLTAICDASEACVSALLGNELDPLPEKV | |
| | | | | LQQRPNANAVRSMEKVMEIHSKYWRCLQRTTSTAGRSLIEAQTCE | |
| | | | | NEEAETVTAMASLSVGVKPAEKRPDEEPMEEEPPL* | |
| SLC25A19 | solute carrier family 25 | 60386 | 321 | MVGYDPKPDGRNNTKFQVAVAGSVSGLVTRALISPFDVIKIRFQLQ | 405. |
| | member 19 | | | HERLSRSDPSAKYHGILQASRQILQEEGPTAFWKGHVPAQILSIGYG | |
| | [Source:HGNC | | | AVQFLSFEMLTELVHRGSVYDAREFSVHFVCGGLAACMATLTVHP | |
| | Symbol; Acc:HGNC: | | | VDVLRTRFAAQGEPKVYNTLRHAVGTMYRSEGPQVFYKGLAPTLI | |
| | 14409] | | | AIFPYAGLQFSCYSSLKHLYKWAIPAEGKKNENLQNLLCGSGAGVI | |
| | | | | SKTLTYPLDLFKKRLQVGGFEHARAAFGQVRRYKGLMDCAKQVL | |
| | | | | QKEGALGFFKGLSPSLLKAALSTGFMFFSYEFFCNVFHCMNRTASQ | |
| | | | | R* | |
| SLC25A19 | solute carrier family 25 | 60386 | 264 | MVGYDPKPDGRNNTKFQVAVAGSVSGLVTRALISPFDVIKIRFQLQ | 406. |
| | member 19 | | | HERLSRSDPSAKYHGILQASRQILQEEGPTAFWKGHVPAQILSIGYG | |
| | [Source:HGNC | | | AVQVYNTLRHAVGTMYRSEGPQVFYKGLAPTLIAIFPYAGLQFSCY | |
| | Symbol; Acc:HGNC: | | | SSLKHLYKWAIPAEGKKNENLQNLLCGSGAGVISKTLTYPLDLFKK | |
| | 14409] | | | RLQVGGFEHARAAFGQVRRYKGLMDCAKQVLQKEGALGFFKGLS | |
| | | | | PSLLKAALSTGFMFFSYEFFCNVFHCMNRTASQR* | |
| SLC25A19 | solute carrier family 25 | 60386 | 153 | MVGYDPKPDGRNNTKFQVAVAGSVSGLVTRALISPFDVIKIRFQLQ | 407. |
| | member 19 | | | HERLSRSDPSAKYHGILQASRQILQEEGPTAFWKGHVPAQILSIGYG | |
| | [Source:HGNC | | | AVQFLSFEMLTELVHRGSVYDAREFSVHFVCGGLAACMATLTVHP | |
| | Symbol; Acc:HGNC: | | | VDVLRTRFAAQGEPK | |
| | 14409] | | | | |
| SLC25A19 | solute carrier family 25 | 60386 | 106 | MVGYDPKPDGRNNTKFQVAVAGSVSGLVTRALISPFDVIKIRFQLQ | 408. |
| | member 19 | | | HERLSRSDPSAKYHGILQASRQILQEEGPTAFWKGHVPAQILSIGYG | |
| | [Source:HGNC | | | AVQFLSFEMLTEL | |
| | Symbol; Acc:HGNC: | | | | |
| | 14409] | | | | |
| SLC25A19 | solute carrier family 25 | 60386 | 68 | XKFQVAVAGSVSGLVTRALISPFDVIKIRFQLQHERLSRSDPSAKYH | 409. |
| | member 19 | | | GILQGSPHERIRGNAGRGLW* | |
| | [Source:HGNC | | | | |
| | Symbol; Acc:HGNC: | | | | |
| | 14409] | | | | |
| SLC25A19 | solute carrier family 25 | 60386 | 85 | MVGYDPKPDGRNNTKFQVAVAGSVSGLVTRALISPFDVIKIRFQLQ | 410. |
| | member 19 | | | HERLSRSDPSAKYHGILQASRQILQEEGPTAFWKGHVPA | |
| | [Source:HGNC | | | | |
| | Symbol; Acc:HGNC: | | | | |
| | 14409] | | | | |
| SLC25A19 | solute carrier family 25 | 60386 | 84 | MLTELVHRGSVYDAREFSVHFVCGGLAACMATLTVHPVDVLRTRF | 411. |
| | member 19 | | | AAQGEPKVYNTLRHAVGTMYRSEGPQVFYKGLAPTLIAI | |
| | [Source:HGNC | | | | |
| | Symbol; Acc:HGNC: | | | | |
| | 14409] | | | | |
| SLC25A19 | solute carrier family 25 | 60386 | 39 | MVGYDPKPDGRNNTKFQVAVAGSVSGLVTRALISPFDVI | 412. |
| | member 19 | | | | |
| | [Source:HGNC | | | | |
| | Symbol; Acc:HGNC: | | | | |
| | 14409] | | | | |
| SLC25A19 | solute carrier family 25 | 60386 | 182 | MVGYDPKPDGRNNTKFQVAVAGSVSGLVTRALISPFDVIKIRFQLQ | 413. |
| | member 19 | | | HERLSRSDPSAKYHGILQASRQILQEEGPTAFWKGHVPAQILSIGYG | |
| | [Source:HGNC | | | AVQFLSFEMLTELVHRGSVYDAREFSVHFVCGGLAACMATLTVHP | |
| | Symbol; Acc:HGNC: | | | VDVLRTRFAAQGEPKVRRYKGLMDCAKQVLQKEGALGFFKGLSP | |
| | 14409] | | | | |
| SLC25A19 | solute carrier family 25 | 60386 | 151 | MVGYDPKPDGRNNTKFQVAVAGSVSGLVTRALISPFDVIKIRFQLQ | 414. |
| | member 19 | | | HERLSRSDPSAKYHGILQASRQILQEEGPTAFWKGHVPAQILSIGYG | |
| | [Source:HGNC | | | AVQFLSFEMLTELVHRGSVYDAREFSVHFVCGGLAACMATLTVHP | |
| | Symbol; Acc:HGNC: | | | VDVLRTRFAAQGE | |
| | 14409] | | | | |

In some embodiments of the methods described herein, increasing expression and/or activity of a cellular factor set forth in Table 1 may comprise increasing expression of the cellular factor or increasing expression of a polynucleotide, for example, an mRNA, encoding the cellular factor in the target cell. In some embodiments of the methods described herein, decreasing expression and/or activity of a cellular factor set forth in Table 1 may comprise decreasing expression of the cellular factor or decreasing expression of a polynucleotide, for example, an mRNA, encoding the cellular factor in the target cell. In some embodiments, expression and/or activity of one or more cellular factors set forth in Table 1 is increased in the target cell. In some embodiments, expression and/or activity of one or more cellular factors set forth in Table 1 is decreased in the target cell. As described in detail further herein, one or more available methods may be used to increase or decrease the expression and/or activity of one or more cellular factors set forth in Table 1.

In some embodiments of the methods described herein, increasing or decreasing expression and/or activity of the one or more cellular factors in Table 1 comprises contacting the target cell with an agent selected from the group consisting of an antibody, a small molecule, a polypeptide, siRNA, microRNA or a drug.

In some embodiments activity of one one or more cellular factors in Table 1 is increased or decreased by contacting the target cell with an antibody. Examples of antibodies that can be used to increase or decrease activity of one one or more cellular factors in Table 1 or Table 2 are set forth in Table 3.

TABLE 3

| | | Antibody | |
|---|---|---|---|
| Antibody Target | Clone | Function | Source |
| anti-BTN3A | 20.1 | Activating | ThermoFisher |
| (CD277) | | | Scientific |
| anti-BTN3A | 103.2 | Blocking | Creative Biolabs |
| (CD277) | | | |
| anti-BTN3A | 849203 | | R&D Systems |
| (CD277) | | | |
| anti-BTN2A | 4.15 | Blocking | WO 2019/057933 Al |
| anti-BTN2A | 5.28 | Blocking | WO 2019/057933 Al |
| anti-BTN2A | 7.28 | Blocking | WO 2019/057933 Al |
| anti-BTN2A | 7.48 | Blocking | WO 2019/057933 Al |
| anti-BTN2A | 8.15 | Blocking | WO 2019/057933 Al |
| anti-BTN2A | 3A1 | | Sigma Aldrich |
| anti-BTN2A | 8.16 | Blocking | WO 2019/057933 Al |
| anti-ICAMI | HCD54 | Blocking | BioLegend |
| anti-ICAMI | YN1/1.7.4 | Blocking | StemCell, Abcam |

In some embodiments, the antibody is a bispecific antibody, wherein the bispecific antibody has specificity for an epitope of the one or more cellular factors of Table 1 expressed by the target cell and specificity for an epitope on the γδ T cell. In some embodiments, the bispecific antibody brings the target cell and the γδ T cell into proximity to increase cytotoxicity of the γδ T cell against the target cell.

In some embodiments, overexpressing a cellular factor set forth in Table 1 may comprise introducing a polynucleotide encoding the cellular factor into the target cell. In other embodiments of the methods described herein, overexpressing a cellular factor set forth in Table 1 may comprise introducing an agent that induces expression of the endogenous gene encoding the cellular factor in the target cell. For example, RNA activation, where short double-stranded RNAs induce endogenous gene expression by targeting promoter sequences, can be used to induce endogenous gene expression (See, for example, Wang et al. “Inducing gene expression by targeting promoter sequences using small activating RNAs,” J. Biol. Methods 2(1): e14 (2015)). In another example, artificial transcription factors containing zinc-finger binding domains can be used to activate or repress expression of endogenous genes. See, for example, Dent et al., “Regulation of endogenous gene expressing using small molecule-controlled engineered zinc-finger protein transcription factors,” Gene Ther. 14(18): 1362-9 (2007).

In some embodiments, inhibiting expression may comprise contacting a polynucleotide encoding the cellular factor, with a target nuclease, a guide RNA (gRNA), an siRNA, an antisense RNA, microRNA (miRNA), or short hairpin RNA (shRNA).

An siRNA, an antisense RNA, a miRNA, or a shRNA may target a sequence comprising at least 10, at least 20, at least 30, at least 40, at least 50, at least 60, at least 70, at least 80, at least 90, or at least 100 contiguous nucleotides. An siRNA may be produced from a short hairpin RNA (shRNA). A shRNA is an artificial RNA molecule with a hairpin turn that can be used to silence target gene expression via the siRNA it produces in cells. See, e.g., Fire et. al., Nature 391:806-811, 1998; Elbashir et al., Nature 411:494-498, 2001; Chakraborty et al., Mol Ther Nucleic Acids 8:132-143, 2017; and Bouard et al., Br. J. Pharmacol. 157:153-165, 2009. Expression of shRNA in cells is typically accomplished by delivery of plasmids or through viral or bacterial vectors. Suitable bacterial vectors include but not limited to adeno-associated viruses (AAVs), adenoviruses, and lentiviruses. After the vector has integrated into the host genome, the shRNA is then transcribed in the nucleus by polymerase II or polymerase III (depending on the promoter used). The resulting pre-shRNA is exported from the nucleus, then processed by a protein called Dicer and loaded into the RNA-induced silencing complex (RISC). The sense strand is degraded by RISC and the antisense strand directs RISC to an mRNA that has a complementary sequence. A protein called Ago2 in the RISC then cleaves the mRNA, or in some cases, represses translation of the mRNA, leading to its destruction and an eventual reduction in the protein encoded by the mRNA. Thus, the shRNA leads to targeted gene silencing.

The shRNA or siRNA may be encoded in a vector. In some embodiments, the vector further comprises appropriate expression control elements known in the art, including, e.g., promoters (e.g., inducible promoters or tissue specific promoters), enhancers, and transcription terminators.

In some embodiments, increasing or decreasing expression and/or activity of one or more cellular factors set forth in Table 1 comprises inhibiting or activating one or more cellular factors in the mevalonate pathway, pyrophosphate metabolism pathway or cholesterol pathway of the target cell. Examples of agents that can be used to inhibit or activate one or more cellular factors in the mevalonate pathway, pyrophosphate metabolism pathway or cholesterol pathway of the target cell include, but are not limited to, the agents set forth in Table 4.

TABLE 4

| Target | Inhibitor |
|---|---|
| Farnesyltransferase | Tipifarnib |
| Farnesyltransferase | Lonafarnib |
| Farnesyltransferase | L-744,832 |
| Farnesyltransferase | BMS-214662 hydrochloride |
| Squalene synthase | Zaragozic acid A trisodium |
| | salt |
| Mevalonate-pyrophosphate | 6-Fluoromevalonate |
| decarboxylase | |
| Geranylgeranyltransferase I | GGTI 298 trifluoroacetate |
| (GGTase I) | salt hydrate |
| Squalene synthase | YM-53601 |
| Farnesyltransferase | FTI-277 trifluoroacetate salt |
| 7-dehydro cholesterol Δ7- | AY 9944 |
| reductase (DHCR7) enzyme | |
| Farnesyl diphosphate synthase | Pamidronate |
| Farnesyl diphosphate synthase | Neridronate |
| Farnesyl diphosphate synthase | Olpadronate |
| Farnesyl diphosphate synthase | Alendronate |
| Farnesyl diphosphate synthase | Ibandronate |
| Farnesyl diphosphate synthase | Risedronate |
| Farnesyl diphosphate synthase | Zoledronate |
| HMGCR | Atorvastatin |
| HMGCR | Cerivastatin |
| HMGCR | Fluvastatin |
| HMGCR | Lovastatin |
| HMGCR | Mevastatin |
| HMGCR | Pitavastatin |
| HMGCR | Pravastatin |
| HMGCR | Rosuvastatin |
| HMGCR | Simvastatin |

In some embodiments, the target cell is a cancer cell, a cell infected with an infectious agent or pathogen, a non-cancerous cell expressing one or more antigens recognized by γδ T cells (for example, one or more cellular factors set forth in Table 1), an osteoclast or an osteoblast, to name a few. Any of the methods provided herein can be performed in vitro, ex vivo or in vivo.

Also provided is a modified γδ T cell comprising a cell-surface ligand that binds to a cellular factor set forth in Table 1. In some embodiments, the cell-surface ligand inhibits activity of a cellular factor set forth in Table 1. In some embodiments, the cell-surface ligand stimulates or increases activity of a cellular factor set forth in Table 1. In some embodiments, the modified γδ T cell comprises a heterologous polynucleotide that encodes a cell-surface protein that binds to a cellular factor set forth in Table 1. In some embodiments, the cell-surface protein an antibody or a fragment thereof that is expressed on the cell surface of the γδ T cell and binds to a cellular factor set forth in Table 1. In some embodiments, the cell surface ligand or protein is a cognate ligand that binds to a cellular factor set forth in Table 1. Populations of the modified cells described herein are also provided.

Methods for modifying cells, for example, target cells or γδ T cells are known in the art. For example, viral vectors such as a gammaretroviral vector can be used to transduce γδ T cells with a polynucleotide encoding a polypeptide, for example, a cell-surface ligand, that binds to a cellular factor set forth in Table 1. Non-viral methods, for example, transposon-based gene transfer can also be used. See, for example, Fisher and Anderson, “Engineering Approaches in Human Gamma Delta T Cells for Cancer immunotherapy,” Front. Immunol. June 2018. Methods employing targeted nucleases for insertion of a polynucleotide can also be used. In some embodiments, the targeted nuclease is selected from the group consisting of an RNA-guided nuclease domain, a transcription activator-like effector nuclease (TALEN), a zinc finger nuclease (ZFN) and a megaTAL (See, for example, Merkert and Martin “Site-Specific Genome Engineering in Human Pluripotent Stem Cells,” Int. J. Mol. Sci. 18(7): 1000 (2016)). In some embodiments, the RNA-guided nuclease is a Cas9 nuclease and the method further comprises introducing into the cell a guide RNA that specifically hybridizes to a target region in the genome of γδ T cell.

As used throughout, a guide RNA (gRNA) sequence is a sequence that interacts with a site-specific or targeted nuclease and specifically binds to or hybridizes to a target nucleic acid within the genome of a cell, such that the gRNA and the targeted nuclease co-localize to the target nucleic acid in the genome of the cell. Each gRNA includes a DNA targeting sequence or protospacer sequence of about 10 to 50 nucleotides in length that specifically binds to or hybridizes to a target DNA sequence in the genome. For example, the DNA targeting sequence is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the gRNA comprises a crRNA sequence and a transactivating crRNA (tracrRNA) sequence. In some embodiments, the gRNA does not comprise a tracrRNA sequence.

Generally, the DNA targeting sequence is designed to complement (e.g., perfectly complement) or substantially complement the target DNA sequence. In some cases, the DNA targeting sequence can incorporate wobble or degenerate bases to bind multiple genetic elements. In some cases, the 19 nucleotides at the 3′ or 5′ end of the binding region are perfectly complementary to the target genetic element or elements. In some cases, the binding region can be altered to increase stability. For example, non-natural nucleotides, can be incorporated to increase RNA resistance to degradation. In some cases, the binding region can be altered or designed to avoid or reduce secondary structure formation in the binding region. In some cases, the binding region can be designed to optimize G-C content. In some cases, G-C content is preferably between about 40% and about 60% (e.g., 40%, 45%, 50%, 55%, 60%).

In some embodiments, the Cas9 nuclease, the guide RNA and the nucleic acid sequence encoding a heterologous polypeptide are introduced into the cell as a ribonucleoprotein complex (RNP)-DNA template complex, wherein the RNP-DNA template complex comprises: (i) the RNP, wherein the RNP comprises the Cas9 nuclease and the guide RNA; and (ii) the DNA template encoding a heterologous polypeptide. In some embodiments, the RNP complex may be introduced into about 1×10⁵ to about 2×10⁶ cells (e.g., 1×10⁵ cells to about 5×10⁵ cells, about 1×10⁵ cells to about 1×10⁶ cells, 1×10⁵ cells to about 1.5×10⁶ cells, 1×10⁵ cells to about 2×10⁶ cells, about 1×10⁶ cells to about 1.5×10⁶ cells, or about 1×10⁶ cells to about 2×10⁶ cells). In some embodiments, the δ T cells are cultured under conditions effective for expanding the population of modified γδ T cells. Also disclosed herein is a population of γδ T cells, in which the genome of at least 20%, 30%, 40%, 50%, 60%, 70%, 80%, 90%, 95%, 99% or greater of the cells comprises a heterologous polynucleotide encoding a cell-surface protein that binds to one or more cellular factors set forth in Table 1.

III. Methods of Treatment

Any of the methods described herein for increasing or decreasing sensitivity of target cells to killing by γδ T cells may be performed in a human subject. Any of the methods and compositions described herein may be used to increase or decrease sensitivity of a target cell to killing by γδ T cells, wherein the target cells are obtained from a human subject. Any of the methods provided herein may be used to treat or prevent a disease (e.g., cancer, an autoimmune disease, an infectious disease, a metabolic disorder or a bone disorder).

Provided herein is a method of increasing sensitivity of target cells to killing by a γδ T cell in a subject in need thereof, comprising increasing expression and/or activity of one or more cellular factors set forth in Table 1, in the target cells of the subject. In some embodiments, the subject has cancer or an infectious disease.

Examples of cancers include, but are not limited to, Chondrosarcoma, Ewing's sarcoma, Malignant fibrous histiocytoma of bone/osteosarcoma, Osteosarcoma, Rhabdomyosarcoma, Heart cancer, Astrocytoma, Brainstem glioma, Pilocytic astrocytoma, Ependymoma, Primitive neuroectodermal tumor, Cerebellar astrocytoma, Cerebral astrocytoma, Glioma, Medulloblastoma, Neuroblastoma, Oligodendroglioma, Pineal astrocytoma, Pituitary adenoma, Visual pathway and hypothalamic glioma, Breast cancer, Invasive lobular carcinoma, Tubular carcinoma, Invasive cribriform carcinoma, Medullary carcinoma, Male breast cancer, Phyllodes tumor, Inflammatory Breast Cancer, Adrenocortical carcinoma, Islet cell carcinoma, Multiple endocrine neoplasia syndrome, Parathyroid cancer, Pheochromocytoma, Thyroid cancer, Merkel cell carcinoma, Uveal melanoma, Retinoblastoma, Anal cancer, Appendix cancer, cholangiocarcinoma, Carcinoid tumor (gastrointestinal), Colon cancer, Extrahepatic bile duct cancer, Gallbladder cancer, Gastric (stomach) cancer, Gastrointestinal carcinoid tumor, Gastrointestinal stromal tumor (GIST), Hepatocellular cancer, Pancreatic cancer (islet cell), Rectal cancer, Bladder cancer, Cervical cancer, Endometrial cancer, Extragonadal germ cell tumor, Ovarian cancer, Ovarian epithelial cancer (surface epithelial-stromal tumor), Ovarian germ cell tumor, Penile cancer, Renal cell carcinoma, Renal pelvis and ureter (transitional cell cancer), Prostate cancer, Testicular cancer, Gestational trophoblastic tumor, Ureter and renal pelvis (transitional cell cancer), Urethral cancer, Uterine sarcoma, Vaginal cancer, Vulvar cancer, Wilms tumor, Esophageal cancer, Head and neck cancer, Nasopharyngeal carcinoma, Oral cancer, Oropharyngeal cancer, Paranasal sinus and nasal cavity cancer, Pharyngeal cancer, Salivary gland cancer, Hypopharyngeal cancer, Acute biphenotypic leukemia, Acute eosinophilic leukemia, Acute lymphoblastic leukemia, Acute myeloid leukemia, Acute myeloid dendritic cell leukemia, AIDS-related lymphoma, Anaplastic large cell lymphoma, Angioimmunoblastic T-cell lymphoma, B-cell prolymphocytic leukemia, Burkitt's lymphoma, Chronic lymphocytic leukemia, Chronic myelogenous leukemia, Cutaneous T-cell lymphoma, Diffuse large B-cell lymphoma, Follicular lymphoma, Hairy cell leukemia, Hepatosplenic T-cell lymphoma, Hodgkin's lymphoma, Intravascular large B-cell lymphoma, Large granular lymphocytic leukemia, Lymphoplasmacytic lymphoma, Lymphomatoid granulomatosis, Mantle cell lymphoma, Marginal zone B-cell lymphoma, Mast cell leukemia, Mediastinal large B cell lymphoma, Multiple myeloma/plasma cell neoplasm, Myelodysplastic syndromes, Mucosa-associated lymphoid tissue lymphoma, Mycosis fungoides, Nodal marginal zone B cell lymphoma, Non-Hodgkin lymphoma, Precursor B lymphoblastic leukemia, Primary central nervous system lymphoma, Primary cutaneous follicular lymphoma, Primary cutaneous immunocytoma, Primary effusion lymphoma, Plasmablastic lymphoma, Sézary syndrome, Splenic marginal zone lymphoma, T-cell prolymphocytic leukemia, Basal cell carcinoma, Squamous cell carcinoma, Skin adnexal tumors (e.g. sebaceous carcinoma), Melanoma, Merkel cell carcinoma, Sarcomas of primary cutaneous origin (e.g. dermatofibrosarcoma protuberans), Lymphomas of primary cutaneous origin (e.g. mycosis fungoides), Bronchial adenomas/carcinoids, Small cell lung cancer, Mesothelioma, Non-small cell lung cancer, Pleuropulmonary blastoma, Laryngeal cancer, Thymoma and thymic carcinoma, Kaposi sarcoma, Epithelioid hemangioendothelioma (EHE), Desmoplastic small round cell tumor and Liposarcoma.

Examples of infectious diseases include bacterial infections, viral infections and parasitic infections such as, for example, malaria (Plasmodium spp.), tuberculosis, listeriosis and cytomegalovirus infection.

Also provided herein is a method of decreasing sensitivity of target cells to killing by a γδ T cell in a subject in need thereof, comprising decreasing expression and/or activity of one or more cellular factors set forth in Table 1, in the target cells of the subject. In some embodiments, the subject has a bone disorder, a metabolic disorder or an autoimmune disorder.

Examples of bone disorders include, but are not limited to, osteoporosis, Paget's disease of bone, fibrous dysplasia of bone, osteogenesis imperfecta and primary by hyperparathyroidism.

Examples of metabolic disorders include, but are not limited to, abnormal cholesterol levels, Gaucher's disease, Fabry disease, Sitosterolemia, Lysosomal acid lipase deficiency and Cerebrotendineous xanthomatosis.

Examples of autoimmune disorders that can be treated or prevented include, but are not limited to, rheumatoid arthritis, Type 1 diabetes, multiple sclerosis, Crohn's disease, systemic lupus erythematosus, scleroderma, Sjogren's syndrome, Addison's disease, ankylosing spondylitis, aplastic anemia, autoimmune hemolytic anemia, autoimmune hepatitis, coeliac disease, dermatomyositis, Goodpasture's syndrome, Graves' disease, Guillain-Barre syndrome. Hashimoto's disease, idiopathic leucopenia, idiopathic thrombocytopenic purpura, male infertility, mixed connective tissue disease, myasthenia gravis, pernicious anemia, phacogenic uveitis, primary biliary cirrhosis, primary myxoedema and Reiter's syndrome.

Any of the methods of treatment described herein can further comprise administering γδ T cells to the subject. In some embodiments, the γδ T cells are autologous γδ T cells or allogeneic γδ T cells. In some embodiments, the γδ T cells are Vγ9Vδ2 T cell. In some embodiments, the γδ T cells comprise a heterologous cell-surface ligand that binds to a cellular factor set forth in Table 1. In some embodiments, the γδ T cells comprise a heterologous polynucleotide that encodes a cell-surface protein that binds to a cellular factor set forth in Table 1. In some embodiments, the cell-surface ligand is a heterologous antibody expressed by the γδ T cells.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to one or more molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Examples

The following examples are provided by way of illustration only and not by way of limitation. Those of skill in the art will readily recognize a variety of non-critical parameters that could be changed or modified to yield essentially the same or similar results.

### Virus Production

The Human Improved Genome-wide Knockout CRISPR Library (Deposited by Kosuke Yusa, Pooled Library #67989, Addgene) was used for these studies. The library targeted 18,010 genes in the human genome with 90,709 guide RNAs (gRNAs). Twelve million HEK293T cells were plated in 15-cm poly-L-Lysine coated dishes 16 hours before transfection and cultured in complete DMEM (5% FBS, 1% pen/strep). Cells were transfected with the gRNA Yusa Library transfer plasmids and 2nd generation lentiviral packaging plasmids, pMD2.G (Addgene, Cat #12259) and psPAX2 (Addgene, Cat #12260) using FugeneHD (Promega) following the manufacturer's protocol. The following day, media was supplemented with ViralBoost Reagent (Alstem). The viral supernatant was collected 48 hours after transfection and spun down at 300×g for 10 minutes, to remove cell debris. To concentrate the lentiviral particles, Lentivirus Precipitation Solution (Alstem) was added to the collected supernatant and refrigerated at 4 C overnight. Then, the virus was concentrated by centrifugation at 1500×g for 30 minutes at 4 C. Finally, each lentiviral pellet was resuspended in 1/100th of the original volume in cold PBS and stored at −80° C.

### Daudi Genome-Wide Knockout

Daudi-Cas9 cells were cultured for at least two weeks in complete RPMI+Blasticidin (2 mM L-glutamine, 10% FCS, 1% Pen/Strep, 5 μg/mL blasticidin). For the genome-wide knockout, we grew 250E6 Daudi cells. The cells were brought to 3E6 cells/mL in cRPMI+Blasticidin and supplemented with 4 μg/mL polybrene and virus added at 1:400 dilution. The cell mixture was spun in 6-well plates at 2.5 mL per well, 300×g, 25° C., 2 hours. At the end of the spin, the cells were transferred into the 37° C. incubator. After resting for 6 hours, the cells were diluted to 300E3 cells/mL and placed at 37° C. Three days after the infection, the cells were split following the standard procedure of seeding the cells at 300E3 cells/mL. The infection frequency was confirmed to be 20-30%. The cells were supplemented with 5 μg/mL puromycin to select only for cells that were successfully infected with the virus. Four days after the puromycin treatment, the cells were checked for infection purity by assaying BFP expression on a flow cytometer. The cells were placed in fresh cRPMI media without puromycin or blasticidin. The cells were passaged every 2-3 days at 300E3 cells/mL, maintaining a large enough pool of cells (>150E6 cells) to have sufficient genome-wide gRNA library representation (>1000× coverage). Genomic DNA was prepared from 50E6 cells and amplified the virally integrated gRNA-encoding region. The amplified library was analyzed by Next Generation Sequencing (NGS) (HiSeq, Illumina) to confirm even distribution of gRNAs from the library in the CRISPR-edited pool of Daudi cells. The results showed good distribution of gRNAs and relative reduction in gRNAs targeted against essential genes. One day prior to the killing assay, the edited Daudi cells were treated with 50 uM zoledronic acid.

### Vγ9Vδ2 T Cell Expansion

Peripheral blood mononuclear cells (PBMCs) from four healthy donors were obtained from TRIMA residuals from apheresis collection. For each donor, an aliquot of PBMCs was analyzed by flow cytometry to assess baseline frequency of Vγ9Vδ2 T cells. Three of the donors had sufficiently high levels of Vγ9Vδ2 T cells (>1% of total live PBMCs). We proceeded with these three donors. The cells were diluted to 1E6 cells/mL in cRPMI supplemented with 5 uM zoledronic acid and 100 U/mL IL-2. The cells were given 100 U/mL of IL-2 every 2-3 days. The cells were cultured for 8 days.

### Vγ9Vδ2 T Cell-Daudi Killing Assay Screen

Eight days after starting the Vγ9Vδ2 T cell expansion, the cells were harvested. Using flow cytometry we confirmed that Vγ9Vδ2 T cells sufficiently expanded (>75% of total live cells). Using a custom γδ T cell negative isolation kit (StemCell Technologies), we isolated γδ T cells for all three donors. The Vγ9Vδ2 T cells were aliquoted into flasks. In parallel, Daudi cells were harvested and washed. For each T cell donor, Vγ9Vδ2 T cells and Daudi cells were mixed at effector:target (E:T) ratios of 1:2 and 1:4. The cells were cultured at 2E6 cells/mL in cRPMI supplemented with 5 uM zoledronic acid and 100 U/mL IL-2. At 24 hours after the start of the co-culture, we harvested Daudi cells by depleting the Vγ9Vδ2 T cells using a CD3 Positive Isolation Kit (StemCell Technologies). The Daudi cells were cultured until the dead cells were depleted from the culture. Using the resulting Daudi cell population, we obtained genomic DNA, amplified the integrated gRNA sequence through two rounds of PCR, and sequenced the libraries by NGS (HiSeq, Illumina).

### Data Analysis

Counts for gRNA libraries were generated using the count command in MAGeCK version 0.5.8 (mageck count --norm-method none). High outlier counts were filtered out before calculating differentially enriched gRNAs between the low and high bins using the mageck test command (mageck test -k countfile -t D6_1-2_S4_L001_R1_001.fastq.gz,D6_1-4_S4_L001_R1_001.fastq.gz,D8_1-4_S5_L001_R1_001.fastq.gz,D9_1-2_S2_L001_R1_001.fastq.gz,D9_1-4_S3_L001_R1_001.fastq.gz -c Pre-Kill_Daudi-Cas9-Yusa_S1_L001_R1_001.fastq.gz --sort-criteria pos -n). We used an FDR<0.10 as a cutoff to call significantly differentially enriched sgRNAs.

### Results

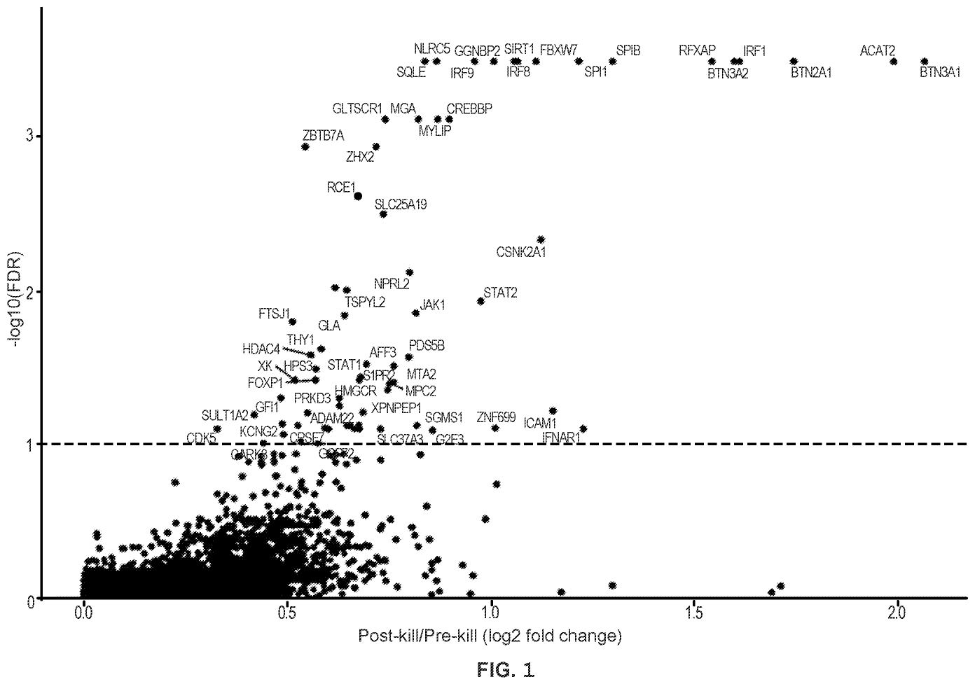

FIG. 1 provides aggregate data of gRNA enrichment among Daudi cells that survived the co-culture with expanded Vγ9Vδ2 T cells from three different healthy donors. The labeled data points above the dashed lines are positively enriched genes with a false-discovery rate (FDR) of less than 0.1. Their biological function is set forth in Table 5.

Table 5 provides functional groupings of some of the significantly enriched (FDR<0.1) gene knockouts (as shown in FIG. 1) across co-culture-based screens performed with expanded Vγ9Vδ2 T cells from three different healthy donors and target Daudi cells. Surviving Daudi cells were analyzed for integrated gRNAs within their genomic DNA. Some of the genes are placed in more than one grouping.

TABLE 5
Functional groupings of significantly enriched genes.

Metabolism/Nutrient
Sensing
NPRL2
MPC2
SIRT1
GLA
ACSL3
SGMS1
CARKD (NAXD)
Transcription
Factors
SPIB
IRF1
SPI1
IRF9
STAT2
STAT1
IRF8
CREBBP
ZBTB7A
MGA
ZHX2
FOXP1
AFF3
RFXAP
NLRC5
FTSJ1
HDAC4
GFI1
CPSF7
ZC3H12A
Mevalonate/Cholesterol
Pathway
ACAT2
SQLE
MYLIP
RCE1
HMGCR
Butyrophilins
BTN3A1
BTN2A1
BTN3A2
Kinase/Phosphatase
CSNK2A1
PRKD3
PRKCE
MTMR8
CDK5
Lysosomal
targeting/Intracellular
trafficking
HPS3
LRBA
Expression Regulation
FBXW7
MTA2
G2E3
GSPT2
Tumor Suppressors/
Cell cycling
TSPYL2
GGNBP2
GLTSCR1 (BICRA)
PDS5B
Channels/Transporters
LRRC8A
LRRC8C
SLC25A19
KCNG2
SLC37A3
Surface Receptors (for
example, immune
receptors) (non-BTN)
IFNAR1
ICAM1
THY1
S1PR2
XK
Misc./Unknown
XPNPEP1
ADAM22
SULT1A2
EFHC2
MOSPD1
ZNF699
DDIT4
ZNF687
PNKD
FAM122A
AMMECR1

1. A method of increasing sensitivity of a target cell to killing by a γδ T cell, comprising:
increasing expression and/or activity of one or more cellular factors set forth in Table 1, in the target cell.

2. The method of claim 1, wherein the sensitivity of the target cell is increased in the presence of the γδ T cell.

3. The method of claim 1, wherein the target cell is a cancer cell.

4. The method of claim 3, wherein the target cell is a cell that is infected with an infectious agent.

5. The method of claim 1, wherein increasing expression and/or activity of the one or more cellular factors in Table 1 comprises contacting the target cell with an agent selected from the group consisting of an antibody, a small molecule, a polypeptide, siRNA, microRNA or a drug.

6. The method of claim 1, wherein increasing expression comprises increasing expression of the cellular factor of Table 1, or increasing expression of a polynucleotide encoding the cellular factor of Table 1.

7. A method of decreasing sensitivity of a target cell to killing by a γδ T cell, comprising:
inhibiting expression and/or activity of one or more cellular factors set forth in Table 1, in the target cell.

8. The method of claim 7, wherein the sensitivity of the target cell is decreased in the presence of the γδ T cell.

9. The method of claim 7, wherein decreasing expression and/or activity of the one or more cellular factors in Table 1 comprises contacting the target cell with an agent is selected from the group consisting of an antibody, a small molecule, a polypeptide, siRNA, microRNA, or a drug.

10. The method of claim 7, wherein decreasing expression comprises reducing expression of the cellular factor, or reducing expression of a polynucleotide encoding the cellular factor.

11. The method of claim 5, wherein the antibody is a bispecific antibody, wherein the bispecific antibody has specificity for an epitope of the one or more cellular factors of Table 1 expressed by the target cell and specificity for an epitope on the γδ T cell.

12. The method of claim 1, wherein the target cell is ex vivo, in vitro or in vivo.

13. The method of claim 1, wherein the sensitivity of the target cell is increased or decreased in a human.

14. The method of claim 13, wherein the human has cancer, an autoimmune disorder or an infectious disease.

15. The method of claim 1, wherein the γδ T cell is a Vγ9Vδ2 T cell.

16. The method of claim 13, wherein the method further comprises administering γδ T cells to the human.

17. The method of claim 16, wherein the γδ T cells are autologous γδ T cells or allogeneic γδ T cells.

18. The method of claim 16, wherein the γδ T cells comprise a heterologous cell-surface protein that binds to a cellular factor set forth in Table 1.

19. The method of claim 1, wherein sensitivity of target cells to killing by a γδ T cell is increased in a subject in need thereof.

20. The method of claim 19, wherein the subject has cancer or an infectious disease.

21. The method of claim 7, wherein sensitivity of target cells to killing by a γδ T cell is decreased in a subject in need thereof.

22. The method of claim 21, wherein the subject has a bone disorder, a metabolic disorder or an autoimmune disease.

23. The method of claim 19, further comprising administering γδ T cells to the subject.

24. The method of claim 23, wherein the γδ T cells are Vγ9Vδ2 T cells.

25. The method of claim 23, wherein the γδ T cells are autologous γδ T cells or allogeneic γδ T cells.

26. The method of claim 23, wherein the γδ T cells comprise a heterologous cell-surface ligand that binds to a cellular factor set forth in Table 1.

27. A modified T cell comprising a heterologous polynucleotide that encodes a cell-surface protein that binds to a cellular factor set forth in Table 1.

28. The modified γδ T cell of claim 27, wherein the cell-surface protein is an antibody.

29. The modified γδ T cell of claim 27, wherein the γδ T cell is a Vγ9Vδ2 T cell.
